(19) United States
(12) Patent Application Publication
(10) Pub. No.: US 2025/0295766 A1
(43) Pub. Date: Sep. 25, 2025

(54) PROGNOSTIC AND THERAPEUTIC METHODS FOR CANCER

(71) Applicant: Genentech, Inc., South San Francisco, CA (US)

(72) Inventors: Xiangnan GUAN, South San Francisco, CA (US); Barzin Y. NABET, Bala Cynwyd, PA (US); Namrata Srivastava PATIL, Sunnyvale, CA (US)

(21) Appl. No.: 18/968,331

(22) Filed: Dec. 4, 2024

Related U.S. Application Data
(63) Continuation of application No. PCT/US2023/067965, filed on Jun. 6, 2023.
(60) Provisional application No. 63/349,896, filed on Jun. 7, 2022.
(60) Provisional application No. 63/380,032, filed on Oct. 18, 2022.
(60) Provisional application No. 63/488,329, filed on Mar. 3, 2023.

(51) Int. Cl. A61K 39/395; A61K 39/00; A61P 35/00; C12Q 1/6886; G01N 33/574

(52) U.S. Cl. CPC A61K 39/39558; A61P 35/00; C12Q 1/6886; G01N 33/57423; A61K 2039/507; C12Q 2600/106; C12Q 2600/158; G01N 2474/20

(57) ABSTRACT

The present invention relates to prognostic and therapeutic methods for the treatment of cancer (e.g., lung cancer, e.g., non-small cell lung cancer (NSCLC)) using expression levels of tumor-associated macrophage (TAM) and regulatory T cell (Treg) genes. In particular, the invention provides methods for patient selection and treatment.

### SEQUENCE LISTING

The instant application contains a Sequence Listing which has been submitted electronically in XML format and is hereby incorporated by reference in its entirety. Said XML copy, created on Nov. 22, 2024, is named 50474-290005_Sequence_Listing_1122_24 and is 33,394 bytes in size.

### FIELD OF THE INVENTION

Provided herein are prognostic and therapeutic methods for the treatment of cancer (e.g., lung cancer, e.g., non-small cell lung cancer (NSCLC)) using expression levels of tumor-associated macrophage (TAM) and regulatory T cell (Treg) genes. In particular, the invention provides methods for patient selection and treatment.

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

Programmed cell death-1/programmed cell death ligand-1 (PD-1/PD-L1) blockade is efficacious across a broad range of malignancies. However, not all patients benefit, and a significant fraction of initial responders eventually relapse. One approach to extend and expand the impact of cancer immunotherapy has been to target additional immune checkpoints. One such co-inhibitory checkpoint is TIGIT (T cell immunoreceptor with Ig and immunoreceptor tyrosine-based inhibitory motif (ITIM) domain).

Non-small cell lung cancer (NSCLC) is the predominant subtype of lung cancer, accounting for approximately 80%-85% of all cases. For advanced disease, the overall five-year survival rate is 2%-4%.

Despite improvements in the first-line treatment of patients with advanced NSCLC that have resulted in longer survival times and reduced disease-related symptoms, nearly all patients experience disease progression. Cancer immunotherapies, in particular, offer the possibility of long-term disease control. In particular, NSCLC patients have been found to benefit from treatment with combinations comprising a PD-1 axis binding antagonist (atezolizumab) and an anti-TIGIT antagonist antibody (tiragolumab).

Thus, there is an unmet need in the field for robust prognostic methods that identify patients likely to benefit from a treatment comprising a PD-1 axis binding antagonist and an anti-TIGIT antagonist antibody for more effective management of the disease.

### SUMMARY OF THE INVENTION

In one aspect, the invention provides a method of identifying an individual having a cancer who may benefit from a treatment comprising a PD-1 axis binding antagonist and an anti-TIGIT antagonist antibody, the method comprising detecting an expression level of each of C1QC, MSR1, MRC1, VSIG4, SPP1, and MARCO in a sample from the individual and determining a tumor-associated macrophage (TAM) signature score therefrom, wherein a TAM signature score that is above a reference TAM signature score identifies the individual as one who may benefit from a treatment comprising a PD-1 axis binding antagonist and an anti-TIGIT antagonist antibody.

In another aspect, the invention provides a method for selecting a therapy for an individual having a cancer, the method comprising detecting an expression level of each of C1QC, MSR1, MRC1, VSIG4, SPP1, and MARCO in a sample from the individual and determining a TAM signature score therefrom, wherein a TAM signature score that is above a reference TAM signature score identifies the individual as one who may benefit from a treatment comprising a PD-1 axis binding antagonist and an anti-TIGIT antagonist antibody.

In some aspects, the individual has a TAM signature score in the sample that is above a reference TAM signature score, and the method further comprises administering to the individual an effective amount of a PD-1 axis binding antagonist and an anti-TIGIT antagonist antibody.

In another aspect, the invention provides a method of treating an individual having a cancer, the method comprising (a) detecting an expression level of each of C1QC, MSR1, MRC1, VSIG4, SPP1, and MARCO in a sample from the individual and determining a TAM signature score therefrom, wherein the TAM signature score is above a reference TAM signature score and thereby identifies the individual as one who may benefit from a treatment comprising a PD-1 axis binding antagonist and an anti-TIGIT antagonist antibody; and (b) administering an effective amount of a PD-1 axis binding antagonist and an anti-TIGIT antagonist antibody to the individual.

In another aspect, the invention provides a method of treating an individual having a cancer, the method comprising administering a PD-1 axis binding antagonist and an anti-TIGIT antagonist antibody to the individual, wherein the individual has been determined to have a TAM signature score that is above a reference TAM signature score, thereby identifying the individual as one who may benefit from a treatment comprising a PD-1 axis binding antagonist and an anti-TIGIT antagonist antibody, and wherein the TAM signature score is based on the expression level of each of C1QC, MSR1, MRC1, VSIG4, SPP1, and MARCO detected in a sample from the individual.

In some aspects, the sample is obtained from the individual prior to treatment with the PD-1 axis binding antagonist and the anti-TIGIT antagonist antibody.

In some aspects, the benefit is an increase in progression-free survival (PFS), objective response rate (ORR), or overall survival (OS).

In some aspects, the reference TAM signature score is a pre-assigned TAM signature score.

In some aspects, the reference TAM signature score is a TAM signature score in a reference population. In some aspects, the TAM signature score in the reference population is a median TAM signature score of the reference population. In some aspects, the reference population is a population of individuals having the cancer.

In some aspects, the TAM signature score is an average of the expression levels of C1QC, MSR1, MRC1, VSIG4, SPP1, and MARCO in the sample from the individual. In some aspects, the TAM signature score is an average of the normalized expression levels of C1QC, MSR1, MRC1, VSIG4, SPP1, and MARCO in the sample from the individual.

In some aspects, the method comprises further detecting the expression level of one or more of ACP5, MCEMP1, CYP27A1, OLR1, GRN, GLIPR2, ARRDC4, APOE, FOLR2, and CTSD in the sample from the individual.

In some aspects, the TAM signature score is an average of the expression levels of C1QC, MSR1, MRC1, VSIG4, SPP1, MARCO, and one or more of ACP5, MCEMP1, CYP27A1, OLR1, GRN, GLIPR2, ARRDC4, APOE, FOLR2, and CTSD in the sample from the individual. In some aspects, the TAM signature score is an average of the normalized expression levels of C1QC, MSR1, MRC1, VSIG4, SPP1, MARCO, and one or more of ACP5, MCEMP1, CYP27A1, OLR1, GRN, GLIPR2, ARRDC4, APOE, FOLR2, and CTSD in the sample from the individual.

In some aspects, the method comprises further detecting the expression level of each of ACP5, MCEMP1, CYP27A1, OLR1, GRN, GLIPR2, ARRDC4, APOE, FOLR2, and CTSD in the sample from the individual and determining therefrom the TAM signature score, wherein the TAM signature score is an average of the expression levels of C1QC, MSR1, MRC1, VSIG4, SPP1, MARCO, ACP5, MCEMP1, CYP27A1, OLR1, GRN, GLIPR2, ARRDC4, APOE, FOLR2, and CTSD in the sample from the individual.

In some aspects, the expression level of one or more of ACP5, MCEMP1, CYP27A1, OLR1, GRN, GLIPR2, ARRDC4, APOE, FOLR2, and CTSD has been detected in the sample from the individual.

In some aspects, the TAM signature score is an average of the expression levels of C1QC, MSR1, MRC1, VSIG4, SPP1, MARCO, and one or more of ACP5, MCEMP1, CYP27A1, OLR1, GRN, GLIPR2, ARRDC4, APOE, FOLR2, and CTSD in the sample from the individual.

In some aspects, the expression level of each of ACP5, MCEMP1, CYP27A1, OLR1, GRN, GLIPR2, ARRDC4, APOE, FOLR2, and CTSD has been detected in the sample from the individual and the TAM signature score has been determined therefrom, wherein the TAM signature score is an average of the expression levels of C1QC, MSR1, MRC1, VSIG4, SPP1, MARCO, ACP5, MCEMP1, CYP27A1, OLR1, GRN, GLIPR2, ARRDC4, APOE, FOLR2, and CTSD in the sample from the individual.

In another aspect, the invention provides a method for monitoring the response of an individual having a cancer to a treatment comprising a PD-1 axis binding antagonist and an anti-TIGIT antagonist antibody, the method comprising detecting an expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 in a sample from the individual at a time point during or after administration of the PD-1 axis binding antagonist and the anti-TIGIT antagonist antibody, wherein an increase in the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 relative to a respective reference expression level is predictive of an individual who is likely to respond to the treatment comprising the PD-1 axis binding antagonist and the anti-TIGIT antagonist antibody.

In some aspects, the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is detected three weeks after the initiation of the treatment comprising the PD-1 axis binding antagonist and the anti-TIGIT antagonist antibody.

In some aspects, the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is detected six weeks after the initiation of the treatment comprising the PD-1 axis binding antagonist and the anti-TIGIT antagonist antibody.

In some aspects, the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is increased relative to a respective reference expression level, thereby predicting that the individual is likely to respond to the treatment comprising the PD-1 axis binding antagonist and the anti-TIGIT antagonist antibody, and the method further comprises administering an additional dose of the PD-1 axis binding antagonist and the anti-TIGIT antagonist antibody to the individual.

In some aspects, the response to treatment is an increase in PFS or OS.

In some aspects, the reference expression level is a baseline expression level from a sample from the individual at a time point prior to the initiation of the treatment comprising the PD-1 axis binding antagonist and the anti-TIGIT antagonist antibody.

In another aspect, the invention provides a method of identifying an individual having a cancer who may benefit from a treatment comprising a PD-1 axis binding antagonist and an anti-TIGIT antagonist antibody, the method comprising detecting an expression level of each of FOXP3, CTLA4, IL110, TNFRSF18, CCR8, IKZF4, and IKZF2 in a sample from the individual and determining a regulatory T cell (Treg) signature score therefrom, wherein a Treg signature score that is above a reference Treg signature score identifies the individual as one who may benefit from a treatment comprising a PD-1 axis binding antagonist and an anti-TIGIT antagonist antibody.

In another aspect, the invention provides a method for selecting a therapy for an individual having a cancer, the method comprising detecting an expression level of each of FOXP3, CTLA4, IL110, TNFRSF18, CCR8, IKZF4, and IKZF2 in a sample from the individual and determining a Treg signature score therefrom, wherein a Treg signature score that is above a reference Treg signature score identifies the individual as one who may benefit from a treatment comprising a PD-1 axis binding antagonist and an anti-TIGIT antagonist antibody.

In some aspects, the individual has a Treg signature score in the sample that is above a reference Treg signature score, and the method further comprises administering to the individual an effective amount of a PD-1 axis binding antagonist and an anti-TIGIT antagonist antibody.

In another aspect, the invention provides a method of treating an individual having a cancer, the method comprising (a) detecting the expression level of each of FOXP3, CTLA4, IL10, TNFRSF18, CCR8, IKZF4, and IKZF2 in a sample from the individual and determining a Treg signature score therefrom, wherein the Treg signature score is above a reference Treg signature score and thereby identifies the individual as one who may benefit from a treatment comprising a PD-1 axis binding antagonist and an anti-TIGIT antagonist antibody; and (b) administering an effective amount of a PD-1 axis binding antagonist and an anti-TIGIT antagonist antibody to the individual.

In another aspect, the invention provides a method of treating an individual having a cancer, the method comprising administering a PD-1 axis binding antagonist and an anti-TIGIT antagonist antibody to the individual, wherein the individual has been determined to have a Treg signature score that is above a reference Treg signature score, thereby identifying the individual as one who may benefit from a treatment comprising a PD-1 axis binding antagonist and an anti-TIGIT antagonist antibody, and wherein the Treg signature score is based on the expression level of each of FOXP3, CTLA4, IL10, TNFRSF18, CCR8, IKZF4, and IKZF2 detected in a sample from the individual.

In some aspects, the sample is obtained from the individual prior to treatment with a PD-1 axis binding antagonist and an anti-TIGIT antagonist antibody.

In some aspects, the benefit is an increase in PFS, ORR, or OS.

In some aspects, the reference Treg signature score is a pre-assigned Treg signature score.

In some aspects, the reference Treg signature score is a Treg signature score in a reference population. In some aspects, the Treg signature score in the reference population is a median Treg signature score of the reference population. In some aspects, the reference population is a population of individuals having the cancer.

In some aspects, the Treg signature score is an average of the expression levels of FOXP3, CTLA4, IL10, TNFRSF18, CCR8, IKZF4, and IKZF2 in the sample from the individual. In some aspects, the Treg signature score is an average of the normalized expression levels of FOXP3, CTLA4, IL10, TNFRSF18, CCR8, IKZF4, and IKZF2 in the sample from the individual.

In some aspects, the expression level is a nucleic acid expression level or a protein expression level.

In some aspects, the expression level is a nucleic acid expression level. In some aspects, the nucleic acid expression level is determined by RNA-seq, RT-qPCR, qPCR, multiplex qPCR or RT-qPCR, microarray analysis, SAGE, MassARRAY technique, ISH, or a combination thereof.

In some aspects, the nucleic acid expression level is an mRNA expression level. In some aspects, the mRNA expression level is determined by RNA-seq.

In some aspects, the expression level is a protein expression level. In some aspects, the protein expression level is determined by mass spectrometry.

In some aspects, the sample is a tissue sample, a tumor sample, a whole blood sample, a plasma sample, a serum sample, or a combination thereof.

In some aspects, the sample is a tissue sample. In some aspects, the tissue sample is a tumor tissue sample. In some aspects, the tumor tissue sample is a biopsy.

In some aspects, the sample is a serum sample.

In some aspects, the sample is an archival sample, a fresh sample, or a frozen sample.

In some aspects, the sample has been determined to have a PD-L1-positive tumor cell fraction by an immunohistochemical (IHC) assay.

In some aspects, the PD-L1-positive tumor cell fraction is determined by positive staining with an anti-PD-L1 antibody, wherein the anti-PD-L1 antibody is SP263, 22C3, SP142, or 28-8.

In some aspects, the PD-L1-positive tumor cell fraction is greater than, or equal to, 50%, as determined by positive staining with the anti-PD-L1 antibody SP263. In some aspects, the PD-L1-positive tumor cell fraction is calculated using the Ventana SP263 IHC assay.

In some aspects, the PD-L1-positive tumor cell fraction is greater than, or equal to, 50%, as determined by positive staining with the anti-PD-L1 antibody 22C3. In some aspects, the PD-L1-positive tumor cell fraction is calculated using the pharmDx 22C3 IHC assay.

In some aspects, the cancer is a lung cancer. In some aspects, the lung cancer is a non-small cell lung cancer (NSCLC).

In some aspects, the anti-TIGIT antagonist antibody comprises the following hypervariable regions (HVRs): (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4); (e) an HVR-L2 comprising the amino acid sequence of WASTRES (SEQ ID NO: 5); and (f) an HVR-L3 comprising the amino acid sequence of QQYYSTPFT (SEQ ID NO: 6).

In some aspects, the anti-TIGIT antagonist antibody further comprises the following light chain variable region FRs: (a) an FR-L1 comprising the amino acid sequence of DIVMTQSPDSLAVSLGERATINC (SEQ ID NO: 7); (b) an FR-L2 comprising the amino acid sequence of WYQQKPGQPPNLLIY (SEQ ID NO: 8); (c) an FR-L3 comprising the amino acid sequence of GVPDRFSGSGSGTDFTLTISSLQAEDVAVYYC (SEQ ID NO: 9); and (d) an FR-L4 comprising the amino acid sequence of FGPGTKVEIK (SEQ ID NO: 10).

In some aspects, the anti-TIGIT antagonist antibody further comprises the following heavy chain variable region FRs: (a) an FR-H1 comprising the amino acid sequence of X₁VQLQQSGPGLVKPSQTLSLTCAISGDSVS (SEQ ID NO: 11), wherein X₁ is Q or E; (b) an FR-H2 comprising the amino acid sequence of WIRQSPSRGLEWLG (SEQ ID NO: 12); (c) an FR-H3 comprising the amino acid sequence of RITINPDTSKNQFSLQLNSVTPEDTAVFYCTR (SEQ ID NO: 13); and (d) an FR-H4 comprising the amino acid sequence of WGQGTLVTVSS (SEQ ID NO: 14).

In some aspects, X₁ is Q. In some aspects, X₁ is E.

In some aspects, the anti-TIGIT antagonist antibody comprises (a) a VH domain comprising an amino acid sequence having at least 95% sequence identity to the amino acid sequence of EVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 17) or QVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 18); (b) a VL domain comprising an amino acid sequence having at least 95% sequence identity to the amino acid sequence of DIVMTQSPDSLAVSLGERATINCKSSQTVLYSSNNKKYLAWYQQKPGQPPNLLIYWASTRESGVPDRFS GSGSGTDFTLTISSLQAEDVAVYYCQQYYSTPFTFGPGTKVEIK (SEQ ID NO: 19); or (c) a VH domain as in (a) and a VL domain as in (b).

In some aspects, the anti-TIGIT antagonist antibody comprises (a) a VH domain comprising the amino acid sequence of SEQ ID NO: 17 or 18; and (b) a VL domain comprising the amino acid sequence of SEQ ID NO: 19.

In some aspects, the anti-TIGIT antagonist antibody comprises (a) a VH domain comprising the amino acid sequence of SEQ ID NO: 17; and (b) a VL domain comprising the amino acid sequence of SEQ ID NO: 19.

In some aspects, the anti-TIGIT antagonist antibody comprises (a) a heavy chain comprising the amino acid sequence of SEQ ID NO: 33; and (b) a light chain comprising the amino acid sequence of SEQ ID NO: 34.

In some aspects, the anti-TIGIT antagonist antibody is a monoclonal antibody.

In some aspects, the anti-TIGIT antagonist antibody is a human antibody.

In some aspects, the anti-TIGIT antagonist antibody is a full-length antibody.

In some aspects, the anti-TIGIT antagonist antibody exhibits effector function.

In some aspects, the anti-TIGIT antagonist antibody comprises an Fc domain that is able to interact with an Fc gamma receptor (FcγR).

In some aspects, the anti-TIGIT antagonist antibody is an IgG class antibody. In some aspects, the IgG class antibody is an IgG1 subclass antibody.

In some aspects, the anti-TIGIT antagonist antibody is tiragolumab.

In some aspects, the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

In some aspects, the anti-TIGIT antagonist antibody is vibostolimab, etigilimab, EOS084448, SGN-TGT, TJ-T6, BGB-A1217, or AB308.

In some aspects, the PD-1 axis binding antagonist is selected from the group consisting of a PD-L1 binding antagonist, a PD-1 binding antagonist, and a PD-L2 binding antagonist.

In some aspects, the PD-1 axis binding antagonist is a PD-L1 binding antagonist.

In some aspects, the PD-L1 binding antagonist inhibits the binding of PD-L1 to one or more of its ligand binding partners. In some aspects, the PD-L1 binding antagonist inhibits the binding of PD-L1 to PD-1, B7-1, or both PD-1 and B7-1.

In some aspects, the PD-L1 binding antagonist is an anti-PD-L1 antagonist antibody. In some aspects, the anti-PD-L1 antagonist antibody is atezolizumab, MDX-1105, durvalumab, avelumab, SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, or HS-636.

In some aspects, the anti-PD-L1 antagonist antibody is atezolizumab.

In some aspects, the anti-PD-L1 antagonist antibody comprises the following HVRs: (a) an HVR-H1 sequence comprising the amino acid sequence of GFTFSDSWIH (SEQ ID NO: 20); (b) an HVR-H2 sequence comprising the amino acid sequence of AWISPYGGSTYYADSVKG (SEQ ID NO: 21); (c) an HVR-H3 sequence comprising the amino acid sequence of RHWPGGFDY (SEQ ID NO: 22); (d) an HVR-L1 sequence comprising the amino acid sequence of RASQDVSTAVA (SEQ ID NO: 23); (e) an HVR-L2 sequence comprising the amino acid sequence of SASFLYS (SEQ ID NO: 24); and (f) an HVR-L3 sequence comprising the amino acid sequence of QQYLYHPAT (SEQ ID NO: 25).

In some aspects, the anti-PD-L1 antagonist antibody comprises (a) a heavy chain variable (VH) domain comprising an amino acid sequence having at least 95% sequence identity to the amino acid sequence of SEQ ID NO: 26; (b) a light chain variable (VL) domain comprising an amino acid sequence having at least 95% sequence identity to the amino acid sequence of SEQ ID NO: 27; or (c) a VH domain as in (a) and a VL domain as in (b).

In some aspects, the anti-PD-L1 antagonist antibody comprises (a) a VH domain comprising the amino acid sequence of SEQ ID NO: 26; and (b) a VL domain comprising the amino acid sequence of SEQ ID NO: 27.

In some aspects, the anti-PD-L1 antagonist antibody comprises (a) a heavy chain comprising the amino acid sequence of SEQ ID NO: 28; and (b) a light chain comprising the amino acid sequence of SEQ ID NO: 29.

In some aspects, the anti-PD-L1 antagonist antibody is a monoclonal antibody.

In some aspects, the anti-PD-L1 antagonist antibody is a humanized antibody.

In some aspects, the anti-PD-L1 antagonist antibody is a full-length antibody.

In some aspects, the anti-PD-L1 antagonist antibody is an antibody fragment that binds PD-L1 selected from the group consisting of Fab, Fab′, Fab′-SH, Fv, scFv, and (Fab′)₂ fragments.

In some aspects, the anti-PD-L1 antagonist antibody is an IgG class antibody. In some aspects, the IgG class antibody is an IgG1 subclass antibody.

In some aspects, the PD-1 axis binding antagonist is a PD-1 binding antagonist. In some aspects, the PD-1 binding antagonist inhibits the binding of PD-1 to one or more of its ligand binding partners. In some aspects, the PD-1 binding antagonist inhibits the binding of PD-1 to PD-L1, PD-L2, or both PD-L1 and PD-L2.

In some aspects, the PD-1 binding antagonist is an anti-PD-1 antagonist antibody. In some aspects, the anti-PD-1 antagonist antibody is nivolumab, pembrolizumab, MEDI-0680, spartalizumab, cemiplimab, BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, or hAb21.

In some aspects, the PD-1 binding antagonist is an Fc fusion protein. In some aspects, the Fc fusion protein is AMP-224.

In some aspects, the individual is a human.

In some aspects, the anti-TIGIT antagonist antibody is capable of Fc-dependent activation of myeloid cells, optionally wherein the myeloid cell is a cell selected from the group consisting of intratumoral type 1 conventional dendritic cells (cDC1s), macrophages, neutrophils, and circulating monocytes.

In some aspects, the anti-TIGIT antagonist antibody is capable of interacting with the Fc gamma receptor (FcγR) on myeloid cells and is capable of inducing CD8+ T cell mobilization in the blood and/or an expansion of proliferating CD8+ T cells within the tumor bed.

In another aspect, the invention provides a method of treating an individual having a cancer, the method comprising administering an anti-TIGIT antagonist antibody to the individual, wherein the anti-TIGIT antagonist antibody is capable of Fc-dependent activation of myeloid cells, optionally wherein the myeloid cell is a cell selected from the group consisting of intratumoral type 1 conventional dendritic cells (cDC1s), macrophages, neutrophils, and circulating monocytes.

In another aspect, the invention provides use of an anti-TIGIT antagonist antibody in the manufacture of a medicament for treating cancer, wherein the anti-TIGIT antagonist antibody is capable of Fc-dependent activation of myeloid cells, optionally wherein the myeloid cell is a cell selected from the group consisting of intratumoral cDC1s, macrophages, neutrophils, and circulating monocytes.

In another aspect, the invention provides a method of treating an individual having a cancer, the method comprising administering an anti-TIGIT antagonist antibody to the individual, wherein the anti-TIGIT antagonist antibody is capable of interacting with the Fc gamma receptor (FcγR) on myeloid cells and is capable of inducing CD8+ T cell mobilization in the blood and/or an expansion of proliferating CD8+ T cells within the tumor bed.

In another aspect, the invention provides use of an anti-TIGIT antagonist antibody in the manufacture of a medicament for treating cancer, wherein the anti-TIGIT antagonist antibody is capable of interacting with the FcγR and is capable of inducing CD8+ T cell mobilization in the blood and/or an expansion of proliferating CD8+ T cells within the tumor bed.

In another aspect, the invention provides a method of identifying an individual having a cancer who may benefit from a treatment comprising a PD-1 axis binding antagonist and an anti-TIGIT antagonist antibody that exhibits effector function, the method comprising detecting an expression level of each of C1QC, MSR1, MRC1, VSIG4, SPP1, and MARCO in a sample from the individual and determining a tumor-associated macrophage (TAM) signature score therefrom, wherein a TAM signature score that is above a reference TAM signature score identifies the individual as one who may benefit from a treatment comprising a PD-1 axis binding antagonist and an anti-TIGIT antagonist antibody that exhibits effector function.

In another aspect, the invention provides a method for selecting a therapy for an individual having a cancer, the method comprising detecting an expression level of each of C1QC, MSR1, MRC1, VSIG4, SPP1, and MARCO in a sample from the individual and determining a TAM signature score therefrom, wherein a TAM signature score that is above a reference TAM signature score identifies the individual as one who may benefit from a treatment comprising a PD-1 axis binding antagonist and an anti-TIGIT antagonist antibody that exhibits effector function.

In some aspects, the individual has a TAM signature score in the sample that is above a reference TAM signature score, and the method further comprises administering to the individual an effective amount of a PD-1 axis binding antagonist and an anti-TIGIT antagonist antibody that exhibits effector function.

In another aspect, the invention provides a method of treating an individual having a cancer, the method comprising (a) detecting an expression level of each of C1QC, MSR1, MRC1, VSIG4, SPP1, and MARCO in a sample from the individual and determining a TAM signature score therefrom, wherein the TAM signature score is above a reference TAM signature score and thereby identifies the individual as one who may benefit from a treatment comprising a PD-1 axis binding antagonist and an anti-TIGIT antagonist antibody that exhibits effector function; and (b) administering an effective amount of a PD-1 axis binding antagonist and an anti-TIGIT antagonist antibody that exhibits effector function to the individual.

In another aspect, the invention provides a method of treating an individual having a cancer, the method comprising administering a PD-1 axis binding antagonist and an anti-TIGIT antagonist antibody that exhibits effector function to the individual, wherein the individual has been determined to have a TAM signature score that is above a reference TAM signature score, thereby identifying the individual as one who may benefit from a treatment comprising a PD-1 axis binding antagonist and an anti-TIGIT antagonist antibody that exhibits effector function, and wherein the TAM signature score is based on the expression level of each of C1QC, MSR1, MRC1, VSIG4, SPP1, and MARCO detected in a sample from the individual.

In another aspect, the invention provides a method for monitoring the response of an individual having a cancer to a treatment comprising a PD-1 axis binding antagonist and an anti-TIGIT antagonist antibody that exhibits effector function, the method comprising detecting an expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 in a sample from the individual at a time point during or after administration of the PD-1 axis binding antagonist and the anti-TIGIT antagonist antibody that exhibits effector function, wherein an increase in the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 relative to a respective reference expression level is predictive of an individual who is likely to respond to the treatment comprising the PD-1 axis binding antagonist and the anti-TIGIT antagonist antibody that exhibits effector function.

In another aspect, the invention provides a method of identifying an individual having a cancer who may benefit from a treatment comprising a PD-1 axis binding antagonist and an anti-TIGIT antagonist antibody that exhibits effector function, the method comprising detecting an expression level of each of FOXP3, CTLA4, IL110, TNFRSF18, CCR8, IKZF4, and IKZF2 in a sample from the individual and determining a regulatory T cell (Treg) signature score therefrom, wherein a Treg signature score that is above a reference Treg signature score identifies the individual as one who may benefit from a treatment comprising a PD-1 axis binding antagonist and an anti-TIGIT antagonist antibody that exhibits effector function.

In another aspect, the invention provides a method for selecting a therapy for an individual having a cancer, the method comprising detecting an expression level of each of FOXP3, CTLA4, IL10, TNFRSF18, CCR8, IKZF4, and IKZF2 in a sample from the individual and determining a Treg signature score therefrom, wherein a Treg signature score that is above a reference Treg signature score identifies the individual as one who may benefit from a treatment comprising a PD-1 axis binding antagonist and an anti-TIGIT antagonist antibody that exhibits effector function.

In some aspects, the individual has a Treg signature score in the sample that is above a reference Treg signature score, and the method further comprises administering to the individual an effective amount of a PD-1 axis binding antagonist and an anti-TIGIT antagonist antibody that exhibits effector function.

In another aspect, the invention provides a method of treating an individual having a cancer, the method comprising (a) detecting the expression level of each of FOXP3, CTLA4, IL10, TNFRSF18, CCR8, IKZF4, and IKZF2 in a sample from the individual and determining a Treg signature score therefrom, wherein the Treg signature score is above a reference Treg signature score and thereby identifies the individual as one who may benefit from a treatment comprising a PD-1 axis binding antagonist and an anti-TIGIT antagonist antibody that exhibits effector function; and (b) administering an effective amount of a PD-1 axis binding antagonist and an anti-TIGIT antagonist antibody that exhibits effector function to the individual.

In another aspect, the invention provides a method of treating an individual having a cancer, the method comprising administering a PD-1 axis binding antagonist and an anti-TIGIT antagonist antibody that exhibits effector function to the individual, wherein the individual has been determined to have a Treg signature score that is above a reference Treg signature score, thereby identifying the individual as one who may benefit from a treatment comprising a PD-1 axis binding antagonist and an anti-TIGIT antagonist antibody that exhibits effector function, and wherein the Treg signature score is based on the expression level of each of FOXP3, CTLA4, IL10, TNFRSF18, CCR8, IKZF4, and IKZF2 detected in a sample from the individual.

In some aspects, the anti-TIGIT antagonist antibody comprises an Fc domain that is able to interact with an Fc gamma receptor (FcγR).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a set of Kaplan-Meier (KM) curves showing overall survival (OS) in non-small cell lung cancer (NSCLC) patients in the biomarker evaluable population (BEP) of the CITYSCAPE trial (GO40290) who were treated with atezolizumab (atezo) and a placebo or tiragolumab (tira) and atezolizumab. Hazard ratio (HR) and 95% confidence interval were determined using a univariate Cox model. Mo: months.

FIG. 1B is a forest plot showing the association between high abundance of the indicated cell types in tumors and objective response rate (ORR) in the BEP of the CITYSCAPE trial. T+A: tiragolumab+atezolizumab. P+A: placebo+atezolizumab. Intratumoral cell types were determined as high or low based on the median signature score cutoffs. Hazard ratio and 95% confidence interval were determined using a univariate Cox model.

FIG. 1C is a set of photomicrographs showing H&E staining and multiplex immunofluorescence (mIF) staining of panCK (green), FoxP3 (white), CD68 (red), and programmed death-ligand 1 (PD-L1) (yellow) in CITYSCAPE patient tumor samples representative of Treg-high, myeloid-high (top); Treg-high, myeloid-low (middle); and Treg-low, myeloid-low (bottom) samples.

FIG. 1D is a set of KM curves showing OS in patients having tumors enriched (solid lines) or not enriched (dashed lines) for tumor-associated macrophages (TAMs) who were treated with placebo+atezolizumab or tiragolumab+atezolizumab. Enrichment was determined by the median cell type signature score cutoffs. Hazard ratio and 95% confidence interval were determined using univariate Cox model.

FIG. 1E is a set of KM curves showing OS in patients having tumors enriched (solid lines) or not enriched (dashed lines) for regulatory T cells (Tregs) who were treated with placebo+atezolizumab or tiragolumab+atezolizumab. Enrichment was determined by the median cell type signature score cutoffs. Hazard ratio and 95% confidence interval were determined using univariate Cox model.

FIG. 1F is a set of KM curves showing OS in patients having tumors enriched (solid lines) or not enriched (dashed lines) for CD16 monocytes who were treated with placebo+atezolizumab or tiragolumab+atezolizumab. Enrichment was determined by the median cell type signature score cutoffs. Hazard ratio and 95% confidence interval were determined using univariate Cox model.

FIG. 1G is a set of KM curves showing OS in patients having tumors enriched (solid lines) or not enriched (dashed lines) for CD8+ T effector cells (tGE8) who were treated with placebo+atezolizumab or tiragolumab+atezolizumab. Enrichment was determined by the median cell type signature score cutoffs. Hazard ratio and 95% confidence interval were determined using univariate Cox model.

FIG. 2A is a pair of charts showing levels of the indicated protein/peptide markers in serum at Cycle 2, Day 1 (C2D1) in the placebo plus atezolizumab arm (left panel) or the atezolizumab plus tiragolumab combination arm (right panel) relative to baseline levels.

FIG. 2B is a heat map showing levels of the significantly increased serum proteins identified in FIG. 2A by their gene expression profiles in each of the indicated cell types based on a public single cell RNAseq (scRNAseq) NSCLC dataset, which suggested myeloid origin of most of the proteins, including NGAL (LCN2), TRFL (LTF), LCAT, VCAM1, APOC4, LYAM1 (SELL), CD5L, MARCO, CAMP, APOE, APOC2, CD163, LYSC (LYZ), APOA2, PERM (MPO), CSF1R, CD44, B2MG (B2M). For protein-gene pairs that have distinct names, the gene names are shown in parentheses in italics.

FIG. 2C is a set of KM curves showing progression-free survival (PFS) in patients with low (dashed lines) or high (solid lines) levels of serum myeloid proteins at C2D1 relative to Cycle 1, Day 1 (C1D1) using a composite of all significantly increased proteins (MARCO, CAMP, CD163, CSF1R, CD5L, NGAL (LCN2), GAPR1, APOC1, APOC2, APOC3, and APOC4), as determined by the median composite score cutoff. Hazard ratio and 95% confidence interval were determined using univariate Cox model.

FIG. 2D is a set of KM curves showing OS in patients with low (dashed lines) or high (solid lines) levels of serum myeloid proteins at C2D1 relative to C1 D1 using a composite of all significantly increased proteins (MARCO, CAMP, CD163, CSF1R, CD5L, NGAL (LCN2), GAPR1, APOC1, APOC2, APOC3, and APOC4), as determined by the median composite score cutoff. Hazard ratio and 95% confidence interval were determined using univariate Cox model.

FIG. 2E is a scatter plot showing the correlation between soluble CD163 (sCD163) levels as detected by enzyme-linked immunosorbent assay (ELISA) and CD163 levels detected by mass spectrometry (Biognosys PQ500™).

FIG. 2F is a set of KM curves showing PFS in patients with low (dashed lines) or high (solid lines) fold-change in sCD163, as determined by the median fold-change cutoff. Hazard ratio and 95% confidence interval were determined using univariate Cox model.

FIG. 2G is a set of KM curves showing OS in patients with low (dashed lines) or high (solid lines) fold-change in sCD163, as determined by the median fold-change cutoff. Hazard ratio and 95% confidence interval were determined using univariate Cox model.

FIG. 3A is a uniform manifold approximation and projection (UMAP) showing single peripheral blood mononuclear cells (PBMCs) from patients treated with tiragolumab+atezolizumab combination therapy colored by cell type (n=407,219). ILC: innate lymphoid cells; MDSC: myeloid-derived suppressor cells.

FIG. 3B is a box plot showing the proportion of PBMCs that were proliferating cells at Cycle 1, Day 1 (C1D1), Cycle 1, Day 15 (C1D15), Cycle 2, Day 1 (C2D1), and Cycle 4, Day 1 (C4D1) of tiragolumab+atezolizumab combination therapy. Boxplot center line, median; box, interquartile range (IQR; the range between the 25th and 75th percentile); whiskers, 1.58×IQR. Mean values per time point are connected by solid black lines. Samples from the same patient at different time points are connected by grey lines. P values shown are calculated by paired two-tailed student's t-test and BH-adjusted.

FIG. 3C is a box plot showing the proportion of CD4+ T cells that were Tregs at C1 D1, C1 D15, C2D1, and C4D1 of tiragolumab+atezolizumab combination therapy. Boxplot center line, median; box, interquartile range; whiskers, 1.58×IQR. Mean values per time point are connected by solid black lines. Samples from the same patient at different time points are connected by grey lines. P values shown are calculated by paired two-tailed student's t-test and BH-adjusted.

FIG. 3D is a set of box plots showing the proportion of total monocytes that were classical monocytes (left) or intermediate monocytes (right) at C1 D1, C1 D15, C2D1, and C4D1 of tiragolumab+atezolizumab combination therapy. Boxplot center line, median; box, interquartile range; whiskers, 1.58×IQR. Mean values per time point are connected by solid black lines. Samples from the same patient at different time points are connected by grey lines. P values shown are calculated by paired two-tailed student's t-test and BH-adjusted.

FIG. 3E is a set of heat maps showing levels of the indicated pathways in samples obtained on-treatment (C1D15, C2D1, C4D1) compared with those obtained at baseline (C1D1) from patients with NSCLC (n=15 pairs) across the indicated immune cell types. Color hue represents false discovery rate (FDR) significance. Red color indicates enrichment in on-treatment samples and blue color indicates enrichment in baseline samples. P values were calculated by nonparametric permutation test, and black asterisks represent FDR<0.05. TNFA, tumor necrosis factor alpha; TGF, transforming growth factor; NFKB, nuclear factor kappa B.

FIG. 4A is a set of growth curve charts showing tumor volume (mm³) over time in BALB/c mice that were implanted with syngeneic CT26 tumors. Tumor cells were allowed to grow for two weeks before treatment with a control IgG2a, anti-PD-L1, and/or anti-T-cell immunoglobulin and ITIM domain (anti-TIGIT) mIgG2a-LALAPG (fragment crystallizable region (Fc)-inactive), mIgG2b, or mIgG2a. Data are representative of one independent experiment with n=10 mice in each group.

FIG. 4B is a set of plots showing the mean fluorescence intensity (MFI) of cell surface major histocompatibility complex II (MHC-II) on tumor-infiltrating dendritic cells (DC), macrophages, and monocytes and a set or representative histograms relating to the monocyte data. DC: *, P=0.0264; *, P=0.0043. Macrophages: *, P=0.0119. Monocytes: *, P=0.0026; *, P=0.0017. Mean+/−SEM with one-way ANOVA and Dunnett's multiple comparisons, with the anti-PD-L1 monotherapy group designated as the control group. Each dot represents data from one mouse and n=5 per group.

FIG. 4C is a plot showing the proportion of tumor-infiltrating CD8+ T cells that were interferon gamma (IFNg)+ and TNFa+ after the indicated treatment and a representative pair of fluorescence-activated cell sorting (FACS) plots showing a gating strategy for identifying such cells. *, P=0.0007. Mean+/−SEM with one-way ANOVA and Dunnett's multiple comparisons, with the anti-PD-L1 monotherapy group designated as the control group.

FIG. 4D is a plot showing the proportion of tumor-infiltrating non-Treg (FoxP3−) CD4+ T cells that were IFNg+ and TNFa+after the indicated treatment and a representative pair of fluorescence-activated cell sorting (FACS) plots showing a gating strategy for identifying such cells. *P=0.0163, ****P<0.0001. Mean+/−SEM with one-way ANOVA and Dunnett's multiple comparisons, with the anti-PD-L1 monotherapy group designated as the control group.

FIG. 4E is a set of plots showing the proportion of total CD45+ cells that were FoxP3-non-Treg CD4+ T cells (left), FoxP3+ Treg CD4+ T cells (middle), or CD8+ T cells (right) after the indicated treatment. *P=0.0115. Mean+/−SEM with one-way ANOVA and Dunnett's multiple comparisons, with the anti-PD-L1 monotherapy group designated as the control group.

FIG. 4F is a plot showing the ratio of ratio of CD8+ T cells to FoxP3+ Treg CD4+ T cells after the indicated treatment. Mean+/−SEM with one-way ANOVA and Dunnett's multiple comparisons, with the anti-PD-L1 monotherapy group designated as the control group.

FIG. 5A is a pair of UMAPs showing tumor-infiltrating lymphocytes (top) and myeloid cells (bottom) from BALB/c mice colored by cell type.

FIG. 5B is a pair of bubble plots showing expression of the indicated marker genes in tumor-infiltrating T and NK cells (left) and myeloid cells (right) as shown in FIG. 5A. Broken y-axis was used to make the y-axis range comparable and for better comparison between treatments. P values are calculated by Wilcoxon rank-sum test.

FIG. 5C is a bubble plot (left) showing the expression of the indicated major histocompatibility complex (MHC) genes across the indicated treatments in all tumor-infiltrating monocytes and macrophages combined and a pair of volcano plots (middle and right) showing gene expression in grouped monocytes and macrophages following treatment with anti-PD-L1+anti-TIGIT IgG2b versus anti-PD-L1 (middle) or anti-PD-L1+anti-TIGIT IgG2a versus anti-PD-L1 (right). Broken y-axis was used to make the y-axis range comparable and for better comparison between treatments. P values are calculated by Wilcoxon rank-sum test.

FIG. 5D is a bubble plot (left) showing the expression of the indicated memory-like and exhaustion genes across the indicated treatments in total tumor-infiltrating CD8+ T cells (combined) and a pair of volcano plots (middle and right) showing gene expression in CD8+ T cells following treatment with anti-PD-L1+anti-TIGIT IgG2b versus anti-PD-L1 (middle) or anti-PD-L1+anti-TIGIT IgG2a versus anti-PD-L1 (right). Broken y-axis was used to make the y-axis range comparable and for better comparison between treatments. P values are calculated by Wilcoxon rank-sum test.

FIG. 5E is a bubble plot (left) showing the expression of the indicated immunosuppressive genes across the indicated treatments in tumor-infiltrating CD4 Tregs and a pair of volcano plots (middle and right) showing gene expression in CD4 Tregs following treatment with anti-PD-L1+anti-TIGIT IgG2b versus anti-PD-L1 (middle) or anti-PD-L1+anti-TIGIT IgG2a versus anti-PD-L1 (right). P values are calculated by Wilcoxon rank-sum test.

FIG. 6A is a UMAP showing peripheral blood cells colored by cell types.

FIG. 6B is a bubble plot showing expression of the indicated marker genes in the indicated cell types as shown in FIG. 6A.

FIG. 6C is a heatmap showing scaled gene expression of marker genes distinguishing classical, non-classical, and intermediate monocytes (top), and the expression patterns of FC gamma receptor (FcγR) (bottom) in the indicated monocyte subsets.

FIG. 6D is a set of volcano plots showing gene expression in classical (left), intermediate (middle), and non-classical (right) monocytes treated with anti-PD-L1+anti-TIGIT-IgG2a versus anti-PD-L1. P-values are calculated by Wilcoxon rank-sum test.

FIG. 7A is a pair of forest plots showing the association between high or low expression of the indicated genes in tumors and PFS (left) or OS (right) in patients treated with tiragolumab+atezolizumab versus placebo+atezolizumab. Hazard ratio and 95% confidence interval were determined using a univariate Cox model.

FIG. 7B is a set of Kaplan-Meier curves comparing PFS between patients having tumors enriched for TAMs and patients having tumors not enriched for TAMs in PD-L1-positive patients from the phase 3 NSCLC OAK study who received atezolizumab monotherapy. Patients were dichotomized by median signature score. Hazard ratio and 95% confidence interval were determined using a univariate Cox model.

FIG. 7C is a set of Kaplan-Meier curves comparing OS between patients having tumors enriched for TAMs and patients having tumors not enriched for TAMs in PD-L1-positive patients from the phase 3 NSCLC OAK study who received atezolizumab monotherapy. Patients were dichotomized by median signature score. Hazard ratio and 95% confidence interval were determined using a univariate Cox model.

FIG. 7D is a set of Kaplan-Meier curves comparing PFS between patients having tumors enriched for Tregs and patients having tumors not enriched for Tregs in PD-L1-positive patients from the phase 3 NSCLC OAK study who received atezolizumab monotherapy. Patients were dichotomized by median signature score. Hazard ratio and 95% confidence interval were determined using a univariate Cox model.

FIG. 7E is a set of Kaplan-Meier curves comparing OS between patients having tumors enriched for Tregs and patients having tumors not enriched for Tregs in PD-L1-positive patients from the phase 3 NSCLC OAK study who received atezolizumab monotherapy. Patients were dichotomized by median signature score. Hazard ratio and 95% confidence interval were determined using a univariate Cox model.

FIG. 8A is a scatter plot showing the correlation between the TAM gene signature score and the proportion of total cells that were CD68+, as quantified by mIF. Two-tailed Pearson correlation.

FIG. 8B is a scatter plot showing the correlation between the Treg gene signature score and the proportion of total cells that were FoxP3+, as quantified by mIF. Two-tailed Pearson correlation.

FIG. 9A is a scatter plot showing S and G2M cell cycle phase scores for individual cells. Cells identified as being in in proliferating or non-proliferating states are identified by color.

FIG. 9B is a bar graph showing the proportion of proliferating cells that were classified as belonging to each of the indicated cell types.

FIG. 9C is a set of box-and-whisker plots showing the proportion of proliferating cells that were CD4+ non-naïve T cells, CD8+ non-naïve T cells, and NK cells at C1 D1, C1 D15, C2D1, and C4D1 of tiragolumab+atezolizumab combination therapy. Boxplot center line, median; box, interquartile range; whiskers, 1.58×IQR. Mean values per time point are connected by solid black lines. Samples from the same patient at different time points are connected by grey lines. P values shown are calculated by paired two-tailed student's t-test and BH-adjusted.

FIG. 9D is a set of box-and-whisker plots showing the proportion of PBMCs that were identified as belonging to the indicated cell types at C1 D1, C1 D15, C2D1, and C4D1 of tiragolumab+atezolizumab combination therapy. Boxplot center line, median; box, interquartile range; whiskers, 1.58×IQR. Mean values per time point are connected by solid black lines. Samples from the same patient at different time points are connected by grey lines. P values shown are calculated by paired two-tailed student's t-test and BH-adjusted.

FIG. 9E is a set of box-and-whisker plots showing the proportion of PBMCs that were identified as belonging to the indicated cell types at C1 D1, C1 D15, C2D1, and C4D1 of tiragolumab+atezolizumab combination therapy in responders (complete response or partial response (CRPR)) and non-responders (stable disease or progressive disease (SDPD)). Boxplot center line, median; box, interquartile range; whiskers, 1.58×IQR. Mean values per time point are connected by solid black lines. Samples from the same patient at different time points are connected by grey lines. Nominal P values derived from two-tailed unpaired Student t-test are shown and red asterisk represents significance levels where * P<0.05.

FIG. 10A is a pair of UMAPs showing tumor-infiltrating T cells and NK cells (top) and myeloid cells (bottom) from BALB/c mice colored by cell type.

FIG. 10B is a pair of bubble plots showing expression of the indicated marker genes in T and NK cells (left) and myeloid cells (right) as shown in FIG. 10A.

FIG. 10C is a bubble plot showing the scaled expression of the indicated MHC and cytokine genes across the indicated treatments in tumor macrophages and monocytes.

FIG. 10D is a bubble plot showing the scaled expression of the indicated memory-like and exhaustion genes across the indicated treatments in total tumor CD8+ T cells.

FIG. 10E is a bubble plot showing the scaled expression of the indicated immunosuppressive genes across the indicated treatments in tumor CD4+ Tregs.

FIG. 10F is a UMAP showing single peripheral blood cells colored by cell types.

FIG. 10G is a bubble plot showing expression of the indicated marker genes in the indicated cell types as shown in FIG. 10F.

FIG. 10H is a heatmap showing scaled gene expression of marker genes distinguishing classical, non-classical, and intermediate monocytes (top), and the expression patterns of FcγR (bottom) in the indicated monocyte subsets.

FIG. 10I is a bubble plot showing scaled expression of the indicated MHC and interferon response genes in non-classical monocytes from the indicated treatment groups.

FIG. 11A is a set of growth curve charts showing tumor volume (mm³) over time in BALB/c mice that were implanted with syngeneic CT26 tumors. Tumor cells were allowed to grow for two weeks before treatment with a control IgG2a, and/or anti-TIGIT mIgG2a-LALAPG, mIgG2b, or mIgG2a. Data are representative of one independent experiment.

FIG. 11B is a set of growth curve charts showing tumor volume (mm³) over time in wild-type (top) or FcγR knockout (bottom) BALB/c mice that were implanted with syngeneic CT26 tumors. Tumor cells were allowed to grow for two weeks before treatment with a control IgG2a or anti-PD-L1 and anti-TIGIT mIgG2a. Data are representative of 1 independent experiment.

FIG. 12A is a pair of plots showing the proportion of gp70+CD226+ T cells in CT26-tumour bearing mice that were TCF1+ and SLAMF6+(memory-like) after treatment with a control antibody or anti-PD-L1 plus anti-TIGIT mIgG2a-LALAPG or mIgG2a antibodies. Statistics are one-way ANOVA with Tukey's multiple comparisons. *, p<0.05; **, p<0.01; ****, p<0.0001.

FIG. 12B is a pair of plots showing the proportion of gp70+CD226+ T cells in CT26-tumour bearing mice that were Tox+(terminally differentiated effector T cells) after treatment with a control antibody or anti-PD-L1 plus anti-TIGIT mIgG2a-LALAPG or mIgG2a antibodies. Statistics are one-way ANOVA with Tukey's multiple comparisons. *, p<0.05; **, p<0.01; ****, p<0.0001.

FIG. 13A is a set of box plots showing the proportion of total PBMCs belonging to the indicated cell types in mice treated with an IgG2a isotype control (B1); aPD-L1 (B2); aTIGIT-IgG2b (B3); aTIGIT-IgG2a (B4); aPD-L1+aTIGIT-IgG2b (B5); or aPD-L1+aTIGIT-IgG2a (B6). Boxplot center line, median; box, interquartile range; whiskers, 1.58×IQR. Normal P values by unpaired two-tailed student's t-test are shown in grey color; adjusted P values by Dunnett's multiple comparison were shown in black color.

FIG. 13B is a set of volcano plots showing gene expression in classical (left), intermediate (middle), and non-classical (right) monocytes from mice treated with anti-PD-L1+anti-TIGIT-IgG2b versus anti-PD-L1. P values are calculated by Wilcoxon rank-sum test.

FIG. 14A is a volcano plot showing relative expression levels of the indicated genes in tumor-infiltrating CD8+ T cells from BALB/c mice implanted with syngeneic CT26 tumors that were treated with an Fc-enabled anti-TIGIT IgG2a antibody and an anti-PD-L1 antibody as compared to mice treated with the anti-PD-L1 antibody alone. T effector memory genes (“memory”) and exhaustion-related genes (“exhaustion”) are indicated by color. FC: fold change relative to anti-PD-L1 monotherapy.

FIG. 14B is a heat map showing the average expression level of the indicated genes (indicated by dot color) and the percent of cells expressing the indicated genes (indicated by dot size) in tumor-infiltrating CD8+ T cells from BALB/c mice implanted with syngeneic CT26 tumors that were treated with a control IgG2a (T1); an anti-PD-L1 antibody (T2); an mIgG2b anti-TIGIT antibody (T3); an mIgG2a anti-TIGIT antibody (T4); an anti-PD-L1 antibody and an mIgG2b anti-TIGIT antibody (T5); or an anti-PD-L1 antibody and an mIgG2a anti-TIGIT antibody (T6). The yellow color indicates high expression level; blue color indicates low expression level. The colors indicate scaled average expression (i.e., average gene expression for cells in a group), where the scaled expression has mean=0 and standard deviation (SD)=1.

FIG. 14C is a volcano plot showing relative expression levels of the indicated genes in tumor-infiltrating CD4+ T cells (Tregs) from BALB/c mice implanted with syngeneic CT26 tumors that were treated with the Fc-enabled anti-TIGIT IgG2a antibody and an anti-PD-L1 antibody as compared to mice treated with the anti-PD-L1 antibody alone. Immune suppressive genes are indicated by color. FC: fold change relative to anti-PD-L1 monotherapy.

FIG. 14D is a heat map showing the average expression level of the indicated genes (indicated by dot color) and the percent of cells expressing the indicated genes (indicated by dot size) in tumor-infiltrating CD4+ T cells (Tregs) from BALB/c mice implanted with syngeneic CT26 tumors that were treated with a control IgG2a (T1); an anti-PD-L1 antibody (T2); an mIgG2b anti-TIGIT antibody (T3); an mIgG2a anti-TIGIT antibody (T4); an anti-PD-L1 antibody and an mIgG2b anti-TIGIT antibody (T5); or an anti-PD-L1 antibody and an mIgG2a anti-TIGIT antibody (T6). The yellow color indicates high expression level; blue color indicates low expression level. The colors indicate scaled average expression (i.e., average gene expression for cells in a group), where the scaled expression has mean=0 and SD=1.

FIG. 14E is a volcano plot showing relative expression levels of the indicated genes in tumor-infiltrating monocytes from BALB/c mice implanted with syngeneic CT26 tumors that were treated with the Fc-enabled anti-TIGIT IgG2a antibody and an anti-PD-L1 antibody as compared to mice treated with the anti-PD-L1 antibody alone. FC: fold change relative to anti-PD-L1 monotherapy. MHC-related genes (“MHC”) and other genes (“others”) are indicated by color.

FIG. 14F is a heat map showing the average expression level of the indicated genes (indicated by dot color) and the percent of cells expressing the indicated genes (indicated by dot size) in tumor-infiltrating monocytes from BALB/c mice implanted with syngeneic CT26 tumors that were treated with a control IgG2a (T1); an anti-PD-L1 antibody (T2); an mIgG2b anti-TIGIT antibody (T3); an mIgG2a anti-TIGIT antibody (T4); an anti-PD-L1 antibody and an mIgG2b anti-TIGIT antibody (T5); or an anti-PD-L1 antibody and an mIgG2a anti-TIGIT antibody (T6). The yellow color indicates high expression level; blue color indicates low expression level. The colors indicate scaled average expression (i.e., average gene expression for cells in a group), where the scaled expression has mean=0 and SD=1.

### DETAILED DESCRIPTION

### I. Overview

The present invention is based at least in part on the surprising discovery that a higher abundance of immunosuppressive cells, particularly tumor-associated macrophages (TAMs) and regulatory T cells (Tregs), are associated with improved objective response rate (ORR), overall survival (OS), and progression-free survival (PFS) for tiragolumab+atezolizumab combination therapy, but not for atezolizumab monotherapy. In particular, in an analysis of gene expression in tumor samples from patients in the Phase 2 CITYSCAPE study (GO30103), above-median TAM and Treg gene signature scores were each found to be associated with improved outcomes of tiragolumab+atezolizumab combination therapy. Further, in an analysis of pre-treatment and on-treatment serum samples (Cycle 2, Day 1 (C2D1) and Cycle 3, Day 1 (C3D1)) collected from CITYSCAPE patients, a comparison of circulating peptides that changed relative to baseline and 3 weeks post-treatment (C2D1) showed a statistically significant increase of myeloid-related protein peptides, such as MARCO (macrophage receptor with collagenous structure), CSF1R, CD163, CAMP, CD5L, and apolipoproteins (APOC2/3/4) in the tiragolumab+atezolizumab combination treatment arm, indicating that myeloid activation is a treatment-specific effect. It is also presently discovered that, surprisingly, an increased level of these myeloid proteins was associated with longer PFS and OS in patients receiving the tiragolumab+atezolizumab combination therapy versus patients receiving atezolizumab monotherapy for OS in patients with > median increase of serum myeloid proteins). The combination treatment thus showed a transient increase in serum myeloid proteins that was differentially associated with improved PFS and OS in the tiragolumab+atezolizumab combination treatment arm, indicating that myeloid cells are expected to play a key role in the enhanced anti-tumor efficacy of tiragolumab+atezolizumab.

Also discovered herein are novel pathways upregulated in monocytes that have not been reported for atezolizumab monotherapy and that are specific to tiragolumab+atezolizumab combination therapy, including the MYC targeting pathway, which has been shown to regulate macrophage polarization.

It is also presently discovered that, surprisingly, tiragolumab Fc domain interactions with the Fcγ receptors are required for the observed myeloid activation.

### II. General Techniques and Definitions

The techniques and procedures described or referenced herein are generally well understood and commonly employed using conventional methodology by those skilled in the art, such as, for example, the widely utilized methodologies described in Sambrook et al., Molecular Cloning: A Laboratory Manual3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X₁.”

The “amount,” “level,” or “expression level,” used herein interchangeably, of a biomarker is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein.

The terms “detecting” and “detection” are used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

The presence and/or expression level/amount of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

The term “complement C1q subcomponent subunit C” or “C1QC,” as used herein, broadly refers to any native C1QC from any mammalian source, including primates (e.g., humans) and rodents (e.g., mice and rats), unless otherwise indicated. The term encompasses full-length C1QC and isolated regions or domains of C1QC, e.g., the C1QC ECD. The term also encompasses naturally occurring variants of C1QC, e.g., splice variants or allelic variants. The amino acid sequence of an exemplary human C1QC is shown under UniProt Accession No. P02747. Minor sequence variations, especially conservative amino acid substitutions of C1QC that do not affect C1QC function and/or activity, are also contemplated by the invention.

The term “macrophage scavenger receptor types I and II” or “MSR1,” as used herein, broadly refers to any native MSR1 from any mammalian source, including primates (e.g., humans) and rodents (e.g., mice and rats), unless otherwise indicated. The term encompasses full-length MSR1 and isolated regions or domains of MSR1, e.g., the MSR1 ECD. The term also encompasses naturally occurring variants of MSR1, e.g., splice variants or allelic variants. The amino acid sequence of an exemplary human MSR1 is shown under UniProt Accession No. P21757. Minor sequence variations, especially conservative amino acid substitutions of MSR1 that do not affect MSR1 function and/or activity, are also contemplated by the invention.

The term “macrophage mannose receptor 1” or “MRC1,” as used herein, broadly refers to any native MRC1 from any mammalian source, including primates (e.g., humans) and rodents (e.g., mice and rats), unless otherwise indicated. The term encompasses full-length MRC1 and isolated regions or domains of MRC1, e.g., the MRC1 ECD. The term also encompasses naturally occurring variants of MRC1, e.g., splice variants or allelic variants. The amino acid sequence of an exemplary human MRC1 is shown under UniProt Accession No. P22897. Minor sequence variations, especially conservative amino acid substitutions of MRC1 that do not affect MRC1 function and/or activity, are also contemplated by the invention.

The term “V-set and immunoglobulin domain-containing protein 4” or “VSIG4,” as used herein, broadly refers to any native VSIG4 from any mammalian source, including primates (e.g., humans) and rodents (e.g., mice and rats), unless otherwise indicated. The term encompasses full-length VSIG4 and isolated regions or domains of VSIG4, e.g., the VSIG4 ECD. The term also encompasses naturally occurring variants of VSIG4, e.g., splice variants or allelic variants. The amino acid sequence of an exemplary human VSIG4 is shown under UniProt Accession No. Q9Y279. Minor sequence variations, especially conservative amino acid substitutions of VSIG4 that do not affect VSIG4 function and/or activity, are also contemplated by the invention.

The term “secreted phosphoprotein 1” or “SPP1,” as used herein, broadly refers to any native SPP1 from any mammalian source, including primates (e.g., humans) and rodents (e.g., mice and rats), unless otherwise indicated. The term encompasses full-length SPP1 and isolated regions or domains of SPP1, e.g., the SPP1 ECD. The term also encompasses naturally occurring variants of SPP1, e.g., splice variants or allelic variants. The amino acid sequence of an exemplary human SPP1 is shown under UniProt Accession No. P10451. Minor sequence variations, especially conservative amino acid substitutions of SPP1 that do not affect SPP1 function and/or activity, are also contemplated by the invention.

The term “macrophage receptor with collagenous structure” or “MARCO,” as used herein, broadly refers to any native MARCO from any mammalian source, including primates (e.g., humans) and rodents (e.g., mice and rats), unless otherwise indicated. The term encompasses full-length MARCO and isolated regions or domains of MARCO, e.g., the MARCO ECD. The term also encompasses naturally occurring variants of MARCO, e.g., splice variants or allelic variants. The amino acid sequence of an exemplary human MARCO is shown under UniProt Accession No. Q9UEW3. Minor sequence variations, especially conservative amino acid substitutions of MARCO that do not affect MARCO function and/or activity, are also contemplated by the invention.

The term “tartrate-resistant acid phosphatase type 5” or “ACP5,” as used herein, broadly refers to any native ACP5 from any mammalian source, including primates (e.g., humans) and rodents (e.g., mice and rats), unless otherwise indicated. The term encompasses full-length ACP5 and isolated regions or domains of ACP5, e.g., the ACP5 ECD. The term also encompasses naturally occurring variants of ACP5, e.g., splice variants or allelic variants. The amino acid sequence of an exemplary human ACP5 is shown under UniProt Accession No. P13686. Minor sequence variations, especially conservative amino acid substitutions of ACP5 that do not affect ACP5 function and/or activity, are also contemplated by the invention.

The term “mast cell-expressed membrane protein 1” or “MCEMP1,” as used herein, broadly refers to any native MCEMP1 from any mammalian source, including primates (e.g., humans) and rodents (e.g., mice and rats), unless otherwise indicated. The term encompasses full-length MCEMP1 and isolated regions or domains of MCEMP1, e.g., the MCEMP1 ECD. The term also encompasses naturally occurring variants of MCEMP1, e.g., splice variants or allelic variants. The amino acid sequence of an exemplary human MCEMP1 is shown under UniProt Accession No. Q81X19. Minor sequence variations, especially conservative amino acid substitutions of MCEMP1 that do not affect MCEMP1 function and/or activity, are also contemplated by the invention.

The term “sterol 27-hydroxylase” or “CYP27A1,” as used herein, broadly refers to any native CYP27A1 from any mammalian source, including primates (e.g., humans) and rodents (e.g., mice and rats), unless otherwise indicated. The term encompasses full-length CYP27A1 and isolated regions or domains of CYP27A1, e.g., the CYP27A1 ECD. The term also encompasses naturally occurring variants of CYP27A1, e.g., splice variants or allelic variants. The amino acid sequence of an exemplary human CYP27A1 is shown under UniProt Accession No. Q02318. Minor sequence variations, especially conservative amino acid substitutions of CYP27A1 that do not affect CYP27A1 function and/or activity, are also contemplated by the invention.

The term “oxidized low-density lipoprotein receptor 1” or “OLR1,” as used herein, broadly refers to any native OLR1 from any mammalian source, including primates (e.g., humans) and rodents (e.g., mice and rats), unless otherwise indicated. The term encompasses full-length OLR1 and isolated regions or domains of OLR1, e.g., the OLR1 ECD. The term also encompasses naturally occurring variants of OLR1, e.g., splice variants or allelic variants. The amino acid sequence of an exemplary human OLR1 is shown under UniProt Accession No. P78380. Minor sequence variations, especially conservative amino acid substitutions of OLR1 that do not affect OLR1 function and/or activity, are also contemplated by the invention.

The term “progranulin” or “GRN,” as used herein, broadly refers to any native GRN from any mammalian source, including primates (e.g., humans) and rodents (e.g., mice and rats), unless otherwise indicated. The term encompasses full-length GRN and isolated regions or domains of GRN, e.g., the GRN ECD. The term also encompasses naturally occurring variants of GRN, e.g., splice variants or allelic variants. The amino acid sequence of an exemplary human GRN is shown under UniProt Accession No. P28799. Minor sequence variations, especially conservative amino acid substitutions of GRN that do not affect GRN function and/or activity, are also contemplated by the invention.

The term “glioma pathogenesis-related protein 2” or “GLIPR2,” as used herein, broadly refers to any native GLIPR2 from any mammalian source, including primates (e.g., humans) and rodents (e.g., mice and rats), unless otherwise indicated. The term encompasses full-length GLIPR2 and isolated regions or domains of GLIPR2, e.g., the GLIPR2 ECD. The term also encompasses naturally occurring variants of GLIPR2, e.g., splice variants or allelic variants. The amino acid sequence of an exemplary human GLIPR2 is shown under UniProt Accession No. Q9H4G4. Minor sequence variations, especially conservative amino acid substitutions of GLIPR2 that do not affect GLIPR2 function and/or activity, are also contemplated by the invention.

The term “arrestin domain-containing protein 4” or “ARRDC4,” as used herein, broadly refers to any native ARRDC4 from any mammalian source, including primates (e.g., humans) and rodents (e.g., mice and rats), unless otherwise indicated. The term encompasses full-length ARRDC4 and isolated regions or domains of ARRDC4, e.g., the ARRDC4 ECD. The term also encompasses naturally occurring variants of ARRDC4, e.g., splice variants or allelic variants. The amino acid sequence of an exemplary human ARRDC4 is shown under UniProt Accession No. Q8NCT1. Minor sequence variations, especially conservative amino acid substitutions of ARRDC4 that do not affect ARRDC4 function and/or activity, are also contemplated by the invention.

The term “apolipoprotein E” or “APOE,” as used herein, broadly refers to any native APOE from any mammalian source, including primates (e.g., humans) and rodents (e.g., mice and rats), unless otherwise indicated. The term encompasses full-length APOE and isolated regions or domains of APOE, e.g., the APOE ECD. The term also encompasses naturally occurring variants of APOE, e.g., splice variants or allelic variants. The amino acid sequence of an exemplary human APOE is shown under UniProt Accession No. P02649. Minor sequence variations, especially conservative amino acid substitutions of APOE that do not affect APOE function and/or activity, are also contemplated by the invention.

The term “folate receptor beta” or “FOLR2,” as used herein, broadly refers to any native FOLR2 from any mammalian source, including primates (e.g., humans) and rodents (e.g., mice and rats), unless otherwise indicated. The term encompasses full-length FOLR2 and isolated regions or domains of FOLR2, e.g., the FOLR2 ECD. The term also encompasses naturally occurring variants of FOLR2, e.g., splice variants or allelic variants. The amino acid sequence of an exemplary human FOLR2 is shown under UniProt Accession No. P14207. Minor sequence variations, especially conservative amino acid substitutions of FOLR2 that do not affect FOLR2 function and/or activity, are also contemplated by the invention.

The term “cathepsin D” or “CTSD,” as used herein, broadly refers to any native CTSD from any mammalian source, including primates (e.g., humans) and rodents (e.g., mice and rats), unless otherwise indicated. The term encompasses full-length CTSD and isolated regions or domains of CTSD, e.g., the CTSD ECD. The term also encompasses naturally occurring variants of CTSD, e.g., splice variants or allelic variants. The amino acid sequence of an exemplary human CTSD is shown under UniProt Accession No. P07339. Minor sequence variations, especially conservative amino acid substitutions of CTSD that do not affect CTSD function and/or activity, are also contemplated by the invention.

The term “cathelicidin antimicrobial peptide” or “CAMP,” as used herein, broadly refers to any native CAMP from any mammalian source, including primates (e.g., humans) and rodents (e.g., mice and rats), unless otherwise indicated. The term encompasses full-length CAMP and isolated regions or domains of CAMP, e.g., the CAMP ECD. The term also encompasses naturally occurring variants of CAMP, e.g., splice variants or allelic variants. The amino acid sequence of an exemplary human CAMP is shown under UniProt Accession No. P49913. Minor sequence variations, especially conservative amino acid substitutions of CAMP that do not affect CAMP function and/or activity, are also contemplated by the invention.

The term “CD5 antigen-like” or “CD5L,” as used herein, broadly refers to any native CD5L from any mammalian source, including primates (e.g., humans) and rodents (e.g., mice and rats), unless otherwise indicated. The term encompasses full-length CD5L and isolated regions or domains of CD5L, e.g., the CD5L ECD. The term also encompasses naturally occurring variants of CD5L, e.g., splice variants or allelic variants. The amino acid sequence of an exemplary human CD5L is shown under UniProt Accession No. 043866. Minor sequence variations, especially conservative amino acid substitutions of CD5L that do not affect CD5L function and/or activity, are also contemplated by the invention.

The term “scavenger receptor cysteine-rich type 1 protein M130” or “CD163,” as used herein, broadly refers to any native CD163 from any mammalian source, including primates (e.g., humans) and rodents (e.g., mice and rats), unless otherwise indicated. The term encompasses full-length CD163 and isolated regions or domains of CD163, e.g., the CD163 ECD. The term also encompasses naturally occurring variants of CD163, e.g., splice variants or allelic variants. The amino acid sequence of an exemplary human CD163 is shown under UniProt Accession No. Q86VB7. Minor sequence variations, especially conservative amino acid substitutions of CD163 that do not affect CD163 function and/or activity, are also contemplated by the invention.

The term “neutrophil gelatinase-associated lipocalin” or “NGAL,” as used herein, broadly refers to any native NGAL from any mammalian source, including primates (e.g., humans) and rodents (e.g., mice and rats), unless otherwise indicated. The term encompasses full-length NGAL and isolated regions or domains of NGAL, e.g., the NGAL ECD. The term also encompasses naturally occurring variants of NGAL, e.g., splice variants or allelic variants. The amino acid sequence of an exemplary human NGAL is shown under UniProt Accession No. P80188. Minor sequence variations, especially conservative amino acid substitutions of NGAL that do not affect NGAL function and/or activity, are also contemplated by the invention.

The term “macrophage colony-stimulating factor 1 receptor” or “CSF1R,” as used herein, broadly refers to any native CSF1R from any mammalian source, including primates (e.g., humans) and rodents (e.g., mice and rats), unless otherwise indicated. The term encompasses full-length CSF1R and isolated regions or domains of CSF1R, e.g., the CSF1R ECD. The term also encompasses naturally occurring variants of CSF1R, e.g., splice variants or allelic variants. The amino acid sequence of an exemplary human CSF1R is shown under UniProt Accession No. P07333. Minor sequence variations, especially conservative amino acid substitutions of CSF1R that do not affect CSF1R function and/or activity, are also contemplated by the invention.

The term “CD44 antigen” or “CD44,” as used herein, broadly refers to any native CD44 from any mammalian source, including primates (e.g., humans) and rodents (e.g., mice and rats), unless otherwise indicated. The term encompasses full-length CD44 and isolated regions or domains of CD44, e.g., the CD44 ECD. The term also encompasses naturally occurring variants of CD44, e.g., splice variants or allelic variants. The amino acid sequence of an exemplary human CD44 is shown under UniProt Accession No. P16070. Minor sequence variations, especially conservative amino acid substitutions of CD44 that do not affect CD44 function and/or activity, are also contemplated by the invention.

The term “apolipoprotein C-II” or “APOC2,” as used herein, broadly refers to any native APOC2 from any mammalian source, including primates (e.g., humans) and rodents (e.g., mice and rats), unless otherwise indicated. The term encompasses full-length APOC2 and isolated regions or domains of APOC2, e.g., the APOC2 ECD. The term also encompasses naturally occurring variants of APOC2, e.g., splice variants or allelic variants. The amino acid sequence of an exemplary human APOC2 is shown under UniProt Accession No. P02655. Minor sequence variations, especially conservative amino acid substitutions of APOC2 that do not affect APOC2 function and/or activity, are also contemplated by the invention.

The term “apolipoprotein C-III” or “APOC3,” as used herein, broadly refers to any native APOC3 from any mammalian source, including primates (e.g., humans) and rodents (e.g., mice and rats), unless otherwise indicated. The term encompasses full-length APOC3 and isolated regions or domains of APOC3, e.g., the APOC3 ECD. The term also encompasses naturally occurring variants of APOC3, e.g., splice variants or allelic variants. The amino acid sequence of an exemplary human APOC3 is shown under UniProt Accession No. P02656. Minor sequence variations, especially conservative amino acid substitutions of APOC3 that do not affect APOC3 function and/or activity, are also contemplated by the invention.

The term “apolipoprotein C-IV” or “APOC4,” as used herein, broadly refers to any native APOC4 from any mammalian source, including primates (e.g., humans) and rodents (e.g., mice and rats), unless otherwise indicated. The term encompasses full-length APOC4 and isolated regions or domains of APOC4, e.g., the APOC4 ECD. The term also encompasses naturally occurring variants of APOC4, e.g., splice variants or allelic variants. The amino acid sequence of an exemplary human APOC4 is shown under UniProt Accession No. P55056. Minor sequence variations, especially conservative amino acid substitutions of APOC4 that do not affect APOC4 function and/or activity, are also contemplated by the invention.

The term “apolipoprotein A-II” or “APOA2,” as used herein, broadly refers to any native APOA2 from any mammalian source, including primates (e.g., humans) and rodents (e.g., mice and rats), unless otherwise indicated. The term encompasses full-length APOA2 and isolated regions or domains of APOA2, e.g., the APOA2 ECD. The term also encompasses naturally occurring variants of APOA2, e.g., splice variants or allelic variants. The amino acid sequence of an exemplary human APOA2 is shown under UniProt Accession No. P02652. Minor sequence variations, especially conservative amino acid substitutions of APOA2 that do not affect APOA2 function and/or activity, are also contemplated by the invention.

The term “lactotransferrin” or “TRFL,” as used herein, broadly refers to any native TRFL from any mammalian source, including primates (e.g., humans) and rodents (e.g., mice and rats), unless otherwise indicated. The term encompasses full-length TRFL and isolated regions or domains of TRFL, e.g., the TRFL ECD. The term also encompasses naturally occurring variants of TRFL, e.g., splice variants or allelic variants. The amino acid sequence of an exemplary human TRFL is shown under UniProt Accession No. P02788. Minor sequence variations, especially conservative amino acid substitutions of TRFL that do not affect TRFL function and/or activity, are also contemplated by the invention.

The term “vascular cell adhesion protein 1” or “VCAM1,” as used herein, broadly refers to any native VCAM1 from any mammalian source, including primates (e.g., humans) and rodents (e.g., mice and rats), unless otherwise indicated. The term encompasses full-length VCAM1 and isolated regions or domains of VCAM1, e.g., the VCAM1 ECD. The term also encompasses naturally occurring variants of VCAM1, e.g., splice variants or allelic variants. The amino acid sequence of an exemplary human VCAM1 is shown under UniProt Accession No. P13686. Minor sequence variations, especially conservative amino acid substitutions of VCAM1 that do not affect VCAM1 function and/or activity, are also contemplated by the invention.

The term “beta-2-microglobulin” or “B2MG,” as used herein, broadly refers to any native B2MG from any mammalian source, including primates (e.g., humans) and rodents (e.g., mice and rats), unless otherwise indicated. The term encompasses full-length B2MG and isolated regions or domains of B2MG, e.g., the B2MG ECD. The term also encompasses naturally occurring variants of B2MG, e.g., splice variants or allelic variants. The amino acid sequence of an exemplary human B2MG is shown under UniProt Accession No. P61769. Minor sequence variations, especially conservative amino acid substitutions of B2MG that do not affect B2MG function and/or activity, are also contemplated by the invention.

The term “forkhead box protein P3” or “FOXP3,” as used herein, broadly refers to any native FOXP3 from any mammalian source, including primates (e.g., humans) and rodents (e.g., mice and rats), unless otherwise indicated. The term encompasses full-length FOXP3 and isolated regions or domains of FOXP3, e.g., the FOXP3 ECD. The term also encompasses naturally occurring variants of FOXP3, e.g., splice variants or allelic variants. The amino acid sequence of an exemplary human FOXP3 is shown under UniProt Accession No. Q9BZS1. Minor sequence variations, especially conservative amino acid substitutions of FOXP3 that do not affect FOXP3 function and/or activity, are also contemplated by the invention.

The term “cytotoxic T-lymphocyte protein 4” or “CTLA4,” as used herein, broadly refers to any native CTLA4 from any mammalian source, including primates (e.g., humans) and rodents (e.g., mice and rats), unless otherwise indicated. The term encompasses full-length CTLA4 and isolated regions or domains of CTLA4, e.g., the CTLA4 ECD. The term also encompasses naturally occurring variants of CTLA4, e.g., splice variants or allelic variants. The amino acid sequence of an exemplary human CTLA4 is shown under UniProt Accession No. P16410. Minor sequence variations, especially conservative amino acid substitutions of CTLA4 that do not affect CTLA4 function and/or activity, are also contemplated by the invention.

The term “interleukin 10” or “IL10,” as used herein, broadly refers to any native IL10 from any mammalian source, including primates (e.g., humans) and rodents (e.g., mice and rats), unless otherwise indicated. The term encompasses full-length IL10 and isolated regions or domains of IL10, e.g., the IL10 ECD. The term also encompasses naturally occurring variants of IL10, e.g., splice variants or allelic variants. The amino acid sequence of an exemplary human IL10 is shown under UniProt Accession No. P22301. Minor sequence variations, especially conservative amino acid substitutions of IL10 that do not affect IL10 function and/or activity, are also contemplated by the invention.

The term “tumor necrosis factor receptor superfamily member 18” or “TNFRSF18,” as used herein, broadly refers to any native TNFRSF18 from any mammalian source, including primates (e.g., humans) and rodents (e.g., mice and rats), unless otherwise indicated. The term encompasses full-length TNFRSF18 and isolated regions or domains of TNFRSF18, e.g., the TNFRSF18 ECD. The term also encompasses naturally occurring variants of TNFRSF18, e.g., splice variants or allelic variants. The amino acid sequence of an exemplary human TNFRSF18 is shown under UniProt Accession No. Q9Y5U5. Minor sequence variations, especially conservative amino acid substitutions of TNFRSF18 that do not affect TNFRSF18 function and/or activity, are also contemplated by the invention.

The term “C-C chemokine receptor type 8” or “CCR8,” as used herein, broadly refers to any native CCR8 from any mammalian source, including primates (e.g., humans) and rodents (e.g., mice and rats), unless otherwise indicated. The term encompasses full-length CCR8 and isolated regions or domains of CCR8, e.g., the CCR8 ECD. The term also encompasses naturally occurring variants of CCR8, e.g., splice variants or allelic variants. The amino acid sequence of an exemplary human CCR8 is shown under UniProt Accession No. P51685. Minor sequence variations, especially conservative amino acid substitutions of CCR8 that do not affect CCR8 function and/or activity, are also contemplated by the invention.

The term “zinc finger protein Eos” or “IKZF4,” as used herein, broadly refers to any native IKZF4 from any mammalian source, including primates (e.g., humans) and rodents (e.g., mice and rats), unless otherwise indicated. The term encompasses full-length IKZF4 and isolated regions or domains of IKZF4, e.g., the IKZF4 ECD. The term also encompasses naturally occurring variants of IKZF4, e.g., splice variants or allelic variants. The amino acid sequence of an exemplary human IKZF4 is shown under UniProt Accession No. Q9H2S9. Minor sequence variations, especially conservative amino acid substitutions of IKZF4 that do not affect IKZF4 function and/or activity, are also contemplated by the invention.

The term “zinc finger protein Helios” or “IKZF2,” as used herein, broadly refers to any native IKZF2 from any mammalian source, including primates (e.g., humans) and rodents (e.g., mice and rats), unless otherwise indicated. The term encompasses full-length IKZF2 and isolated regions or domains of IKZF2, e.g., the IKZF2 ECD. The term also encompasses naturally occurring variants of IKZF2, e.g., splice variants or allelic variants. The amino acid sequence of an exemplary human IKZF2 is shown under UniProt Accession No. Q9UKS7. Minor sequence variations, especially conservative amino acid substitutions of IKZF2 that do not affect IKZF2 function and/or activity, are also contemplated by the invention.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 30), as well as any form of TIGIT that results from processing in the cell (e.g., processed human TIGIT without a signal sequence, having the amino acid sequence of SEQ ID NO: 31). The term also encompasses naturally occurring variants of TIGIT, e.g., splice variants or allelic variants. The amino acid sequence of an exemplary human TIGIT may be found under UniProt Accession Number Q495A1.

The term “PD-L1” or “Programmed Cell Death Ligand 1” refers herein to any native PD-L1 from any vertebrate source, including mammals such as primates (e.g., humans) and rodents (e.g., mice and rats), unless otherwise indicated. PD-L1 is also known in the art as CD274 molecule, CD274 antigen, B7 homolog 1, PDCD1 Ligand 1, PDCD1LG1, PDCD1 L1, B7H1, PDL1, programmed death ligand 1, B7-H1, and B7-H. The term also encompasses naturally occurring variants of PD-L1, e.g., splice variants, or allelic variants. The amino acid sequence of an exemplary human PD-L1 may be found under UniProt Accession Number Q9NZQ7 (SEQ ID NO: 32).

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments (e.g., antigen-binding fragments), fragments or amino acid sequence variants of native polypeptides, peptides, antisense oligonucleotides, small organic molecules, etc. Methods for identifying antagonists of a polypeptide may comprise contacting a polypeptide with a candidate antagonist molecule and measuring a detectable change in one or more biological activities normally associated with the polypeptide.

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function (e.g., proliferation, cytokine production, target cell killing). As used herein, a PD-1 axis binding antagonist includes a PD-1 binding antagonist, a PD-L1 binding antagonist, and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is pembrolizumab (formerly lambrolizumab (MK-3475)) described herein. In another specific aspect, a PD-1 binding antagonist is AMP-224 described herein.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is atezolizumab described herein (e.g., MPDL3280A). In another specific aspect, an anti-PD-L1 antibody is MDX-1105 described herein. In still another specific aspect, an anti-PD-L1 antibody is MEDI4736 described herein.

As used herein, the term “atezolizumab” refers to anti-PD-L1 antagonist antibody having the International Nonproprietary Names for Pharmaceutical Substances (INN) List 112 (WHO Drug Information, Vol. 28, No. 4, 2014, p. 488), or the CAS Registry Number 1380723-44-3.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to restore a functional response by T-cells (e.g., proliferation, cytokine production, target cell killing) from a dysfunctional state to antigen stimulation.

For example, an anti-TIGIT antagonist antibody may block signaling through PVR without impacting PVR-CD226 interaction. It will be understood by one of ordinary skill in the art that in some instances, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one embodiment, the extent of binding of an anti-TIGIT antagonist antibody to an unrelated, non-TIGIT protein is less than about 10% of the binding of the antibody to TIGIT as measured, e.g., by a radioimmunoassay (RIA). In certain embodiments, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-TIGIT antagonist antibody binds to an epitope of TIGIT that is conserved among TIGIT from different species or an epitope on TIGIT that allows for cross-species reactivity. In one embodiment, the anti-TIGIT antagonist antibody is tiragolumab.

As used herein, “tiragolumab” is a fully human IgG1/kappa MAb-derived in Open Monoclonal Technology (OMT) rats that binds TIGIT and comprises the heavy chain sequence of SEQ ID NO: 33 and the light chain sequence of SEQ ID NO: 34. Tiragolumab comprises two N-linked glycosylation sites (N306) in the Fc domain. Tiragolumab is also described in WHO Drug Information (International Nonproprietary Names for Pharmaceutical Substances), Proposed INN: List 117, Vol. 31, No. 2, published Jun. 9, 2017 (see page 343).

As used herein, “administering” is meant a method of giving a dosage of a compound (e.g., an anti-TIGIT antagonist antibody or a PD-1 axis binding antagonist (e.g., an anti-PD-L1 antibody)) or a composition (e.g., a pharmaceutical composition, e.g., a pharmaceutical composition including an anti-TIGIT antibody and/or a PD-1 axis binding antagonist (e.g., an anti-PD-L1 antibody)) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

As used herein, “systemic treatment” refers to a treatment that travels through the bloodstream and is capable of contacting multiple organ systems upon a single administration. The term “systemic treatment” is well understood by those skilled in the art and is equivalent to systemic therapy.

A “fixed” or “flat” dose of a therapeutic agent (e.g., an anti-TIGIT antagonist antibody or a PD-1 axis binding antagonist (e.g., an anti-PD-L1 antibody) herein refers to a dose that is administered to a patient without regard for the weight or body surface area (BSA) of the patient. The fixed or flat dose is therefore not provided as a mg/kg dose or a mg/m2 dose, but rather as an absolute amount of the therapeutic agent (e.g., mg).

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “in conjunction with” refers to administration of one treatment modality in addition to another treatment modality. As such, “in conjunction with” refers to administration of one treatment modality before, during, or after administration of the other treatment modality to the individual.

A “disorder” or “disease” is any condition that would benefit from treatment including, but not limited to, disorders that are associated with some degree of abnormal cell proliferation, e.g., cancer, e.g., lung cancer, e.g., non-small cell lung cancer (NSCLC).

The term “dysfunction,” in the context of immune dysfunction, refers to a state of reduced immune responsiveness to antigenic stimulation.

The term “dysfunctional,” as used herein, also includes refractory or unresponsive to antigen recognition, specifically, impaired capacity to translate antigen recognition into downstream T-cell effector functions, such as proliferation, cytokine production (e.g., gamma interferon) and/or target cell killing.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include, but are not limited to lung cancer, such as non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung, or squamous cell cancer of the lung (e.g., epithelial squamous cell cancer (e.g., squamous carcinoma of the lung); and small cell lung cancer (SCLC), which includes extensive stage SCLC (ES-SCLC). Additional examples of cancer are gastric cancer or stomach cancer, including gastrointestinal cancer, gastrointestinal stromal cancer, or gastroesophageal junction cancer; esophageal cancer; colon cancer; rectal cancer; colorectal cancer; cancer of the peritoneum; hepatocellular cancer; pancreatic cancer; glioblastoma; cervical cancer; ovarian cancer; liver cancer; bladder cancer (e.g., urothelial bladder cancer (UBC), muscle invasive bladder cancer (MIBC), and BCG-refractory non-muscle invasive bladder cancer (NMIBC)); cancer of the urinary tract; hepatoma; breast cancer (e.g., HER2+ breast cancer and triple-negative breast cancer (TNBC), which are estrogen receptors (ER−), progesterone receptors (PR−), and HER2 (HER2−) negative); endometrial or uterine carcinoma; salivary gland carcinoma; kidney or renal cancer (e.g., renal cell carcinoma (RCC)); prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL)); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “anti-cancer therapy” refers to a therapy useful in treating cancer (e.g., lung cancer, e.g., NSCLC). Examples of anti-cancer therapeutic agents include, but are limited to, e.g., immunomodulatory agents (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), an anti-TIGIT antagonist antibody, or a PD-1 axis binding antagonist (e.g., an anti-PD-L1 antibody), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), chemotherapeutic agents, growth inhibitory agents, cytotoxic agents, agents used in radiation therapy, anti-angiogenesis agents, apoptotic agents, anti-tubulin agents, and other agents to treat cancer. Combinations thereof are also included in the invention.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anti-cancer agents disclosed below.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors (e.g., an anaplastic lymphoma kinase (Alk) inhibitor, such as AF-802 (also known as CH-5424802 or alectinib)); (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; inhibitors of insulin receptor tyrosine kinases, including anaplastic lymphoma kinase (Alk) inhibitors, such as AF-802 (also known as CH-5424802 or alectinib), ASP3026, X396, LDK378, AP26113, crizotinib (XALKORI®), and ceritinib (ZYKADIA®); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFa) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/P2 blockers such as Anti-lymphotoxin alpha (LTa); radioactive isotopes (e.g., At211, 1131, 1125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

An “effective amount” of a compound, for example, an anti-TIGIT antagonist antibody or a PD-1 axis binding antagonist (e.g., anti-PD-L1 antibody), or a composition (e.g., pharmaceutical composition) thereof, is at least the minimum amount required to achieve the desired therapeutic result, such as a measurable increase in overall survival or progression-free survival of a particular disease or disorder (e.g., cancer, e.g., lung cancer (e.g., NSCLC)). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease (e.g., reduction or delay in cancer-related pain, symptomatic skeletal-related events (SSE), reduction in symptoms per the European Organization for Research and Treatment of Cancer Quality-of-Life Questionnaire (EORTC QLQ-C30, e.g., fatigue, nausea, vomiting, pain, dyspnea, insomnia, appetite loss, constipation, diarrhea, or general level of physical emotional, cognitive, or social functioning), reduction in pain as measured by, e.g., the 10-point pain severity (measured at its worst) numerical rating scale (NRS), and/or reduction in symptoms associated with lung cancer per the health-related quality of life (HRQoL) questionnaire as assessed by symptoms in lung cancer (SILC) scale (e.g., time to deterioration (TTD) in cough dyspenea and chest pain), increasing the quality of life of those suffering from the disease, decreasing the dose of other medications required to treat the disease, enhancing effect of another medication such as via targeting, delaying the progression of the disease (e.g. progression-free survival or radiographic progression-free survival (rPFS); delay of unequivocal clinical progression (e.g., cancer-related pain progression, symptomatic skeletal-related event, deterioration in Eastern Cooperative Group Oncology Group (ECOG) Performance Status (PS) (e.g., how the disease affects the daily living abilities of the patient), and/or initiation of next systemic anti-cancer therapy), and/or delaying time to lung-specific antigen progression), and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Individual response” or “response” can be assessed using any endpoint indicating a benefit to the subject, including, without limitation, (1) inhibition, to some extent, of disease progression (e.g., progression of cancer, e.g., lung cancer (e.g., NSCLC)), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the disease or disorder (e.g., cancer, e.g., lung cancer (e.g., NSCLC)); (6) increase or extension in the length of survival, including overall survival and progression-free survival; and/or (9) decreased mortality at a given point of time following treatment.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “objective response rate” (ORR) refers to the sum of complete response (CR) rate and partial response (PR) rate.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); resulting in an objective response (including a complete response or a partial response); or improving signs or symptoms of cancer.

As used herein, “survival” refers to the patient remaining alive, and includes overall survival as well as progression-free survival.

As used herein, “overall survival” (OS) refers to the percentage of subjects in a group who are alive after a particular duration of time, e.g., 1 year or 5 years from the time of diagnosis or treatment.

As used herein, “progression-free survival” (PFS) refers to the length of time during and after treatment during which the disease being treated (e.g., cancer, e.g., lung cancer (e.g., NSCLC)) does not get worse. Progression-free survival may include the amount of time patients have experienced a complete response or a partial response, as well as the amount of time patients have experienced stable disease.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “delaying progression” of a disorder or disease means to defer, hinder, slow, retard, stabilize, and/or postpone development of the disease or disorder (e.g., cancer, e.g., lung cancer (e.g., NSCLC)). This delay can be of varying lengths of time, depending on the history of the disease and/or subject being treated. As is evident to one skilled in the art, a sufficient or significant delay can, in effect, encompass prevention, in that the subject does not develop the disease. For example, in a late stage cancer, development of central nervous system (CNS) metastasis, may be delayed.

By “extending survival” is meant increasing overall or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

As used herein, “hazard ratio” or “HR” is a statistical definition for rates of events. For the purpose of the invention, hazard ratio is defined as representing the probability of an event (e.g., PFS or OS) in the experimental (e.g., treatment) group/arm divided by the probability of an event in the control group/arm at any specific point in time. An HR with a value of 1 indicates that the relative risk of an endpoint (e.g., death) is equal in both the “treatment” and “control” groups; a value greater than 1 indicates that the risk is greater in the treatment group relative to the control group; and a value less than 1 indicates that the risk is greater in the control group relative to the treatment group. “Hazard ratio” in progression-free survival analysis (i.e., PFS HR) is a summary of the difference between two progression-free survival curves, representing the reduction in the risk of death on treatment compared to control, over a period of follow-up. “Hazard ratio” in overall survival analysis (i.e., OS HR) is a summary of the difference between two overall survival curves, representing the reduction in the risk of death on treatment compared to control, over a period of follow-up.

As used herein, the “Ventana SP263 IHC assay” (also referred to herein as the Ventana SP263 CDx assay) is conducted according to the Ventana PD-L1 (SP263) Assay package insert (Tucson, AZ: Ventana Medical Systems, Inc.), which is incorporated herein by reference in its entirety.

As used herein, the “Ventana SP142 IHC assay” is conducted according to the Ventana PD-L1 (SP142) Assay package insert (Tucson, AZ: Ventana Medical Systems, Inc.), which is incorporated herein by reference in its entirety.

As used herein, the “pharmDx 22C3 IHC assay” is conducted according to the PD-L1 IHC 22C3 pharmDx package insert (Carpinteria, CA: Dako, Agilent Pathology Solutions), which is incorporated herein by reference in its entirety.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+ T lymphocytes and/or CD4+ T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

The term “biomarker,” as used herein, refers to an indicator, e.g., predictive, diagnostic, and/or prognostic, which can be detected in a sample. In some embodiments, a biomarker is a gene. Biomarkers include, but are not limited to, polypeptides, polynucleotides (e.g., DNA, and/or RNA), polynucleotide copy number alterations (e.g., DNA copy numbers), polypeptide and polynucleotide modifications (e.g., posttranslational modifications), carbohydrates, and/or glycolipid-based molecular markers.

The term “antibody” includes monoclonal antibodies (including full-length antibodies which have an immunoglobulin Fc region), antibody compositions with polyepitopic specificity, multispecific antibodies (e.g., bispecific antibodies), diabodies, and single-chain molecules, as well as antibody fragments, including antigen-binding fragments, such as Fab, F(ab′)₂, and Fv. The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).
   Unless otherwise indicated, the CDRs are determined according to Kabat et al., supra. One of skill in the art will understand that the CDR designations can also be determined according to Chothia, supra, McCallum, supra, or any other scientifically accepted nomenclature system.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen-binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′-fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3 and IgG4. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Da\tilde{e}ron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Affinity” refers to the strength of the sum total of non-covalent interactions between a single binding site of a molecule (e.g., an antibody) and its binding partner (e.g., an antigen, e.g., TIGIT or PD-L1). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat. Acad. Sci. USA, 103:3557-3562 (2006) regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “subject” or “individual” means a mammal, including, but not limited to, a human or non-human mammal, such as a bovine, equine, canine, ovine, or feline. In some embodiments, the subject is a human. Patients are also subjects herein.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “tumor sample,” “disease sample,” and variations thereof refers to any sample obtained from a subject of interest that would be expected or is known to contain the cellular and/or molecular entity that is to be characterized. In some embodiments, the sample is a tumor tissue sample (e.g., a lung cancer sample (e.g., a NSCLC sample)). Other samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, stool, tumor lysates, and tissue culture medium, tissue extracts such as homogenized tissue, cellular extracts, and combinations thereof.

The terms “tissue sample” and “cell sample” mean a collection of similar cells obtained from a tissue of a subject or individual. The source of the tissue or cell sample may be solid tissue as from a fresh, frozen, and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a diseased tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of a subject who is not the subject. In even another embodiment, a reference sample, reference cell, reference tissue, control sample, control cell, or control tissue is obtained from an untreated tissue and/or cell of the body of an individual who is not the subject.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of an active ingredient contained therein to be effective, and which contains no additional components which are unacceptably toxic to a subject to which the formulation would be administered.

### III. Prognostic Methods and Assays

a. Tumor-Associated Macrophage (TAM) Genes and Gene Signatures
Methods of Identifying Individuals Who May Benefit from Treatment

### (i) TAM Genes

In one aspect, the invention provides a method of identifying an individual having a cancer (e.g., a lung cancer, e.g., a non-small cell lung cancer (NSCLC)) who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), the method comprising detecting an expression level of one or more of tumor-associated macrophage (TAM) genes complement C1q subcomponent subunit C (C1QC), macrophage scavenger receptor types I and II (MSR1), Macrophage mannose receptor 1 (MRC1), V-set and immunoglobulin domain-containing protein 4 (VSIG4), secreted phosphoprotein 1 (SPP1), and macrophage receptor with collagenous structure (MARCO) (e.g., one, two, three, four, five, or all six of C1QC, MSR1, MRC1, VSIG4, SPP1, and MARCO) in a sample from the individual, wherein an expression level of one or more of C1QC, MSR1, MRC1, VSIG4, SPP1, and MARCO that is above a respective reference expression level identifies the individual as one who may benefit from a treatment comprising a PD-1 axis binding antagonist and an anti-TIGIT antagonist antibody.

### (ii) TAM Signature Scores

In another aspect, the invention provides a method of identifying an individual having a cancer (e.g., a lung cancer, e.g., a NSCLC) who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), the method comprising detecting an expression level of at least two of C1QC, MSR1, MRC1, VSIG4, SPP1, and MARCO (e.g., two, three, four, five, or all six of C1QC, MSR1, MRC1, VSIG4, SPP1, and MARCO) in a sample from the individual and determining a tumor-associated macrophage (TAM) signature score therefrom, wherein a TAM signature score that is above a reference TAM signature score identifies the individual as one who may benefit from a treatment comprising a PD-1 axis binding antagonist and an anti-TIGIT antagonist antibody.

In another aspect, the invention provides a method of identifying an individual having a cancer (e.g., a lung cancer, e.g., a NSCLC) who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), the method comprising detecting an expression level of each of C1QC, MSR1, MRC1, VSIG4, SPP1, and MARCO in a sample from the individual and determining a TAM signature score therefrom, wherein a TAM signature score that is above a reference TAM signature score identifies the individual as one who may benefit from a treatment comprising a PD-1 axis binding antagonist and an anti-TIGIT antagonist antibody.

In some aspects, the individual has a TAM signature score in the sample that is above a reference TAM signature score, and the method further comprises administering to the individual an effective amount of a PD-1 axis binding antagonist and an anti-TIGIT antagonist antibody. Exemplary methods for determining a TAM signature score and exemplary reference TAM signature scores are provided below. Exemplary PD-1 axis binding antagonists, anti-TIGIT antagonist antibodies, and methods of treatment comprising these agents are provided in Section IV.

In some aspects of any of the methods provided herein, the cancer is a lung cancer, e.g., a NSCLC, a small cell lung cancer (SCLC), or a lung carcinoid tumor. In some aspects, the individual is a human.

### Methods of Selecting a Therapy

### (i) TAM Genes

In another aspect, the invention provides a method for selecting a therapy for an individual having a cancer (e.g., a lung cancer, e.g., a NSCLC), the method comprising detecting an expression level of one or more of tumor-associated macrophage (TAM) genes C1QC, MSR1, MRC1, VSIG4, SPP1, and MARCO (e.g., one, two, three, four, five, or all six of C1QC, MSR1, MRC1, VSIG4, SPP1, and MARCO) in a sample from the individual, wherein an expression level of one or more of C1QC, MSR1, MRC1, VSIG4, SPP1, and MARCO that is above a respective reference expression level identifies the individual as one who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)).

### (ii) TAM Signature Scores

In another aspect, the invention provides a method for selecting a therapy for an individual having a cancer (e.g., a lung cancer, e.g., a NSCLC), the method comprising detecting an expression level of at least two of C1QC, MSR1, MRC1, VSIG4, SPP1, and MARCO (e.g., two, three, four, five, or all six of C1QC, MSR1, MRC1, VSIG4, SPP1, and MARCO) in a sample from the individual and determining a TAM signature score therefrom, wherein a TAM signature score that is above a reference TAM signature score identifies the individual as one who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)).

In another aspect, the invention provides a method for selecting a therapy for an individual having a cancer (e.g., a lung cancer, e.g., a NSCLC), the method comprising detecting an expression level of each of C1QC, MSR1, MRC1, VSIG4, SPP1, and MARCO in a sample from the individual and determining a TAM signature score therefrom, wherein a TAM signature score that is above a reference TAM signature score identifies the individual as one who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)).

In some aspects, the individual has a TAM signature score in the sample that is above a reference TAM signature score, and the method further comprises administering to the individual an effective amount of a PD-1 axis binding antagonist and an anti-TIGIT antagonist antibody.

### Methods of Treatment

### (i) TAM Genes

In another aspect, the invention provides a method of treating an individual having a cancer (e.g., a lung cancer, e.g., a NSCLC), the method comprising (a) detecting an expression level of one or more of tumor-associated macrophage (TAM) genes C1QC, MSR1, MRC1, VSIG4, SPP1, and MARCO (e.g., one, two, three, four, five, or all six of C1QC, MSR1, MRC1, VSIG4, SPP1, and MARCO) in a sample from the individual, wherein the expression level of one or more of C1QC, MSR1, MRC1, VSIG4, SPP1, and MARCO is above a respective reference expression level and thereby identifies the individual as one who may benefit from a treatment comprising a PD-1 axis binding antagonist and an anti-TIGIT antagonist antibody; and (b) administering an effective amount of a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual.

In another aspect, the invention provides a method of treating an individual having a cancer (e.g., a lung cancer, e.g., a NSCLC), the method comprising administering a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual, wherein the individual has been determined to have an expression level of one or more of C1QC, MSR1, MRC1, VSIG4, SPP1, and MARCO that is above a respective reference expression level, thereby identifying the individual as one who may benefit from a treatment comprising a PD-1 axis binding antagonist and an anti-TIGIT antagonist antibody.

### (ii) TAM Signature Scores

In another aspect, the invention provides a method of treating an individual having a cancer (e.g., a lung cancer, e.g., a NSCLC), the method comprising (a) detecting an expression level of at least two of C1QC, MSR1, MRC1, VSIG4, SPP1, and MARCO (e.g., two, three, four, five, or all six of C1QC, MSR1, MRC1, VSIG4, SPP1, and MARCO) in a sample from the individual and determining a TAM signature score therefrom, wherein the TAM signature score is above a reference TAM signature score and thereby identifies the individual as one who may benefit from a treatment comprising a PD-1 axis binding antagonist and an anti-TIGIT antagonist antibody; and (b) administering an effective amount of a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual.

In another aspect, the invention provides a method of treating an individual having a cancer (e.g., a lung cancer, e.g., a NSCLC), the method comprising (a) detecting an expression level of each of C1QC, MSR1, MRC1, VSIG4, SPP1, and MARCO in a sample from the individual and determining a TAM signature score therefrom, wherein the TAM signature score is above a reference TAM signature score and thereby identifies the individual as one who may benefit from a treatment comprising a PD-1 axis binding antagonist and an anti-TIGIT antagonist antibody; and (b) administering an effective amount of a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual.

In another aspect, the invention provides a method of treating an individual having a cancer (e.g., a lung cancer, e.g., a NSCLC), the method comprising administering a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual, wherein the individual has been determined to have a TAM signature score that is above a reference TAM signature score, thereby identifying the individual as one who may benefit from a treatment comprising a PD-1 axis binding antagonist and an anti-TIGIT antagonist antibody, and wherein the TAM signature score is based on the expression level of at least two of C1QC, MSR1, MRC1, VSIG4, SPP1, and MARCO (e.g., two, three, four, five, or all six of C1QC, MSR1, MRC1, VSIG4, SPP1, and MARCO) detected in a sample from the individual.

In another aspect, the invention provides a method of treating an individual having a cancer (e.g., a lung cancer, e.g., a NSCLC), the method comprising administering a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual, wherein the individual has been determined to have a TAM signature score that is above a reference TAM signature score, thereby identifying the individual as one who may benefit from a treatment comprising a PD-1 axis binding antagonist and an anti-TIGIT antagonist antibody, and wherein the TAM signature score is based on the expression level of each of C1QC, MSR1, MRC1, VSIG4, SPP1, and MARCO detected in a sample from the individual.

### Benefit

An individual who benefits from receiving treatment with a PD-1 axis binding antagonist and an anti-TIGIT antagonist antibody may experience, for example, a delay or prevention in the occurrence or recurrence of a cancer (e.g., a lung cancer, e.g., a NSCLC), alleviation of symptoms of the cancer, diminishment of any direct or indirect pathological consequences of the cancer, prevention of metastasis, decrease in the rate of disease progression, amelioration or palliation of the disease state, or remission or improved prognosis.

In some aspects, the benefit achieved by the treatment comprising a PD-1 axis binding antagonist and an anti-TIGIT antagonist antibody is an increase in progression-free survival (PFS) (e.g., an increase in the duration of PFS experienced by an individual treated according to the method or an increase in the average PFS of a population of individuals treated according to the method), an increase in objective response rate (ORR) (e.g., an increase in the ORR of a population of individuals treated according to the method), and/or an increase in overall survival (OS) (e.g., an increase in the duration of OS experienced by an individual treated according to the method or an increase in the average OS of a population of individuals treated according to the method).

An increased PFS, ORR, and/or OS may be determined by comparison to, e.g., an untreated reference individual and/or a reference population of individuals; a reference individual and/or a reference population of individuals who have received a control treatment, such as one or more previously approved treatments or marketed products for treatment of the cancer; and/or a reference individual and/or a reference population of individuals who have been treated with a PD-1 axis binding antagonist (e.g., atezolizumab) or an anti-TIGIT antagonist antibody (e.g., tiragolumab) as a monotherapy. In some aspects, the increased PFS, ORR, and/or OS is determined relative to a reference individual and/or a reference population of individuals having cancer that have been treated with a treatment comprising a PD-1 axis binding antagonist and an anti-TIGIT antagonist antibody (e.g., atezolizumab and tiragolumab), wherein the reference individual and/or each individual in the reference population has a TAM signature score that is at or below a reference TAM signature score and/or has an expression level of one or more of C1QC, MSR1, MRC1, VSIG4, SPP1, and MARCO that is at or below a respective reference expression level. Reference TAM signature scores are described herein and may, for example, be a median TAM signature score of a reference population of individuals having cancer (e.g., a lung cancer, e.g., a NSCLC) or a median expression level of one or more of C1QC, MSR1, MRC1, VSIG4, SPP1, and MARCO in a reference population of individuals having cancer (e.g., a lung cancer, e.g., a NSCLC).

The skilled person is readily able to decide whether a given clinical outcome is improved in accordance with the invention. For example, “improved” in this context means that the clinical outcome resulting from the treatment of an individual having an expression level of one or more of C1QC, MSR1, MRC1, VSIG4, SPP1, and MARCO that is above a respective reference expression level or having a TAM signature score that is above a reference TAM signature score with a treatment comprising a PD-1 axis binding antagonist and an anti-TIGIT antagonist antibody (e.g., atezolizumab and tiragolumab) is at least 3% higher, at least 5% higher, at least 7% higher, at least 10% higher, at least 15% higher, at least 20% higher, at least 25% higher, at least 30% higher, at least 40% higher, at least 50% higher, at least 75% higher, at least 100% higher, or at least 120% higher, as compared to the clinical outcome resulting from a comparator treatment as described above.

For example, in some aspects, the duration of PFS or OS experienced by an individual treated according to the method or the average PFS or OS of a population of individuals treated according to the method is increased by at least 3%, at least 5%, at least 7%, at least 10%, at least 15%, at least 20%, at least 25%, at least 30%, at least 40%, at least 50%, at least 75%, at least 100%, or at least 120%.

In another example, in some aspects, the ORR of a population of individuals treated according to the method is increased by at least 3%, at least 5%, at least 7%, at least 10%, at least 15%, at least 20%, at least 25%, at least 30%, at least 40%, at least 50%, at least 75%, at least 100%, or at least 120%.

The time at which the clinical outcome/clinical endpoint is assessed can readily be determined by the skilled person. In principle, it is determined at a timepoint when the difference in the clinical outcome/clinical endpoint between the two treatments becomes evident. This time may, for example, be at least 1 month, at least 2 months, at least 3 months, at least 6 months, at least 12 months, at least 18 months, at least 24 months, at least 30 months, at least 36 months, at least 42 months, or at least 48 months after the beginning of the treatment.

### Samples

An expression level of one or more of C1QC, MSR1, MRC1, VSIG4, SPP1, and MARCO and/or a TAM signature score may be determined from any suitable sample. Exemplary sample types include, without limitation, a tissue sample, a tumor sample, a whole blood sample, a plasma sample, a serum sample, and combinations thereof. Samples may be fresh, archival, or frozen.

In some aspects, the sample is a tissue sample, e.g., a tumor tissue sample. In some aspects, the tumor tissue sample is a biopsy. In some aspects in which the cancer is a lung cancer (e.g., a NSCLC), the sample is a biopsy of the lung cancer.

In some aspects, the sample is obtained from the individual prior to treatment with the PD-1 axis binding antagonist and the anti-TIGIT antagonist antibody, e.g., is obtained immediately prior to the first administration of the PD-1 axis binding antagonist and the anti-TIGIT antagonist antibody, or is obtained at least one day, at least one week, or at least one month prior to the first administration of the PD-1 axis binding antagonist and the anti-TIGIT antagonist antibody.

### Tam Signature Scores

In some aspects, determining a TAM signature score in the sample from the individual comprises calculating the average of the expression levels of at least two of C1QC, MSR1, MRC1, VSIG4, SPP1, and MARCO in the sample from the individual. Thus, in some aspects, the TAM signature score is an average of the expression levels (e.g., an average of the normalized expression levels) of at least two of C1QC, MSR1, MRC1, VSIG4, SPP1, and MARCO in the sample from the individual.

In some aspects, determining a TAM signature score in the sample from the individual comprises calculating the average of the expression levels of each of C1QC, MSR1, MRC1, VSIG4, SPP1, and MARCO in the sample from the individual. Thus, in some aspects, the TAM signature score is an average of the expression levels (e.g., an average of the normalized expression levels) of each of C1QC, MSR1, MRC1, VSIG4, SPP1, and MARCO in the sample from the individual.

### Expression Levels

The expression levels of C1QC, MSR1, MRC1, VSIG4, SPP1, and/or MARCO detected in the methods provided herein may be, e.g., nucleic acid expression levels or protein expression levels.

In some aspects, the expression levels are nucleic acid expression levels, e.g., mRNA expression levels. Nucleic acid expression levels may be detected using any suitable method known in the art, e.g., may be determined by RNA-seq, reverse transcriptase quantitative PCR (RT-qPCR), quantitative PCR (qPCR), real-time PCR, quantitative real-time PCR (qRT-PCR), multiplex qPCR or RT-qPCR, microarray analysis, SAGE, MassARRAY technique, in situ hybridization (ISH), or a combination thereof. Other amplification-based methods include, for example, transcript-mediated amplification (TMA), strand displacement amplification (SDA), nucleic acid sequence based amplification (NASBA), and signal amplification methods such as bDNA.

In some instances, nucleic acid expression levels of the genes described herein may be measured by sequencing-based techniques, such as, for example, RNA-seq, serial analysis of gene expression (SAGE), high-throughput sequencing technologies (e.g., massively parallel sequencing), and Sequenom MassARRAY® technology. Nucleic acid expression levels also may be measured by, for example, NanoString nCounter, and high-coverage expression profiling (HiCEP). Additional protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Other methods for detecting nucleic acid levels of the genes described herein include protocols which examine or detect mRNAs, such as target mRNAs, in a tissue or cell sample by microarray technologies.

Other methods to detect nucleic acid expression levels of the genes described herein include electrophoresis, Northern and Southern blot analyses, in situ hybridization (e.g., single or multiplex nucleic acid in situ hybridization), RNAse protection assays, and microarrays (e.g., Illumina BEADARRAY™ technology; Beads Array for Detection of Gene Expression (BADGE)).

In some aspects, the expression level is a protein expression level, e.g., a protein expression level determined by mass spectrometry, Western blot, ELISA, immunoprecipitation, immunohistochemistry, immunofluorescence, radioimmunoassay, dot blotting, immunodetection methods, surface plasmon resonance, optical spectroscopy, mass spectrometry, or HPLC.

### Normalization of Expression Levels

In some aspects, the expression levels of C1QC, MSR1, MRC1, VSIG4, SPP1, and/or MARCO are normalized expression levels, e.g., the TAM signature score is an average of the normalized expression levels of the one or more genes in the sample from the individual.

In some aspects, the TAM signature score is an average of the normalized expression levels of C1QC, MSR1, MRC1, VSIG4, SPP1, and MARCO in the sample from the individual.

The detected expression level of a gene may be normalized using any one of the standard normalization methods known in the art. One of skill in the art will appreciate that the normalization method used may depend on the gene expression methodology used (e.g., one or more housekeeping genes may be used for normalization in the context of an RT-qPCR methodology, but a whole genome or substantially whole genome may be used as a normalization baseline in the context of an RNA-seq methodology). For example, the detected expression level of each gene assayed can be normalized for differences in the amount of the gene(s) assayed, variability in the quality of the samples used, and/or variability between assay runs.

In some instances, normalization may be accomplished by detecting expression of certain one or more normalizing gene(s), including reference gene(s) (e.g., a housekeeping gene (e.g., β-actin)). For example, in some instances, the nucleic acid expression levels detected using the methods described herein may be normalized to the expression level of one or more reference genes (e.g., one, two, three, four, five, six, seven, eight, nine, or more reference genes, e.g., a housekeeping gene (e.g., β-actin)). Alternatively, normalization can be based on the average signal or median signal of all of the assayed genes. On a gene-by-gene basis, a measured normalized amount of an mRNA can be compared to the amount found in a reference expression level. The presence and/or expression level/amount measured in a particular subject sample to be analyzed will fall at some percentile within this range, which can be determined by methods well known in the art.

In other instances, to determine an expression level, the detected expression level of each assayed gene is not normalized.

Any statistical approaches known in the art may be used to determine the expression level of each gene. For example, the expression level may reflect the median expression level, median normalized expression level, or mean expression level, or mean normalized expression level.

In some aspects, the TAM signature score is a numerical value that reflects the aggregated Z-score expression level for the combination of genes assayed (e.g., the combination of two or more of C1QC, MSR1, MRC1, VSIG4, SPP1, and MARCO, e.g., the combination of all six of C1QC, MSR1, MRC1, VSIG4, SPP1, and MARCO).

### Detection of Additional Genes

Any of the methods provided above may further comprise detecting additional genes in the sample from the individual, e.g., may comprise detecting an expression level of at least one of C1QC, MSR1, MRC1, VSIG4, SPP1, and MARCO and one or more additional genes. In some aspects, the method comprises further detecting the expression level of one or more of tartrate-resistant acid phosphatase type 5 (ACP5), mast cell-expressed membrane protein 1 (MCEMP1), sterol 27-hydroxylase (CYP27A1), oxidized low-density lipoprotein receptor 1 (OLR1), progranulin (GRN), glioma pathogenesis-related protein 2 (GLIPR2), arrestin domain-containing protein 4 (ARRDC4), apolipoprotein E (APOE), folate receptor beta (FOLR2), and cathepsin D (CTSD) in the sample from the individual. In some instances, the method comprises further detecting the expression level of one, two, three, four, five, six, seven, eight, nine, or all ten of ACP5, MCEMP1, CYP27A1, OLR1, GRN, GLIPR2, ARRDC4, APOE, FOLR2, and CTSD.

In some aspects, determining a TAM signature score in the sample from the individual comprises further detecting the expression level of one, two, three, four, five, six, seven, eight, nine, or all ten of ACP5, MCEMP1, CYP27A1, OLR1, GRN, GLIPR2, ARRDC4, APOE, FOLR2, and CTSD.

For example, in some aspects, the TAM signature score is an average (e.g., a normalized average) of the expression levels of C1QC, MSR1, MRC1, VSIG4, SPP1, MARCO, and one or more of ACP5, MCEMP1, CYP27A1, OLR1, GRN, GLIPR2, ARRDC4, APOE, FOLR2, and CTSD in the sample from the individual. Measurement and normalization of expression levels may be performed as described above. For example, in some aspects, the TAM signature score is a numerical value that reflects the aggregated Z-score expression level for the combination of one or more of C1QC, MSR1, MRC1, VSIG4, SPP1, and MARCO and one or more of ACP5, MCEMP1, CYP27A1, OLR1, GRN, GLIPR2, ARRDC4, APOE, FOLR2, and CTSD.

In some aspects, the method comprises further detecting the expression level of each of ACP5, MCEMP1, CYP27A1, OLR1, GRN, GLIPR2, ARRDC4, APOE, FOLR2, and CTSD in the sample from the individual and determining therefrom the TAM signature score, wherein the TAM signature score is an average of the expression levels of C1QC, MSR1, MRC1, VSIG4, SPP1, MARCO, ACP5, MCEMP1, CYP27A1, OLR1, GRN, GLIPR2, ARRDC4, APOE, FOLR2, and CTSD in the sample from the individual.

In some aspects in which the individual has been determined to have a TAM signature score that is above a reference TAM signature score, thereby identifying the individual as one who may benefit from a treatment comprising a PD-1 axis binding antagonist and an anti-TIGIT antagonist antibody, the expression level of one or more of ACP5, MCEMP1, CYP27A1, OLR1, GRN, GLIPR2, ARRDC4, APOE, FOLR2, and CTSD has been detected in the sample from the individual.

In some aspects in which the individual has been determined to have a TAM signature score that is above a reference TAM signature score, thereby identifying the individual as one who may benefit from a treatment comprising a PD-1 axis binding antagonist and an anti-TIGIT antagonist antibody, the expression level of each of ACP5, MCEMP1, CYP27A1, OLR1, GRN, GLIPR2, ARRDC4, APOE, FOLR2, and CTSD has been detected in the sample from the individual and the TAM signature score has been determined therefrom, wherein the TAM signature score is an average of the expression levels of C1QC, MSR1, MRC1, VSIG4, SPP1, MARCO, ACP5, MCEMP1, CYP27A1, OLR1, GRN, GLIPR2, ARRDC4, APOE, FOLR2, and CTSD in the sample from the individual.

### Reference Expression Levels and TAM Signature Scores

The terms “reference expression level” and “reference TAM signature score” refer to an expression level or a TAM signature score against which another expression level or TAM signature score is compared, e.g., to make a diagnostic, predictive, prognostic, and/or therapeutic determination.

In some aspects, the reference expression level or reference TAM signature score is a pre-assigned reference expression level or reference TAM signature score.

In some aspects, the reference expression level or reference TAM signature score is an expression level or a TAM signature score in a reference population (e.g., a population of individuals having the cancer, e.g., a population of individuals having a lung cancer (e.g., a NSCLC)).

In some aspects, the expression level or TAM signature score in the reference population is a median expression level or TAM signature score of the reference population.

In other aspects, the expression level or TAM signature score in the reference population is a mean expression level or TAM signature score of the reference population.

In still other aspects, the expression level or TAM signature score is defined as the 25^{th} percentile, the 26^{th} percentile, the 27^{th} percentile, the 28^{th} percentile, the 29th percentile, the 30^{th} percentile, the 31^{st} percentile, the 32^{nd} percentile, the 33^{rd} percentile, the 34^{th} percentile, the 35^{th} percentile, the 36^{th} percentile, the 37^{th} percentile, the 38^{th} percentile, the 39^{th} percentile, the 40^{th} percentile, the 41^{st} percentile, the 42^{nd} percentile, the 43^{rd} percentile, the 44^{th} percentile, the 45^{th} percentile, the 46^{th} percentile, the 47^{th} percentile, the 48^{th} percentile, the 49^{th} percentile, the 50^{th} percentile, the 51^{st} percentile, the 52^{nd} percentile, the 53^{rd} percentile, the 54^{th} percentile, the 55^{th} percentile, the 56^{th} percentile, the 57^{th} percentile, the 58^{th} percentile, the 59^{th} percentile, the 60^{th} percentile, the 61^{st} percentile, the 62^{nd} percentile, the 63^{rd} percentile, the 64^{th} percentile, the 65^{th} percentile, the 66^{th} percentile, the 67^{th} percentile, the 68^{th} percentile, the 69^{th} percentile, the 70^{th} percentile, the 71^{st} percentile, the 72^{nd} percentile, the 73^{rd} percentile, the 74^{th} percentile, the 75^{th} percentile, the 76^{th} percentile, the 77^{th} percentile, the 78^{th} percentile, the 79^{th} percentile, the 80^{th} percentile, the 81^{st} percentile, the 82^{nd} percentile, the 83^{rd} percentile, the 84^{th} percentile, the 85^{th} percentile, the 86^{th} percentile, the 87^{th} percentile, the 88^{th} percentile, the 89^{th} percentile, the 90^{th} percentile, the 91^{st} percentile, the 92^{nd} percentile, the 93^{rd} percentile, the 94^{th} percentile, the 95^{th} percentile, the 96^{th} percentile, the 97^{th} percentile, the 98^{th} percentile, or the 99^{th} percentile of expression levels or TAM signature scores in the reference population.

In some instances, the reference expression level or reference TAM signature score is a cut-off value that significantly separates a first and a second subset of individuals who have been treated with a PD-1 axis binding antagonist and an anti-TIGIT antagonist antibody (e.g., atezolizumab and tiragolumab) in the same reference population based on a significant difference between an individual's responsiveness to treatment with the PD-1 axis binding antagonist and the anti-TIGIT antagonist antibody above the cut-off value or at or below the cut-off value. In some aspects, the individual's responsiveness to treatment with the PD-1 axis binding antagonist and the anti-TIGIT antagonist antibody is significantly improved relative to the individual's responsiveness to treatment with the PD-1 axis binding antagonist and the anti-TIGIT antagonist antibody at or above the cut-off value.

### PD-L1 Status

In some aspects, the expression level of PD-L1 has been assessed in the sample from a subject described herein. In some aspects, the sample has been determined to have a PD-L1-positive tumor cell fraction (e.g., by an immunohistochemical (IHC) assay, e.g., by positive staining with an anti-PD-L1 antibody, wherein the anti-PD-L1 antibody is SP263, 22C3, SP142, or 28-8).

In some aspects, the PD-L1-positive tumor cell fraction is greater than, or equal to, 50%, as determined by positive staining with the anti-PD-L1 antibody SP263 (e.g., as calculated using the Ventana SP263 IHC assay).

In some aspects, the PD-L1-positive tumor cell fraction is greater than, or equal to, 50%, as determined by positive staining with the anti-PD-L1 antibody 22C3 (e.g., as calculated using the pharmDx 22C3 IHC assay).

Exemplary methods for assessing the expression level of PD-L1 are provided in Section III(E).

### B. Myeloid Markers in On-Treatment Serum Samples

### Methods of Monitoring Response to Treatment

In another aspect, the invention provides a method for monitoring the response of an individual having a cancer (e.g., a lung cancer, e.g., a NSCLC) to a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), the method comprising detecting an expression level (e.g., a gene expression level, e.g., a protein expression level or a nucleic acid expression level) of one or more of myeloid markers macrophage receptor with collagenous structure (MARCO), cathelicidin antimicrobial peptide (CAMP), CD5 antigen-like (CD5L), scavenger receptor cysteine-rich type 1 protein M130 (CD163), neutrophil gelatinase-associated lipocalin (NGAL), macrophage colony-stimulating factor 1 receptor (CSF1R), CD44 antigen (CD44), apolipoprotein C-II (APOC2), apolipoprotein C-III (APOC3), apolipoprotein C-IV (APOC4), apolipoprotein A-II (APOA2), apolipoprotein E (APOE), lactotransferrin (TRFL), vascular cell adhesion protein 1 (VCAM1), PERM, beta-2-microglobulin (B2MG), LYSC, LYAM1, LCAT, and LIRA3 in a sample from the individual at a time point during or after administration of the PD-1 axis binding antagonist and the anti-TIGIT antagonist antibody, wherein an increase in the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 (e.g., 1, 2, 3, 4, 5, 6, 7, 8, 9, 10, 11, 12, 13, 14, 15, 16, 17, 18, 19, or all 20 of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3) relative to a respective reference expression level is predictive of an individual who is likely to respond to the treatment comprising the PD-1 axis binding antagonist and the anti-TIGIT antagonist antibody.

In some aspects, the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is increased relative to a respective reference expression level, thereby predicting that the individual is likely to respond to the treatment comprising the PD-1 axis binding antagonist and the anti-TIGIT antagonist antibody, and the method further comprises administering an additional dose of the PD-1 axis binding antagonist and the anti-TIGIT antagonist antibody to the individual.

In some aspects, an increase in the expression level (e.g., gene expression level, e.g., protein expression level or nucleic acid expression level) of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is defined as an increase of at least 1.2-fold (e.g., an increase of 1.3-fold, 1.4-fold, 1.5-fold, 1.6-fold, 1.7-fold, 1.8-fold, 1.9-fold, 2-fold, or more than 2-fold) relative to a reference level. For example, in some aspects, an increased expression level is an expression level that is increased by between 1.2-fold and 5-fold (e.g., increased by between 1.2-fold and 4-fold, between 1.2-fold and 3-fold, between 1.2-fold and 2-fold, between 1.2-fold and 1.9-fold, between 1.2-fold and 1.7-fold, or between 1.2-fold and 1.5-fold).

In some aspects, the response to treatment is an increase in progression-free survival (PFS) or overall survival (OS).

In some aspects of any of the methods provided herein, the cancer is a lung cancer, e.g., a NSCLC. In some aspects, the individual is a human.

### Expression Levels

The expression levels of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 detected in the methods provided herein may be gene expression levels, e.g., nucleic acid expression levels or protein expression levels (e.g., protein levels determined by mass spectrometry). Exemplary methods for detecting and normalizing nucleic acid expression levels are provided in Section IIIA, above.

In some aspects, the expression level is a protein expression level, e.g., a protein expression level determined by mass spectrometry, Western blot, ELISA, immunoprecipitation, immunohistochemistry, immunofluorescence, radioimmunoassay, dot blotting, immunodetection methods, surface plasmon resonance, optical spectroscopy, mass spectrometry, or HPLC. In some aspects, the protein expression level is measured in a serum sample.

In other aspects, the expression levels are nucleic acid expression levels, e.g., mRNA expression levels. In one aspect, the expression levels of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 are detected in cells from a blood sample from the individual, e.g., are detected in peripheral blood mononuclear cells (PBMCs) derived from a blood sample from the individual.

### Samples

An expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 may be determined from any suitable sample. Exemplary sample types include, without limitation, a tissue sample, a tumor sample, a whole blood sample, a plasma sample, a serum sample, and combinations thereof. Samples may be fresh, archival, or frozen.

In some aspects, the sample is a serum sample.

The sample (e.g., serum sample) may be collected from the individual, and the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 may be detected therein, at any suitable time point following the first administration of the treatment comprising the PD-1 axis binding antagonist and the anti-TIGIT antagonist antibody to the individual. For example, the sample may be collected at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days (e.g., 1-10 days, 5-15 days, 10-20 days, 15-25 days, 20-30 days, 25-35 days, 30-40 days, 35-45 days, 40-50 days, 45-55 days, or 50-60 days) after the initiation of the treatment comprising the PD-1 axis binding antagonist and the anti-TIGIT antagonist antibody. In other examples, the sample is collected about one week, about two weeks, about three weeks, about four weeks, about five weeks, about six weeks, about seven weeks, about eight weeks, or more than eight weeks after the initiation of the treatment comprising the PD-1 axis binding antagonist and the anti-TIGIT antagonist antibody (e.g., is collected one, two, three, four, five, six, seven, eight, or more than eight weeks after the initiation of the treatment comprising the PD-1 axis binding antagonist and the anti-TIGIT antagonist antibody).

In some aspects, the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is detected three weeks after the initiation of the treatment comprising the PD-1 axis binding antagonist and the anti-TIGIT antagonist antibody (e.g., the sample (e.g., serum sample) is collected from the individual three weeks after the initiation of the treatment comprising the PD-1 axis binding antagonist and the anti-TIGIT antagonist antibody and the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is detected therein).

In some aspects, the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is detected six weeks after the initiation of the treatment comprising the PD-1 axis binding antagonist and the anti-TIGIT antagonist antibody (e.g., the sample (e.g., serum sample) is collected from the individual six weeks after the initiation of the treatment comprising the PD-1 axis binding antagonist and the anti-TIGIT antagonist antibody and the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 is detected therein).

### Reference Expression Levels

The term “reference expression level” refers to an expression level (e.g., a protein expression level) against which another expression level is compared, e.g., to make a diagnostic, predictive, prognostic, and/or therapeutic determination.

In some aspects, the reference expression level is a baseline expression level from a sample from the individual at a time point prior to the initiation of the treatment comprising the PD-1 axis binding antagonist and the anti-TIGIT antagonist antibody (e.g., a sample collected from the individual obtained immediately prior to the first administration of the PD-1 axis binding antagonist and the anti-TIGIT antagonist antibody or obtained from the individual at least one day, at least one week, or at least one month prior to the first administration of the PD-1 axis binding antagonist and the anti-TIGIT antagonist antibody.

In some aspects, the reference expression level is a pre-assigned reference expression level.

In some aspects, the reference expression level is an expression level in a reference population (e.g., a population of individuals having the cancer, e.g., a population of individuals having a lung cancer (e.g., a NSCLC)).

In some aspects, the expression level in the reference population is a median expression level of the reference population.

In other aspects, the expression level in the reference population is a mean expression level of the reference population.

In still other aspects, the expression level is defined as the 25^{th} percentile, the 26^{th} percentile, the 27^{th} percentile, the 28^{th} percentile, the 29^{th} percentile, the 30^{th} percentile, the 31^{st} percentile, the 32^{nd} percentile, the 33^{rd} percentile, the 34^{th} percentile, the 35^{th} percentile, the 36^{th} percentile, the 37^{th} percentile, the 38^{th} percentile, the 39^{th} percentile, the 40^{th} percentile, the 41^{st} percentile, the 42^{nd} percentile, the 43^{rd} percentile, the 44^{th} percentile, the 45^{th} percentile, the 46^{th} percentile, the 47^{th} percentile, the 48^{th} percentile, the 49^{th} percentile, the 50^{th} percentile, the 51^{st} percentile, the 52^{nd} percentile, the 53^{rd} percentile, the 54^{th} percentile, the 55^{th} percentile, the 56^{th} percentile, the 57^{th} percentile, the 58^{th} percentile, the 59^{th} percentile, the 60^{th} percentile, the 61^{st} percentile, the 62^{nd} percentile, the 63^{rd} percentile, the 64^{th} percentile, the 65^{th} percentile, the 66^{th} percentile, the 67^{th} percentile, the 68^{th} percentile, the 69^{th} percentile, the 70^{th} percentile, the 71^{st} percentile, the 72^{nd} percentile, the 73^{rd} percentile, the 74^{th} percentile, the 75^{th} percentile, the 76^{th} percentile, the 77^{th} percentile, the 78^{th} percentile, the 79^{th} percentile, the 80^{th} percentile, the 81^{st} percentile, the 82^{nd} percentile, the 83^{rd} percentile, the 84^{th} percentile, the 85^{th} percentile, the 86^{th} percentile, the 87^{th} percentile, the 88^{th} percentile, the 89^{th} percentile, the 90^{th} percentile, the 91^{st} percentile, the 92^{nd} percentile, the 93^{rd} percentile, the 94^{th} percentile, the 95^{th} percentile, the 96^{th} percentile, the 97^{th} percentile, the 98^{th} percentile, or the 99^{th} percentile of expression levels in the reference population.

In some instances, the reference expression level is a cut-off value that significantly separates a first and a second subset of individuals who have been treated with a PD-1 axis binding antagonist and an anti-TIGIT antagonist antibody (e.g., atezolizumab and tiragolumab) in the same reference population based on a significant difference between an individual's responsiveness to treatment with the PD-1 axis binding antagonist and the anti-TIGIT antagonist antibody above the cut-off value or at or below the cut-off value. In some aspects, the individual's responsiveness to treatment with the PD-1 axis binding antagonist and the anti-TIGIT antagonist antibody is significantly improved relative to the individual's responsiveness to treatment with the PD-1 axis binding antagonist and the anti-TIGIT antagonist antibody at or above the cut-off value.

### PD-L1 Status

In some aspects, the expression level of PD-L1 has been assessed in the sample from a subject described herein. In some aspects, the sample has been determined to have a PD-L1-positive tumor cell fraction (e.g., by an immunohistochemical (IHC) assay, e.g., by positive staining with an anti-PD-L1 antibody, wherein the anti-PD-L1 antibody is SP263, 22C3, SP142, or 28-8).

In some aspects, the PD-L1-positive tumor cell fraction is greater than, or equal to, 50%, as determined by positive staining with the anti-PD-L1 antibody SP263 (e.g., as calculated using the Ventana SP263 IHC assay).

In some aspects, the PD-L1-positive tumor cell fraction is greater than, or equal to, 50%, as determined by positive staining with the anti-PD-L1 antibody 22C3 (e.g., as calculated using the pharmDx 22C3 IHC assay).

Exemplary methods for assessing the expression level of PD-L1 are provided in Section III(E).

### C. Regulatory T Cell (Treg) Genes and Signatures

Methods of Identifying Individuals Who May Benefit from Treatment

### (i) Treg Genes

In one aspect, the invention provides a method of identifying an individual having a cancer (e.g., a lung cancer, e.g., a non-small cell lung cancer (NSCLC)) who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), the method comprising detecting an expression level of one or more of Treg-associated genes forkhead box protein P3 (FOXP3), cytotoxic T-lymphocyte protein 4 (CTLA4), interleukin 10 (IL10), tumor necrosis factor receptor superfamily member 18 (TNFRSF18), C-C chemokine receptor type 8 (CCR8), zinc finger protein Eos (IKZF4), and zinc finger protein Helios (IKZF2) (e.g., one, two, three, four, five, six, or all seven of FOXP3, CTLA4, IL10, TNFRSF18, CCR8, IKZF4, and IKZF2) in a sample from the individual, wherein an expression level of one or more of FOXP3, CTLA4, IL10, TNFRSF18, CCR8, IKZF4, and IKZF2 that is above a respective reference expression level identifies the individual as one who may benefit from a treatment comprising a PD-1 axis binding antagonist and an anti-TIGIT antagonist antibody.

### (ii) Treg Signature Scores

In another aspect, the invention provides a method of identifying an individual having a cancer (e.g., a lung cancer, e.g., a NSCLC) who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), the method comprising detecting an expression level of at least two of FOXP3, CTLA4, IL10, TNFRSF18, CCR8, IKZF4, and IKZF2 (e.g., two, three, four, five, six, or all seven of FOXP3, CTLA4, IL10, TNFRSF18, CCR8, IKZF4, and IKZF2) in a sample from the individual and determining a regulatory T cell (Treg) signature score therefrom, wherein a Treg signature score that is above a reference Treg signature score identifies the individual as one who may benefit from a treatment comprising a PD-1 axis binding antagonist and an anti-TIGIT antagonist antibody.

In another aspect, the invention provides a method of identifying an individual having a cancer (e.g., a lung cancer, e.g., a NSCLC) who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), the method comprising detecting an expression level of each of FOXP3, CTLA4, IL110, TNFRSF18, CCR8, IKZF4, and IKZF2 in a sample from the individual and determining a Treg signature score therefrom, wherein a Treg signature score that is above a reference Treg signature score identifies the individual as one who may benefit from a treatment comprising a PD-1 axis binding antagonist and an anti-TIGIT antagonist antibody.

In some aspects, the individual has a Treg signature score in the sample that is above a reference Treg signature score, and the method further comprises administering to the individual an effective amount of a PD-1 axis binding antagonist and an anti-TIGIT antagonist antibody. Exemplary methods for determining a Treg signature score and exemplary reference Treg signature scores are provided below.

Exemplary PD-1 axis binding antagonists, anti-TIGIT antagonist antibodies, and methods of treatment comprising these agents are provided in Section IV.

In some aspects of any of the methods provided herein, the cancer is a lung cancer, e.g., a NSCLC. In some aspects, the individual is a human.

### Methods of Selecting a Therapy

### (i) Treg Genes

In another aspect, the invention provides a method for selecting a therapy for an individual having a cancer (e.g., a lung cancer, e.g., a NSCLC), the method comprising detecting an expression level of one or more of Treg-associated genes FOXP3, CTLA4, IL110, TNFRSF18, CCR8, IKZF4, and IKZF2 (e.g., one, two, three, four, five, six, or all seven of FOXP3, CTLA4, IL110, TNFRSF18, CCR8, IKZF4, and IKZF2) in a sample from the individual, wherein an expression level of one or more of FOXP3, CTLA4, IL10, TNFRSF18, CCR8, IKZF4, and IKZF2 that is above a respective reference expression level identifies the individual as one who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)).

### (ii) Treg Signature Scores

In another aspect, the invention provides a method for selecting a therapy for an individual having a cancer (e.g., a lung cancer, e.g., a NSCLC), the method comprising detecting an expression level of at least two of FOXP3, CTLA4, IL10, TNFRSF18, CCR8, IKZF4, and IKZF2 (e.g., one, two, three, four, five, six, or all seven of FOXP3, CTLA4, IL10, TNFRSF18, CCR8, IKZF4, and IKZF2) in a sample from the individual and determining a Treg signature score therefrom, wherein a Treg signature score that is above a reference Treg signature score identifies the individual as one who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)).

In another aspect, the invention provides a method for selecting a therapy for an individual having a cancer (e.g., a lung cancer, e.g., a NSCLC), the method comprising detecting an expression level of each of FOXP3, CTLA4, IL10, TNFRSF18, CCR8, IKZF4, and IKZF2 in a sample from the individual and determining a Treg signature score therefrom, wherein a Treg signature score that is above a reference Treg signature score identifies the individual as one who may benefit from a treatment comprising a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)).

In some aspects, the individual has a Treg signature score in the sample that is above a reference Treg signature score, and the method further comprises administering to the individual an effective amount of a PD-1 axis binding antagonist and an anti-TIGIT antagonist antibody.

### Methods of Treatment

### (i) Treg Genes

In another aspect, the invention provides a method of treating an individual having a cancer (e.g., a lung cancer, e.g., a NSCLC), the method comprising (a) detecting an expression level of one or more of Treg-associated genes FOXP3, CTLA4, IL110, TNFRSF18, CCR8, IKZF4, and IKZF2 (e.g., one, two, three, four, five, six, or all seven of FOXP3, CTLA4, IL110, TNFRSF18, CCR8, IKZF4, and IKZF2) in a sample from the individual, wherein the expression level of one or more of FOXP3, CTLA4, IL110, TNFRSF18, CCR8, IKZF4, and IKZF2 is above a respective reference expression level and thereby identifies the individual as one who may benefit from a treatment comprising a PD-1 axis binding antagonist and an anti-TIGIT antagonist antibody; and (b) administering an effective amount of a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual.

In another aspect, the invention provides a method of treating an individual having a cancer (e.g., a lung cancer, e.g., a NSCLC), the method comprising administering a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual, wherein the individual has been determined to have an expression level of one or more of FOXP3, CTLA4, IL10, TNFRSF18, CCR8, IKZF4, and IKZF2 that is above a respective reference expression level, thereby identifying the individual as one who may benefit from a treatment comprising a PD-1 axis binding antagonist and an anti-TIGIT antagonist antibody.

### (ii) Treg Signature Scores

In another aspect, the invention provides a method of treating an individual having a cancer (e.g., a lung cancer, e.g., a NSCLC), the method comprising (a) detecting an expression level of at least two of FOXP3, CTLA4, I10, TNFRSF18, CCR8, IKZF4, and IKZF2 (e.g., two, three, four, five, six, or all seven of FOXP3, CTLA4, IL10, TNFRSF18, CCR8, IKZF4, and IKZF2) in a sample from the individual and determining a Treg signature score therefrom, wherein the Treg signature score is above a reference Treg signature score and thereby identifies the individual as one who may benefit from a treatment comprising a PD-1 axis binding antagonist and an anti-TIGIT antagonist antibody; and (b) administering an effective amount of a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual.

In another aspect, the invention provides a method of treating an individual having a cancer (e.g., a lung cancer, e.g., a NSCLC), the method comprising (a) detecting an expression level of each of FOXP3, CTLA4, IL10, TNFRSF18, CCR8, IKZF4, and IKZF2 in a sample from the individual and determining a Treg signature score therefrom, wherein the Treg signature score is above a reference Treg signature score and thereby identifies the individual as one who may benefit from a treatment comprising a PD-1 axis binding antagonist and an anti-TIGIT antagonist antibody; and (b) administering an effective amount of a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual.

In another aspect, the invention provides a method of treating an individual having a cancer (e.g., a lung cancer, e.g., a NSCLC), the method comprising administering a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual, wherein the individual has been determined to have a Treg signature score that is above a reference Treg signature score, thereby identifying the individual as one who may benefit from a treatment comprising a PD-1 axis binding antagonist and an anti-TIGIT antagonist antibody, and wherein the Treg signature score is based on the expression level of at least two of FOXP3, CTLA4, IL10, TNFRSF18, CCR8, IKZF4, and IKZF2 (e.g., two, three, four, five, six, or all seven of FOXP3, CTLA4, IL10, TNFRSF18, CCR8, IKZF4, and IKZF2) detected in a sample from the individual.

In another aspect, the invention provides a method of treating an individual having a cancer (e.g., a lung cancer, e.g., a NSCLC), the method comprising administering a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) to the individual, wherein the individual has been determined to have a Treg signature score that is above a reference Treg signature score, thereby identifying the individual as one who may benefit from a treatment comprising a PD-1 axis binding antagonist and an anti-TIGIT antagonist antibody, and wherein the Treg signature score is based on the expression level of each of FOXP3, CTLA4, IL10, TNFRSF18, CCR8, IKZF4, and IKZF2 detected in a sample from the individual.

### Benefit

In some aspects, the benefit achieved by the treatment comprising a PD-1 axis binding antagonist and an anti-TIGIT antagonist antibody is an increase in progression-free survival (PFS) (e.g., an increase in the duration of PFS experienced by an individual treated according to the method or an increase in the average PFS of a population of individuals treated according to the method), an increase in objective response rate (ORR) (e.g., an increase in the ORR of a population of individuals treated according to the method), and/or an increase in overall survival (OS) (e.g., an increase in the duration of OS experienced by an individual treated according to the method or an increase in the average OS of a population of individuals treated according to the method).

An increased PFS, ORR, and/or OS may be determined by comparison to, e.g., an untreated reference individual and/or a reference population of individuals; a reference individual and/or a reference population of individuals who have received a control treatment, such as one or more previously approved treatments or marketed products for treatment of the cancer; and/or a reference individual and/or a reference population of individuals who have been treated with a PD-1 axis binding antagonist (e.g., atezolizumab) or an anti-TIGIT antagonist antibody (e.g., tiragolumab) as a monotherapy. In some aspects, the increased PFS, ORR, and/or OS is determined relative to a reference individual and/or a reference population of individuals having cancer that have been treated with a treatment comprising a PD-1 axis binding antagonist and an anti-TIGIT antagonist antibody (e.g., atezolizumab and tiragolumab) wherein the reference individual and/or each individual in the reference population has a Treg signature score that is at or below a reference Treg signature score and/or has an expression level of one or more of FOXP3, CTLA4, IL110, TNFRSF18, CCR8, IKZF4, and IKZF2 that is at or below a respective reference expression level. Reference Treg signature scores are described herein and may, for example, be a median Treg signature score of a reference population of individuals having cancer (e.g., a lung cancer, e.g., a NSCLC) or a median expression level of one or more of FOXP3, CTLA4, IL110, TNFRSF18, CCR8, IKZF4, and IKZF2 in a reference population of individuals having cancer (e.g., a lung cancer, e.g., a NSCLC).

Exemplary methods for determining whether a given clinical outcome is improved in accordance with the invention are provided in Section III(A).

### Samples

An expression level of one or more of FOXP3, CTLA4, IL10, TNFRSF18, CCR8, IKZF4, and IKZF2 and/or a Treg signature score may be determined from any suitable sample. Exemplary sample types include, without limitation, a tissue sample, a tumor sample, a whole blood sample, a plasma sample, a serum sample, and combinations thereof. Samples may be fresh, archival, or frozen.

In some aspects, the sample is a tissue sample, e.g., a tumor tissue sample. In some aspects, the tumor tissue sample is a biopsy. In some aspects in which the cancer is a lung cancer (e.g., a NSCLC), the sample is a biopsy of the lung cancer.

In some aspects, the sample is obtained from the individual prior to treatment with the PD-1 axis binding antagonist and the anti-TIGIT antagonist antibody, e.g., is obtained immediately prior to the first administration of the PD-1 axis binding antagonist and the anti-TIGIT antagonist antibody or is obtained at least one day, at least one week, or at least one month prior to the first administration of the PD-1 axis binding antagonist and the anti-TIGIT antagonist antibody.

### Treg Signature Scores

In some aspects, determining a Treg signature score in the sample from the individual comprises calculating the average of the expression levels of at least two of FOXP3, CTLA4, IL110, TNFRSF18, CCR8, IKZF4, and IKZF2 in the sample from the individual. Thus, in some aspects, the Treg signature score is an average of the expression levels (e.g., an average of the normalized expression levels) of at least two of FOXP3, CTLA4, IL10, TNFRSF18, CCR8, IKZF4, and IKZF2 in the sample from the individual.

In some aspects, determining a Treg signature score in the sample from the individual comprises calculating the average of the expression levels of each of FOXP3, CTLA4, IL10, TNFRSF18, CCR8, IKZF4, and IKZF2 in the sample from the individual. Thus, in some aspects, the TAM signature score is an average of the expression levels (e.g., an average of the normalized expression levels) of each of FOXP3, CTLA4, IL10, TNFRSF18, CCR8, IKZF4, and IKZF2 in the sample from the individual.

### Expression Levels

The expression levels of FOXP3, CTLA4, IL10, TNFRSF18, CCR8, IKZF4, and/or IKZF2 detected in the methods provided herein may be, e.g., nucleic acid expression levels or protein expression levels (e.g., protein levels determined by mass spectrometry).

In some aspects, the expression levels are nucleic acid expression levels, e.g., mRNA expression levels. Nucleic acid expression levels may be detected using any suitable method known in the art, e.g., may be determined by RNA-seq, RT-qPCR, qPCR, real-time PCR, quantitative real-time PCR (qRT-PCR), multiplex qPCR or RT-qPCR, microarray analysis, SAGE, MassARRAY technique, in situ hybridization (ISH), or a combination thereof. Further exemplary methods for measuring nucleic acid expression levels are provided in Section III(A).

In some aspects, the expression level is a protein expression level, e.g., a protein expression level determined by mass spectrometry, Western blot, ELISA, immunoprecipitation, immunohistochemistry, immunofluorescence, radioimmunoassay, dot blotting, immunodetection methods, surface plasmon resonance, optical spectroscopy, mass spectrometry, or HPLC.

### Normalization of Expression Levels

In some aspects, the expression levels of FOXP3, CTLA4, IL10, TNFRSF18, CCR8, IKZF4, and/or IKZF2 are normalized expression levels, e.g., the Treg signature score is an average of the normalized expression levels of the one or more genes in the sample from the individual.

In some aspects, the Treg signature score is an average of the normalized expression levels of FOXP3, CTLA4, IL10, TNFRSF18, CCR8, IKZF4, and IKZF2 in the sample from the individual.

Exemplary methods for normalizing the detected expression level of a gene are provided in Section III(A).

In some aspects, the Treg signature score is a numerical value that reflects the aggregated Z-score expression level for the combination of genes assayed (e.g., the combination of two or more of FOXP3, CTLA4, IL10, TNFRSF18, CCR8, IKZF4, and IKZF2, e.g., the combination of all seven of FOXP3, CTLA4, IL10, TNFRSF18, CCR8, IKZF4, and IKZF2).

### Reference Expression Levels and Treg Signature Scores

The terms “reference expression level” and “reference Treg signature score” refer to an expression level or a Treg signature score against which another expression level or Treg signature score is compared, e.g., to make a diagnostic, predictive, prognostic, and/or therapeutic determination.

In some aspects, the reference expression level or reference Treg signature score is a pre-assigned reference expression level or reference Treg signature score.

In some aspects, the reference expression level or reference Treg signature score is an expression level or a Treg signature score in a reference population (e.g., a population of individuals having the cancer, e.g., a population of individuals having a lung cancer (e.g., a NSCLC)).

In some aspects, the expression level or Treg signature score in the reference population is a median expression level or Treg signature score of the reference population.

In other aspects, the expression level or Treg signature score in the reference population is a mean expression level or Treg signature score of the reference population.

In still other aspects, the expression level or Treg signature score is defined as the 25^{th} percentile, the 26^{th} percentile, the 27^{th} percentile, the 28^{th} percentile, the 29^{th} percentile, the 30^{th} percentile, the 31^{st} percentile, the 32^{nd} percentile, the 33^{rd} percentile, the 34^{th} percentile, the 35^{th} percentile, the 36^{th} percentile, the 37^{th} percentile, the 38^{th} percentile, the 39^{th} percentile, the 40^{th} percentile, the 41^{st} percentile, the 42^{nd} percentile, the 43^{rd} percentile, the 44^{th} percentile, the 45^{th} percentile, the 46^{th} percentile, the 47^{th} percentile, the 48^{th} percentile, the 49^{th} percentile, the 50^{th} percentile, the 51^{st} percentile, the 52^{nd} percentile, the 53^{rd} percentile, the 54^{th} percentile, the 55^{th} percentile, the 56^{th} percentile, the 57^{th} percentile, the 58^{th} percentile, the 59^{th} percentile, the 60^{th} percentile, the 61^{st} percentile, the 62^{nd} percentile, the 63^{rd} percentile, the 64^{th} percentile, the 65^{th} percentile, the 66^{th} percentile, the 67^{th} percentile, the 68^{th} percentile, the 69^{th} percentile, the 70^{th} percentile, the 71^{st} percentile, the 72^{nd} percentile, the 73^{rd} percentile, the 74^{th} percentile, the 75^{th} percentile, the 76^{th} percentile, the 77^{th} percentile, the 78^{th} percentile, the 79^{th} percentile, the 80^{th} percentile, the 81^{st} percentile, the 82^{nd} percentile, the 83^{rd} percentile, the 84^{th} percentile, the 85^{th} percentile, the 86^{th} percentile, the 87^{th} percentile, the 88^{th} percentile, the 89^{th} percentile, the 90^{th} percentile, the 91^{st} percentile, the 92^{nd} percentile, the 93^{rd} percentile, the 94^{th} percentile, the 95^{th} percentile, the 96^{th} percentile, the 97^{th} percentile, the 98^{th} percentile, or the 99^{th} percentile of expression levels or Treg signature scores in the reference population.

In some instances, the reference expression level or reference Treg signature score is a cut-off value that significantly separates a first and a second subset of individuals who have been treated with a PD-1 axis binding antagonist and an anti-TIGIT antagonist antibody (e.g., atezolizumab and tiragolumab) in the same reference population based on a significant difference between an individual's responsiveness to treatment with the PD-1 axis binding antagonist and the anti-TIGIT antagonist antibody above the cut-off value or at or below the cut-off value. In some aspects, the individual's responsiveness to treatment with the PD-1 axis binding antagonist and the anti-TIGIT antagonist antibody is significantly improved relative to the individual's responsiveness to treatment with the PD-1 axis binding antagonist and the anti-TIGIT antagonist antibody at or above the cut-off value.

### PD-L1 Status

In some aspects, the expression level of PD-L1 has been assessed in the sample from a subject described herein. In some aspects, the sample has been determined to have a PD-L1-positive tumor cell fraction (e.g., by an immunohistochemical (IHC) assay, e.g., by positive staining with an anti-PD-L1 antibody, wherein the anti-PD-L1 antibody is SP263, 22C3, SP142, or 28-8).

In some aspects, the PD-L1-positive tumor cell fraction is greater than, or equal to, 50%, as determined by positive staining with the anti-PD-L1 antibody SP263 (e.g., as calculated using the Ventana SP263 IHC assay).

In some aspects, the PD-L1-positive tumor cell fraction is greater than, or equal to, 50%, as determined by positive staining with the anti-PD-L1 antibody 22C3 (e.g., as calculated using the pharmDx 22C3 IHC assay).

Exemplary methods for assessing the expression level of PD-L1 are provided in Section III(E).

### D. Assessment of TIGIT Expression

In some aspects, the expression of TIGIT is assessed in an individual described herein. The methods provided herein may include determining the expression level of TIGIT in a biological sample (e.g., a tumor sample) obtained from the individual. In other examples, the expression level of TIGIT in a biological sample (e.g., a tumor sample) obtained from the individual has been determined prior to initiation of treatment or after initiation of treatment. TIGIT expression may be determined using any suitable approach. Any suitable tumor sample may be used, e.g., a formalin-fixed and paraffin-embedded (FFPE) tumor sample, an archival tumor sample, a fresh tumor sample, or a frozen tumor sample.

For example, TIGIT expression may be determined in terms of the percentage of a tumor sample comprised by tumor-infiltrating immune cells expressing a detectable expression level of TIGIT, as the percentage of tumor-infiltrating immune cells in a tumor sample expressing a detectable expression level of TIGIT, and/or as the percentage of tumor cells in a tumor sample expressing a detectable expression level of TIGIT. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the individual, for example, as assessed by IHC using an anti-TIGIT antagonist antibody. Any suitable anti-TIGIT antagonist antibody may be used. In some examples, the anti-TIGIT antagonist antibody is 10A7 (WO 2009/126688A3; U.S. Pat. No. 9,499,596). In other examples, the anti-TIGIT antagonist antibody is the anti-human TIGIT rabbit monoclonal antibody clone SP410 (Roche Tissue Diagnostics, Pleasanton, CA). In some aspects, the TIGIT antagonist antibody (e.g., SP410) is detected using the VENTANA OptiView DAB IHC Detection Kit on the automated VENTANA BenchMark ULTRA platform.

### E. Assessment of PD-L1 Expression

In some aspects, the expression of PD-L1 is assessed in an individual described herein. The methods provided herein may include determining the expression level of PD-L1 in a biological sample (e.g., a tumor sample) obtained from the individual. In other examples, the expression level of PD-L1 in a biological sample (e.g., a tumor sample) obtained from the individual has been determined prior to initiation of treatment or after initiation of treatment. PD-L1 expression may be determined using any suitable approach. For example, PD-L1 expression may be determined as described in U.S. Patent Application Publication Nos. US20180030138A1 and US20180037655A1. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the individual, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263. In some examples, the anti-PD-L1 antibody is 22C3. In some examples, the anti-PD-L1 antibody is 28-8.

In some examples, a tumor sample obtained from the individual has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the individual has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some aspects, a tumor sample obtained from the individual has a detectable expression level of PD-L1 in tumor-infiltrating immune cells that comprise 5%-19% of the tumor sample (e.g., TIC 5%-19%); e.g., has a PD-L1 expression level that is PD-L1 low. In some aspects, a tumor sample obtained from the individual has a detectable expression level of PD-L1 in tumor-infiltrating immune cells that comprise 220% of the tumor sample (e.g., TIC 220%); e.g., has a PD-L1 expression level that is PD-L1 high. In some embodiments, tumor samples that have been determined to have a TIC of greater than, or equal to, 5% are comparable to a CPS of greater than, or equal to, 1.

In some examples, tumor samples may be scored for PD-L1 positivity in tumor-infiltrating immune cells and/or in tumor cells according to the criteria for diagnostic assessment shown in Table 1 and/or Table 2, respectively.

TABLE 1
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any intensity in tumor-infiltrating immune | |
| cells covering <1% of tumor area occupied by tumor cells, associated intratumoral | |
| stroma, and contiguous peri-tumoral desmoplastic stroma | |
| Presence of discernible PD-L1 staining of any intensity in tumor-infiltrating immune | IC1 |
| cells covering ≥1% to <5% of tumor area occupied by tumor cells, associated | |
| intratumoral stroma, and contiguous peri-tumoral desmoplastic stroma | |
| Presence of discernible PD-L1 staining of any intensity in tumor-infiltrating immune | IC2 |
| cells covering ≥5% to <10% of tumor area occupied by tumor cells, associated | |
| intratumoral stroma, and contiguous peri-tumoral desmoplastic stroma | |
| Presence of discernible PD-L1 staining of any intensity in tumor-infiltrating immune | IC3 |
| cells covering ≥10% of tumor area occupied by tumor cells, associated intratumoral | |
| stroma, and contiguous peri-tumoral desmoplastic stroma | |

TABLE 2
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any intensity in <1% of tumor cells | |
| Presence of discernible PD-L1 staining of any intensity in ≥1% to <5% of tumor cells | TC1 |
| Presence of discernible PD-L1 staining of any intensity in ≥5% to <50% of tumor cells | TC2 |
| Presence of discernible PD-L1 staining of any intensity in ≥50% of tumor cells | TC3 |

In some instances, in any of the methods, uses, or compositions for use described herein, the individual has a PD-L1-selected tumor (e.g., a proportion of tumor area occupied by PD-L1 expressing tumor-infiltrating immune cells (ICs) is greater than or equal to 5% in the tumor sample as determined by an IHC with the SP142 antibody). In some instances, the PD-L1-selected tumor is a tumor that has been determined to have a proportion of tumor area occupied by PD-L1 expressing immune cells (ICs) greater than or equal to 5% by an immunohistochemical (IHC) assay. In some instances, the IHC assay uses the anti-PD-L1 antibody SP142, SP263, 22C3, or 28-8. In some instances, the IHC assay uses anti-PD-L1 antibody SP142. In some instances, the IHC assay uses anti-PD-L1 antibody SP263. In some instances, the IHC assay uses anti-PD-L1 antibody 22C3. In some instances, the IHC assay uses anti-PD-L1 antibody 22C3. In some instances, the IHC assay uses anti-PD-L1 antibody 28-8.

In some instances, the IC score has been determined to be greater than, or equal to, 5% (e.g., as determined using the Ventana (SP142) PD-L1 IHC assay). In some instances, the IC score has been determined to be 2 or 3 (e.g., as determined using the Ventana (SP142) PD-L1 IHC assay). In some instances, the IC score has been determined to be greater than, or equal to, 1% (e.g., as determined using the Ventana (SP142) PD-L1 IHC assay). In some instances, the IC score has been determined to be greater than, or equal to, 10% (e.g., as determined using the Ventana (SP142) PD-L1 IHC assay). In some instances, the IC score has been determined to be greater than, or equal to, 1% and less than 50% (e.g., as determined using the Ventana (SP142) PD-L1 IHC assay). In some instances, the IC score has been determined to be greater than, or equal to, 1% and less than 30% (e.g., as determined using the Ventana (SP142) PD-L1 IHC assay).

In some instances, in any of the methods, uses, or compositions for use described herein, a tumor sample obtained from the individual has a detectable protein expression level of PD-L1. In some instances, the detectable protein expression level of PD-L1 has been determined by an IHC assay. In some instances, the IHC assay uses anti-PD-L1 antibody SP142. In some instances, the tumor sample has been determined to have a detectable expression level of PD-L1 in tumor-infiltrating immune cells that comprise greater than, or equal to, 5% of the tumor sample. In some instances, the tumor sample has been determined to have a detectable expression level of PD-L1 in tumor-infiltrating immune cells that comprise greater than, or equal to, 1% of the tumor sample. In some instances, the tumor sample has been determined to have a detectable expression level of PD-L1 in tumor-infiltrating immune cells that comprise greater than, or equal to, 1% and less than 5% of the tumor sample. In some instances, the tumor sample has been determined to have a detectable expression level of PD-L1 in tumor-infiltrating immune cells that comprise greater than, or equal to, 5% and less than 10% of the tumor sample. In some instances, the tumor sample has been determined to have a detectable expression level of PD-L1 in tumor-infiltrating immune cells that comprise greater than, or equal to, 10% of the tumor sample. In some instances, the tumor sample has been determined to have a detectable expression level of PD-L1 in greater than, or equal to, 1% of the tumor cells in the tumor sample. In some instances, the tumor sample has been determined to have a detectable expression level of PD-L1 in greater than, or equal to, 1% and less than 5% of the tumor cells in the tumor sample. In some instances, the tumor sample has been determined to have a detectable expression level of PD-L1 in greater than, or equal to, 5% and less than 50% of the tumor cells in the tumor sample. In some instances, the tumor sample has been determined to have a detectable expression level of PD-L1 in greater than, or equal to, 50% of the tumor cells in the tumor sample.

In some instances, in any of the methods, uses, or compositions for use described herein, the individual has a PD-L1-selected tumor (e.g., a PD-L1 “high”-selected tumor (e.g., a PD-L1 tumor proportion score (TPS) greater than or equal to 50% in a tumor sample as determined by an IHC with the SP263 antibody)). In some instances, the PD-L1-selected tumor is a PD-L1 “high”-selected tumor. In some instances, the PD-L1-selected tumor is a tumor that has been determined to have TPS greater than or equal to 50% by an immunohistochemical (IHC) assay. In some instances, the IHC assay uses the anti-PD-L1 antibody SP263, SP142, 22C3, or 28-8. In some instances, the IHC assay uses anti-PD-L1 antibody SP263. In some instances, the IHC assay uses anti-PD-L1 antibody SP142. In some instances, the IHC assay uses anti-PD-L1 antibody 22C3. In some instances, the TPS has been determined to be greater than, or equal to, 50% (e.g., as determined using the Ventana (SP263) PD-L1 IHC assay). In some instances, the TPS has been determined to be less than 50% (e.g., as determined using the Ventana (SP263) PD-L1 IHC assay). In some instances, the TPS has been determined to be greater than, or equal to, 1% (e.g., as determined using the Ventana (SP263) PD-L1 IHC assay). In some instances, the TPS has been determined to be greater than, or equal to, 1% and less than 50% (e.g., as determined using the Ventana (SP263) PD-L1 IHC assay).

In some instances, in any of the methods, uses, or compositions for use described herein, a tumor sample obtained from the individual has a detectable protein expression level of PD-L1. In some instances, the detectable protein expression level of PD-L1 has been determined by an IHC assay. In some instances, the IHC assay uses anti-PD-L1 antibody SP263. In some instances, the tumor sample has been determined to have a PD-L1-positive tumor cell fraction greater than, or equal to, 50% of the tumor sample. In some instances, the tumor sample has been determined to have a PD-L1-positive tumor cell fraction less than 50% of the tumor sample. In some instances, the tumor sample has been determined to have a PD-L1-positive tumor cell fraction greater than, or equal to, 1% and less than 50% of the tumor sample.

In some instances, the IHC assay uses the anti-PD-L1 antibody 22C3. In some instances, the IHC assay is the pharmDx 22C3 IHC assay. In some instances, the PD-L1-positive tumor cell fraction is greater than, or equal to, 50% as determined by positive staining with the anti-PD-L1 antibody 22C3. In some embodiments, the tumor sample has been determined to have a combined positive score (CPS) of greater than, or equal to, 10 or a tumor proportion score (TPS) of greater than or equal to 1% in the tumor sample, e.g., as determined using the anti-PD-L1 antibody 22C3 as part of the pharmDx 22C3 IHC assay.

In some embodiments, the tumor sample has been determined to have a CPS of greater than, or equal to, 10 or a TPS of greater than or equal to 1% and less than 50% in the tumor sample, e.g., as determined using the anti-PD-L1 antibody 22C3 as part of the pharmDx 22C3 IHC assay. In some embodiments, the tumor sample has been determined to have a CPS of greater than, or equal to, 20 or a TPS of greater than or equal to 50% in the tumor sample, e.g., as determined using the anti-PD-L1 antibody 22C3 as part of the pharmDx 22C3 IHC assay. In some embodiments, tumor samples that have been determined to have a CPS of greater than, or equal to, 1 are comparable to a TIC of greater than, or equal to, 5%.

In some instances, the IHC assay uses the anti-PD-L1 antibody 28-8. In some instances, the IHC assay is the pharmDx 28-8 IHC assay. In some instances, the PD-L1-positive tumor cell fraction is greater than, or equal to, 50% as determined by positive staining with the anti-PD-L1 antibody 28-8.

In some instances, in any of the methods, uses, or compositions for use described herein, a tumor sample obtained from the individual has a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

### IV. Exemplary Anti-Tigit Antagonist Antibodies and PD-1 Axis Binding Antagonists

Exemplary anti-TIGIT antagonist antibodies and PD-1 axis binding antagonists useful for treating an individual (e.g., a human) having a cancer in accordance with the methods, uses, and compositions for use of the invention are described herein.

### A. Exemplary Anti-TIGIT Antagonist Antibodies

The invention provides anti-TIGIT antagonist antibodies useful for treating cancer in a subject (e.g., a human).

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibody includes at least one, two, three, four, five, or six HVRs selected from: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and/or (f) an HVR-L3 comprising the amino acid sequence of QQYYSTPFT (SEQ ID NO: 6), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 1-6.

In some instances, anti-TIGIT antagonist antibodies may include (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4); (e) an HVR-L2 comprising the amino acid sequence of WASTRES (SEQ ID NO: 5); and (f) an HVR-L3 comprising the amino acid sequence of QQYYSTPFT (SEQ ID NO: 6). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, EVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 17) or an amino acid sequence having at least 90% sequence identity (e.g., at least 91%, 92%, 93%, 94%, 95%, 96%, 97%, 98%, or 99% sequence identity) to, or the sequence of, QVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSS (SEQ ID NO: 18); and/or a VL domain comprising an amino acid sequence having at least 90% sequence identity (e.g., at least 91%, 92%, 93%, 94%, 95%, 96%, 97%, 98%, or 99% sequence identity) to, or the sequence of, DIVMTQSPDSLAVSLGERATINCKSSQTVLYSSNNKKYLAWYQQKPGQPPNLLIYWASTRESGVPDRFS GSGSGTDFTLTISSLQAEDVAVYYCQQYYSTPFTFGPGTKVEIK (SEQ ID NO: 19). In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 17 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 17 and a VL domain comprising the amino acid sequence of SEQ ID NO: 19. In some instances, the anti-TIGIT antagonist antibody has a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 18 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising the amino acid sequence of SEQ ID NO: 18 and a VL domain comprising the amino acid sequence of SEQ ID NO: 19.

In some instances, the anti-TIGIT antagonist antibody includes a heavy chain and a light chain sequence, wherein: (a) the heavy chain comprises the amino acid sequence: EVQLQQSGPGLVKPSQTLSLTCAISGDSVSSNSAAWNWIRQSPSRGLEWLGKTYYRFKWYSDYAVSVK GRITINPDTSKNQFSLQLNSVTPEDTAVFYCTRESTTYDLLAGPFDYWGQGTLVTVSSASTKGPSVFPLA PSSKSTSGGTAALGCLVKDYFPEPVTVSWNSGALTSGVHTFPAVLQSSGLYSLSSVVTVPSSSLGTQTYI CNVNHKPSNTKVDKKVEPKSCDKTHTCPPCPAPELLGGPSVFLFPPKPKDTLMISRTPEVTCVVVDVSHE DPEVKFNWYVDGVEVHNAKTKPREEQYNSTYRVVSVLTVLHQDWLNGKEYKCKVSNKALPAPIEKTISK AKGQPREPQVYTLPPSREEMTKNQVSLTCLVKGFYPSDIAVEWESNGQPENNYKTTPPVLDSDGSFFLY SKLTVDKSRWQQGNVFSCSVMHEALHNHYTQKSLSLSPGK (SEQ ID NO: 33); and (b) the light chain comprises the amino acid sequence: DIVMTQSPDSLAVSLGERATINCKSSQTVLYSSNNKKYLAWYQQKPGQPPNLLIYWASTRESGVPDRFS GSGSGTDFTLTISSLQAEDVAVYYCQQYYSTPFTFGPGTKVEIKRTVAAPSVFIFPPSDEQLKSGTASVVC LLNNFYPREAKVQWKVDNALQSGNSQESVTEQDSKDSTYSLSSTLTLSKADYEKHKVYACEVTHQGLSS PVTKSFNRGEC (SEQ ID NO: 34). In some aspects, the anti-TIGIT antagonist antibody comprises (a) a heavy chain comprising the amino acid sequence of SEQ ID NO: 33; and (b) a light chain comprising the amino acid sequence of SEQ ID NO: 34.

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and/or an FR-L4 comprising the amino acid sequence of FGPGTKVEIK (SEQ ID NO: 10), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 7-10. In some instances, for example, the antibody further comprises an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10).

In some instances, the anti-TIGIT antagonist antibody further comprises at least one, two, three, or four of the following heavy chain variable region FRs: an FR-H1 comprising the amino acid sequence of X₁VQLQQSGPGLVKPSQTLSLTCAISGDSVS (SEQ ID NO: 11), wherein X₁ is E or Q; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 11-14. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-15. In some instances, the anti-TIGIT antagonist antibody includes an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14). In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-14 and 16. In some instances, the anti-TIGIT antagonist antibody includes an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a K_{D} of about 10 nM or lower and cyno TIGIT with a K_{D} of about 10 nM or lower (e.g., binds human TIGIT with a K_{D} of about 0.1 nM to about 1 nM and cyno TIGIT with a K_{D} of about 0.5 nM to about 1 nM, e.g., binds human TIGIT with a K_{D} of about 0.1 nM or lower and cyno TIGIT with a K_{D} of about 0.5 nM or lower).

In some instances, the anti-TIGIT antagonist antibody specifically binds TIGIT and inhibits or blocks TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an IC50 value of 10 nM or lower (e.g., 1 nM to about 10 nM). In some instances, the anti-TIGIT antagonist antibody specifically binds TIGIT and inhibits or blocks TIGIT interaction with PVR, without impacting PVR-CD226 interaction. In some instances, the antagonist antibody inhibits or blocks binding of cyno TIGIT to cyno PVR with an IC50 value of 50 nM or lower (e.g., 1 nM to about 50 nM, e.g., 1 nM to about 5 nM). In some instances, the anti-TIGIT antagonist antibody inhibits and/or blocks the interaction of CD226 with TIGIT. In some instances, the anti-TIGIT antagonist antibody inhibits and/or blocks the ability of TIGIT to disrupt CD226 homodimerization.

In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and (f) an HVR-L3 comprising the amino acid sequence of QQYYSTPFT (SEQ ID NO: 6). The methods described herein may also include administering an isolated anti-TIGIT antagonist antibody that binds to the same epitope as an anti-TIGIT antagonist antibody described above.

In some aspects, the anti-TIGIT antagonist antibody is an antibody having intact Fc-mediated effector function (e.g., tiragolumab, vibostolimab, etigilimab, EOS084448, or TJ-T6) or enhanced effector function (e.g., SGN-TGT).

In some aspects, the anti-TIGIT antagonist antibody comprises an Fc domain that is able to interact with (e.g., activate) an Fc gamma receptor (FcγR). In some aspects, the anti-TIGIT antagonist antibody comprises an Fc domain that is able to interact with (e.g., activate) the FcγR of one or more myeloid cell types (e.g., one or more of cDC1s, macrophages, neutrophils, and circulating monocytes).

In some aspects, the anti-TIGIT antagonist antibody is capable of Fc-dependent activation of one or more myeloid cell types, e.g., one or more of intratumoral type 1 conventional dendritic cells (cDC1s), macrophages, neutrophils, and circulating monocytes.

In some aspects, the anti-TIGIT antagonist antibody is capable of interacting with the FcγR of one or more myeloid cell types (e.g., one or more of cDC1s, macrophages, neutrophils, and circulating monocytes) and is capable of inducing CD8+ T cell mobilization in the blood and/or an expansion of proliferating CD8+ T cells within the tumor bed.

In some aspects, the anti-TIGIT antagonist antibody is capable of interacting with (e.g., upregulating) the MYC targeting pathway. In some aspects, the invention comprises detecting a level (e.g., a gene expression level, e.g., a protein level or a nucleic acid level) of one or more members of the MYC targeting pathway (e.g., MYC) in the sample from the individual, e.g., comprises detecting a level of one or more members of the MYC targeting pathway in a sample from the individual at a time point during or after administration of the PD-1 axis binding antagonist and the anti-TIGIT antagonist antibody and comparing the detected level to reference expression level, e.g., a baseline expression level from a sample from the individual at a time point prior to the initiation of the treatment comprising the PD-1 axis binding antagonist and the anti-TIGIT antagonist antibody.

In some aspects, the anti-TIGIT antagonist antibody is an IgG1 class antibody, e.g., tiragolumab, vibostolimab, etigilimab, BGB-A1217, SGN-TGT, EOS084448 (EOS-448), TJ-T6, or AB308. Tiragolumab is an anti-TIGIT antibody monoclonal antibody (mAb) with a IgG1/kappa Fc.

In other aspects, the anti-TIGIT antagonist antibody is an IgG4 class antibody.

The anti-TIGIT antagonist antibodies (e.g., tiragolumab) useful in this invention, including compositions containing such antibodies, may be used in combination with a PD-1 axis binding antagonist (e.g., PD-L1 binding antagonists (e.g., anti-PD-L1 antagonist antibodies, e.g., atezolizumab), PD-1 binding antagonists (e.g., anti-PD-1 antagonist antibodies, e.g., pembrolizumab), and PD-L2 binding antagonists (e.g., anti-PD-L2 antagonist antibodies)).

In some embodiments, the anti-TIGIT antagonist antibody functions to inhibit TIGIT signaling. In some embodiments, the anti-TIGIT antagonist antibody inhibits the binding of TIGIT to its binding partners. Exemplary TIGIT binding partners include CD155 (PVR), CD112 (PVRL2 or Nectin-2), and CD113 (PVRL3 or Nectin-3). In some embodiments, the anti-TIGIT antagonist antibody is capable of inhibiting binding between TIGIT and CD155. In some embodiments, the anti-TIGIT antagonist antibody may inhibit binding between TIGIT and CD112. In some embodiments, the anti-TIGIT antagonist antibody inhibits binding between TIGIT and CD113. In some embodiments, the anti-TIGIT antagonist antibody inhibits TIGIT-mediated cellular signaling in immune cells. In some embodiments, the anti-TIGIT antagonist antibody inhibits TIGIT by depleting regulatory T cells (e.g., when engaging a FcγR).

In some embodiments, the anti-TIGIT antibody is a monoclonal antibody. In some embodiments, the anti-TIGIT antibody is an antibody fragment selected from the group consisting of Fab, Fab′-SH, Fv, scFv, and (Fab′)₂ fragments. In some embodiments, the anti-TIGIT antibody is a humanized antibody. In some embodiments, the anti-TIGIT antibody is a human antibody. In some embodiments, the anti-TIGIT antibody described herein binds to human TIGIT. In some embodiments, the anti-TIGIT antibody is an Fc fusion protein.

In some embodiments, the anti-TIGIT antibody is selected from the group consisting of tiragolumab (MTIG7192A, RG6058 or R07092284), vibostolimab (MK-7684), EOS884448 (EOS-448), SEA-TGT (SGN-TGT)), BGB-A1217, IB1939, M6223, AB308, AB154, TJ-T6, MG1131, NB6253, HLX301, HLX53, SL-9258 (TIGIT-Fc-LIGHT), STW264, and YBL-012. In some embodiments, the anti-TIGIT antibody is selected from the group consisting of tiragolumab (MTIG7192A, RG6058 or R07092284), vibostolimab (MK-7684), EOS-448, and SEA-TGT (SGN-TGT). The anti-TIGIT antibody may be tiragolumab (MTIG7192A, RG6058 or R07092284).

Non-limiting examples of anti-TIGIT antibodies that are useful for the methods disclosed herein, and methods for making thereof are described in PCT Pub. Nos. WO2018183889A1, WO2019129261A1, WO2016106302A9, WO2018033798A1, WO2020020281A1, WO2019023504A1, WO2017152088A1, WO2016028656A1, WO2017030823A2, WO2018204405A1, WO2019152574A1, and WO2020041541A2; U.S. Pat. Nos. 10,189,902, 10,213,505, 10,124,061, 10,537,633, and 10,618,958; and U.S. Pub. Nos. 2020/0095324, 2019/0112375, 2018/0371083, and 2020/0062859, each of which is incorporated herein by reference in its entirety. Additional non-limiting examples of anti-TIGIT antibodies, useful for the methods of disclosed herein, and methods for making thereof are described in PCT Pub. Nos. WO2018204363A1, WO2018047139A1, WO2019175799A2, WO2018022946A1, WO2015143343A2, WO2018218056A1, WO2019232484A1, WO2019079777A1, WO2018128939A1, WO2017196867A1, WO2019154415A1, WO2019062832A1, WO2018234793A3, WO2018102536A1, WO2019137548A1, WO2019129221A1, WO2018102746A1, WO2018160704A9, WO2020041541A2, WO2019094637A9, WO2017037707A1, WO2019168382A1, WO2006124667A3, WO2017021526A1, WO2017184619A2, WO2017048824A1, WO2019032619A9, WO2018157162A1, WO2020176718A1, WO2020047329A1, WO2020047329A1, WO2018220446A9; U.S. Pat. Nos. 9,617,338, 9,567,399, 10,604,576, and 9,994,637; and Pub. Nos. US 2018/0355040, US 2019/0175654, US 2019/0040154, US 2019/0382477, US 2019/0010246, US 2020/0164071, US 2020/0131267, US 2019/0338032, US 2019/0330351, US 2019/0202917, US 2019/0284269, US 2018/0155422, US 2020/0040082, US 2019/0263909, US 2018/0185480, US 2019/0375843, US 2017/0037133, US 2019/0077869, US 2019/0367579, US 2020/0222503, US 2020/0283496, CN109734806A, and CN110818795A, each of which is incorporated herein by reference in its entirety.

The anti-TIGIT antibodies useful in the methods disclosed herein include BGB-A1217, M6223, IB1939, EOS-448, vibostolimab (MK-7684), and SEA-TGT (SGN-TGT). Additional anti-TIGIT antibodies useful in the methods disclosed herein include AGEN1307; AGEN1777; antibody clones pab2197 and pab2196 (Agenus Inc.); antibody clones TBB8, TDC8, 3TB3, 5TB10, and D1Y1A (Anhui Anke Biotechnology Group Co. Ltd.), antibody clones MAB1, MAB2, MAB3, MAB4, MAB5, MAB6, MAB 7, MAB8, MAB9, MAB 10, MAB 11, MAB 12, MAB13, MAB 14, MAB 15, MAB 16, MAB 17, MAB 18, MAB19, MAB20, MAB21 (Astellas Pharma/Potenza Therapeutics), antibody clones hu1217-1-1 and hu1217-2-2 (BeiGene), antibody clones 4D4 and 19G (Brigham & Women's Hospital), antibody clones 11G11, 10D7, 15A6, 22G2, TIGIT G2a, and TIGIT G1 D265A, including such antibodies with modified heavy chain constant regions (Bristol-Myers Squibb); antibody clones 10A7, CPA.9.086, CPA.9.083.H4(S241P), CPA.9.086.H4(S241P), CHA.9.547.7.H4(S241P) and CHA.9.547.13.H4(S241P) (Compugen); anti-PVRIG/anti-TIGIT bispecific antibodies (Compugen), antibody clones 315293, 328189, 350426, 326504, and 331672 (Fred Hutchinson Cancer Research Center); antibody clones T-01, T-02, T-03, T-04, T-05, T-06, T-07, T-08, T-09, and T-10 (Gensun BioPharma Inc.); antibody clones 1H6, 2B111, 3A10, 4A5, 4A9, 4H5, 6A2, 6B7, 7F4, 8E1, 8G3, 9F4, 9G6, 10C1, 10F10, 11G4, 12B7, 12C8, 15E9, 16C11, 16D6, and 16E10 (Hefei Ruida Immunological Drugs Research Institute Co. Ltd.); antibody clones h3C5H1, h3C5H2, h3C5H3, h3C5H4, h3C5H3-1, h3C5H3-2, h3C5H3-3, h3C5L1, and h3C5L2 (IGM Biosciences Inc.); antibody clones 90D9, 101E1, 116H8, 118A12, 131A12, 143B6, 167F7, 221F11, 222H4, 327C9, 342A9, 344F2, 349H6, and 350D10 (I-Mab Biopharma); antibody clones ADI-27238, ADI-30263, ADI-30267, ADI-30268, ADI-27243, ADI-30302, ADI-30336, ADI-27278, ADI-30193, ADI-30296, ADI-27291, ADI-30283, ADI-30286, ADI-30288, ADI27297, ADI-30272, ADI-30278, ADI-27301, ADI-30306, and ADI-30311 (Innovent Biologics, Inc.); antibody clones 26518, 29478, 26452, 29487, 29489, 31282,26486,29494,29499,26521, 29513, 26493,29520,29523,29527,31288,32919,32931, 26432, and 32959 (iTeos Therapeutics); antibody clones m1707, m1708, m1709, m1710, m1711, h1707, h1708, h1709, h1710, and h1711 (Jiangsu Hengrui Medicine Co. Ltd.); antibody clones TIG1, TIG2, and TIG3 (JN Biosciences LLC); antibody clones (e.g., KY01, KY02, KY03, KY04, KY05, KY06, KY07, KY08, KY09, KY10, K11, K12, K13, K14, K15, K16, K17, K18, K19, K20, K21, K22, K23 Kymab TIGIT (Antibody 2), and Tool TIGIT (Antibody 4) (Kymab Limited); bispecific antibodies 1D05/in-house anti-TIGIT with 1D05 (anti-PD-L1) Native variable domain and Kymab TIGIT antigen binding site (ABS) domain (Bispecific 1), In-house anti-TIGIT/1D05 with Kymab TIGIT Native variable domain and 1D05 ABS domain (Bispecific 2), Tool anti-TIGIT/Tool anti-PD-L1 with Toon anti-TIGIT Native variable domain and Tool anti-PD-L1 ABS domain (Bispecific 3), Tool anti-PD-L1/Tool anti-TIGIT with Tool anti-PD-L1 Native variable domain and Tool anti-TIGIT ABS domain (Bispecific 4) (Kymab Limited); antibody clones and clone variants 14D7, 26B10, Hu14D7, Hu26B10, 14A6, Hu14A6, 28H5, 31C6, Hu31C6, 25G10, MBS43, 37D10, 18G10, 11A11, c18G10, and LB155.14A6. G2. A8 (Merck); etigilimab (OMP-313M32) (Mereo BioPharma); antibody clones 64G1E9B4, 100C4E7D11, 83G5H11C12, 92E9D4B4, 104G12E12G2, 121C2F10B5, 128E3F10F3F2, 70A11A8E6, 11D8E124A, 16F10H12C11, 8F2D8E7, 48B5G4E12,139E2C2D2, 128E3G7F5, AS19584, AS19852, AS19858, AS19886, AS19887, AS19888, AS20160, AS19584VH26, AS19584VH29, AS19584VH30, AS19584VH31, AS19886VH5, AS19886VH8, AS19886VH9, AS19886VH10, AS19886VH19, AS19886VH20, AS19584VH28-Fc, AS19886VH5-Fc, AS19886VH8-Fc, AS19584-Fc, and AS19886-Fc (Nanjing Legend Biotechnology Co. Ltd.); antibody clones ARE clones: Ab58, Ab69, Ab75, Ab133, Ab177, Ab122, Ab86, Ab180, Ab83, Ab26, Ab20, Ab147, Ab12, Ab66, Ab176, Ab96, Ab123, Ab109, Ab149, Ab34, Ab61, Ab64, Ab105, Ab108, Ab178, Ab166, Ab29, Ab135, Ab171, Ab194, Ab184, Ab164, Ab183, Ab158, Ab55, Ab136, Ab39, Ab159, Ab151, Ab139, Ab107, Ab36, Ab193, Ab115, Ab106, Ab13f8, Ab127, Ab165, Ab155, Ab19, Ab6, Ab187, Ab179, Ab65, Ab114, Ab102, Ab94, Ab163, Ab110, Ab80, Ab92, Ab117, Ab162, Ab121, Ab195, Ab84, Ab161, Ab198, Ab24, Ab98, Ab116, Ab174, Ab196, Ab51, Ab91, Ab185, Ab23, Ab7, Ab95, Ab100, Ab140, Ab145, Ab150, Ab168, Ab54, Ab77, Ab43, Ab160, Ab82, Ab189, Ab17, Ab103, Ab18, Ab130, Ab132, Ab134, Ab144; ARG Clones: Ab2, Ab47, Ab49, Ab31, Ab53, Ab40, Ab5, Ab9, Ab48, Ab4, Ab10, Ab37, Ab33, Ab42, Ab45; ARV Clones: Ab44, Ab97, Ab81, Ab188, Ab186, Ab62, Ab57, Ab192, Ab73, Ab60, Ab28, Ab32, Ab78, Ab14, Ab152, Ab72, Ab137, Ab128, Ab169, Ab87, Ab74, Ab172, Ab153, Ab120, Ab13, Ab113, Ab16, Ab56, Ab129, Ab50, Ab90, Ab99, Ab3, Ab148, Ab124, Ab22, Ab41, Ab119, Ab157, Ab27, Ab15, Ab191, Ab190, Ab79, Ab181, Ab146, Ab167, Ab88, Ab199, Ab71, Ab85, Ab59, Ab141, Ab68, Ab143, Ab46, Ab197, Ab175, Ab156, Ab63, Ab11, Ab182, Ab89, Ab8, Ab101, Ab25, Ab154, Ab21, Ab111, Ab118, Ab173, Ab38, Ab76, Ab131, Ab1, Ab67, Ab70, Ab170, Ab30, Ab93, Ab142, Ab104, Ab112, Ab35, Ab126, and Ab125 (Rigel Pharmaceuticals, Inc.); CASC-674 (Seattle Genetics); antibody clones 2, 2C, 3, 5, 13, 13A, 13B, 13C, 13D, 14, 16, 16C, 16D, 16E, 18, 21, 22, 25, 25A, 25B, 25C, 25D, 25E, 27, 54, 13 IgG2a afucosylated, 13 hIgG1 wild-type, and 13 LALA-PG (Seattle Genetics); JS006 (Shanghai Junshi Biosciences Ltd.); anti-TIGIT Fc antibody and bispecific antibody PD1×TIGIT (Xencor), antibody clone VSIG9 #1 (Vsig9.01) and 258-CS1 #4 (#4) (Yissum Research Development Company of The Hebrew University Of Jerusalem Ltd.); YH29143 (Yuhan Co, Ltd.); antibody clones S02, S03, S04, S05, S06, S11, S12, S14, S19, S32, S39, S43, S62, S64, F01, F02, F03, F04, 32D7, 101H3, 10A7, and 1F4 (Yuhan Co, Ltd.); anti-zB7R1 clones 318.4.1.1 (E9310), 318.28.2.1 (E9296), 318.39.1.1 (E9311), 318.59.3.1 (E9400), and 318.77.1.10 (ZymoGenetics, Inc).

In some embodiments, the anti-TIGIT antibody is selected from the group consisting of tiragolumab, BGB-A1217, M6223, IB1939, EOS884448 (EOS-448), vibostolimab (MK-7684), and SEA-TGT (SGN-TGT). ASP874 (PTZ-201) is an anti-TIGIT monoclonal antibody described in PCT Pub. No. WO2018183889A1 and US Pub. No. 2020/0095324. BGB-A1217 is an anti-TIGIT antibody as described in PCT Pub. No. WO2019129261A1. IB1939 is an anti-TIGIT antibody as described in PCT Pub. No. WO2020020281A1. EOS884448 (EOS-448) is an anti-TIGIT antibody described in PCT Pub. No. WO2019023504A1. Vibostolimab (MK-7684) is an anti-TIGIT antibody described in PCT Pub. Nos. WO2016028656A1, WO2017030823A2, WO2018204405A1, and/or WO2019152574A1, U.S. Pat. No. 10,618,958, and US Pub. No. 2018/0371083. SEA-TGT (SGN-TGT) is an anti-TIGIT antibody as described in PCT Pub. No. WO2020041541A2 and US Pub. No. 2020/0062859.

In some embodiments, the anti-TIGIT antagonist antibody is tiragolumab (CAS Registry Number: 1918185-84-8). Tiragolumab (Genentech) is also known as MTIG7192A, RG6058 or R07092284. Tiragolumab is an anti-TIGIT antagonistic monoclonal antibody described in PCT Pub. No. WO2003072305A8, WO2004024068A3, WO2004024072A3, WO2009126688A2, WO2015009856A2, WO2016011264A1, WO2016109546A2, WO2017053748A2, and WO2019165434A1, and US Pub. Nos. 2017/0044256, 2017/0037127, 2017/0145093, 2017/260594, 2017/0088613, 2018/0186875, 2019/0119376 and U.S. Pat. No. 9,873,740B2, U.S. Ser. No. 10/626,174B2, U.S. Ser. No. 10/611,836B2, U.S. Pat. No. 9,499,596B2, U.S. Pat. No. 8,431,350B2, U.S. Ser. No. 10/047,158B2, and U.S. Ser. No. 10/017,572B2.

In some embodiments, the anti-TIGIT antibody comprises at least one, two, three, four, five, or six complementarity determining regions (CDRs) of any of the anti-TIGIT antibodies disclosed herein. In some embodiments, the anti-TIGIT antibody comprises the six CDRs of any of the anti-TIGIT antibodies disclosed herein. In some embodiments, the anti-TIGIT antibody comprises the six CDRs of any one of the antibodies selected from the group consisting of tiragolumab, BGB-A1217, M6223, IB1939, EOS884448 (EOS-448), vibostolimab (MK-7684), and SEA-TGT (SGN-TGT).

In some embodiments, the anti-TIGIT antibody comprises a heavy chain and a light chain, wherein the heavy chain comprises a heavy chain variable region (VH) sequence of any one of the anti-TIGIT antibodies disclosed herein and the light chain comprises a light chain variable region (VL) of the same antibody. In some embodiments, the anti-TIGIT antibody comprises the VH and VL of an anti-TIGIT antibody selected from the group consisting of tiragolumab, BGB-A1217, M6223, IB1939, EOS884448 (EOS-448), vibostolimab (MK-7684), and SEA-TGT (SGN-TGT).

In some embodiments, the anti-TIGIT antibody comprises the heavy chain and the light chain of any of the anti-TIGIT antibodies disclosed herein. In some embodiments, the anti-TIGIT antibody comprises the heavy chain and the light chain of an anti-TIGIT antibody selected from the group consisting of tiragolumab, BGB-A1217, M6223, IB1939, EOS884448 (EOS-448), vibostolimab (MK-7684), and SEA-TGT (SGN-TGT).

In some embodiments, an anti-TIGIT antagonist antibody (according to any of the embodiments described herein may incorporate any of the features, singly or in combination, as described in Section IV(C) below.

### B. PD-1 Axis Binding Antagonists

Provided herein are methods for treating a cancer in a subject (e.g., a human) comprising administering to the subject an effective amount of a PD-1 axis binding antagonist. PD-1 axis binding antagonists may include PD-L1 binding antagonists, PD-1 binding antagonists, and PD-L2 binding antagonists. Any suitable PD-1 axis binding antagonist may be used.

### 1. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

Atezolizumab is a PD-L1-targeting monoclonal antibody (mAb) approved as first-line (1 L) monotherapy for patients with metastatic non-small cell lung cancer (NSCLC) whose tumors have high PD-L1 expression and as an adjuvant treatment for patients with resected Stage II-IIIA NSCLC.

In some instances, the anti-PD-L1 antibody (e.g., atezolizumab) includes at least one, two, three, four, five, or six HVRs selected from: (a) an HVR-H1 sequence is GFTFSDSWIH (SEQ ID NO: 20); (b) an HVR-H2 sequence is AWISPYGGSTYYADSVKG (SEQ ID NO: 21); (c) an HVR-H3 sequence is RHWPGGFDY (SEQ ID NO: 22), (d) an HVR-L1 sequence is RASQDVSTAVA (SEQ ID NO: 23); (e) an HVR-L2 sequence is SASFLYS (SEQ ID NO: 24); and (f) an HVR-L3 sequence is QQYLYHPAT (SEQ ID NO: 25).

In some instances, the anti-PD-L1 antibody comprises:
(a) an HVR-H1, HVR-H2, and HVR-H3 sequence of SEQ ID NO: 20, SEQ ID NO: 21 and SEQ ID NO: 22, respectively, and
(b) an HVR-L1, HVR-L2, and HVR-L3 sequence of SEQ ID NO: 23, SEQ ID NO: 24 and SEQ ID NO: 25, respectively.

In some instances, the anti-PD-L1 antibody (e.g., atezolizumab) comprises a heavy chain and a light chain sequence, wherein: (a) the heavy chain variable (VH) region sequence comprises the amino acid sequence: EVQLVESGGGLVQPGGSLRLSCAASGFTFSDSWIHWVRQAPGKGLEWVAWISPYGGSTYYADSVKGR FTISADTSKNTAYLQMNSLRAEDTAVYYCARRHWPGGFDYWGQGTLVTVSS (SEQ ID NO: 26); and (b) the light chain variable (VL) region sequence comprises the amino acid sequence: DIQMTQSPSSLSASVGDRVTITCRASQDVSTAVAWYQQKPGKAPKLLIYSASFLYSGVPSRFSGSGSGT DFTLTISSLQPEDFATYYCQQYLYHPATFGQGTKVEIKR (SEQ ID NO: 27).

In some instances, the anti-PD-L1 antibody (e.g., atezolizumab) comprises a heavy chain and a light chain sequence, wherein: (a) the heavy chain comprises the amino acid sequence: EVQLVESGGGLVQPGGSLRLSCAASGFTFSDSWIHWVRQAPGKGLEWVAWISPYGGSTYYADSVKGR FTISADTSKNTAYLQMNSLRAEDTAVYYCARRHWPGGFDYWGQGTLVTVSSASTKGPSVFPLAPSSKST SGGTAALGCLVKDYFPEPVTVSWNSGALTSGVHTFPAVLQSSGLYSLSSVVTVPSSSLGTQTYICNVNHK PSNTKVDKKVEPKSCDKTHTCPPCPAPELLGGPSVFLFPPKPKDTLMISRTPEVTCVVVDVSHEDPEVKF NWYVDGVEVHNAKTKPREEQYASTYRVVSVLTVLHQDWLNGKEYKCKVSNKALPAPIEKTISKAKGQPR EPQVYTLPPSREEMTKNQVSLTCLVKGFYPSDIAVEWESNGQPENNYKTTPPVLDSDGSFFLYSKLTVD KSRWQQGNVFSCSVMHEALHNHYTQKSLSLSPG (SEQ ID NO: 28); and (b) the light chain comprises the amino acid sequence: DIQMTQSPSSLSASVGDRVTITCRASQDVSTAVAWYQQKPGKAPKLLIYSASFLYSGVPSRFSGSGSGT DFTLTISSLQPEDFATYYCQQYLYHPATFGQGTKVEIKRTVAAPSVFIFPPSDEQLKSGTASVVCLLNNFY PREAKVQWKVDNALQSGNSQESVTEQDSKDSTYSLSSTLTLSKADYEKHKVYACEVTHQGLSSPVTKSF NRGEC (SEQ ID NO: 29).

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 26); (b) a VL domain comprising an amino acid sequence comprising having at least 95% sequence identity (e.g., at least 95%, 96%, 97%, 98%, or 99% sequence identity) to, or the sequence of (SEQ ID NO: 27); or (c) a VH domain as in (a) and a VL domain as in (b). In one embodiment, the anti-PD-L1 antibody comprises atezolizumab, which comprises: (a) the heavy chain amino acid sequence of SEQ ID NO: 28, and (b) the light chain amino acid sequence of SEQ ID NO: 29.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### 2. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings).

ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal Fc-mediated effector function. In a still further specific aspect, the minimal Fc-mediated effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### 3. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

The PD-1 axis binding antagonists (e.g., atezolizumab) useful in this invention, including compositions containing such molecules, may be used in combination with an anti-TIGIT antagonist antibody.

In a further aspect, a PD-1 axis binding antagonist is a PD-1 axis binding antagonist antibody according to any of the above instances may incorporate any of the features, singly or in combination, as described in Section IV(C) below.

### C. Antibody Formats and Properties

### 1. Antibody Affinity

In certain instances, an anti-TIGIT antagonist antibody, PD-1 axis binding antagonist antibody (e.g., anti-PD-L1 antagonist antibody or anti-PD-1 antagonist antibody), anti-VEGF antibody, and/or anti-IL-6R antibody) provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one instance, K_{D} is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20©) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, K_{D} is measured using a BIACORE® surface plasmon resonance assay. For example, an assay using a BIACORE©-2000 or a BIACORE ©-3000 (BIAcore, Inc., Piscataway, NJ) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10⁶M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain instances, an anti-TIGIT antagonist antibody and/or PD-1 axis binding antagonist antibody (e.g., anti-PD-L1 antagonist antibody or anti-PD-1 antagonist antibody) provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, MA; see, e.g., U.S. Pat. No. 6,248,516B1).

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain instances, an anti-TIGIT antagonist antibody and/or PD-1 axis binding antagonist antibody (e.g., anti-PD-L1 antagonist antibody or anti-PD-1 antagonist antibody) provided herein is a chimeric antibody. Certain chimeric antibodies are described, e.g., in U.S. Pat. No. 4,816,567; and Morrison et al. Proc. Nat. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain instances, an anti-TIGIT antagonist antibody and/or PD-1 axis binding antagonist antibody (e.g., anti-PD-L1 antagonist antibody or anti-PD-1 antagonist antibody) provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):1 85-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Anti-TIGIT antagonist antibody and/or PD-1 axis binding antagonist antibodies (e.g., anti-PD-L1 antagonist antibodies or anti-PD-1 antagonist antibodies) of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (e.g., from human) to provide a single source of antibodies to a wide range of non-self and also self antigens without any immunization as described by Griffiths et al., EMBO J, 12: 725-734 (1993). Finally, naïve libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Anti-TIGIT antagonist antibody and/or PD-1 axis binding antagonist antibodies (e.g., anti-PD-L1 antagonist antibodies) or antibody fragments isolated from human antibody libraries are considered human antibodies or human antibody fragments herein.

### 6. Antibody Variants

In certain instances, amino acid sequence variants of the anti-TIGIT antagonist antibodies and/or PD-1 axis binding antagonist antibodies (e.g., anti-PD-L1 antagonist antibodies or anti-PD-1 antagonist antibodies) of the invention are contemplated. As described in detail herein, anti-TIGIT antagonist antibodies and PD-1 axis binding antagonist antibodies (e.g., anti-PD-L1 antagonist antibodies) may be optimized based on desired structural and functional properties. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

### I. Substitution, Insertion, and Deletion Variants

In certain instances, anti-TIGIT antagonist antibody and/or PD-1 axis binding antagonist antibody (e.g., anti-PD-L1 antagonist antibody or anti-PD-1 antagonist antibody) variants having one or more amino acid substitutions are provided. Sites of interest for substitutional mutagenesis include the HVRs and FRs. Conservative substitutions are shown in Table 3 under the heading of “preferred substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 3
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen-contacting residues in the HVRs. In certain instances of the variant VH and VL sequences provided above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### II. Glycosylation Variants

In certain instances, anti-TIGIT antagonist antibodies and/or PD-1 axis binding antagonist antibodies (e.g., anti-PD-L1 antagonist antibodies or anti-PD-1 antagonist antibodies) of the invention can be altered to increase or decrease the extent to which the antibody is glycosylated. Addition or deletion of glycosylation sites to anti-TIGIT antagonist antibody and/or PD-1 axis binding antagonist antibody (e.g., anti-PD-L1 antagonist antibody) of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention are made in order to create antibody variants with certain improved properties.

In one instance, anti-TIGIT antagonist antibody and/or PD-1 axis binding antagonist antibody (e.g., anti-PD-L1 antagonist antibody or anti-PD-1 antagonist antibody) variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In view of the above, in some instances, the methods of the invention involve administering to the subject in the context of a fractionated, dose-escalation dosing regimen an anti-TIGIT antagonist antibody (e.g., an anti-TIGIT antagonist antibody disclosed herein, e.g., tiragolumab) and/or PD-1 axis binding antagonist antibody (e.g., anti-PD-L1 antagonist antibody (e.g., atezolizumab) or anti-PD-1 antagonist antibody) variant that comprises an aglycosylation site mutation. In some instances, the aglycosylation site mutation reduces effector function of the antibody. In some instances, the aglycosylation site mutation is a substitution mutation. In some instances, the PD-1 axis binding antagonist antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred instance, the substitution mutation is N297A.

Anti-TIGIT antagonist antibody and/or PD-1 axis binding antagonist antibody (e.g., anti-PD-L1 antagonist antibody or anti-PD-1 antagonist antibody) variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### III. Fc Region Variants

In certain instances, one or more amino acid modifications are introduced into the Fc region of an anti-TIGIT antagonist (e.g., an anti-TIGIT antagonist antibody disclosed herein, e.g., tiragolumab) antibody and/or PD-1 axis binding antagonist antibody (e.g., anti-PD-L1 antagonist antibody (e.g., atezolizumab) or anti-PD-1 antagonist antibody) of the invention, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the invention contemplates a PD-1 axis binding antagonist antibody (e.g., anti-PD-L1 antagonist antibody variant) that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FγRIII only, whereas monocytes express FcγRI, FcγRII, and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT™ non-radioactive cytotoxicity assay for flow cytometry (CellTechnology, Inc. Mountain View, CA; and CYTOTOX 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies (e.g., PD-1 axis binding antagonist antibodies) with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the PD-1 axis binding antagonist antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcγRIII (Sondermann et al.: Nature 406, 267-273 (20 Jul. 2000)). In certain instances, the antibody comprises at least one further amino acid substitution. In one instance, the further amino acid substitution is S228P, E233P, L234A, L235A, L235E, N297A, N297D, or P331S, and still in another instance the at least one further amino acid substitution is L234A and L235A of the human IgG1 Fc region or S228P and L235E of the human IgG4 Fc region (see e.g., US 2012/0251531), and still in another instance the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instance, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424, or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects, the anti-PD-L1 antagonist antibody (e.g., atezolizumab) comprises an Fc region comprising an N297G mutation (EU numbering).

In some instances, the anti-TIGIT antagonist antibody (e.g., an anti-TIGIT antagonist antibody disclosed herein, e.g., tiragolumab) and/or PD-1 axis binding antagonist antibody (e.g., anti-PD-L1 antagonist antibody (e.g., atezolizumab) or anti-PD-1 antagonist antibody) comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some instances, at least one of the one or more heavy chain constant domains is paired with another heavy chain constant domain. In some instances, the CH3₁ and CH3₂ domains each comprise a protuberance or cavity, and wherein the protuberance or cavity in the CH3₁ domain is positionable in the cavity or protuberance, respectively, in the CH3₂ domain. In some instances, the CH3₁ and CH3₂ domains meet at an interface between said protuberance and cavity. In some instances, the CH2₁ and CH2₂ domains each comprise a protuberance or cavity, and wherein the protuberance or cavity in the CH2₁ domain is positionable in the cavity or protuberance, respectively, in the CH2₂ domain. In other instances, the CH2₁ and CH2₂ domains meet at an interface between said protuberance and cavity. In some instances, the anti-TIGIT antagonist antibody (e.g., an anti-TIGIT antagonist antibody disclosed herein, e.g., tiragolumab) and/or anti-PD-L1 antagonist antibody (e.g., atezolizumab) is an IgG1 antibody.

### IV. Cysteine Engineered Antibody Variants

In certain instances, it is desirable to create cysteine engineered anti-TIGIT antagonist antibodies and/or PD-1 axis binding antagonist antibodies (e.g., anti-PD-L1 antagonist antibodies or anti-PD-1 antagonist antibodies), e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

### V. Antibody Derivatives

In certain instances, an anti-TIGIT antagonist antibody of the invention (e.g., an anti-TIGIT antagonist antibody (e.g., tiragolumab) or a variant thereof) and/or PD-1 axis binding antagonist antibody (e.g., anti-PD-L1 antagonist antibody of the invention (e.g., atezolizumab or a variant thereof)) provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Recombinant Production Methods

Anti-TIGIT antagonist antibodies (e.g., an anti-TIGIT antagonist antibody disclosed herein, e.g., tiragolumab) and/or PD-1 axis binding antagonist antibodies (e.g., anti-PD-L1 antagonist antibodies (e.g., atezolizumab) or anti-PD-1 antagonist antibodies) of the invention may be produced using recombinant methods and compositions, for example, as described in U.S. Pat. No. 4,816,567, which is incorporated herein by reference in its entirety.

For recombinant production of an anti-TIGIT antagonist antibody and/or PD-1 axis binding antagonist antibody (e.g., anti-PD-L1 antagonist antibody or anti-PD-1 antagonist antibody), nucleic acid encoding an antibody, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### Immunoconjugates

Also provided are immunoconjugates comprising an anti-TIGIT antagonist antibody as disclosed herein, e.g., tiragolumab and/or PD-1 axis binding antagonist (e.g., anti-PD-L1 antagonist antibody (e.g., atezolizumab) or anti-PD-1 antagonist antibody (e.g., pembrolizumab)) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes, for use in the methods or uses described herein.

In some instances, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an anti-TIGIT antagonist antibody as described herein (e.g., tiragolumab) or a PD-1 axis binding antagonist (e.g., an anti-PD-L1 antagonist antibody (e.g., atezolizumab)) conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an anti-TIGIT antagonist antibody as described herein (e.g., tiragolumab) and/or a PD-1 axis binding antagonist (e.g., an anti-PD-L1 antagonist antibody) as described herein (e.g., atezolizumab) conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker, or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

ADCs not comprising an anti-TIGIT antagonist antibody or a PD-1 axis binding antagonist may also be used in the methods described herein. In some instances, the ADC is enfortumab vedotin or sacituzumab govitecan.

### D. Methods of Delivery

The compositions utilized in the methods described herein (e.g., immune checkpoint inhibitors) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some aspects, an immune checkpoint inhibitor (e.g., a PD-1 axis binding antagonist, e.g., atezolizumab and/or an anti-TIGIT antagonist antibody, e.g., tiragolumab) is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Immune checkpoint inhibitors (e.g., a PD-1 axis binding antagonist or an anti-TIGIT antagonist antibody) described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immune checkpoint inhibitor need not be, but is optionally formulated with and/or administered concurrently with one or more agents currently used to prevent or treat the disorder in question, e.g., one or more of the agents provided herein. The effective amount of such other agents depends on the amount of the immune checkpoint inhibitor present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of a cancer, e.g., a lung cancer (e.g., a NSCLC), the appropriate dosage of an immune checkpoint inhibitor, e.g., a PD-1 axis binding antagonist, an anti-TIGIT antagonist antibody, or any combination thereof, described herein (when used alone or in combination with one or more other additional therapeutic agents) will depend on the type of disease to be treated, the severity and course of the disease, whether the PD-L1 axis binding antagonist and/or anti-TIGIT antagonist antibody is administered for preventive or therapeutic purposes, previous therapy, the patient's clinical history and response to the PD-L1 axis binding antagonist and/or anti-TIGIT antagonist antibody, and the discretion of the attending physician. The immune checkpoint inhibitor is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the immune checkpoint inhibitor). An initial higher loading dose, followed by one or more lower doses, may be administered. However, other dosage regimens may be useful. The progress of this therapy is easily monitored by conventional techniques and assays.

### E. Dosing

### L. Dosing of Anti-TIGIT Antagonist Antibodies

As a general proposition, the therapeutically effective amount of an anti-TIGIT antagonist antibody (e.g., an anti-TIGIT antagonist antibody as disclosed herein, e.g., tiragolumab) administered to a human will be in the range of about 0.01 to about 50 mg/kg of patient body weight, whether by one or more administrations. In some embodiments, the therapeutically effective amount of an anti-TIGIT antagonist antibody (e.g., an anti-TIGIT antagonist antibody as disclosed herein, e.g., tiragolumab) administered to a human is in the range of 0.01 to 50 mg/kg of patient body weight, whether by one or more administrations.

In some exemplary embodiments, the anti-TIGIT antagonist antibody (e.g., an anti-TIGIT antagonist antibody as disclosed herein, e.g., tiragolumab) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In exemplary embodiments, the anti-TIGIT antagonist antibody (e.g., an anti-TIGIT antagonist antibody as disclosed herein, e.g., tiragolumab) is administered in a dose of 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, every two weeks, every three weeks, or every four weeks, for example.

In some instances, the anti-TIGIT antagonist antibody (e.g., an anti-TIGIT antagonist antibody as disclosed herein, e.g., tiragolumab) is administered on about Day 1 (e.g., Day −3, Day −2, Day −1, Day 1, Day 2, or Day 3) of a dosing cycle.

In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject). Such dosing regimens can be utilized in treatments for subjects having relatively low body weight (e.g., 40 kg or less (e.g., from 5 kg to 40 kg, from 15 kg to 40 kg, or from 5 kg to 15 kg)) and have been developed through biostimulation studies based on extrapolations of pharmacokinetic parameters estimated from adult data.

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body weight. In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between about 10 mg to about 1000 mg every three weeks (e.g., about 300 mg every three weeks); (b) greater than 15 kg and less than or equal to 40 kg, and the anti-TIGIT antagonist antibody is administered at a dose of between about 10 mg to about 1000 mg every three weeks (e.g., about 400 mg every three weeks); or (c) greater than 40 kg, and the anti-TIGIT antagonist antibody is administered at a dose of between about 30 mg to about 1200 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between about 250 mg to about 350 mg every three weeks (e.g., about 300 mg every three weeks); (b) greater than 15 kg and less than or equal to 40 kg, and the anti-TIGIT antagonist antibody is administered at a dose of between about 350 mg to about 450 mg every three weeks (e.g., about 400 mg every three weeks); or (c) greater than 40 kg, and the anti-TIGIT antagonist antibody is administered at a dose of between about 550 mg to about 650 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of about 300 mg every three weeks; (b) greater than 15 kg and less than or equal to 40 kg, and the anti-TIGIT antagonist antibody is administered at a dose of about 400 mg every three weeks; or (c) greater than 40 kg, and the anti-TIGIT antagonist antibody is administered at a dose of about 600 mg every three weeks. In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between 10 mg to 1000 mg every three weeks (e.g., 300 mg every three weeks); (b) greater than 15 kg and less than or equal to 40 kg, and the anti-TIGIT antagonist antibody is administered at a dose of between 10 mg to 1000 mg every three weeks (e.g., 400 mg every three weeks); or (c) greater than 40 kg, and the anti-TIGIT antagonist antibody is administered at a dose of between 30 mg to 1200 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between 250 mg to 350 mg every three weeks (e.g., 300 mg every three weeks); (b) greater than 15 kg and less than or equal to 40 kg, and the anti-TIGIT antagonist antibody is administered at a dose of between 350 mg to 450 mg every three weeks (e.g., 400 mg every three weeks); or (c) greater than 40 kg, and the anti-TIGIT antagonist antibody is administered at a dose of between 550 mg to 650 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of 300 mg every three weeks; (b) greater than 15 kg and less than or equal to 40 kg, and the anti-TIGIT antagonist antibody is administered at a dose of 400 mg every three weeks; or (c) greater than 40 kg, and the anti-TIGIT antagonist antibody is administered at a dose of 600 mg every three weeks.

In some instances, the effective amount of the anti-TIGIT antagonist antibody (e.g., an anti-TIGIT antagonist antibody as disclosed herein, e.g., tiragolumab) is a dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W) for subject with a body weight greater than 40 kg (e.g., 40.5 kg, 41 kg, 42 kg, 43 kg, 44 kg, 45 kg, 46 kg, 47 kg, 48 kg, 49 kg, 50 kg, 51 kg, 52 kg, 53 kg, 54 kg, 55 kg, 56 kg, 57 kg, 58 kg, 59 kg, 60 kg, 61 kg, 62 kg, 63 kg, 64 kg, 65 kg, 66 kg, 67 kg, 68 kg, 69 kg, 70 kg, 75 kg, 80 kg, 85 kg, 90 kg, 95 kg, 100 kg, 110 kg, 120 kg, 130 kg, 140 kg, 150 kg or more). In some instances, the effective amount of the anti-TIGIT antagonist antibody (e.g., an anti-TIGIT antagonist antibody as disclosed herein, e.g., tiragolumab) is a dose of about 600 mg every three weeks for subject with a body weight greater than 40 kg. In some instances, the effective amount of the anti-TIGIT antagonist antibody (e.g., an anti-TIGIT antagonist antibody as disclosed herein, e.g., tiragolumab) is a dose of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W) for subject with a body weight greater than 40 kg (e.g., 40.5 kg, 41 kg, 42 kg, 43 kg, 44 kg, 45 kg, 46 kg, 47 kg, 48 kg, 49 kg, 50 kg, 51 kg, 52 kg, 53 kg, 54 kg, 55 kg, 56 kg, 57 kg, 58 kg, 59 kg, 60 kg, 61 kg, 62 kg, 63 kg, 64 kg, 65 kg, 66 kg, 67 kg, 68 kg, 69 kg, 70 kg, 75 kg, 80 kg, 85 kg, 90 kg, 95 kg, 100 kg, 110 kg, 120 kg, 130 kg, 140 kg, 150 kg or more). In some instances, the effective amount of the anti-TIGIT antagonist antibody (e.g., an anti-TIGIT antagonist antibody as disclosed herein, e.g., tiragolumab) is a dose of 600 mg every three weeks for subject with a body weight greater than 40 kg.

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 700 mg, e.g., between about 250 mg to about 600 mg, e.g., between about 300 mg to about 500 mg, e.g., between about 350 mg to about 450 mg, e.g., between about 390 mg to about 410 mg, e.g., about 400 mg) every three weeks (Q3W) for subject with a body weight greater than 15 kg and less than or equal to 40 kg (e.g., 15.1 kg, 15.2 kg, 15.3 kg, 15.4 kg, 15.5 kg, 16 kg, 17 kg, 18 kg, 19 kg, 20 kg, 21 kg, 22 kg, 23 kg, 24 kg, 25 kg, 26 kg, 27 kg, 28 kg, 29 kg, 30 kg, 31 kg, 32 kg, 33 kg, 34 kg, 35 kg, 36 kg, 37 kg, 38 kg, 39 kg, or 39.5 kg). In some instances, the effective amount of the anti-TIGIT antagonist antibody (e.g., an anti-TIGIT antagonist antibody as disclosed herein, e.g., tiragolumab) is a dose of about 400 mg every three weeks (e.g., 400 mg±10 mg, e.g., 400±6 mg, e.g., 400±5 mg, e.g., 400±3 mg, e.g., 400±1 mg, e.g., 400±0.5 mg, e.g., 400 mg every three weeks) for subject with a body weight greater than 15 kg and less than or equal to 40 kg. In some instances, the effective amount of the anti-TIGIT antagonist antibody (e.g., an anti-TIGIT antagonist antibody as disclosed herein, e.g., tiragolumab) is a dose of between 10 mg to 1000 mg (e.g., between 20 mg to 1000 mg, e.g., between 50 mg to 900 mg, e.g., between 100 mg to 850 mg, e.g., between 200 mg to 700 mg, e.g., between 250 mg to 600 mg, e.g., between 300 mg to 500 mg, e.g., between 350 mg to 450 mg, e.g., between 390 mg to 410 mg, e.g., 400 mg) every three weeks (Q3W) for subject with a body weight greater than 15 kg and less than or equal to 40 kg (e.g., 15.1 kg, 15.2 kg, 15.3 kg, 15.4 kg, 15.5 kg, 16 kg, 17 kg, 18 kg, 19 kg, 20 kg, 21 kg, 22 kg, 23 kg, 24 kg, 25 kg, 26 kg, 27 kg, 28 kg, 29 kg, 30 kg, 31 kg, 32 kg, 33 kg, 34 kg, 35 kg, 36 kg, 37 kg, 38 kg, 39 kg, or 39.5 kg). In some instances, the effective amount of the anti-TIGIT antagonist antibody (e.g., an anti-TIGIT antagonist antibody as disclosed herein, e.g., tiragolumab) is a dose of 400 mg every three weeks (e.g., 400 mg±10 mg, e.g., 400±6 mg, e.g., 400±5 mg, e.g., 400±3 mg, e.g., 400±1 mg, e.g., 400±0.5 mg, e.g., 400 mg every three weeks) for subject with a body weight greater than 15 kg and less than or equal to 40 kg.

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 10 mg to about 900 mg, e.g., between about 50 mg to about 900 mg, e.g., between about 100 mg to about 750 mg, e.g., between about 100 mg to about 600 mg, e.g., between about 150 mg to about 500 mg, e.g., between about 200 mg to about 400 mg, e.g., between about 250 mg to about 350 mg, e.g., between about 290 mg to about 310 mg, e.g., about 300 mg) every three weeks (Q3W) for subject with a body weight less than or equal to 15 kg (e.g., 0.5 kg, 1 kg, 1.5 kg, 2.0 kg, 2.5 kg, 3.0 kg, 3.5 kg, 4.0 kg, 4.5 kg, 5.0 kg, 5.5 kg, 6.0 kg, 6.5 kg, 7.0 kg, 7.5 kg, 8.0 kg, 8.5 kg, 9.0 kg, 9.5 kg, 10.0 kg, 10.5 kg, 11.0 kg, 11.5 kg, 12.0 kg, 12.5 kg, 13.0 kg, 13.5 kg, 14.0 kg, 14.5 kg, or 15.0 kg).

In some instances, the effective amount of the anti-TIGIT antagonist antibody (e.g., an anti-TIGIT antagonist antibody as disclosed herein, e.g., tiragolumab) is a dose of about 300 mg every three weeks (e.g., 300 mg±10 mg, e.g., 300±6 mg, e.g., 300±5 mg, e.g., 300±3 mg, e.g., 300±1 mg, e.g., 300±0.5 mg, e.g., 300 mg every three weeks) for subject with a body weight less than or equal to 15 kg. In some instances, the effective amount of the anti-TIGIT antagonist antibody (e.g., an anti-TIGIT antagonist antibody as disclosed herein, e.g., tiragolumab) is a dose of between 10 mg to 1000 mg (e.g., between 10 mg to 900 mg, e.g., between 50 mg to 900 mg, e.g., between 100 mg to 750 mg, e.g., between 100 mg to 600 mg, e.g., between 150 mg to 500 mg, e.g., between 200 mg to 400 mg, e.g., between 250 mg to 350 mg, e.g., between 290 mg to 310 mg, e.g., 300 mg) every three weeks (Q3W) for subject with a body weight less than or equal to 15 kg (e.g., 0.5 kg, 1 kg, 1.5 kg, 2.0 kg, 2.5 kg, 3.0 kg, 3.5 kg, 4.0 kg, 4.5 kg, 5.0 kg, 5.5 kg, 6.0 kg, 6.5 kg, 7.0 kg, 7.5 kg, 8.0 kg, 8.5 kg, 9.0 kg, 9.5 kg, 10.0 kg, 10.5 kg, 11.0 kg, 11.5 kg, 12.0 kg, 12.5 kg, 13.0 kg, 13.5 kg, 14.0 kg, 14.5 kg, or 15.0 kg). In some instances, the effective amount of the anti-TIGIT antagonist antibody (e.g., an anti-TIGIT antagonist antibody as disclosed herein, e.g., tiragolumab) is a dose of 300 mg every three weeks (e.g., 300 mg±10 mg, e.g., 300±6 mg, e.g., 300±5 mg, e.g., 300±3 mg, e.g., 300±1 mg, e.g., 300±0.5 mg, e.g., 300 mg every three weeks) for subject with a body weight less than or equal to 15 kg.

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body surface area. In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between about 10 mg to about 1000 mg every three weeks (e.g., about 300 mg every three weeks); (b) greater than 0.5 m² and less than or equal to 0.75 m², and the anti-TIGIT antagonist antibody is administered at a dose of between about 10 mg to about 1000 mg every three weeks (e.g., about 350 mg every three weeks); (c) greater than 0.75 m² and less than or equal to 1.25 m², and the anti-TIGIT antagonist antibody is administered at a dose of between about 10 mg to about 1000 mg every three weeks (e.g., about 450 mg every three weeks); or (d) greater than 1.25 m², and the anti-TIGIT antagonist antibody is administered at a dose of between about 30 mg to about 1200 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between about 250 mg to about 350 mg every three weeks (e.g., about 300 mg every three weeks); (b) greater than 0.5 m² and less than or equal to 0.75 m², and the anti-TIGIT antagonist antibody is administered at a dose of between about 300 mg to about 400 mg every three weeks (e.g., about 350 mg every three weeks); or (c) greater than 0.75 m² and less than or equal to 1.25 m², and the anti-TIGIT antagonist antibody is administered at a dose of between about 400 mg to about 500 mg every three weeks (e.g., about 450 mg every three weeks); or (d) greater than 1.25 m², and the anti-TIGIT antagonist antibody is administered at a dose of between about 550 mg to about 650 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of about 300 mg every three weeks; (b) greater than 0.5 m² and less than or equal to 0.75 m², and the anti-TIGIT antagonist antibody is administered at a dose of about 400 mg every three weeks; (c) greater than 0.75 m² and less than or equal to 1.25 m², and the anti-TIGIT antagonist antibody is administered at a dose of 450 mg every three weeks; or (d) greater than 1.25 m², and the anti-TIGIT antagonist antibody is administered at a dose of about 600 mg every three weeks. In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body surface area. In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between 10 mg to 1000 mg every three weeks (e.g., 300 mg every three weeks); (b) greater than 0.5 m² and less than or equal to 0.75 m², and the anti-TIGIT antagonist antibody is administered at a dose of between 10 mg to 1000 mg every three weeks (e.g., 350 mg every three weeks); (c) greater than 0.75 m² and less than or equal to 1.25 m², and the anti-TIGIT antagonist antibody is administered at a dose of between 10 mg to 1000 mg every three weeks (e.g., 450 mg every three weeks); or (d) greater than 1.25 m², and the anti-TIGIT antagonist antibody is administered at a dose of between 30 mg to 1200 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between 250 mg to 350 mg every three weeks (e.g., 300 mg every three weeks); (b) greater than 0.5 m² and less than or equal to 0.75 m², and the anti-TIGIT antagonist antibody is administered at a dose of between 300 mg to 400 mg every three weeks (e.g., 350 mg every three weeks); or (c) greater than 0.75 m² and less than or equal to 1.25 m², and the anti-TIGIT antagonist antibody is administered at a dose of between 400 mg to 500 mg every three weeks (e.g., 450 mg every three weeks); or (d) greater than 1.25 m², and the anti-TIGIT antagonist antibody is administered at a dose of between 550 mg to 650 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of 300 mg every three weeks; (b) greater than 0.5 m² and less than or equal to 0.75 m², and the anti-TIGIT antagonist antibody is administered at a dose of 400 mg every three weeks; (c) greater than 0.75 m² and less than or equal to 1.25 m², and the anti-TIGIT antagonist antibody is administered at a dose of 450 mg every three weeks; or (d) greater than 1.25 m², and the anti-TIGIT antagonist antibody is administered at a dose of 600 mg every three weeks.

In some instances, the effective amount of the anti-TIGIT antagonist antibody (e.g., an anti-TIGIT antagonist antibody as disclosed herein, e.g., tiragolumab) is a dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W) for subject with a body surface area greater than 1.25 m² (e.g., 1.25 m², 1.35 m², 1.45 m², 1.50 m², 1.55 m², 1.60 m², 1.65 m², 1.70 m², 1.75 m², 1.80 m², 1.85 m², 1.90 m², 1.95 m², 2.0 m², 2.1 m², 2.2 m², 2.3 m², 2.4 m², 2.5 m², 2.6 m², 2.7 m², 2.8 m², 2.9 m², 3.0 m² or more). In some instances, the effective amount of the anti-TIGIT antagonist antibody (e.g., an anti-TIGIT antagonist antibody as disclosed herein, e.g., tiragolumab) is a dose of about 600 mg every three weeks for subject with a body surface area greater than 1.25 m². In some instances, the effective amount of the anti-TIGIT antagonist antibody (e.g., an anti-TIGIT antagonist antibody as disclosed herein, e.g., tiragolumab) is a dose of between 30 mg to 1200 mg (e.g., between 30 mg to 1100 mg, e.g., between 60 mg to 1000 mg, e.g., between 100 mg to 900 mg, e.g., between 200 mg to 800 mg, e.g., between 300 mg to 800 mg, e.g., between 400 mg to 800 mg, e.g., between 400 mg to 750 mg, e.g., between 450 mg to 750 mg, e.g., between 500 mg to 700 mg, e.g., between 550 mg to 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W) for subject with a body surface area greater than 1.25 m² (e.g., 1.25 m², 1.35 m², 1.45 m², 1.50 m², 1.55 m², 1.60 m², 1.65 m², 1.70 m², 1.75 m², 1.80 m², 1.85 m², 1.90 m², 1.95 m², 2.0 m², 2.1 m², 2.2 m², 2.3 m², 2.4 m², 2.5 m², 2.6 m², 2.7 m², 2.8 m², 2.9 m², 3.0 m² or more). In some instances, the effective amount of the anti-TIGIT antagonist antibody (e.g., an anti-TIGIT antagonist antibody as disclosed herein, e.g., tiragolumab) is a dose of 600 mg every three weeks for subject with a body surface area greater than 1.25 m².

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 700 mg, e.g., between about 250 mg to about 600 mg, e.g., between about 300 mg to about 500 mg, e.g., between about 400 mg to about 500 mg, e.g., between about 440 mg to about 460 mg, e.g., about 450 mg) every three weeks (Q3W) for subject with a body surface area greater than 0.75 m² and less than or equal to 1.25 m² (e.g., 0.76 m², 0.77 m², 0.78 m², 0.79 m², 0.80 m², 0.82 m², 0.84 m², 0.86 m², 0.88 m², 0.90 m², 0.95 m², 1.0 m², 1.05 m², 1.10 m², 1.15 m², 1.20 m², or 1.25 m²). In some instances, the effective amount of the anti-TIGIT antagonist antibody (e.g., an anti-TIGIT antagonist antibody as disclosed herein, e.g., tiragolumab) is a dose of about 450 mg every three weeks (e.g., 450 mg±10 mg, e.g., 450±6 mg, e.g., 450±5 mg, e.g., 450±3 mg, e.g., 450±1 mg, e.g., 450±0.5 mg, e.g., 450 mg every three weeks) for subject with a body surface area greater than 0.75 m² and less than or equal to 1.25 m².

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 20 mg to about 1000 mg, e.g., between about 50 mg to about 900 mg, e.g., between about 100 mg to about 850 mg, e.g., between about 200 mg to about 700 mg, e.g., between about 250 mg to about 600 mg, e.g., between about 300 mg to about 500 mg, e.g., between about 300 mg to about 400 mg, e.g., between about 340 mg to about 360 mg, e.g., about 350 mg) every three weeks (Q3W) for subject with a body surface area greater than 0.5 m² and less than or equal to 0.75 m² (e.g., 0.51 m², 0.52 m², 0.53 m², 0.54 m², 0.55 m², 0.56 m², 0.57 m², 0.58 m², 0.59 m², 0.60 m², 0.61 m², 0.62 m², 0.63 m², 0.64 m², 0.65 m², 0.66 m², 0.67 m², 0.68 m², 0.69 m², 0.70 m², 0.71 m², 0.72 m², 0.73 m², 0.74 m², or 0.75 m²). In some instances, the effective amount of the anti-TIGIT antagonist antibody (e.g., an anti-TIGIT antagonist antibody as disclosed herein, e.g., tiragolumab) is a dose of about 350 mg every three weeks (e.g., 350 mg±10 mg, e.g., 350±6 mg, e.g., 350±5 mg, e.g., 350±3 mg, e.g., 350±1 mg, e.g., 350±0.5 mg, e.g., 350 mg every three weeks) for subject with a body surface area greater than 0.5 m² and less than or equal to 0.75 m².

In some instances, the effective amount of the anti-TIGIT antagonist antibody (e.g., an anti-TIGIT antagonist antibody as disclosed herein, e.g., tiragolumab) is a dose of between about 10 mg to about 1000 mg (e.g., between about 10 mg to about 900 mg, e.g., between about 50 mg to about 900 mg, e.g., between about 100 mg to about 750 mg, e.g., between about 100 mg to about 600 mg, e.g., between about 150 mg to about 500 mg, e.g., between about 200 mg to about 400 mg, e.g., between about 250 mg to about 350 mg, e.g., between about 290 mg to about 310 mg, e.g., about 300 mg) every three weeks (Q3W) for subject with a body surface area less than or equal to 0.5 m² (e.g., 0.02 m², 0.04 m², 0.06 m², 0.08 m², 0.1 m², 0.15 m², 0.20 m², 0.25 m², 0.30 m², 0.35 m², 0.40 m², 0.45 m², or 0.50 m²). In some instances, the effective amount of the anti-TIGIT antagonist antibody (e.g., an anti-TIGIT antagonist antibody as disclosed herein, e.g., tiragolumab) is a dose of about 300 mg every three weeks (e.g., 300 mg±10 mg, e.g., 300±6 mg, e.g., 300±5 mg, e.g., 300±3 mg, e.g., 300±1 mg, e.g., 300±0.5 mg, e.g., 300 mg every three weeks) for subject with a body surface area less than or equal to 0.5 m².

In some instances, the effective amount of the anti-TIGIT antagonist antibody (e.g., an anti-TIGIT antagonist antibody as disclosed herein, e.g., tiragolumab) is a dose (e.g., a fixed dose) of between about 10 mg to about 1000 mg (e.g., between about 20 mg to about 1000 mg, e.g., between about 50 mg to about 900 mg, e.g., between about 100 mg to about 850 mg, e.g., between about 200 mg to about 800 mg, e.g., between about 300 mg to about 600 mg, e.g., between about 400 mg to about 500 mg, e.g., between about 405 mg to about 450 mg, e.g., between about 410 mg to about 430 mg, e.g., about 420 mg) every two weeks (Q2W). In some instances, the effective amount of the anti-TIGIT antagonist antibody (e.g., an anti-TIGIT antagonist antibody as disclosed herein, e.g., tiragolumab) is a dose of about 420 mg every two weeks (e.g., 420 mg±10 mg, e.g., 420±6 mg, e.g., 420±5 mg, e.g., 420±3 mg, e.g., 420±1 mg, e.g., 420±0.5 mg, e.g., 420 mg every two weeks). In some instances, the method comprises administering to the subject or population of subjects the anti-TIGIT antagonist antibody at a dose of about 300 mg to about 600 mg every two weeks. In some instances, the method comprises administering to the subject or population of subjects the anti-TIGIT antagonist antibody at a dose of 300 mg to 600 mg every two weeks. In some instances, the method comprises administering to the subject or population of subjects the anti-TIGIT antagonist antibody (e.g., tiragolumab) at a dose of about 420 every two weeks. In some instances, the method comprises administering to the subject or population of subjects the anti-TIGIT antagonist antibody (e.g., tiragolumab) at a dose of 420 every two weeks. In some instances, the dose of the anti-TIGIT antagonist antibody (e.g., tiragolumab) is a fixed dose.

In some instances, the effective amount of the anti-TIGIT antagonist antibody (e.g., an anti-TIGIT antagonist antibody as disclosed herein, e.g., tiragolumab) is a dose (e.g., a fixed dose) of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks (Q3W). In some instances, the effective amount of the anti-TIGIT antagonist antibody (e.g., an anti-TIGIT antagonist antibody as disclosed herein, e.g., tiragolumab) is a dose of about 600 mg every three weeks. In some instances, the method comprises administering to the subject or population of subjects the anti-TIGIT antagonist antibody (e.g., tiragolumab) at a dose of about 600 every three weeks. In some instances, the method comprises administering to the subject or population of subjects the anti-TIGIT antagonist antibody (e.g., tiragolumab) at a dose of 600 mg every three weeks. In some instances, the dose of the anti-TIGIT antagonist antibody (e.g., tiragolumab) is a fixed dose.

In some instances, the effective amount of the anti-TIGIT antagonist antibody (e.g., an anti-TIGIT antagonist antibody as disclosed herein) is a dose of between about 200 mg to about 2000 mg (e.g., between about 200 mg to about 2000 mg, e.g., between about 400 mg to about 1900 mg, e.g., between about 500 mg to about 1800 mg, e.g., between about 600 mg to about 1700 mg, e.g., between about 700 mg to about 1400 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., about 1200 mg, e.g., 1200 mg±10 mg, e.g., 1200±6 mg, e.g., 1200±5 mg, e.g., 1200±3 mg, e.g., 1200±1 mg, e.g., 1200±0.5 mg, e.g., 1200 mg) every three weeks (Q3W). In some instances, the effective amount of the anti-TIGIT antagonist antibody (e.g., an anti-TIGIT antagonist antibody as disclosed herein) is a dose of about 600 mg every three weeks. In some instances, the effective amount of the anti-TIGIT antagonist antibody (e.g., an anti-TIGIT antagonist antibody as disclosed herein) is a dose of 600 mg every three weeks.

In some instances, the effective amount of the anti-TIGIT antagonist antibody (e.g., an anti-TIGIT antagonist antibody as disclosed herein, e.g., tiragolumab) is a dose of between about 200 mg to about 2000 mg (e.g., between about 200-300 mg, between about 300-400 mg, between about 400-500 mg, between about 500-600 mg, between about 600-700 mg, between about 700-800 mg, between about 800-900 mg, between about 900-1000 mg, between about 1000-1100 mg, between about 1100-1200 mg, between about 1200-1300 mg, between about 1300-1400 mg, between about 1400-1500 mg, between about 1500-1600 mg, between about 1600-1700 mg, between about 1700-1800 mg, between about 1800-1900 mg, or between about 1900-2000 mg, e.g., between about 200 mg to about 1600 mg, e.g., between about 250 mg to about 1600 mg, e.g., between about 300 mg to about 1600 mg, e.g., between about 400 mg to about 1500 mg, e.g., between about 500 mg to about 1400 mg, e.g., between about 600 mg to about 1200 mg, e.g., between about 700 mg to about 1100 mg, e.g., between about 800 mg to about 1000 mg, e.g., between about 800 mg to about 900 mg, e.g., about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600 mg, about 1650 mg, about 1700 mg, about 1750 mg, about 1800 mg, about 1850 mg, about 1900 mg, about 1950 mg, or about 2000 mg, e.g., about 800, about 810, about 820, about 830, about 840, about 850, about 860, about 870, about 880, about 890, or about 900 mg) every four weeks (Q4W). In some instances, the effective amount of the anti-TIGIT antagonist antibody (e.g., an anti-TIGIT antagonist antibody as disclosed herein, e.g., tiragolumab) is about 700 mg to about 1000 mg every four weeks. In some instances, the effective amount of the anti-TIGIT antagonist antibody (e.g., an anti-TIGIT antagonist antibody as disclosed herein, e.g., tiragolumab) is 700 mg to 1000 mg every four weeks. In some instances, the effective amount of the anti-TIGIT antagonist antibody (e.g., an anti-TIGIT antagonist antibody as disclosed herein, e.g., tiragolumab) is about 840 mg every four weeks. In some instances, the effective amount of the anti-TIGIT antagonist antibody (e.g., an anti-TIGIT antagonist antibody as disclosed herein, e.g., tiragolumab) is 840 mg every four weeks. The 840 mg Q4W dosing regimen is supported by results from PK modeling and simulation and exposure-safety analyses. Briefly, the average concentration following the 840 mg Q4W dosing regimen is similar to that of the 600 mg every 3 weeks dosing regimen, which was evaluated in previous studies. The Cmax of the 840 mg Q4W dosing regimen was simulated to be 28% higher at steady state, relative to the Cmax for the 600 mg every 3 weeks dosing regimen, but falls within the range of observed exposure of the highest administered dose in the clinic (1200 mg every 3 weeks). A preliminary analysis of the tiragolumab exposure-safety relationship based on previous observations (tiragolumab doses of 2-1200 mg every 3 weeks administered as monotherapy or in combination with atezolizumab 1200 mg every 3 weeks) suggest that tiragolumab exhibits a flat exposure-safety relationship. In summary, the 840 mg Q4W dosing regimen can provide comparable safety and efficacy as the 600 mg every-3-weeks dosing regimen, given that the predicted exposure is within the range of observed efficacious exposures and tiragolumab exhibits a flat exposure-safety relationship.

In some instances, the effective amount of the anti-TIGIT antagonist antibody (e.g., an anti-TIGIT antagonist antibody as disclosed herein, e.g., tiragolumab) is a dose of between about 200 mg to about 2000 mg (e.g., between about 200 mg to about 2000 mg, e.g., between about 400 mg to about 1900 mg, e.g., between about 500 mg to about 1800 mg, e.g., between about 600 mg to about 1700 mg, e.g., between about 700 mg to about 1400 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg (e.g., between 200 mg to 2000 mg, e.g., between 400 mg to 1900 mg, e.g., between 500 mg to 1800 mg, e.g., between 600 mg to 1700 mg, e.g., between 700 mg to 1400 mg, e.g., between 800 mg to 1600 mg, e.g., between 900 mg to 1500 mg, e.g., between 1000 mg to 1400 mg, e.g., between 1050 mg to 1350 mg, e.g., between 1100 mg to 1300 mg, e.g., between 1150 mg to 1250 mg, e.g., between 1175 mg to 1225 mg, e.g., between 1190 mg to 1210 mg), e.g., about 1200 mg, e.g., 1200 mg±10 mg, e.g., 1200±6 mg, e.g., 1200±5 mg, e.g., 1200±3 mg, e.g., 1200±1 mg, e.g., 1200±0.5 mg, e.g., 1200 mg) every four weeks (Q4W). In some instances, the effective amount of anti-TIGIT antagonist antibody (e.g., an anti-TIGIT antagonist antibody as disclosed herein, e.g., tiragolumab) is a dose of about 840 mg every four weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every four weeks). In some instances, the effective amount of anti-TIGIT antagonist antibody (e.g., an anti-TIGIT antagonist antibody as disclosed herein, e.g., tiragolumab) is a dose of 840 mg every four weeks. In some instances, the dose of the anti-TIGIT antagonist antibody (e.g., tiragolumab) is a fixed dose.

In some instances, the effective amount of the anti-TIGIT antagonist antibody (e.g., an anti-TIGIT antagonist antibody as disclosed herein) is a dose of about 1200 mg every four weeks.

In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., anti-PD-L1 antagonist antibody (e.g., atezolizumab) or anti-PD-1 antagonist antibody (e.g., pembrolizumab)) may be reduced as compared to a standard dose of the anti-TIGIT antagonist antibody administered as a monotherapy.

In some instances, the anti-TIGIT antagonist antibody (e.g., an anti-TIGIT antagonist antibody as disclosed herein, e.g., tiragolumab) is administered intravenously. Alternatively, in some embodiments, the anti-TIGIT antagonist antibody (e.g., an anti-TIGIT antagonist antibody as disclosed herein, e.g., tiragolumab) is administered subcutaneously. In some instances, tiragolumab is administered to the patient intravenously at a dose of about 420 mg every 2 weeks, about 600 mg every 3 weeks, or about 840 mg of every 4 weeks. In some instances, tiragolumab is administered to the patient intravenously at a dose of 420 mg every 2 weeks, 600 mg every 3 weeks, or 840 mg of every 4 weeks.

In some instances, a subject is administered a total of 1 to 20 doses of an anti-TIGIT antagonist antibody (e.g., an anti-TIGIT antagonist antibody as disclosed herein, e.g., tiragolumab), e.g., 1, 2, 3, 4, 5, 6, 7, 8, 9, 10, 11, 12, 13, 14, 15, 16, 17, 18, 19, or 20 doses. In some instances, a subject is administered a total of 1 to 50 doses of an anti-TIGIT antagonist antibody (e.g., an anti-TIGIT antagonist antibody as disclosed herein, e.g., tiragolumab), e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

ii. Dosing of PD-1 Axis Binding Antagonists

As a general proposition, the therapeutically effective amount of a PD-1 axis binding antagonist (e.g., atezolizumab) administered to a human will be in the range of about 0.01 to about 50 mg/kg of patient body weight, whether by one or more administrations.

In some exemplary embodiments, the PD-1 axis binding antagonist is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In some exemplary embodiments, the PD-1 axis binding antagonist is administered in a dose of 0.01 to 45 mg/kg, 0.01 to 40 mg/kg, 0.01 to 35 mg/kg, 0.01 to 30 mg/kg, 0.01 to 25 mg/kg, 0.01 to 20 mg/kg, 0.01 to 15 mg/kg, 0.01 to 10 mg/kg, 0.01 to 5 mg/kg, or 0.01 to 1 mg/kg administered daily, weekly, every two weeks, every three weeks, or every four weeks, for example.

In some instances, the PD-1 axis binding antagonist is administered on about Day 1 (e.g., Day −3, Day −2, Day −1, Day 1, Day 2, or Day 3) of a dosing cycle.

In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose (e.g., a fixed dose) of between about 20 mg to about 1600 mg (e.g., between about 40 mg to about 1500 mg, e.g., between about 200 mg to about 1400 mg, e.g., between about 300 mg to about 1400 mg, e.g., between about 400 mg to about 1400 mg, e.g., between about 500 mg to about 1300 mg, e.g., between about 600 mg to about 1200 mg, e.g., between about 700 mg to about 1100 mg, e.g., between about 800 mg to about 1000 mg, e.g., between about 800 mg to about 900 mg, e.g., about 800, about 810, about 820, about 830, about 840, about 850, about 860, about 870, about 880, about 890, or about 900 mg) every two weeks (Q2W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose (e.g., a fixed dose) of between 20 mg to 1600 mg (e.g., between 40 mg to 1500 mg, e.g., between 200 mg to 1400 mg, e.g., between 300 mg to 1400 mg, e.g., between 400 mg to 1400 mg, e.g., between 500 mg to 1300 mg, e.g., between 600 mg to 1200 mg, e.g., between 700 mg to 1100 mg, e.g., between 800 mg to 1000 mg, e.g., between 800 mg to 900 mg, e.g., 800, 810, 820, 830, 840, 850, 860, 870, 880, 890, or 900 mg) every two weeks (Q2W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of about 840 mg every two weeks (e.g., 840 mg±10 mg, e.g., 840±6 mg, e.g., 840±5 mg, e.g., 840±3 mg, e.g., 840±1 mg, e.g., 840±0.5 mg, e.g., 840 mg every two weeks). In some instances, the effective amount of atezolizumab is a dose of about 840 mg every two weeks.

In some instances, the effective amount of the PD-1 axis binding antagonist (e.g., anti-PD-1 antagonist antibody (e.g., pembrolizumab) or anti-PD-L1 antagonist antibody (e.g., atezolizumab)) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 5 mg/kg to about 15 mg/kg, e.g., between about 7.5 mg/kg to about 12.5 mg/kg, e.g., about 10±2 mg/kg, about 10±1 mg/kg, about 10±0.5 mg/kg, about 10±0.2 mg/kg, or about 10±0.1 mg/kg, e.g., about 10 mg/kg) every two weeks. In some instances, the effective amount of the PD-1 axis binding antagonist (e.g., anti-PD-1 antagonist antibody (e.g., pembrolizumab) or anti-PD-L1 antagonist antibody (e.g., atezolizumab)) is a dose of between about 0.01 mg/kg to about 10 mg/kg of the subject's body weight (e.g., between about 0.1 mg/kg to about 10 mg/kg, e.g., between about 0.5 mg/kg to about 10 mg/kg, e.g., between about 1 mg/kg to about 10 mg/kg, e.g., between about 2.5 mg/kg to about 10 mg/kg, e.g., between about 5 mg/kg to about 10 mg/kg, e.g., between about 7.5 mg/kg to about 10 mg/kg, e.g., between about 8 mg/kg to about 10 mg/kg, e.g., between about 9 mg/kg to about 10 mg/kg, e.g., between about 9.5 mg/kg to about 10 mg/kg, e.g., about 10±1 mg/kg, e.g., about 10±0.5 mg/kg, e.g., about 10±0.2 mg/kg, e.g., about 10±0.1 mg/kg, e.g., about 10 mg/kg) every two weeks. In some instances, the effective amount of the PD-1 axis binding antagonist (e.g., anti-PD-1 antagonist antibody (e.g., pembrolizumab) or anti-PD-L1 antagonist antibody (e.g., atezolizumab)) is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 5 mg/kg to 15 mg/kg, e.g., between 7.5 mg/kg to 12.5 mg/kg, e.g., 10±2 mg/kg, 10±1 mg/kg, 10±0.5 mg/kg, 10±0.2 mg/kg, or 10±0.1 mg/kg, e.g., 10 mg/kg) every two weeks. In some instances, the effective amount of the PD-1 axis binding antagonist (e.g., anti-PD-1 antagonist antibody (e.g., pembrolizumab) or anti-PD-L1 antagonist antibody (e.g., atezolizumab)) is a dose of between 0.01 mg/kg to 10 mg/kg of the subject's body weight (e.g., between 0.1 mg/kg to 10 mg/kg, e.g., between 0.5 mg/kg to 10 mg/kg, e.g., between 1 mg/kg to 10 mg/kg, e.g., between 2.5 mg/kg to 10 mg/kg, e.g., between 5 mg/kg to 10 mg/kg, e.g., between 7.5 mg/kg to 10 mg/kg, e.g., between 8 mg/kg to 10 mg/kg, e.g., between 9 mg/kg to 10 mg/kg, e.g., between 9.5 mg/kg to 10 mg/kg, e.g., 10±1 mg/kg, e.g., 10±0.5 mg/kg, e.g., 10±0.2 mg/kg, e.g., 10±0.1 mg/kg, e.g., 10 mg/kg) every two weeks. In some instances, the effective amount of pembrolizumab is a dose of about 10 mg/kg every two weeks. In some instances, the effective amount of pembrolizumab is a dose of 10 mg/kg every two weeks.

In some instances, the effective amount of the PD-1 axis binding antagonist (e.g., anti-PD-L1 antagonist antibody (e.g., atezolizumab)) to treat a subject having a cancer is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the PD-1 axis binding antagonist (e.g., anti-PD-L1 antagonist antibody (e.g., atezolizumab)) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of the PD-1 axis binding antagonist (e.g., anti-PD-L1 antagonist antibody (e.g., atezolizumab)) to treat a subject having a cancer is a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) every three weeks. In some instances, the effective amount of the PD-1 axis binding antagonist (e.g., anti-PD-L1 antagonist antibody (e.g., atezolizumab)) is a dose of between 0.01 mg/kg to 15 mg/kg of the subject's body weight (e.g., between 0.1 mg/kg to 15 mg/kg, e.g., between 0.5 mg/kg to 15 mg/kg, e.g., between 1 mg/kg to 15 mg/kg, e.g., between 2.5 mg/kg to 15 mg/kg, e.g., between 5 mg/kg to 15 mg/kg, e.g., between 7.5 mg/kg to 15 mg/kg, e.g., between 10 mg/kg to 15 mg/kg, e.g., between 12.5 mg/kg to 15 mg/kg, e.g., between 14 mg/kg to 15 mg/kg, e.g., 15±1 mg/kg, e.g., 15±0.5 mg/kg, e.g., 15±0.2 mg/kg, e.g., 15±0.1 mg/kg, e.g., 15 mg/kg) every three weeks. In some instances, the effective amount of PD-1 axis binding antagonist (e.g., anti-PD-L1 antagonist antibody (e.g., atezolizumab)) is a dose of about 15 mg/kg administered every three weeks. In some instances, the effective amount of PD-1 axis binding antagonist (e.g., anti-PD-L1 antagonist antibody (e.g., atezolizumab)) is a dose of about 15 mg/kg administered every three weeks with a maximum dose of 1200 mg every three weeks. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist administered as a monotherapy. In some embodiments, the PD-1 axis binding antagonist (e.g., anti-PD-L1 antagonist antibody (e.g., atezolizumab)) is administered at a maximum dose of 1200 mg every three weeks.

In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between about 80 mg to about 2000 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200 mg) every three weeks (Q3W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of about 1200 mg every three weeks (e.g., 1200 mg±10 mg, e.g., 1200±6 mg, e.g., 1200±5 mg, e.g., 1200±3 mg, e.g., 1200±1 mg, e.g., 1200±0.5 mg, e.g., 1200 mg every three weeks). In some instances, the effective amount of atezolizumab is a dose of 1200 mg every three weeks.

In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between about 10 mg and about 800 mg (e.g., between about 10 mg and about 800 mg, e.g., between about 20 mg and about 700 mg, e.g., between about 50 mg and about 600 mg, e.g., between about 75 mg and about 500 mg, e.g., between about 100 mg and about 400 mg, e.g., between about 100 mg and about 300 mg, e.g., between about 125 mg and about 275 mg, e.g., between about 150 mg and about 250 mg, e.g., between about 175 mg and about 225 mg, e.g., between about 190 mg and about 210 mg, e.g., about 200 mg±10 mg, e.g., 200 mg±7.5 mg, e.g., 200 mg±5 mg, e.g., 200±2.5 mg, e.g., 200±1.0 mg, e.g., 200±0.5 mg, e.g., 200 mg) every three weeks (Q3W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of about 200 mg every three weeks (e.g., 200 mg±10 mg, e.g., 200±6 mg, e.g., 200±5 mg, e.g., 200±3 mg, e.g., 200±1 mg, e.g., 200±0.5 mg, e.g., 200 mg every three weeks). In some instances, the effective amount of the anti-PD-1 antagonist antibody (e.g., pembrolizumab)) is a dose of about 200 mg every three weeks (e.g., 200 mg±10 mg, e.g., 200±6 mg, e.g., 200±5 mg, e.g., 200±3 mg, e.g., 200±1 mg, e.g., 200±0.5 mg, e.g., 200 mg every three weeks). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between 10 mg and 800 mg (e.g., between 10 mg and 800 mg, e.g., between 20 mg and 700 mg, e.g., between 50 mg and 600 mg, e.g., between 75 mg and 500 mg, e.g., between 100 mg and 400 mg, e.g., between 100 mg and 300 mg, e.g., between 125 mg and 275 mg, e.g., between 150 mg and 250 mg, e.g., between 175 mg and 225 mg, e.g., between 190 mg and 210 mg, e.g., 200 mg±10 mg, e.g., 200 mg±7.5 mg, e.g., 200 mg±5 mg, e.g., 200±2.5 mg, e.g., 200±1.0 mg, e.g., 200±0.5 mg, e.g., 200 mg) every three weeks (Q3W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of 200 mg every three weeks (e.g., 200 mg±10 mg, e.g., 200±6 mg, e.g., 200±5 mg, e.g., 200±3 mg, e.g., 200±1 mg, e.g., 200±0.5 mg, e.g., 200 mg every three weeks). In some instances, the effective amount of the anti-PD-1 antagonist antibody (e.g., pembrolizumab)) is a dose of 200 mg every three weeks (e.g., 200 mg±10 mg, e.g., 200±6 mg, e.g., 200±5 mg, e.g., 200±3 mg, e.g., 200±1 mg, e.g., 200±0.5 mg, e.g., 200 mg every three weeks). In some instances, the effective amount of pembrolizumab is a dose of about 200 mg every three weeks. In some instances, the effective amount of pembrolizumab is a dose of 200 mg every three weeks.

In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between about 80 mg to about 3000 mg (e.g., between about 80-200 mg, between about 200-400 mg, between about 400-600 mg, between about 600-800 mg, between about 800-1000 mg, between about 1000-1200 mg, between about 1200-1400 mg, between about 1400-1600 mg, between about 1600-1800 mg, between about 1800-2000 mg, between about 2200-2400 mg, between about 2400-2600 mg, between about 2600-2800 mg, or between about 2800-3000 mg, e.g., between about 100 mg and about 3000 mg, e.g., between about 200 mg and about 2900 mg, e.g., between about 500 mg to about 2800 mg, e.g., between about 600 mg to about 2700 mg, e.g., between about 650 mg to about 2600 mg, e.g., between about 700 mg to about 2500 mg, e.g., between about 1000 mg to about 2400 mg, e.g., between about 1100 mg to about 2300 mg, e.g., between about 1200 mg to about 2200 mg, e.g., between about 1300 mg to about 2100 mg, e.g., between about 1400 mg to about 2000 mg, e.g., between about 1500 mg to about 1900 mg, e.g., between about 1600 mg to about 1800 mg, e.g., between about 1620 mg to about 1700 mg, e.g., between about 1640 mg to about 1690 mg, e.g., between about 1660 mg to about 1680 mg, about 1680 mg, e.g., about 80 mg, about 200 mg, about 400 mg, about 600 mg, about 800 mg, about 1000 mg, about 1200 mg, about 1400 mg, about 1600 mg, about 1800 mg, about 2000 mg, about 2200 mg, about 2400 mg, about 2600 mg, about 2800 mg, or about 3000 mg, e.g., about 1600 mg, about 1610 mg, about 1620 mg, about 1630 mg, about 1640 mg, about 1650 mg, about 1660 mg, about 1670 mg, about 1680 mg, about 1690 mg, or about 1700 mg) every four weeks (Q4W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of between 500 mg to 3000 mg (e.g., between 500 mg to 2800 mg, e.g., between 600 mg to 2700 mg, e.g., between 650 mg to 2600 mg, e.g., between 700 mg to 2500 mg, e.g., between 1000 mg to 2400 mg, e.g., between 1100 mg to 2300 mg, e.g., between 1200 mg to 2200 mg, e.g., between 1300 mg to 2100 mg, e.g., between 1400 mg to 2000 mg, e.g., between 1500 mg to 1900 mg, e.g., between 1600 mg to 1800 mg, e.g., between 1620 mg to 1700 mg, e.g., between 1640 mg to 1690 mg, e.g., between 1660 mg to 1680 mg, 1680 mg, e.g., 1600 mg, 1610 mg, 1620 mg, 1630 mg, 1640 mg, 1650 mg, 1660 mg, 1670 mg, 1680 mg, 1690 mg, or 1700 mg) every four weeks (Q4W). In some instances, the effective amount of the PD-1 axis binding antagonist (e.g., anti-PD-L1 antagonist antibody (e.g., atezolizumab) or anti-PD-1 antagonist antibody (e.g., pembrolizumab)) is a dose of 1680 mg every four weeks (e.g., 1680 mg±10 mg, e.g., 1680±6 mg, e.g., 1680±5 mg, e.g., 1680±3 mg, e.g., 1680±1 mg, e.g., 1680±0.5 mg, e.g., 1680 mg every four weeks). In some instances, the effective amount of atezolizumab is a dose of about 1680 mg every four weeks. In some instances, the effective amount of atezolizumab is a dose of 1680 mg every four weeks.

In some instances, the effective amount of an anti-PD-1 antagonist antibody (e.g., pembrolizumab) is a dose of between about 50 mg to about 2000 mg (e.g., between about 50-100 mg, between about 100-250 mg, between about 250-500 mg, between about 500-750 mg, between about 750-1000 mg, between about 1000-1250 mg, between about 1250-1500 mg, between about 1500-1750 mg, or between about 1750-2000 mg, e.g., between about 100 mg to about 1000 mg, between about 120 mg to about 900 mg, between about 150 mg to about 800 mg, between about 200 mg to about 700 mg, between about 250 mg to about 600 mg, between about 300 mg to about 500 mg, or between about 350 mg to about 450 mg, e.g., between about 50 mg to about 100 mg, between about 100 mg to about 200 mg, between about 200 mg to about 300 mg, between about 300 mg to about 400 mg, between about 400 mg to about 500 mg, between about 500 mg to about 600 mg, between about 600 mg to about 700 mg, between about 700 mg to about 800 mg, or between about 800 mg to about 1000 mg, e.g., about 50 mg, about 100 mg, about 250 mg, about 500 mg, about 750 mg, about 1000 mg, about 1250 mg, about 1500 mg, about 1750 mg, or about 2000 mg, e.g., about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, e.g., 400 mg) every six weeks (Q6W). In some instances, the effective amount of an anti-PD-1 antagonist antibody (e.g., pembrolizumab) is a dose of between 50 mg to 2000 mg (e.g., between 100 mg to 1000 mg, between 120 mg to 900 mg, between 150 mg to 800 mg, between 200 mg to 700 mg, between 250 mg to 600 mg, between 300 mg to 500 mg, or between 350 mg to 450 mg, e.g., between 50 mg to 100 mg, between 100 mg to 200 mg, between 200 mg to 300 mg, between 300 mg to 400 mg, between 400 mg to 500 mg, between 500 mg to 600 mg, between 600 mg to 700 mg, between 700 mg to 800 mg, or between 800 mg to 1000 mg, e.g., 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg, e.g., 400 mg) every six weeks (Q6W). In some instances, the effective amount of the anti-PD-1 antagonist antibody (e.g., pembrolizumab) is a dose of about 400 mg every six weeks (e.g., 400 mg±10 mg, e.g., 400±6 mg, e.g., 400±5 mg, e.g., 400±3 mg, e.g., 400±1 mg, e.g., 400±0.5 mg, e.g., 400 mg every six weeks). In some instances, the dose of the PD-1 axis binding antagonist is a fixed dose. In some instances, the effective amount of pembrolizumab is a dose of (e.g., a fixed dose) about 400 mg every six weeks. In some instances, the effective amount of pembrolizumab is a dose (e.g., a fixed dose) of 400 mg every six weeks.

In some instances, the dose of the PD-1 axis binding antagonist (e.g., anti-PD-L1 antagonist antibody (e.g., atezolizumab) or anti-PD-1 antagonist antibody (e.g., pembrolizumab)) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the PD-1 axis binding antagonist administered as a monotherapy.

In some instances, the PD-1 axis binding antagonist (e.g., anti-PD-L1 antagonist antibody (e.g., atezolizumab) or anti-PD-1 antagonist antibody (e.g., pembrolizumab)) is administered intravenously. Alternatively, in some embodiments, the PD-1 axis binding antagonist (e.g., anti-PD-L1 antagonist antibody (e.g., atezolizumab) or anti-PD-1 antagonist antibody (e.g., pembrolizumab)) is administered subcutaneously. In some instances, atezolizumab is administered to the patient intravenously at a dose of about 840 mg every 2 weeks, about 1200 mg every 3 weeks, or about 1680 mg every 4 weeks. In some instances, atezolizumab is administered to the patient intravenously at a dose of 840 mg every 2 weeks, 1200 mg every 3 weeks, or 1680 mg every 4 weeks.

In some instances, a subject is administered a total of 1 to 20 doses of a PD-1 axis binding antagonist, e.g., 1, 2, 3, 4, 5, 6, 7, 8, 9, 10, 11, 12, 13, 14, 15, 16, 17, 18, 19, or 20 doses. In some instances, a subject is administered a total of 1 to 50 doses of a PD-1 axis binding antagonist, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

iii. Dosing Cycles for Anti-TIGIT Antagonist Antibodies and PD-1 Axis Binding Antagonists

In any of the methods and uses of the invention, the anti-TIGIT antagonist antibody (e.g., an anti-TIGIT antagonist antibody as disclosed herein, e.g., tiragolumab) and/or the PD-1 axis binding antagonist (e.g., anti-PD-L1 antagonist antibody (e.g., atezolizumab)) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) continue until there is a loss of clinical benefit (e.g., confirmed disease progression, drug resistance, death, or unacceptable toxicity). In some instances, the length of each dosing cycle is about 7 to 42 days (e.g., 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 41 days, 42 days). In some instances, the length of each dosing cycle is about 14 days. In some instances, the length of each dosing cycle is about 21 days. In some instances, the length of each dosing cycle is about 28 days. In some instances, the length of each dosing cycle is about 42 days. In some instances, the length of each dosing cycle is about 7 days. In some instances, the anti-TIGIT antagonist antibody (e.g., an anti-TIGIT antagonist antibody as disclosed herein, e.g., tiragolumab) is administered on about Day 1 (e.g., Day 1±3 days) of each dosing cycle. In some instances, the anti-TIGIT antagonist antibody (e.g., an anti-TIGIT antagonist antibody as disclosed herein, e.g., tiragolumab) is administered on about Day 15 (e.g., Day 15±3 days) of each dosing cycle. In some instances, the anti-TIGIT antagonist antibody (e.g., an anti-TIGIT antagonist antibody as disclosed herein, e.g., tiragolumab) is administered on about Day 22 (e.g., Day 22±3 days) of each dosing cycle. In some instances, the anti-TIGIT antagonist antibody (e.g., an anti-TIGIT antagonist antibody as disclosed herein, e.g., tiragolumab) is administered on about Day 29 (e.g., Day 29±3 days) of each dosing cycle. For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 of each 21-day cycle (i.e., at a dose of about 600 mg every three weeks). For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 and Day 15 of each 28-day cycle (i.e., at a dose of about 420 mg every two weeks). For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1, Day 15, and Day 29 of each 42-day cycle (i.e., at a dose of about 420 mg every two weeks). For example, the anti-TIGIT antagonist antibody (e.g., an anti-TIGIT antagonist antibody as disclosed herein, e.g., tiragolumab) is administered intravenously at a dose (e.g., a fixed dose) of about 600 mg on Day 1 and Day 22 of each 42-day cycle (i.e., at a dose of about 600 mg every three weeks). In some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is administered on about Day 1 (e.g., Day 1±3 days) of each dosing cycle. In some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is administered on about Day 15 (e.g., Day 15±3 days) of each dosing cycle. For example, the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). For example, the PD-1 axis binding antagonist (e.g., anti-PD-L1 antagonist antibody (e.g., atezolizumab)) is administered intravenously at a dose of about 1200 mg on Day 1 and Day 15 of each 28-day cycle (i.e., at a dose of about 840 mg every two weeks). In some examples, the anti-TIGIT antagonist antibody (e.g., an anti-TIGIT antagonist antibody as disclosed herein, e.g., tiragolumab) is administered intravenously at a dose (e.g., a fixed dose) of 600 mg on Day 1 of each 21-day cycle (i.e., at a dose of 600 mg every three weeks). In some instances, the PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) is administered on Day 1 (e.g., Day 1±3 days) of each dosing cycle. For example, the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks).

In some instances, the anti-TIGIT antagonist antibody (e.g., an anti-TIGIT antagonist antibody as disclosed herein, e.g., tiragolumab) and the PD-1 axis binding antagonist (e.g., anti-PD-L1 antagonist antibody (e.g., atezolizumab)) are administered on about Day 1 (e.g., Day 1±3 days) of each dosing cycle.

In some instances, the anti-TIGIT antagonist antibody (e.g., an anti-TIGIT antagonist antibody as disclosed herein, e.g., tiragolumab) is administered intravenously at a dose of about 600 mg on Day 1 of each 21-day cycle (i.e., at a dose of about 600 mg every three weeks) and the PD-1 axis binding antagonist (e.g., anti-PD-L1 antagonist antibody (e.g., atezolizumab)) is administered intravenously at a dose of about 1200 mg on Day 1 of each 21-day cycle (i.e., at a dose of about 1200 mg every three weeks). In some instances, the anti-TIGIT antagonist antibody (e.g., an anti-TIGIT antagonist antibody as disclosed herein, e.g., tiragolumab) is administered intravenously at a dose of 600 mg on Day 1 of each 21-day cycle (i.e., at a dose of 600 mg every three weeks) and the PD-1 axis binding antagonist (e.g., anti-PD-L1 antagonist antibody (e.g., atezolizumab)) is administered intravenously at a dose of 1200 mg on Day 1 of each 21-day cycle (i.e., at a dose of 1200 mg every three weeks).

In other instances, the anti-TIGIT antagonist antibody (e.g., an anti-TIGIT antagonist antibody as disclosed herein, e.g., tiragolumab) is administered intravenously at a dose of about 420 mg on Day 1 of each 14-day cycle (i.e., at a dose of about 420 mg every two weeks) and the PD-1 axis binding antagonist (e.g., anti-PD-L1 antagonist antibody (e.g., atezolizumab)) is administered intravenously at a dose of about 840 mg on Day 1 of each 14-day cycle (i.e., at a dose of about 840 mg every two weeks).

In some instances, the anti-TIGIT antagonist antibody (e.g., an anti-TIGIT antagonist antibody as disclosed herein, e.g., tiragolumab) is administered intravenously at a dose of 420 mg on Day 1 of each 14-day cycle (i.e., at a dose of about 420 mg every two weeks) and the PD-1 axis binding antagonist (e.g., anti-PD-L1 antagonist antibody (e.g., atezolizumab)) is administered intravenously at a dose of 840 mg on Day 1 of each 14-day cycle (i.e., at a dose of 840 mg every two weeks).

In other instances, the anti-TIGIT antagonist antibody (e.g., an anti-TIGIT antagonist antibody as disclosed herein, e.g., tiragolumab) is administered intravenously at a dose of about 840 mg on Day 1 of each 28-day cycle (i.e., at a dose of about 840 mg every four weeks) and the PD-1 axis binding antagonist (e.g., anti-PD-L1 antagonist antibody (e.g., atezolizumab)) is administered intravenously at a dose of about 1680 mg on Day 1 of each 28-day cycle (i.e., at a dose of about 1680 mg every four weeks). In some instances, the anti-TIGIT antagonist antibody (e.g., an anti-TIGIT antagonist antibody as disclosed herein, e.g., tiragolumab) is administered intravenously at a dose of 840 mg on Day 1 of each 28-day cycle (i.e., at a dose of 840 mg every four weeks) and the PD-1 axis binding antagonist (e.g., anti-PD-L1 antagonist antibody (e.g., atezolizumab)) is administered intravenously at a dose of 1680 mg on Day 1 of each 28-day cycle (i.e., at a dose of 1680 mg every four weeks).

In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., an anti-PD-L1 antagonist antibody (e.g., atezolizumab) or an anti-PD-1 antagonist antibody (e.g., MDX-1106 (nivolumab) or MK-3475 (pembrolizumab, previously known as lambrolizumab))) may be reduced as compared to a standard dose of the anti-TIGIT antagonist antibody administered as a monotherapy. In some instances, the dose of the anti-TIGIT antagonist antibody (e.g., an anti-TIGIT antagonist antibody as disclosed herein, e.g., tiragolumab) administered in a combination therapy (e.g., a combination treatment with a PD-1 axis binding antagonist (e.g., anti-PD-L1 antagonist antibody (e.g., atezolizumab)), with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel or nab-paclitaxel), and/or a topoisomerase II inhibitor (e.g., doxorubicin))) and/or G-CSF or GM-CSF, may be reduced as compared to a standard dose of the anti-TIGIT antagonist antibody administered as a monotherapy.

In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein (e.g., tiragolumab) may be reduced as compared to a standard dose of the anti-PD-L1 antagonist antibody administered as a monotherapy. In some instances, the dose of the PD-1 axis binding antagonist (e.g., anti-PD-L1 antagonist antibody (e.g., atezolizumab)) administered in a combination therapy (e.g., a combination treatment with an anti-TIGIT antagonist antibody, such as an anti-TIGIT antagonist antibody disclosed herein, e.g., tiragolumab), with or without one or more chemotherapeutic agents (e.g., a platinum-based chemotherapeutic agent (e.g., carboplatin or cisplatin) and/or a non-platinum-based chemotherapeutic agent (e.g., an alkylating agent (e.g., cyclophosphamide), a taxane (e.g., paclitaxel, e.g., nab-paclitaxel), and/or a topoisomerase II inhibitor (e.g., doxorubicin))) and/or G-CSF or GM-CSF may be reduced as compared to a standard dose of the PD-1 axis binding antagonist administered as a monotherapy.

iv. Intravenous Infusion and Subcutaneous Administration of Anti-TIGIT Antagonist Antibodies and PD-1 Axis Binding Antagonists

In some instances, the anti-TIGIT antagonist antibody (e.g., an anti-TIGIT antagonist antibody as disclosed herein, e.g., tiragolumab) is administered intravenously. Alternatively, in some embodiments, the anti-TIGIT antagonist antibody (e.g., an anti-TIGIT antagonist antibody as disclosed herein, e.g., tiragolumab) is administered subcutaneously. In some instances, the PD-1 axis binding antagonist (e.g., anti-PD-L1 antagonist antibody (e.g., atezolizumab)) is administered intravenously. Alternatively, in some embodiments, the PD-1 axis binding antagonist (e.g., anti-PD-L1 antagonist antibody (e.g., atezolizumab)) is administered subcutaneously.

In some instances, the anti-TIGIT antagonist antibody (e.g., an anti-TIGIT antagonist antibody as disclosed herein, e.g., tiragolumab) is administered to the subject or population of subjects by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes). In some instances, the anti-TIGIT antagonist antibody (e.g., an anti-TIGIT antagonist antibody as disclosed herein, e.g., tiragolumab) is administered to the subject or population of subjects by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the PD-1 axis binding antagonist (e.g., anti-PD-L1 antagonist antibody (e.g., atezolizumab) is administered to the subject by intravenous infusion over about 60±15 minutes (e.g., about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes).

In some instances, the PD-1 axis binding antagonist (e.g., anti-PD-L1 antagonist antibody (e.g., atezolizumab)) is administered to the subject by intravenous infusion over about 30±10 minutes (e.g., about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, or about 40 minutes).

v. Administration Order and Observation Periods

In some instances in which both an anti-TIGIT antagonist antibody and PD-1 axis binding antagonist are administered to a subject or population of subjects, the anti-TIGIT antagonist antibody (e.g., an anti-TIGIT antagonist antibody as disclosed herein, e.g., tiragolumab) is administered to the subject before the PD-1 axis binding antagonist (e.g., anti-PD-L1 antagonist antibody (e.g., atezolizumab) or anti-PD-1 antagonist antibody (e.g., pembrolizumab)).

In some instances, for example, following administration of the anti-TIGIT antagonist antibody and before administration of the PD-1 axis binding antagonist the method includes an intervening first observation period. In some instances, for example, following administration of the anti-TIGIT antagonist antibody, the PD-1 axis binding antagonist (e.g., anti-PD-L1 antagonist antibody (e.g., atezolizumab)) is administered to the subject. In some instances, the anti-TIGIT antagonist antibody (e.g., an anti-TIGIT antagonist antibody as disclosed herein, e.g., tiragolumab) is first administered to the subject and the PD-1 axis binding antagonist (e.g., anti-PD-L1 antagonist antibody (e.g., atezolizumab)) is administered to the subject following administration of the anti-TIGIT antagonist antibody (e.g., an anti-TIGIT antagonist antibody as disclosed herein, e.g., tiragolumab).

In some instances, the method further includes a second observation period following administration of the PD-1 axis binding antagonist (e.g., anti-PD-L1 antagonist antibody (e.g., atezolizumab)).

In some instances, the method includes both a first observation period following administration of the anti-TIGIT antagonist antibody and second observation period following administration of the PD-1 axis binding antagonist (e.g., anti-PD-L1 antagonist antibody (e.g., atezolizumab)). In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody, the PD-1 axis binding antagonist (e.g., anti-PD-L1 antagonist antibody (e.g., atezolizumab)) during the first or second observation periods. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody or the PD-1 axis binding antagonist (e.g., anti-PD-L1 antagonist antibody (e.g., atezolizumab)) during the first or second.

In some instances, the PD-1 axis binding antagonist (e.g., anti-PD-L1 antagonist antibody (e.g., atezolizumab) or anti-PD-1 antagonist antibody (e.g., pembrolizumab)) is administered to the subject or population of subjects before the anti-TIGIT antagonist antibody (e.g., an anti-TIGIT antagonist antibody as disclosed herein, e.g., tiragolumab). In some instances, for example, following administration of the PD-1 axis binding antagonist (e.g., anti-PD-L1 antagonist antibody (e.g., atezolizumab)) and before administration of the anti-TIGIT antagonist antibody (e.g., an anti-TIGIT antagonist antibody as disclosed herein, e.g., tiragolumab), the method includes an intervening first observation period.

In some instances, the method further includes a second observation period following administration of the anti-TIGIT antagonist antibody (e.g., an anti-TIGIT antagonist antibody as disclosed herein, e.g., tiragolumab).

In some instances, the method includes both a first observation period following administration of the PD-1 axis binding antagonist (e.g., anti-PD-L1 antagonist antibody (e.g., atezolizumab)) and second observation period following administration of the anti-TIGIT antagonist antibody (e.g., an anti-TIGIT antagonist antibody as disclosed herein, e.g., tiragolumab). In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the PD-1 axis binding antagonist (e.g., anti-PD-L1 antagonist antibody (e.g., atezolizumab)) or the anti-TIGIT antagonist antibody (e.g., an anti-TIGIT antagonist antibody as disclosed herein, e.g., tiragolumab), during the first or second observation periods.

In instances in which the first and second observation periods are each about 30 minutes in length, the method may include recording the subject's vital signs (e.g., pulse rate, respiratory rate, blood pressure, and temperature) at about 15±10 minutes after administration of the PD-1 axis binding antagonist (e.g., anti-PD-L1 antagonist antibody (e.g., atezolizumab)), the anti-TIGIT antagonist antibody (e.g., an anti-TIGIT antagonist antibody as disclosed herein, e.g., tiragolumab), during the first or second observation periods.

In some instances, the method further includes administration of a VEGF antagonist (e.g., an anti-VEGF antibody (e.g., bevacizumab)). In some instances, the VEGF antagonist (e.g., an anti-VEGF antibody (e.g., bevacizumab)) is administered after the PD-1 axis binding antagonist (e.g., anti-PD-L1 antagonist antibody (e.g., atezolizumab) or anti-PD-1 antagonist antibody (e.g., pembrolizumab)) and the anti-TIGIT antagonist antibody (e.g., an anti-TIGIT antagonist antibody as disclosed herein, e.g., tiragolumab). In some instances, the VEGF antagonist (e.g., an anti-VEGF antibody (e.g., bevacizumab)) is administered after the second observation period following administration of the anti-TIGIT antagonist antibody (e.g., an anti-TIGIT antagonist antibody as disclosed herein, e.g., tiragolumab) or the PD-1 axis binding antagonist (e.g., anti-PD-L1 antagonist antibody (e.g., atezolizumab) or anti-PD-1 antagonist antibody (e.g., pembrolizumab)).

In some instances, the method further includes a third observation period following administration of the VEGF antagonist (e.g., an anti-VEGF antibody (e.g., bevacizumab)). In some instances, the third observation period is between about 30 minutes to about 120 minutes in length. In some instances, the first observation period, the second observation period, and the third observation period are each between about 30 minutes to about 120 minutes in length.

vi. Combination Dosing of Anti-TIGIT Antagonist Antibodies and PD-1 Axis Binding Antagonists

In some instances, a dose of an effective amount of an anti-TIGIT antagonist antibody (e.g., an anti-TIGIT antagonist antibody as disclosed herein, e.g., tiragolumab) is administered with a dose of an effective amount of a PD-1 axis binding antagonist (e.g., anti-PD-L1 antagonist antibody (e.g., atezolizumab)) in a combination therapy (e.g., a combination treatment of an anti-TIGIT antagonist antibody (e.g., an anti-TIGIT antagonist antibody as disclosed herein, e.g., tiragolumab) with a PD-1 axis binding antagonist (e.g., anti-PD-L1 antagonist antibody (e.g., atezolizumab)), e.g., for treatment of a subject having a cancer. In some instances, an anti-TIGIT antagonist antibody is administered every two weeks as described in Section IV(E)(i) herein and a PD-1 axis binding antagonist is administered every two weeks as described in Section IV(E)(ii) herein. In some instances, an anti-TIGIT antagonist antibody is administered every two weeks as described in Section IV(E)(i) herein and a PD-1 axis binding antagonist is administered every three weeks as described in Section IV(E)(ii) herein. In some instances, an anti-TIGIT antagonist antibody is administered every two weeks as described in Section IV(E)(i) herein and a PD-1 axis binding antagonist is administered every four weeks as described in Section IV(E)(ii) herein. In some instances, an anti-TIGIT antagonist antibody is administered every two weeks as described in Section IV(E)(i) herein and a PD-1 axis binding antagonist is administered every six weeks as described in Section IV(E)(ii) herein. In some instances, an anti-TIGIT antagonist antibody is administered every three weeks as described in Section IV(E)(i) herein and a PD-1 axis binding antagonist is administered every two weeks as described in Section IV(E)(ii) herein. In some instances, an anti-TIGIT antagonist antibody is administered every three weeks as described in Section IV(E)(i) herein and a PD-1 axis binding antagonist is administered every three weeks as described in Section IV(E)(ii) herein. In some instances, an anti-TIGIT antagonist antibody is administered every three weeks as described in Section IV(E)(i) herein and a PD-1 axis binding antagonist is administered every four weeks as described in Section IV(E)(ii) herein. In some instances, an anti-TIGIT antagonist antibody is administered every three weeks as described in Section IV(E)(i) herein and a PD-1 axis binding antagonist is administered every six weeks as described in Section IV(E)(ii) herein. In some instances, an anti-TIGIT antagonist antibody is administered every four weeks as described in Section IV(E)(i) herein and a PD-1 axis binding antagonist is administered every two weeks as described in Section IV(E)(ii) herein. In some instances, an anti-TIGIT antagonist antibody is administered every four weeks as described in Section IV(E)(i) herein and a PD-1 axis binding antagonist is administered every three weeks as described in Section IV(E)(ii) herein. In some instances, an anti-TIGIT antagonist antibody is administered every four weeks as described in Section IV(E)(i) herein and a PD-1 axis binding antagonist is administered every four weeks as described in Section IV(E)(ii) herein. In some instances, an anti-TIGIT antagonist antibody is administered every four weeks as described in Section IV(E)(i) herein and a PD-1 axis binding antagonist is administered every six weeks as described in Section IV(E)(ii) herein. In some instances, an anti-TIGIT antagonist antibody is administered every two, three, or four weeks as described in Section IV(E)(i) herein and a PD-1 axis binding antagonist is administered every two, three, four, or six weeks as described in Section IV(E)(ii) herein.

In some instances, the dose of the anti-TIGIT antagonist antibody (e.g., an anti-TIGIT antagonist antibody as disclosed herein, e.g., tiragolumab) is a dose of about 600 mg every three weeks. In some instances, the dose of the anti-TIGIT antagonist antibody (e.g., an anti-TIGIT antagonist antibody as disclosed herein, e.g., tiragolumab) is a dose of 600 mg every three weeks. In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject) and a PD-1 axis binding antagonist (e.g., an anti-PD-L1 antagonist antibody, such as atezolizumab) at a dose from about 0.01 mg/kg to about 50 mg/kg (e.g., about 15 mg/kg) up to 1200 mg, e.g., every three weeks. In some instances, the anti-TIGIT antagonist antibody (e.g., an anti-TIGIT antagonist antibody as disclosed herein, e.g., tiragolumab) is administered (e.g., every three weeks) in a tiered dosing regimen (e.g., dosing based on body weight (BW) or body surface area (BSA) of a subject) and a PD-1 axis binding antagonist (e.g., an anti-PD-L1 antagonist antibody, such as atezolizumab) at a dose from 0.01 mg/kg to 50 mg/kg (e.g., 15 mg/kg) up to 1200 mg, e.g., every three weeks. Such dosing regimens can be utilized in treatments for subjects having relatively low body weight (e.g., 40 kg or less (e.g., from 5 kg to 40 kg, from 15 kg to 40 kg, or from 5 kg to 15 kg)) and have been developed through biostimulation studies based on extrapolations of pharmacokinetic parameters estimated from adult data. In some instances, the dose of the anti-TIGIT antagonist antibody (e.g., an anti-TIGIT antagonist antibody as disclosed herein, e.g., tiragolumab) is a tiered dose based on a subject's body weight (e.g., body weight (BW)>40 kg: 600 mg, BW>15 kg and ≤40 kg: 400 mg, and BW≤15 kg: 300 mg). In some instances, the dose of the PD-1 axis binding antagonist (e.g., anti-PD-L1 antagonist antibody (e.g., atezolizumab)) is a dose based on a subject's body weight (e.g., 15 mg/kg). In some instances, the dose of the PD-1 axis binding antagonist (e.g., anti-PD-L1 antagonist antibody (e.g., atezolizumab)) is a dose based on a subject's body surface area (e.g., body surface area (BSA)>1.25 m²: 600 mg, BSA>0.75 m² and ≤1.25 m²: 450 mg, BSA>0.5 m² and ≤0.75 m²: 350 mg, and BSA≤0.5 m²: 300 mg). In some instances, the dose (e.g., about 600 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body weight (e.g., 15 mg/kg) every three weeks. In some instances, the tiered dose (e.g., body weight (BW)>40 kg: 600 mg, BW>15 kg and ≤40 kg: 400 mg, and BW S 15 kg: 300 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body weight (e.g., 15 mg/kg) every three weeks. In some instances, the tiered dose (e.g., body weight (BW)>40 kg: 600 mg, BW>15 kg and ≤40 kg: 400 mg, and BW≤15 kg: 300 mg) of the anti-TIGIT antagonist antibody (e.g., an anti-TIGIT antagonist antibody as disclosed herein, e.g., tiragolumab) is administered in combination with a dose of the PD-1 axis binding antagonist (e.g., anti-PD-L1 antagonist antibody (e.g., atezolizumab)) based on a subject's body surface area (e.g., BSA>1.25 m²: 600 mg, BSA>0.75 m² and ≤1.25 m²: 450 mg, BSA>0.5 m² and ≤0.75 m²: 350 mg, and BSA≤0.5 m²: 300 mg) every three weeks. In some embodiments, the PD-1 axis binding antagonist (e.g., anti-PD-L1 antagonist antibody (e.g., atezolizumab)) is administered at a maximum dose of 1200 mg every three weeks. In some instances, the combination therapy is administered with one or more chemotherapeutic agents (e.g., a platinum-based chemotherapeutic agent (e.g., carboplatin or cisplatin) and/or a non-platinum-based chemotherapeutic agent (e.g., an antimetabolite (e.g., pemetrexed or gemcitabine)).

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body weight, wherein the subject has a body weight of (a) less than or equal to 15 kg, and the anti-TIGIT antagonist antibody is administered at a dose of between about 10 mg to about 1000 mg every three weeks (e.g., about 300 mg every three weeks); (b) greater than 15 kg and less than or equal to 40 kg, and the anti-TIGIT antagonist antibody is administered at a dose of between about 10 mg to about 1000 mg every three weeks (e.g., about 400 mg every three weeks); or (c) greater than 40 kg, and the anti-TIGIT antagonist antibody is administered at a dose of between about 30 mg to about 1200 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between about 250 mg to about 350 mg every three weeks (e.g., about 300 mg every three weeks); (b) greater than 15 kg and less than or equal to 40 kg, and the anti-TIGIT antagonist antibody is administered at a dose of between about 350 mg to about 450 mg every three weeks (e.g., about 400 mg every three weeks); or (c) greater than 40 kg, and the anti-TIGIT antagonist antibody is administered at a dose of between about 550 mg to about 650 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of about 300 mg every three weeks; (b) greater than 15 kg and less than or equal to 40 kg, and the anti-TIGIT antagonist antibody is administered at a dose of about 400 mg every three weeks; or (c) greater than 40 kg, and the anti-TIGIT antagonist antibody is administered at a dose of about 600 mg every three weeks. In some instances, a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) is administered in combination with a tiered dose based on a subject's body weight of the anti-TIGIT antagonist antibody (e.g., an anti-TIGIT antagonist antibody as disclosed herein, e.g., tiragolumab), wherein the subject has a body weight of (a) less than or equal to 15 kg, and the anti-TIGIT antagonist antibody is administered at a dose of between about 10 mg to about 1000 mg every three weeks (e.g., about 300 mg every three weeks); (b) greater than 15 kg and less than or equal to 40 kg, and the anti-TIGIT antagonist antibody is administered at a dose of between about 10 mg to about 1000 mg every three weeks (e.g., about 400 mg every three weeks); or (c) greater than 40 kg, and the anti-TIGIT antagonist antibody is administered at a dose of between about 30 mg to about 1200 mg every three weeks (e.g., about 600 mg every three weeks). In some instances, a subject with a body weight of less than or equal to 15 kg is administered a dose of between about 10 mg to about 1000 mg every three weeks (e.g., about 300 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body weight of greater than 15 kg and less than or equal to 40 kg is administered a dose of between about 10 mg to about 1000 mg every three weeks (e.g., about 400 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body weight of greater than 40 kg is administered a dose of between about 30 mg to about 1200 mg every three weeks (e.g., about 600 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks.

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body weight, wherein the subject has a body weight of (a) less than or equal to 15 kg, and the anti-TIGIT antagonist antibody is administered at a dose of between 10 mg to 1000 mg every three weeks (e.g., 300 mg every three weeks); (b) greater than 15 kg and less than or equal to 40 kg, and the anti-TIGIT antagonist antibody is administered at a dose of between 10 mg to 1000 mg every three weeks (e.g., 400 mg every three weeks); or (c) greater than 40 kg, and the anti-TIGIT antagonist antibody is administered at a dose of between 30 mg to 1200 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of between 250 mg to 350 mg every three weeks (e.g., 300 mg every three weeks); (b) greater than 15 kg and less than or equal to 40 kg, and the anti-TIGIT antagonist antibody is administered at a dose of between 350 mg to 450 mg every three weeks (e.g., 400 mg every three weeks); or (c) greater than 40 kg, and the anti-TIGIT antagonist antibody is administered at a dose of between 550 mg to 650 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body weight, wherein the subject has a body weight of (a) less than or equal to 15 kg, and the anti-TIGIT antagonist antibody is administered at a dose of 300 mg every three weeks; (b) greater than 15 kg and less than or equal to 40 kg, and the anti-TIGIT antagonist antibody is administered at a dose of 400 mg every three weeks; or (c) greater than 40 kg, and the anti-TIGIT antagonist antibody is administered at a dose of 600 mg every three weeks. In some instances, a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) is administered in combination with a tiered dose based on a subject's body weight of the anti-TIGIT antagonist antibody (e.g., an anti-TIGIT antagonist antibody as disclosed herein, e.g., tiragolumab), wherein the subject has a body weight of (a) less than or equal to 15 kg, and the anti-TIGIT antagonist antibody is administered at a dose of between 10 mg to 1000 mg every three weeks (e.g., 300 mg every three weeks); (b) greater than 15 kg and less than or equal to 40 kg, and the anti-TIGIT antagonist antibody is administered at a dose of between 10 mg to 1000 mg every three weeks (e.g., 400 mg every three weeks); or (c) greater than 40 kg, and the anti-TIGIT antagonist antibody is administered at a dose of between 30 mg to 1200 mg every three weeks (e.g., 600 mg every three weeks). In some instances, a subject with a body weight of less than or equal to 15 kg is administered a dose of between 10 mg to 1000 mg every three weeks (e.g., 300 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body weight of greater than 15 kg and less than or equal to 40 kg is administered a dose of between 10 mg to 1000 mg every three weeks (e.g., 400 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body weight of greater than 40 kg is administered a dose of between 30 mg to 1200 mg every three weeks (e.g., 600 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks.

In some instances, the effective amount of the anti-TIGIT antagonist antibody (e.g., an anti-TIGIT antagonist antibody as disclosed herein, e.g., tiragolumab) to treat a subject having a cancer is a tiered dose based on a subject's body surface area. In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between about 10 mg to about 1000 mg every three weeks (e.g., about 300 mg every three weeks); (b) greater than 0.5 m² and less than or equal to 0.75 m², and the anti-TIGIT antagonist antibody is administered at a dose of between about 10 mg to about 1000 mg every three weeks (e.g., about 350 mg every three weeks); (c) greater than 0.75 m² and less than or equal to 1.25 m², and the anti-TIGIT antagonist antibody is administered at a dose of between about 10 mg to about 1000 mg every three weeks (e.g., about 450 mg every three weeks); or (d) greater than 1.25 m², and the anti-TIGIT antagonist antibody is administered at a dose of between about 30 mg to about 1200 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between about 250 mg to about 350 mg every three weeks (e.g., about 300 mg every three weeks); (b) greater than 0.5 m² and less than or equal to 0.75 m², and the anti-TIGIT antagonist antibody is administered at a dose of between about 300 mg to about 400 mg every three weeks (e.g., about 350 mg every three weeks); or (c) greater than 0.75 m² and less than or equal to 1.25 m², and the anti-TIGIT antagonist antibody is administered at a dose of between about 400 mg to about 500 mg every three weeks (e.g., about 450 mg every three weeks); or (d) greater than 1.25 m², and the anti-TIGIT antagonist antibody is administered at a dose of between about 550 mg to about 650 mg every three weeks (e.g., about 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of about 300 mg every three weeks; (b) greater than 0.5 m² and less than or equal to 0.75 m², and the anti-TIGIT antagonist antibody is administered at a dose of about 400 mg every three weeks; (c) greater than 0.75 m² and less than or equal to 1.25 m², and the anti-TIGIT antagonist antibody is administered at a dose of 450 mg every three weeks; or (d) greater than 1.25 m², and the anti-TIGIT antagonist antibody is administered at a dose of about 600 mg every three weeks.

In some instances, a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) is administered in combination with a tiered dose based on a subject's body surface area of the anti-TIGIT antagonist antibody (e.g., an anti-TIGIT antagonist antibody as disclosed herein, e.g., tiragolumab), wherein the subject has a body surface area of (a) less than or equal to 0.5 m², and the anti-TIGIT antagonist antibody is administered at a dose of between about 10 mg to about 1000 mg every three weeks (e.g., about 300 mg every three weeks); (b) greater than 0.5 m² and less than or equal to 0.75 m², and the anti-TIGIT antagonist antibody is administered at a dose of between about 10 mg to about 1000 mg every three weeks (e.g., about 350 mg every three weeks); (c) greater than 0.75 m² and less than or equal to 1.25 m², and the anti-TIGIT antagonist antibody is administered at a dose of between about 10 mg to about 1000 mg every three weeks (e.g., about 450 mg every three weeks); or (d) greater than 1.25 m², and the anti-TIGIT antagonist antibody is administered at a dose of between about 30 mg to about 1200 mg every three weeks (e.g., about 600 mg every three weeks). In some instances, a subject with a body surface area of less than or equal to 0.5 m² is administered a dose of between about 10 mg to about 1000 mg every three weeks (e.g., about 300 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body surface area of greater than 0.5 m² and less than or equal to 0.75 m² is administered a dose of between about 10 mg to about 1000 mg every three weeks (e.g., about 350 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body surface area of greater than 0.75 m² and less than or equal to 1.25 m² is administered a dose of between about 10 mg to about 1000 mg every three weeks (e.g., about 450 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks. In some instances, a subject with a body surface area of greater than 1.25 m² is administered a dose of between about 30 mg to about 1200 mg every three weeks (e.g., about 600 mg every three weeks) of the anti-TIGIT antagonist antibody (e.g., an anti-TIGIT antagonist antibody as disclosed herein, e.g., tiragolumab) and a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) of the PD-1 axis binding antagonist (e.g., anti-PD-L1 antagonist antibody (e.g., atezolizumab)) every three weeks.

In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between 10 mg to 1000 mg every three weeks (e.g., 300 mg every three weeks); (b) greater than 0.5 m² and less than or equal to 0.75 m², and the anti-TIGIT antagonist antibody is administered at a dose of between 10 mg to 1000 mg every three weeks (e.g., 350 mg every three weeks); (c) greater than 0.75 m² and less than or equal to 1.25 m², and the anti-TIGIT antagonist antibody is administered at a dose of between 10 mg to 1000 mg every three weeks (e.g., 450 mg every three weeks); or (d) greater than 1.25 m², and the anti-TIGIT antagonist antibody is administered at a dose of between 30 mg to 1200 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of between 250 mg to 350 mg every three weeks (e.g., 300 mg every three weeks); (b) greater than 0.5 m² and less than or equal to 0.75 m², and the anti-TIGIT antagonist antibody is administered at a dose of between 300 mg to 400 mg every three weeks (e.g., 350 mg every three weeks); or (c) greater than 0.75 m² and less than or equal to 1.25 m², and the anti-TIGIT antagonist antibody is administered at a dose of between 400 mg to 500 mg every three weeks (e.g., 450 mg every three weeks); or (d) greater than 1.25 m², and the anti-TIGIT antagonist antibody is administered at a dose of between 550 mg to 650 mg every three weeks (e.g., 600 mg every three weeks). In some instances, the effective amount of the anti-TIGIT antagonist antibody (e.g., an anti-TIGIT antagonist antibody as disclosed herein, e.g., tiragolumab) is a tiered dose based on a subject's body surface area, wherein the subject has a body surface area of (a) less than or equal to 0.5 m², and the anti-TIGIT antagonist antibody is administered at a dose of 300 mg every three weeks; (b) greater than 0.5 m² and less than or equal to 0.75 m², and the anti-TIGIT antagonist antibody is administered at a dose of 400 mg every three weeks; (c) greater than 0.75 m² and less than or equal to 1.25 m², and the anti-TIGIT antagonist antibody is administered at a dose of 450 mg every three weeks; or (d) greater than 1.25 m², and the anti-TIGIT antagonist antibody is administered at a dose of 600 mg every three weeks.

In some instances, a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) is administered in combination with a tiered dose based on a subject's body surface area of the anti-TIGIT antagonist antibody (e.g., an anti-TIGIT antagonist antibody as disclosed herein, e.g., tiragolumab), wherein the subject has a body surface area of (a) less than or equal to 0.5 m², and the anti-TIGIT antagonist antibody is administered at a dose of between 10 mg to 1000 mg every three weeks (e.g., 300 mg every three weeks); (b) greater than 0.5 m² and less than or equal to 0.75 m², and the anti-TIGIT antagonist antibody is administered at a dose of between 10 mg to 1000 mg every three weeks (e.g., 350 mg every three weeks); (c) greater than 0.75 m² and less than or equal to 1.25 m², and the anti-TIGIT antagonist antibody is administered at a dose of between 10 mg to 1000 mg every three weeks (e.g., 450 mg every three weeks); or (d) greater than 1.25 m², and the anti-TIGIT antagonist antibody is administered at a dose of between 30 mg to 1200 mg every three weeks (e.g., 600 mg every three weeks). In some instances, a subject with a body surface area of less than or equal to 0.5 m² is administered a dose of between 10 mg to 1000 mg every three weeks (e.g., 300 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body surface area of greater than 0.5 m² and less than or equal to 0.75 m² is administered a dose of between 10 mg to 1000 mg every three weeks (e.g., 350 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body surface area of greater than 0.75 m² and less than or equal to 1.25 m² is administered a dose of between 10 mg to 1000 mg every three weeks (e.g., 450 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks. In some instances, a subject with a body surface area of greater than 1.25 m² is administered a dose of between 30 mg to 1200 mg every three weeks (e.g., 600 mg every three weeks) of the anti-TIGIT antagonist antibody (e.g., an anti-TIGIT antagonist antibody as disclosed herein, e.g., tiragolumab) and a dose of between 0.01 mg/kg to 50 mg/kg of the subject's body weight (e.g., between 0.01 mg/kg to 45 mg/kg, e.g., between 0.1 mg/kg to 40 mg/kg, e.g., between 1 mg/kg to 35 mg/kg, e.g., between 2.5 mg/kg to 30 mg/kg, e.g., between 5 mg/kg to 25 mg/kg, e.g., between 10 mg/kg to 20 mg/kg, e.g., between 12.5 mg/kg to 15 mg/kg, e.g., 15±2 mg/kg, 15±1 mg/kg, 15±0.5 mg/kg, 15±0.2 mg/kg, or 15±0.1 mg/kg, e.g., 15 mg/kg) of the PD-1 axis binding antagonist (e.g., anti-PD-L1 antagonist antibody (e.g., atezolizumab)) every three weeks.

### F. Additional Therapeutic Agents

In some aspects, the PD-1 axis binding antagonist and the anti-TIGIT antagonist antibody are used with one or more additional therapeutic agents, e.g., a combination therapy. In some aspects, the composition comprising the PD-1 axis binding antagonist and/or the anti-TIGIT antagonist antibody further comprises the additional therapeutic agent. In another aspect, the additional therapeutic agent is delivered in a separate composition. The one or more additional therapeutic agents may comprise, e.g., an immunomodulatory agent, an anti-neoplastic agent, a chemotherapeutic agent, a growth inhibitory agent, an anti-angiogenic agent, a radiation therapy, a cytotoxic agent, a cell-based therapy, or a combination thereof.

Combination therapies as described above encompass combined administration (wherein two or more therapeutic agents are included in the same or separate formulations) and separate administration (wherein administration of a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)) can occur prior to, simultaneously, and/or following, administration of the additional therapeutic agent or agents). In one aspect, administration of the PD-1 axis binding antagonist and the anti-TIGIT antagonist antibody and administration of an additional therapeutic agent occur within about one month, or within about one, two or three weeks, or within about one, two, three, four, five, or six days, of each other.

i. Chemotherapeutic Agents

In some aspects, the additional therapeutic agent is a chemotherapeutic agent. A chemotherapeutic agent is a chemical compound useful in the treatment of cancer. Exemplary chemotherapeutic agents include, but are not limited to erlotinib (TARCEVA®, Genentech/OSI Pharm.), anti-hormonal agents that act to regulate or inhibit hormone action on tumors such as anti-estrogens and selective estrogen receptor modulators (SERMs), antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), or trastuzumab (HERCEPTIN®, Genentech), EGFR inhibitors (EGFR antagonists), tyrosine kinase inhibitors, and chemotherapeutic agents also include non-steroidal anti-inflammatory drugs (NSAIDs) with analgesic, antipyretic and anti-inflammatory effects.

### V. Pharmaceutical Compositions, Formulations, and Kits

In another aspect of the invention, an article of manufacture or kit containing materials useful for the prognostic assessment and/or treatment of individuals is provided.

In some instances, such articles of manufacture or kits can be used to identify an individual having a cancer (e.g., a lung cancer (e.g., a NSCLC)) who may benefit from treatment with a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)). Such articles of manufacture or kits may include (a) reagents for determining the expression level of one or more genes and (b) instructions for using the reagents to identify an individual having a cancer (e.g., a lung cancer (e.g., a NSCLC) who may benefit from a treatment comprising a PD-1 axis binding antagonist and an anti-TIGIT antagonist antibody.

Any of the articles of manufacture or kits described may include a carrier means being compartmentalized to receive in close confinement one or more container means such as vials, tubes, and the like, each of the container means comprising one of the separate elements to be used in the method. Where the article of manufacture or kit utilizes nucleic acid hybridization to detect the target nucleic acid, the kit may also have containers containing nucleotide(s) for amplification of the target nucleic acid sequence and/or a container comprising a reporter-means, such as an enzymatic, fluorescent, or radioisotope label.

In some aspects, the article of manufacture or kit includes the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above. For example, the article of manufacture or kit may further include a container including a pharmaceutically-acceptable buffer, such as bacteriostatic water for injection (BWFI), phosphate-buffered saline, Ringer's solution, and dextrose solution.

The articles of manufacture or kits described herein may have a number of aspects. In one aspect, the article of manufacture or kit includes a container, a label on said container, and a composition contained within said container, wherein the composition includes one or more polynucleotides that hybridize to a complement of a locus described herein under stringent conditions, and the label on said container indicates that the composition can be used to evaluate the presence of a gene listed herein (e.g., C1QC, MSR1, MRC1, VSIG4, SPP1, MARCO, ACP5, MCEMP1, CYP27A1, OLR1, GRN, GLIPR2, ARRDC4, APOE, FOLR2, CTSD, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, LIRA3, FOXP3, CTLA4, IL10, TNFRSF18, CCR8, IKZF4, and IKZF2) in a sample, and wherein the kit includes instructions for using the polynucleotide(s) for evaluating the presence of the gene RNA or DNA in a particular sample type.

For oligonucleotide-based articles of manufacture or kits, the article of manufacture or kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The article of manufacture or kit can also include, e.g., a buffering agent, a preservative, or a protein stabilizing agent. The article of manufacture or kit can further include components necessary for detecting the detectable label (e.g., an enzyme or a substrate). The article of manufacture or kit can further include components necessary for analyzing the sequence of a sample (e.g., a restriction enzyme or a buffer). The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

### A. Tumor-Associated Macrophage (TAM) Genes and TAM Signature Scores

In some aspects, the invention provides articles of manufacture or kits can be used to identify an individual having a cancer (e.g., lung cancer (e.g., a NSCLC)) who may benefit from treatment with a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), wherein the articles of manufacture or kits may include (a) reagents for determining an expression level of one or more of C1QC, MSR1, MRC1, VSIG4, SPP1, and MARCO (e.g., one, two, three, four, five, or all six of C1QC, MSR1, MRC1, VSIG4, SPP1, and MARCO), and optionally reagents for determining an expression level of one or more of ACP5, MCEMP1, CYP27A1, OLR1, GRN, GLIPR2, ARRDC4, APOE, FOLR2, and CTSD (e.g., one, two, three, four, five, six, seven, eight, nine, or all ten of ACP5, MCEMP1, CYP27A1, OLR1, GRN, GLIPR2, ARRDC4, APOE, FOLR2, and CTSD) from a sample from an individual and (b) instructions for using the reagents to identify an individual having a cancer (e.g., a lung cancer (e.g., a NSCLC)) who may benefit from a treatment comprising a PD-1 axis binding antagonist and an anti-TIGIT antagonist antibody.

In some aspects, the invention provides articles of manufacture or kits can be used to identify an individual having a cancer (e.g., lung cancer (e.g., a NSCLC)) who may benefit from treatment with a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), wherein the articles of manufacture or kits may include (a) reagents for determining the tumor-associated macrophage (TAM) signature of a sample from an individual and (b) instructions for using the reagents to identify an individual having a cancer (e.g., a lung cancer (e.g., a NSCLC)) who may benefit from a treatment comprising a PD-1 axis binding antagonist and an anti-TIGIT antagonist antibody. The reagents for determining the TAM signature may include reagents for determining an expression level of one or more of C1QC, MSR1, MRC1, VSIG4, SPP1, and MARCO (e.g., one, two, three, four, five, or all six of C1QC, MSR1, MRC1, VSIG4, SPP1, and MARCO), and may further include reagents for determining an expression level of one or more of ACP5, MCEMP1, CYP27A1, OLR1, GRN, GLIPR2, ARRDC4, APOE, FOLR2, and CTSD (e.g., one, two, three, four, five, six, seven, eight, nine, or all ten of ACP5, MCEMP1, CYP27A1, OLR1, GRN, GLIPR2, ARRDC4, APOE, FOLR2, and CTSD).

In some aspects, such articles of manufacture or kits include a PD-1 axis binding antagonist and/or an anti-TIGIT antagonist antibody (e.g., a PD-1 axis binding antagonist as disclosed in Section IV herein (e.g., atezolizumab) and/or an anti-TIGIT antagonist antibody as disclosed in Section IV herein (e.g., tiragolumab)), for treating an individual with a cancer (e.g., a lung cancer (e.g., a NSCLC)), wherein the article of manufacture or kit includes (a) a PD-1 axis binding antagonist and/or an anti-TIGIT antagonist antibody (e.g., both a PD-1 axis binding antagonist and an anti-TIGIT antagonist antibody) and (b) a package insert including instructions for administration of the PD-1 axis binding antagonist and/or the anti-TIGIT antagonist antibody to an individual having a cancer (e.g., a lung cancer (e.g., a NSCLC)), wherein, prior to treatment, (i) the TAM signature score of a sample from the individual has been determined and the TAM signature score is above a reference TAM signature score, or (ii) the expression level of one, two, three, four, five, or all six of C1QC, MSR1, MRC1, VSIG4, SPP1, and MARCO in a sample from the individual has been determined and the expression level of one, two, three, four, five, or all six of C1QC, MSR1, MRC1, VSIG4, SPP1, and MARCO in the sample is above a threshold expression level. In some aspects, the expression level of one, two, three, four, five, six, seven, eight, nine, or all ten of ACP5, MCEMP1, CYP27A1, OLR1, GRN, GLIPR2, ARRDC4, APOE, FOLR2, and CTSD in a sample from the individual has been determined and the expression level of one, two, three, four, five, six, seven, eight, nine, or all ten of ACP5, MCEMP1, CYP27A1, OLR1, GRN, GLIPR2, ARRDC4, APOE, FOLR2, and CTSD in the sample is above a threshold expression level.

### B. Myeloid Markers in On-Treatment Serum Samples

In some aspects, the invention provides articles of manufacture or kits can be used to identify an individual having a cancer (e.g., lung cancer (e.g., a NSCLC)) who is likely to respond to treatment with a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), wherein the articles of manufacture or kits may include (a) reagents for determining an expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 (e.g., 1, 2, 3, 4, 5, 6, 7, 8, 9, 10, 11, 12, 13, 14, 15, 16, 17, 18, 19, or all 20 of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3) from a sample from an individual and (b) instructions for using the reagents to identify an individual having a cancer (e.g., a lung cancer (e.g., a NSCLC)) who is likely to respond to a treatment comprising a PD-1 axis binding antagonist and an anti-TIGIT antagonist antibody.

In some aspects, such articles of manufacture or kits include a PD-1 axis binding antagonist and/or an anti-TIGIT antagonist antibody (e.g., a PD-1 axis binding antagonist as disclosed in Section IV herein (e.g., atezolizumab) and/or an anti-TIGIT antagonist antibody as disclosed in Section IV herein (e.g., tiragolumab)), for treating an individual with a cancer (e.g., a lung cancer (e.g., a NSCLC)), wherein the article of manufacture or kit includes (a) a PD-1 axis binding antagonist and/or an anti-TIGIT antagonist antibody (e.g., both a PD-1 axis binding antagonist and an anti-TIGIT antagonist antibody) and (b) a package insert including instructions for administration of the PD-1 axis binding antagonist and/or the anti-TIGIT antagonist antibody to an individual having a cancer (e.g., a lung cancer (e.g., a NSCLC)), wherein, prior to treatment, the expression level of 1, 2, 3, 4, 5, 6, 7, 8, 9, 10, 11, 12, 13, 14, 15, 16, 17, 18, 19, or all 20 of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 in a sample from the individual has been determined and the expression level of 1, 2, 3, 4, 5, 6, 7, 8, 9, 10, 11, 12, 13, 14, 15, 16, 17, 18, 19, or all 20 of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 in the sample is above a threshold expression level.

### C. Regulatory T Cell (Treg) Genes and Treg Signature Scores

In some aspects, the invention provides articles of manufacture or kits can be used to identify an individual having a cancer (e.g., lung cancer (e.g., a NSCLC)) who may benefit from treatment with a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), wherein the articles of manufacture or kits may include (a) reagents for determining an expression level of one or more of FOXP3, CTLA4, IL110, TNFRSF18, CCR8, IKZF4, and IKZF2 (e.g., one, two, three, four, five, six, or all seven of FOXP3, CTLA4, IL10, TNFRSF18, CCR8, IKZF4, and IKZF2) from a sample from an individual and (b) instructions for using the reagents to identify an individual having a cancer (e.g., a lung cancer (e.g., a NSCLC)) who may benefit from a treatment comprising a PD-1 axis binding antagonist and an anti-TIGIT antagonist antibody.

In some aspects, the invention provides articles of manufacture or kits can be used to identify an individual having a cancer (e.g., lung cancer (e.g., a NSCLC)) who may benefit from treatment with a PD-1 axis binding antagonist and an anti-TIGIT antagonist antibody (e.g., a PD-1 axis binding antagonist as disclosed in Section IV herein (e.g., atezolizumab) and an anti-TIGIT antagonist antibody as disclosed in Section IV herein (e.g., tiragolumab)), wherein the articles of manufacture or kits may include (a) reagents for determining the regulatory T cell (Treg) signature of a sample from an individual and (b) instructions for using the reagents to identify an individual having a cancer (e.g., a lung cancer (e.g., a NSCLC)) who may benefit from a treatment comprising a PD-1 axis binding antagonist and an anti-TIGIT antagonist antibody. The reagents for determining the Treg signature may include reagents for determining an expression level of one or more of FOXP3, CTLA4, IL10, TNFRSF18, CCR8, IKZF4, and IKZF2 (e.g., one, two, three, four, five, six, or all seven of FOXP3, CTLA4, IL110, TNFRSF18, CCR8, IKZF4, and IKZF2).

In some aspects, such articles of manufacture or kits include a PD-1 axis binding antagonist and/or an anti-TIGIT antagonist antibody (e.g., a PD-1 axis binding antagonist as disclosed in Section IV herein (e.g., atezolizumab) and/or an anti-TIGIT antagonist antibody as disclosed in Section IV herein (e.g., tiragolumab)), for treating an individual with a cancer (e.g., a lung cancer (e.g., a NSCLC)), wherein the article of manufacture or kit includes (a) a PD-1 axis binding antagonist and/or an anti-TIGIT antagonist antibody (e.g., both a PD-1 axis binding antagonist and an anti-TIGIT antagonist antibody) and (b) a package insert including instructions for administration of the PD-1 axis binding antagonist and/or the anti-TIGIT antagonist antibody to an individual having a cancer (e.g., a lung cancer (e.g., a NSCLC)), wherein, prior to treatment, (i) the Treg signature score of a sample from the individual has been determined and the Treg signature score is above a reference Treg signature score, or (ii) the expression level of one, two, three, four, five, six, or all seven of FOXP3, CTLA4, IL10, TNFRSF18, CCR8, IKZF4, and IKZF2 in a sample from the individual has been determined and the expression level of one, two, three, four, five, six, or all seven of FOXP3, CTLA4, IL10, TNFRSF18, CCR8, IKZF4, and IKZF2 in the sample is above a threshold expression level.

### VI. Examples

The following are examples of the methods of the invention. It is understood that various other embodiments may be practiced, given the general description provided above.

### Example 1. TIGIT and PD-L1 Co-Blockade Relieves Myeloid Cell-Mediated Immunosuppression

a. Overview of Present Study

TIGIT is a co-inhibitory receptor and immune checkpoint associated with T cell and natural killer (NK) cell dysfunction in cancer. Tiragolumab is an anti-TIGIT antibody with an active, IgG1/kappa Fc. In a randomized double-blind phase 2 clinical trial in non-small cell lung cancer (NSCLC), tiragolumab+atezolizumab (anti-PD-L1) combination treatment demonstrated significant improvement relative to atezolizumab alone. However, the mechanisms underlying efficacy of this combination are not well understood.

CITYSCAPE is a randomized Phase 2 study that evaluated the efficacy of first-line (1 L) tiragolumab plus atezolizumab versus atezolizumab monotherapy in patients with PD-L1-positive NSCLC (tumor proportion score (TPS) 21%). The atezolizumab+tiragolumab combination treatment demonstrated superior clinical benefit, with an objective response rate (ORR) of 31%, as compared to a 16% ORR observed with atezolizumab plus placebo monotherapy, and an improvement in both progression-free survival (PFS) (hazard ratio (HR) 0.62, 95% confidence interval (CI): 0.42-0.91) and overall survival (OS) (HR 0.69, 95% CI: 0.44-1.07) in the intent-to-treat (ITT) population (Cho et al., Lancet Oncology, 23: 781-792, 2022).

The present Examples provide the first clinical biomarker analyses of TIGIT and PD-(L)1 antibody combination immunotherapy. Both baseline presence of intratumoral macrophages and on-treatment myeloid cell activation are associated with clinical benefit of tiragolumab+atezolizumab combination therapy, but not with benefit of atezolizumab monotherapy. Reverse translation of these findings in mouse models demonstrates that anti-TIGIT can exploit and modulate tumor-resident macrophages and other myeloid cells by FcγR engagement, remodeling the tumor microenvironment and improving the quality of the anti-tumor T cell response. These data identify a novel, clinically relevant mechanism of action for tiragolumab and suggest that Fc strategy is an important consideration for development of antibodies against TIGIT and other immune checkpoints.

### B. Methods

i. Study Design, Patient Cohort, and Response Assessment

The study was performed using tissue and peripheral samples from the open label, randomized Phase 1b GO30103 (NCT02794571; Bendell et al., Cancer Research, 80: Abstract CT302, 2010) and Phase 2 CITYSCAPE (NCT01903993; Cho et al., Lancet Oncology, 23: 781-792, 2022) trials. Patients were required to have tissue sent to a central laboratory before study entry, and samples were processed at the time of screening. Patients in the Phase Ib study received escalating doses of tiragolumab alone or in combination with 1200 mg atezolizumab (Q3W intravenous (IV) dosing). CITYSCAPE evaluated atezolizumab with tiragolumab versus atezolizumab with placebo in chemotherapy-naïve patients with locally advanced or metastatic NSCLC. Patients received either 1200 mg atezolizumab and/or 600 mg tiragolumab every 3 weeks (Q3W IV dosing) until disease progression or loss of clinical benefit. Protocol approval was obtained from independent ethics committees for each participating site for both studies, and an independent data monitoring committee reviewed the safety data. Patient outcome was characterized as response (complete/partial response (CR/PR)) or non-response (stable/progressive disease (SD/PD)).

ii. Clinical Tumor Collection and Bulk RNA-Seq Sequencing

Tumor biopsies at baseline were collected from patients enrolled in the CITYSCAPE trial. Whole transcriptome profiles were generated for n=105 patients using TruSeq RNA Access technology (ILLUMINA®).

iii. Multiplex Immunofluorescence

Multiplex immunofluorescence (mIF) was performed on a Ventana Discovery ULTRA autostainer. Following antigen retrieval with Cell Conditioning (CC1) solution (Ventana; 950-124), samples were incubated with the anti-FOXP3 rabbit monoclonal antibody SP97 (Abcam; ab99963), the anti-pan-cytokeratin mouse monoclonal antibody AE1/AE3 (Abcam, ab27988), the anti-CD68 rabbit monoclonal antibody SP251 (Spring Bioscience, M5510), and the anti-PD-L1 rabbit monoclonal antibody SP263 (Ventana; 790-4905) and were counterstained with DAPI (ThermoFisher Scientific; D3106). Whole stained slide images were then aligned using ULTISTACKER™ software (Ultivue, Cambridge, MA USA).

iv. Mass Spectroscopy and ELISA

Serum samples were collected from patients enrolled in CITYSCAPE at C1 D1 and C2D1. Samples were depleted of high-abundance proteins using Agilent MARS Human-14 multi affinity removal column connected to a DIONEX™ Ultimate 3000 RS pump (Thermo Scientific) according to manufacturer instructions. The PQ500™ panel had reference peptides (Biognosys) added to each sample.

Trypsinized serum was subjected to Hyper Reaction Monitoring (HRM™)/Data Independent Acquisition (DIA) liquid chromatography-mass spectrometry (LCMS) measurements along with reference peptides using an HRM™/DIA method, consisting of one full range MS1 scan and 29 MS2 segments, as described in Bruderer et al., Mol. Cell Proteomics, 18: 1242-1254, 2019.

HRM™/DIA mass spectrometric data were analyzed using SPECTRONAUT™ software (Biognosys, version 14.10) and normalized using local regression normalization (Callister et al. al., J Proteome Res, 5(2): 277-286, 2006). The mass spectrometric data were searched using SPECTROMINE™ (Biognosys, version 2.5), with a false discovery rate on peptide and protein level set to 1%. Two separate spectral libraries were created from DDA data and DIRECTDIA™ data from HRM™/DIA data. Low quality protein levels were filtered based on Q-values (cutoff 0.01) and the batch effect corrected using combat (Leek et al., PLoS Genet., 3: 1724-1735, 2007). The limma (Ritchie et al, Nucleic Acids Res., 43: e47, 2015) was used to test a difference of log scaled protein levels. PQ500™ assay panel data was used for clinical efficacy analysis. A composite was calculated at each time point (C1D1 and C2D1) by averaging scaled PQ500™ values of all significantly increased proteins (MARCO, CAMP, CD163, CSF1R, CD5L, NGAL (LCN2), GAPR1, APOC1, APOC2, APOC3, and APOC4).

Human CD163 Immunoassay from R&D Systems (cat number DC1630) was qualified in procured human serum samples and then used to measure soluble CD163 from patient serum samples in duplicate.

v. PBMC Sample Collection, RNA-Seq 10× Genomics Library Construction, and Sequencing

Peripheral blood mononuclear cells (PBMCs) were collected from patients enrolled in the phase 1b NSCLC study of tiragolumab plus atezolizumab (G030103). A total of 16 patients had available samples from cycle 1 day 1 (C1D1), cycle 1 day 15 (C1D15, two weeks post-treatment), cycle 2 day 1 (C2D1, 3 weeks post-treatment), and cycle 4 day 1 (C4D1, 9 weeks post-treatment). Frozen PBMCs were thawed, washed 2× in RPMI 2% FCS, treated with the ACK lysis buffer (Lonza) to remove red blood cells (RBCs), and briefly incubated with DAPI. 300,000 cells were then sorted on a DAPI negative gate, stained for 30 minutes at room temperature with a custom panel of 138 Total-Seq-C antibodies (Biolegend; Stoeckius et al., Nature Methods, 14: 865-868, 2017), and washed 3 times using the HT1000 laminar wash system (Curiox). Cells were then counted using the CELLACA™ MX High-throughput Automated Cell Counter (Nexcelom), pooled from 5 samples, and loaded on the 10× Chromium Next GEM Chip G Kit using a superloading strategy. TCR CDR3 sequences were enriched using human V(D)J T cell enrichment. Libraries were prepared according to the manufacturer's protocol (10× Genomics) and sequenced on a NovaSeq 6000 System using the S4 2×150 kit (Illumina).

vi. In Vivo Mouse Tumor Models

The CT26 murine colon carcinoma cell line was obtained from the American Type Culture Collection (ATCC; Manassas, VA). Cells were cultured in Roswell Park Memorial Institute (RPMI) 1640 medium plus 2 mM L-glutamine with 10% fetal bovine serum (HYCLONE™; Waltham, MA). Cells in log-phase growth were centrifuged, washed once with Hank's balanced salt solution (HBSS), counted, and resuspended in 50% HBSS and 50% MATRIGEL™ (BD Biosciences; San Jose, CA). 1×10⁵ cells were inoculated subcutaneously into the right unilateral flank of each mouse

After approximately 10 days, mice bearing tumors of 150-200 mm³ were randomized into treatment groups on the basis of tumor size and treated with anti-mouse PD-L1 (clone 6E11, isotype IgG2A LALAPG, 10 mg/kg), anti-mouse TIGIT (clone 10A7, isotypes IgG2a, mIgG2b, and IgG2a LALAPG, 10 mg/kg), and/or anti-gp120 control antibodies (IgG2A isotype, to total 35 mg/kg overall antibody dosing).

In tumor growth inhibition experiments, antibodies were administered three times per week for 2 weeks; the first dose was administered intravenously and all subsequent doses were administered by intraperitoneal injection. Animals were continuously monitored, and mice were euthanized via asphyxiation when any of the following endpoints were met: study termination, tumor burden 22,000 mm3, tumor ulceration, body weight loss≥20%, or moribund appearance. Tumor burden was measured by caliper, and tumor volumes were calculated using the modified ellipsoid formula ½×(length×width2). In scRNA-seq experiments, antibodies were administered once intravenously. Seventy-two hours after treatment, mice were euthanized via asphyxiation.

vii. Ex Vivo Flow Cytometry of Mouse Tumors

Tumors were minced and digested with collagenase/DNase, filtered, and resuspended in single cell suspension for flow cytometry (FACS) staining. Fluorophore-conjugated antibodies against the indicated surface markers were used to stain single cell solutions of tumors, splenocytes, LN cells, and peripheral blood cells. For intracellular staining, cells were first surface stained for lineage markers, fixed, permeabilized and stained with antibodies against IFNγ, TNFa, FOXP3 or Ki67. Stained cells were analyzed using a FACSYMPHONY™ flow cytometer, and further data analysis was carried out using FlowJo software.

viii. Mouse RNA-Seq 10× Genomics Library Preparation and Sequencing

Blood and tumors were harvested from mice and subject to single cell suspension preparation by enzymatic dissociation and/or red blood cell lysis as needed. Cells within each tissue and treatment group were hashtagged (BIOLEGEND® TOTALSEQ™-C), pooled from different mice, and labeled with fluorescent anti-CD45 and a viability dye. Live CD45+ cells were sorted and cell numbers determined by VI-CELL™ XR cell counter (Beckman Coulter). A total of 20,000 CD45+ cells were processed following 10× Genomics' demonstrated protocol (CG000330_Chromium Next GEM Single Cell 5-v2 Cell Surface Protein UserGuide_RevA) to generate 5′ single-cell RNA-seq and hashed libraries. Both libraries were sequenced in a NovaSeq S4 sequencer (Illumina) with the specification based on 1 Ox Genomics recommendation and as follows: 5′ single-cell RNA-seq libraries were sequenced at 40,000 reads/cells and hashed libraries at 2,000/cells.

Ix. Gene Expression Analysis

All transcriptome profiles were generated using ILLUMINA® TRUSEQ™ RNA Access technology. Alignment of RNAseq reads to ribosomal RNA sequences was performed to remove ribosomal reads. The National Cancer Institute (NCI) Build 38 human reference genome was then used to align the remaining reads using Genomic Short-read Nucleotide Alignment Program (GSNAP) version 2013-10-10, wherein a maximum of two mismatches per 75 base sequence (parameters: ‘-M 2 -n 10 -B 2 -I 1-N 1-w 200000 -E 1--pairmax-rna=200000 --clip-overlap) was allowed (Wu et al., Methods Mol. Biol., 1418: 283-334, 2016). Transcript annotation was based on the Ensembl genes database (release 77). To quantify gene expression levels, the number of reads mapped to the exons of each RefSeq gene was calculated in a strand-specific manner using the functionality provided by the R package Genomic Alignments (Bioconductor) (Lawrence et al., PLoS Comput. Biol., 9: el 003118, 2013).

x. Public scRNA-Seq Processing and Myeloid Cell Signatures

The scRNA-seq dataset for human lung tumors that was reported in Lambrechts et al., Nat. Med., 24: 1277-1289, 2018 was obtained as loom files from E-MTAB-6149. Data were converted to a Seurat object and analyzed using the Seurat R package (version 3.2.2) following the standard workflow (Stuart et al., Cell, 177: 1888-1902.e1821, 2019). Myeloid cells were retrieved and analyzed to define cell subtypes. As reported previously, cells were removed if there were either <201 unique molecular identifiers (UMIs), >6,000 or <101 expressed genes, or >10% UMIs derived from the mitochondrial genome. The filtered gene expression matrix was normalized using the NormalizeData with default parameters. The data were then scaled and the effects of variation of UMI counts and percent mitochondrial contents were regressed out (ScaleData). Principal component analysis was performed on the scaled data cut to the top 2,000 variable genes defined by FindVariableFeatures with default parameters. To integrate different samples, the harmony (v1.0) package (Korsunsky et al., Nature Methods, 16: 1289-1296, 2019) was used and the top 20 principal components were used as input for the RunHarmony function with default parameters. Cell clusters were defined by FindClusters using a resolution of 0.5 and annotated using canonical marker genes curated before (Cheng et al., Cell, 184: 792-809.e723, 2021).

To derive the TAM signatures, markers for each myeloid cluster were defined by comparing cells in a particular myeloid cell cluster to every other cluster in a pairwise fashion. To guarantee myeloid specific expression of markers, only marker genes that were not expressed by non-myeloid cells in an independent dataset (Kim et al., Nat. Commun., 11: 2285, 2020), including stromal, tumor, and non-myeloid immune cells, were retained. Three previously described macrophage populations (Cheng et al., Cell, 184: 792-809.e723, 2021) characterized by their immunosuppressive characteristics were grouped as TAMs. The signature genes from each defined macrophage cluster were combined and the resultant signature was derived (MARCO, ACP5, VSIG4, MRC1, MSR1, MCEMP1, CYP27A1, OLR1, GRN, GLIPR2, ARRDC4, C1QC, APOE, FOLR2, CTSD, SPP1).

xi. Preprocessing of Human PBMC scRNA-Seq Data

The scRNA-seq reads were aligned to the human transcriptome (GRCh38) and UMI counts were quantified to generate a gene-barcode matrix using the Cell Ranger pipeline (10× Genomics, version cellranger-5.0.1). CITE-seq antibody expression matrices were generated using the Cell Ranger pipeline (10× Genomics, version cellranger-5.0.1). TCR reads were aligned to the GRCh38 reference genome and consensus TCR annotation was performed using the Cell Ranger vdj pipeline (10× Genomics, version cellranger-5.0.1). To assign cells to their respective samples of origin, cells were demultiplexed with a modified HTOdemux function from the Seurat package, whereby the negative cluster was defined by minimal non-zero expression.

xii. Cluster Analysis of Human PBMC Immune Cells

The preprocessed gene expression matrix generated by the Cell Ranger pipeline was imported into Seurat (version 3.2.2) for downstream analysis. As a quality control step, genes that were expressed in less than 10 cells were removed and cells were filtered based on the number of detected genes, number of detected UMIs, house-keeping gene expression and percentage of mitochondrial gene expression. Cells that expressed less than 10 house-keeping genes were removed. For UMIs, detected genes and mitochondrial gene expression, cutoffs were defined as the more conservative value between a hard predefined cutoff (UMI: lower 1,000—upper 20,000; Genes: lower 200—upper 5,000; mitochondrial gene expression: 10%) and a dataset specific cutoff computed using interquartile ranges. In addition, RBC and platelet contaminants were removed via automated filtering algorithms. The filtered gene expression matrix (17,804 genes×407,219 cells) was normalized using the NormalizeData function (normalization.method=“LogNormalize” and scale.factor=10,000). Surface proteins were normalized using the centered-log ratio (CLR) method. Variable genes were identified using the FindVariableFeatures function with default parameters. Prior to dimension reduction, the data were scaled, and the effects of variation in UMI counts and percentage mitochondrial contents were regressed out (the ScaleData function). Principal component analysis was then performed on the scaled data cut to the variable genes. Batch effects were mitigated using the Harmony (version 1.0) package (Korsunsky et al., Nature Methods, 16: 1289-1296, 2019). Shared nearest neighbors were computed, and cells were then clustered using graph community clustering methods. A uniform manifold approximation and projection (UMAP) was generated using the RunUMAP function. Cells were annotated using a cell type classifier taking into account RNA, surface proteins and TCR sequences, and further validated and refined by using immunai's curated in-house signatures. Multi-omic data was further utilized to remove low quality cells and previously undetected doublets (e.g., cells which express both CD8 and CD4 protein tags, cells which express both a high B cell signature and have a detected TCR).

xiii. Identification of Proliferating Cells in Human PBMCs

To identify proliferating cells from the scRNA-seq data, cell proliferation scores at S and G2M phases were calculated using the CellCycleScoring function from Seurat. Proliferating cells were called based on S phase score or G2M phase scores 20.22 or S phase scores 20.22.

xiv. Pseudo-Bulk Differential Gene Expression Analysis of Human PBMCs

Differential gene expression (DEG) tests were performed by pseudo-bulk analysis, in which gene counts were aggregated (summed) for each sample and cell type. Samples per cell type that had <10 cells were removed. Differential expression analysis was performed with the limma-voom R package (version 3.44.3) (Lindau et al., Immunology, 138: 105-115, 2013) for each cell type independently. Patient ID was added as a covariate to the design formula to consider the paired design. Patients without matching pre- and on-treatment samples were removed. The moderated t-statistics from limma DEG tests were used as a pre-ranked gene list input for pathway enrichment analysis, which was performed using the fgsea R package (version 1.14.0) (Sergushichev, bioRxiv, 060012, 2016). In this analysis, the Hallmark gene set collected from MSigDB (version 7.2) was used.

xv. Preprocessing of Mouse scRNA-Seq Data

The gene expression FASTQ files were aligned to the mouse transcriptome (mm10) and UMI counts were quantified to generate a gene-barcode matrix using the Cell Ranger pipeline (10× Genomics, version cellranger-6.1.1). Antibody-derived tag (ADT) expression FASTQ files were generated using the Cell Ranger pipeline (10× Genomics, version cellranger-6.1.1). The exported gene expression and ADT expression matrices were imported into the Seurat package for downstream analysis. ADT data were normalized with centered log-ratio transformation and HTODemux function was used to assign mouse-of-origin for each singlet cell, and annotate doublets and singlets.

xvi. Clustering Analysis of Mouse scRNA-Seq Data

Two batches of mouse scRNA-seq data were generated and analyzed separately following the standard Seurat workflow as described above. Throughout the analysis, the absence of batch effects introduced by samples or other technical factors was confirmed, and thus batch effect removal was not performed in the data. Cells were annotated by canonical marker genes and high expression marker genes in the cluster compared with the other cells.

Specifically, for the scRNA-seq data generated from mice treated with isotype control, aTIGIT-LALAPG, aTIGIT-IgG2b, and aTIGIT-IgG2a, singlets and negative cells were used for downstream analysis. Cells collected from the peripheral blood were kept using the following filtering: mitochondrial % counts<5%, 1,000<UMI counts<15,000, and 500<gene counts<3,500, resulting in a total of 26,174 cells. The first 25 PCs and a resolution of 1 were used for dimensionality reduction and clustering, and clusters with similar marker gene expression were combined. Cells collected from the tumor were kept using the following filtering: mitochondrial % counts<5%, 1,000<UMI counts<25,000, and 500<gene counts<6,000. The top 25 PCs were used for dimension reduction and all cells were clustered at a resolution of 0.6 to define the broader myeloid cells (n=5,352) and T/NK lymphocytes (n=21,407). Further clustering analysis was performed on the myeloid cells (top 20 PCs and a resolution of 0.9) and T/NK lymphocytes (top 23 PCs and a resolution of 0.9).

For the scRNA-seq data generated from mice treated with isotype control, aPD-L1, aTIGIT-IgG2b, and aTIGIT-IgG2a, aPD-L1+aTIGIT-IgG2b, and aPD-L1+aTIGIT-IgG2a, and singlets were used for downstream analysis. Cells collected from the peripheral blood were kept using the following filtering: mitochondrial % counts<5%, 1,000<UMI counts<20,000, and 500<gene counts<4,500, resulting in a total of 55,368 cells. The first 25 PCs and a resolution of 1 were used for dimensionality reduction and clustering, and clusters with similar marker gene expression were combined. Cells collected from the tumor were kept using the following filtering: mitochondrial % counts<5%, 1,000<UMI counts<25,000, and 500<gene counts<6,000. The top 25 PCs for were used for dimension reduction all cells were clustered at a resolution of 0.1 to define the broader myeloid cells (n=4,261) and T/NK lymphocytes (n=35,358). Further clustering analysis was performed on the myeloid cells (top 20 PCs and a resolution of 0.9) and T/NK lymphocytes (top 20 PCs and a resolution of 0.6).

xvii. Differential Gene Expression Analysis and Cluster Marker Genes of Mouse scRNA-Seq Data

Differential gene expression analysis was performed using the Wilcoxon rank-sum test implemented in Seurat. FindMarkers function was used to define the differentially expressed genes (DEGs) between cells from each treatment group. Volcano plots and bubble plots were used to visualize genes that were differentially expressed in each treatment group. Marker genes for each cluster were identified by comparing cells in one cluster to cells in all other clusters using FindAllMarkers function.

xvii. Statistical Analysis

Survival outcomes, overall survival and progression free survival were analyzed by the Kaplan-Meier method. Univariate Cox regressions were implemented to estimate HRs and 95% CIs. Statistical details of experiments, number of repeats performed, and statistical tests used can be found in the Brief Description of the Drawings.

### Example 2. Tumor-Resident Macrophages, Regulatory T Cells, and Effector T Cells Correlate with Tiragolumab Plus Atezolizumab Outcome

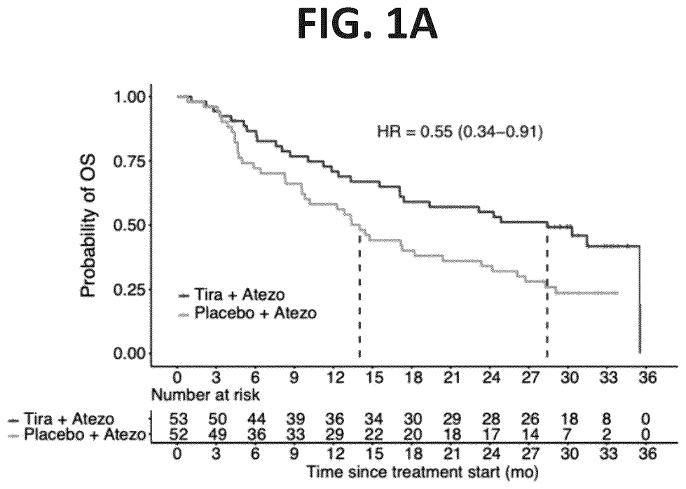

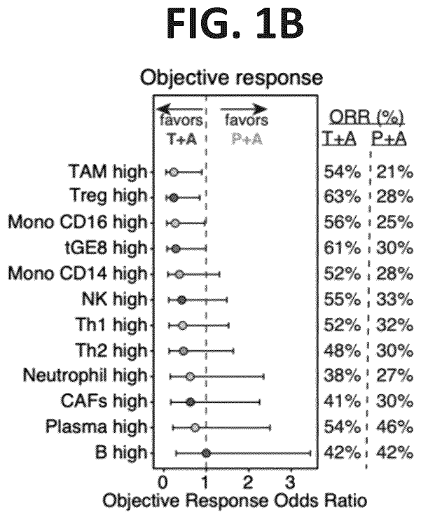

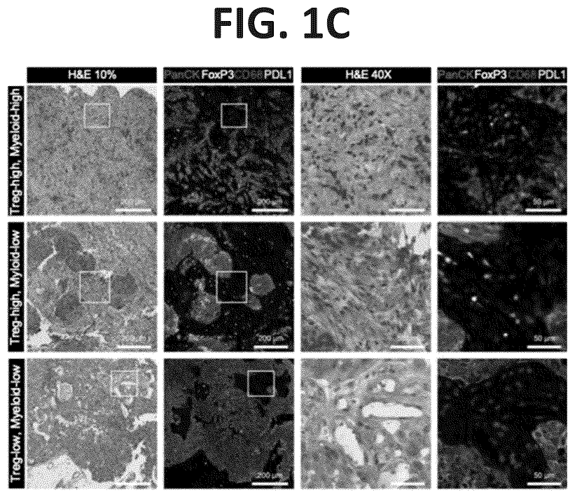

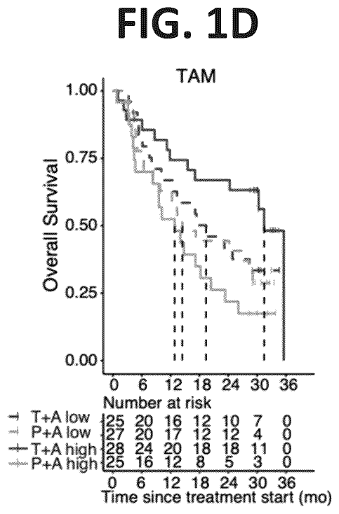

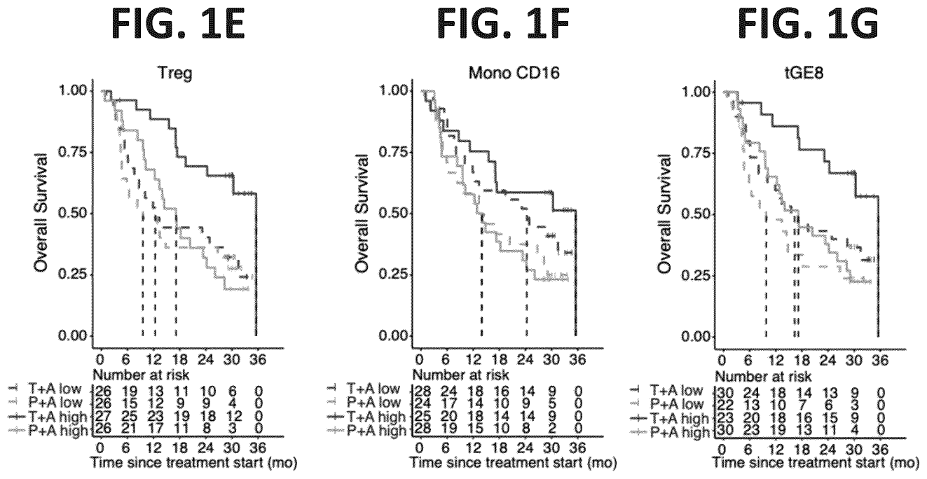

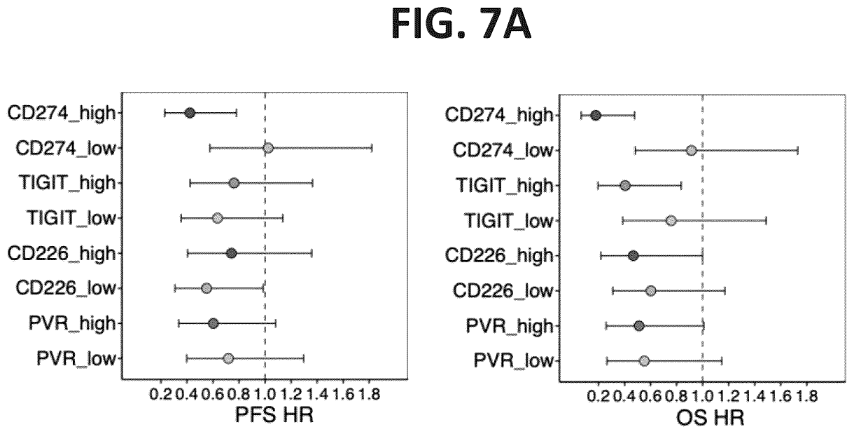

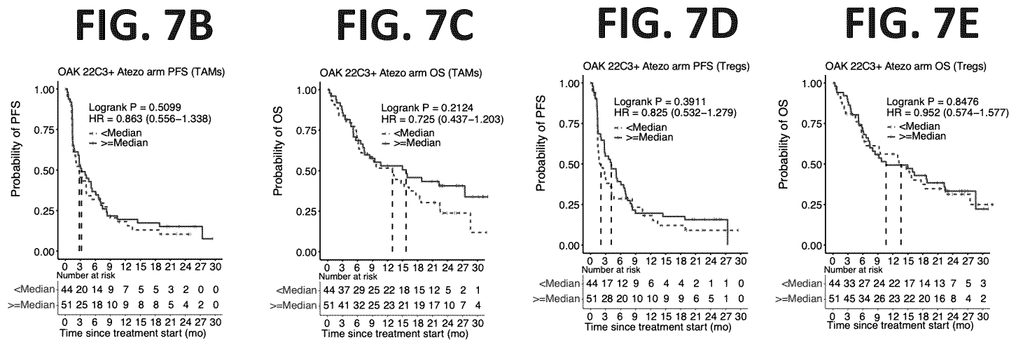

Bulk RNA sequencing (RNA-seq) of available pretreatment tumor samples from patients enrolled in the CITYSCAPE trial was performed. This biomarker-evaluable population (BEP, n=105) displayed comparable baseline demographics to the ITT population (n=135; Table 4), and similar benefits of tiragolumab plus atezolizumab therapy with a BEP overall survival (OS) (unstratified) HR of 0.55 (95% C, 0.34-0.91; FIG. 1A) (Cho et al., Lancet Oncology, 23: 781-792, 2022). Consistent with the results of post-hoc analysis of PD-L1 immunohistochemistry (Cho et al., Lancet Oncology, 23: 781-792, 2022), high CD274 gene expression was associated with improved PFS and OS in the tiragolumab plus atezolizumab arm compared with the placebo plus atezolizumab arm (PFS HR, 0.42 (95% CI, 0.23-0.78); 05 HR, 0.18 (95% CI, 0.06-0.48)) (FIG. 7A).

TABLE 4
Patient demographics

| Intent To Treat Population (ITT) | | Biomarker Evaluable Population (BEP) | |
|---|---|---|---|
| (n = 135) | | (n = 105) | |
| Tiragolumab plus | Placebo plus | Tiragolumab plus | Placebo plus |
| atezolizumab | atezolizumab | atezolizumab | atezolizumab |
| (n = 67) | (n = 68) | (n = 53) | (n = 52) |

Age

| | | | | |
|---|---|---|---|---|
| Median | 68 | 68 | 68 | 68 |
| age, years | | | | |
| Range | 41-84 | 40-83 | 41-84 | 40-83 |
| ≥65 years | 39 (58%) | 40 (59%) | 31 (58%) | 29 (56%) |
| <65 years | 28 (42%) | 28 (41%) | 22 (42%) | 23 (44%) |

Sex, n

| | | | | |
|---|---|---|---|---|
| Male | 39 (58%) | 48 (71%) | 32 (60%) | 38 (73%) |
| Female | 28 (42%) | 20 (29%) | 21 (40%) | 14 (27%) |

Race, n

| | | | | |
|---|---|---|---|---|
| White | 42 (63%) | 40 (59%) | 35 (66%) | 32 (62%) |
| Asian | 18 (27%) | 23 (34%) | 13 (25%) | 17 (33%) |
| Multiple | — | 1 (2%) | — | 1 (2%) |
| Unknown | 7 (10%) | 4 (6%) | 5 (9%) | 2 (4%) |

ECOG PS at baseline, n

| | | | | |
|---|---|---|---|---|
| 0 | 20 (30%) | 19 (28%) | 13 (25%) | 14 (27%) |
| 1 | 47 (70%) | 48 (71%) | 40 (75%) | 37 (71%) |
| 2 | — | 1 (2%) | — | 1 (2%) |

Histology, n

| | | | | |
|---|---|---|---|---|
| Non- | 40 (60%) | 40 (59%) | 31 (58%) | 30 (58%) |
| squamous | | | | |
| Squamous | 27 (40%) | 28 (41%) | 22 (42%) | 22 (42%) |

PD-L1, n

| | | | | |
|---|---|---|---|---|
| TPS 1-49% | 38 (57%) | 39 (57%) | 29 (55%) | 28 (54%) |
| TPS ≥50% | 29 (43%) | 29 (43%) | 24 (45%) | 24 (46%) |

Tobacco use, n

| | | | | |
|---|---|---|---|---|
| Never | 7 (10%) | 7 (10%) | 6 (11%) | 4 (8%) |
| Previous | 41 (61%) | 44 (65%) | 29 (55%) | 36 (69%) |
| Current | 19 (28%) | 17 (25%) | 18 (34%) | 12 (23%) |

BEP, biomarker-evaluable population; ECOG PS, Eastern Cooperative Oncology Group performance status; ITT, intent-to-treat; PD-L1, programmed death-ligand 1; TPS, tumor proportion score.

To investigate the mechanisms underlying the tiragolumab plus atezolizumab combination benefit, patients were stratified based on intratumoral leukocyte and stromal cell gene expression signatures and the association of each signature with clinical outcome was evaluated. These gene signatures were either derived from NSCLC scRNA-seq datasets (Lambrechts et al., Nat. Med., 24: 1277-1289, 2018; Kim et al., Nat. Commun., 11: 2285, 2020) or have been described previously (Bagaev et al., Cancer Cell, 39: 845-865, 2020; Mariathasan et al., Nature, 554: 544-548, 2018). Consistent with their central role in immunotherapy and with studies of other checkpoint inhibitors, CD8+ effector T cells were associated with an improved ORR in patients treated with tiragolumab plus atezolizumab (FIG. 1B). Unexpectedly, higher abundance of tumor-associated macrophages (TAMs) and regulatory T cells (Tregs), which function as immunosuppressive cells in the tumor microenvironment, were also associated with improved ORR with the combination regimen relative to the control arm (FIG. 1B).

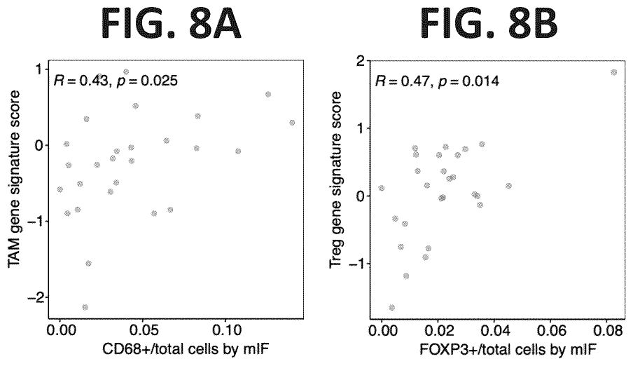

Pretreatment tumor samples (n=22) were evaluated by multiplex immunofluorescence staining for pan-cytokeratin (PanCK, tumor marker), FoxP3 (Treg marker), CD68 (macrophage marker), and PD-L1 to confirm the TAM and Treg transcriptional findings. Abundant CD68+ cells and FoxP3+ cells were detected in samples with high TAM and Treg signatures measured by bulk RNA-seq (FIG. 1C), which also exhibited positive correlations with cell counts measured by multiplex immunofluorescence staining (FIGS. 8A and 8B).

Kaplan-Meier analysis showed that increased TAMs and Tregs in the tumor were associated with improved OS for the combination treatment, but not for atezolizumab monotherapy: OS HR, 0.35 (95% CI, 0.17-0.73) for TAMs and 0.31 (95% CI, 0.14-0.67) for Tregs (FIGS. 1D and 1E). Compared with TAMs, all monocytes, particularly CD16-high non-classical monocytes, exhibited a positive but weaker association with tiragolumab plus atezolizumab survival (FIGS. 1B-1F). Increased CD8+ effector T cells were positively associated with treatment benefit in both arms (FIG. 1G). In contrast, B cells and plasma cells, which we and others have identified as associated with clinical benefit following atezolizumab and other checkpoint inhibitors (Patil et al., Cancer Cell, 40: 289-300e.284, 2022), were not associated with improved ORR for tiragolumab plus atezolizumab treatment relative to the control arm (FIG. 1B).

TAMs and Treg signatures were also analyzed in a similar patient population (PD-L1-positive (TPS≥21%) NSCLC) in a larger independent dataset from the phase 3 OAK study (Rittmeyer et al., Lancet, 389: 255-265, 2017). Consistent with the atezolizumab plus placebo results in CITYSCAPE, TAMs and Treg signatures were not associated with improved PFS or OS with atezolizumab monotherapy in OAK (FIGS. 7B-7E).

Together, these data indicate that tiragolumab plus atezolizumab combination treatment efficacy was selectively and counterintuitively associated with TAMs and tumor Tregs, in addition to typical correlates of checkpoint inhibitor responsiveness such as CD8+ effector T cells and PD-L1 expression. It is thus hypothesized that tiragolumab functions as both a canonical checkpoint inhibitor as well as through a differentiated, non-canonical mechanism of action.

### Example 3. Myeloid Cell Activation Following Tiragolumab Plus Atezolizumab Treatment

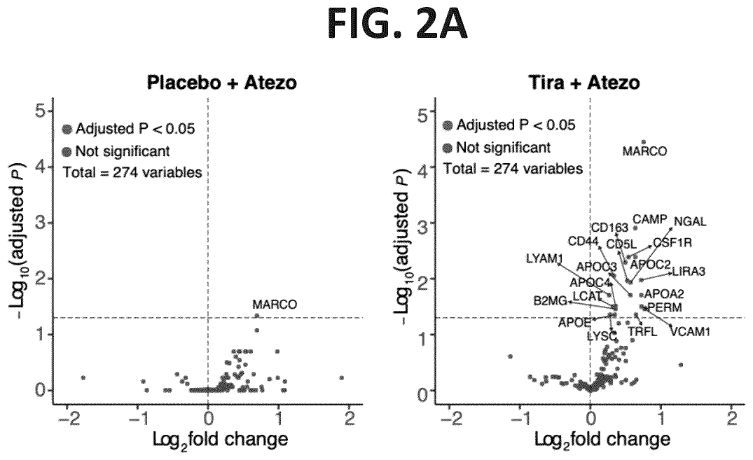

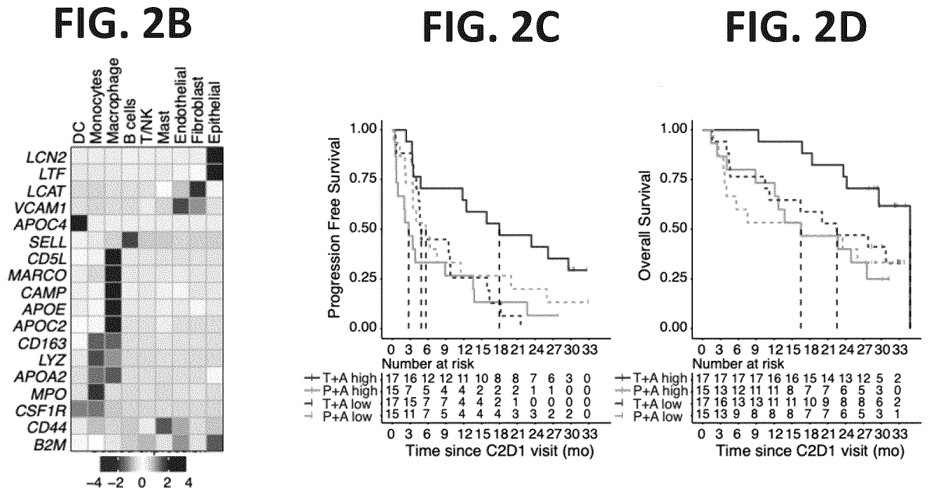

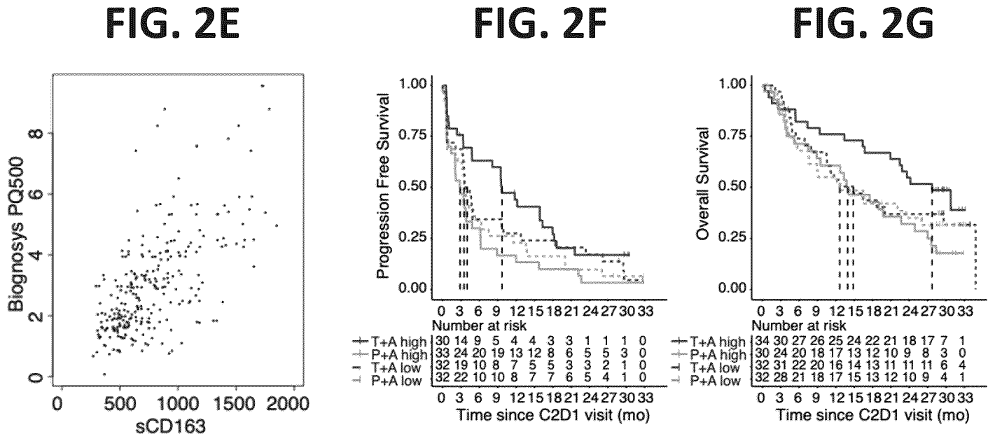

Next, longitudinally collected peripheral serum samples were utilized to identify specific on-treatment signals associated with combination treatment in the CITYSCAPE trial. Mass spectrometry was performed to profile serum proteins present on cycle 1 day 1 (C1D1 or baseline), and cycle 2 day 1 (C2D1 or 3 weeks post-treatment) from serum samples of CITYSCAPE patients (n=64). A comparison of circulating peptides at C2D1 versus baseline showed a statistically significant increase (adjusted p value<0.05) of peptides derived from myeloid-expressed proteins such as macrophage receptor with collagenous structure (MARCO), CSF1R, CD163, CAMP, CD5L, and apolipoproteins (APOC1/2/3) in the tiragolumab plus atezolizumab treatment patients but not in the placebo plus atezolizumab patients (FIG. 2A). The myeloid-specific expression patterns of genes encoding those upregulated proteins were confirmed using a public NSCLC scRNA-seq gene expression data (FIG. 2B).

To understand the kinetics of these proteins in the context of clinical outcomes, a composite was generated of all the significantly modulated proteins (n=11) using their C2D1 fold-changes relative to baseline, and Kaplan-Meier survival analysis for PFS and OS was performed (FIGS. 2C and 2D). In patients who had a greater increase of these myeloid proteins at three weeks, it was observed that patients treated with tiragolumab plus atezolizumab combination showed longer PFS and OS than patients receiving placebo plus atezolizumab (PFS HR, 0.32 (95% CI, 0.14-0.72); OS HR, 0.30 (95% CI, 0.11-0.81)), suggesting that myeloid cell activation may be an important combination treatment-specific mechanism of response.

Although little is known about the presence of most of these proteins in serum, circulating serum soluble CD163 is an established marker of monocyte and tissue macrophage activation and is a haemoglobin-haptoglobin scavenger receptor expressed exclusively on monocytes and macrophages (Dige et al., Scand. J. Immunol., 80: 417-423, 2014; Davis et al., Cytometry B. Clin. Cytom., 63: 16-22, 2005). Soluble CD163 was measured in available serum samples from CITYSCAPE patients (n=127) using a sCD163 ELISA. sCD163 levels by ELISA were correlated with CD163 detected by mass spectrometry in patients who had both sets of data (FIG. 2E). Using the C2D1 fold-changes relative to baseline, Kaplan-Meier survival analysis for PFS and OS showed that in patients with greater elevation in sCD163, tiragolumab plus atezolizumab combination conferred improved PFS and OS than placebo plus atezolizumab (PFS HR, 0.47, (95% CI, 0.29-0.80); OS HR, 0.49, (95% CI, 0.29-0.91)) (FIGS. 2F and 2G).

### Example 4. Peripheral Monocyte Activation and Treg Reduction Following Tiragolumab Plus Atezolizumab Treatment

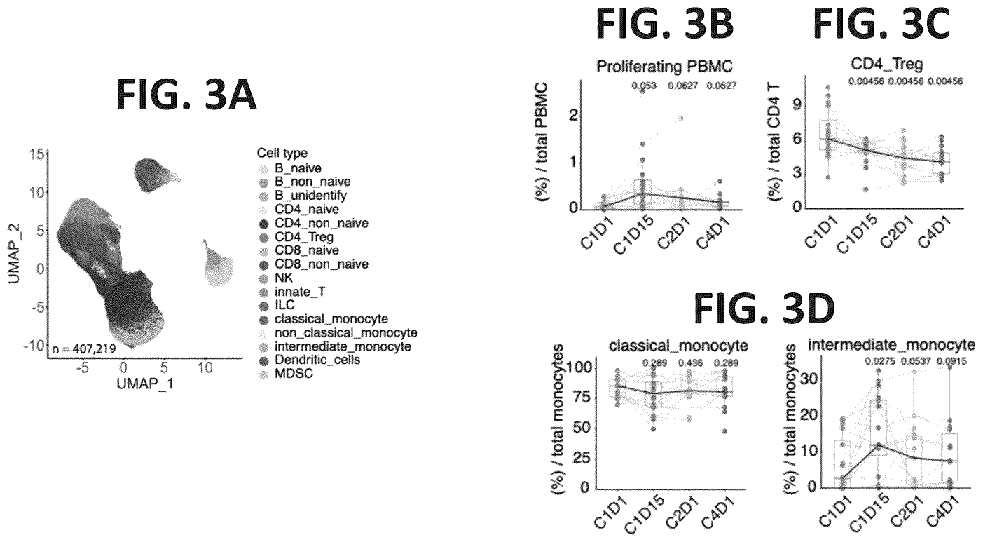

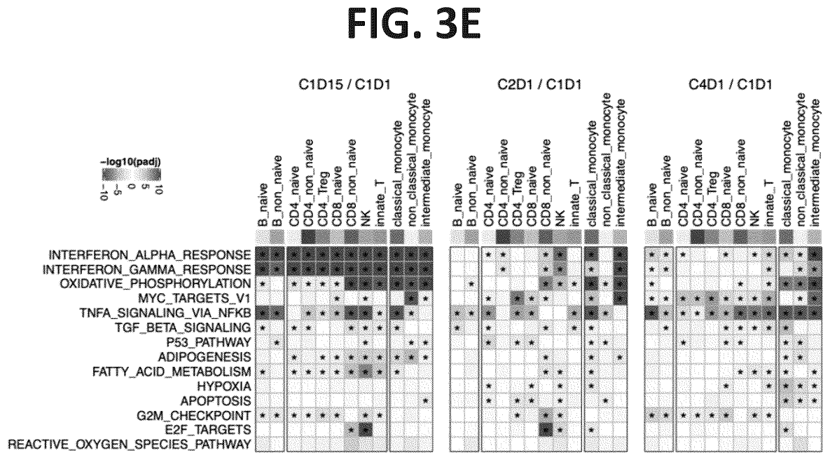

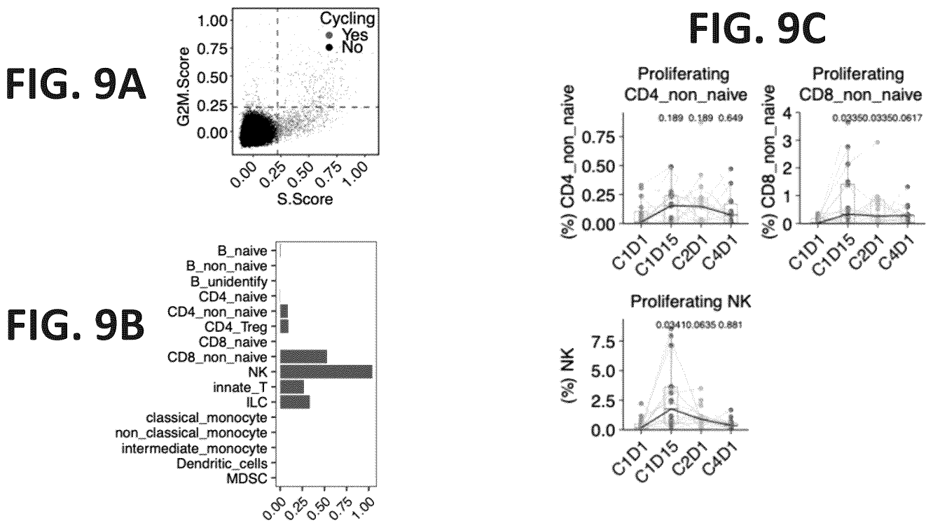

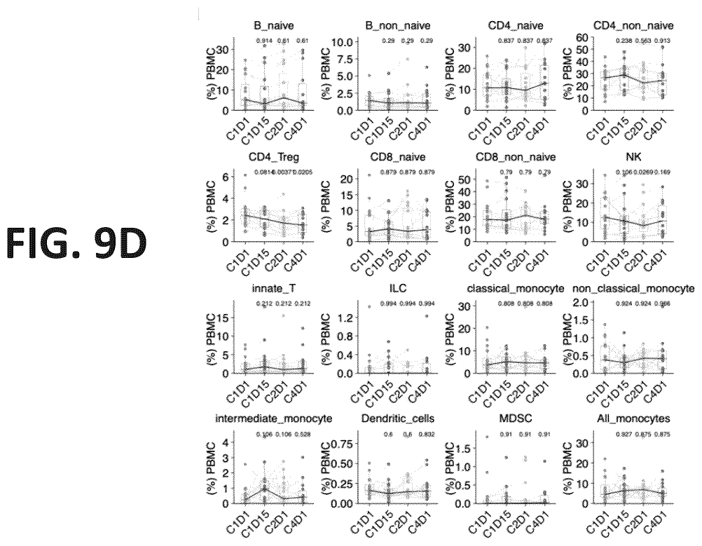

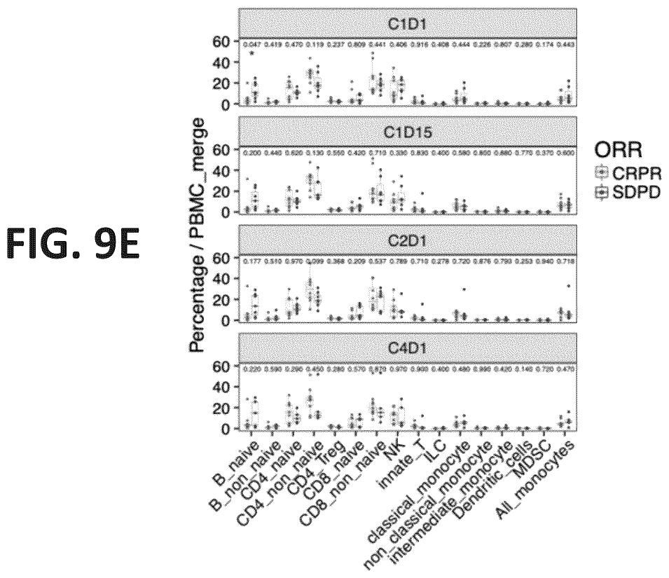

The effects of tiragolumab plus atezolizumab therapy on peripheral blood mononuclear cells (PBMCs) collected at cycle 1 day 1 (C1D1), cycle 1 day 15 (C1D15, two weeks post treatment), cycle 2 day 1 (C2D1, 3 weeks post treatment), and cycle 4 day 1 (C4D1, 9 weeks post treatment) from patients in the phase 1b NSCLC study of tiragolumab plus atezolizumab (GO30103; Bendell et al., Cancer Research, 80: Abstract CT302, 2010) were evaluated. Using scRNA-seq and CITE-seq, transcriptional profiles of 407,219 immune cells were obtained and annotated (FIG. 3A). Increased proliferation of peripheral cells was observed at C1 D15 (FIGS. 3B and 9A), especially in the subsets of CD8 non-naïve cells and natural killer (NK) cells (FIGS. 9B and 9C). The proportions of major cell types as a fraction of PBMCs were not altered over the treatment (FIG. 9D) or between responders and non-responders at each timepoint (FIG. 9E). The proportion of circulating Tregs decreased under treatment when evaluated as a fraction of total CD4+ T cells (FIG. 3C). Interestingly, intermediate monocytes increased at C1 D15, while classical monocytes appeared to decrease when evaluated as a fraction of total monocytes (FIG. 3D).

Gene set enrichment analysis comparing changes at C1 D15 relative to baseline (C1D1) using the hallmark gene set (Liberzon et al., Cell Syst., 1: 417-425, 2015) showed a broad interferon (IFN) response in all cell types, and then at C2D1 the response appeared to become more specific. Increased interferon signaling was observed in non-naïve CD8+ and CD4+ T cells, NK cells, and monocytes, in line with previous observations for atezolizumab monotherapy (FIG. 3E) (Herbst et al., Nature, 515: 563-567, 2014; Bar et al., J. C. I. Insight, 5: e129353, 2020). Novel pathways upregulated in monocytes were also observed, including the interferon response, oxidative phosphorylation pathway, and the MYC targeting pathway that has been shown to regulate macrophage polarization (Pello et al., Blood, 119: 411-421, 2012). Overall, the scRNA-seq showed not just T/NK cell activation, but also monocyte activation in the periphery following combination treatment.

### Example 5. Anti-TIGIT Monotherapy Remodels Immune Cells in the Tumor Immune Microenvironment (TME) and Peripheral Blood Through FcγR

Because on-treatment tumor biopsies were not available in the CITYSCAPE trial, preclinical models were used to assess the effects of anti-TIGIT treatment on immune cells. Preclinical models provided the additional advantage of allowing investigation of non-canonical mechanisms of action of anti-TIGIT, such as those mediated through Fc-FcγR interactions. Specifically, scRNA-seq was performed on tumor-infiltrating and blood leukocytes in CT26 tumor-bearing mice treated with mouse surrogate TIGIT mAbs bearing varying Fc domains: mIgG2a-LALAPG (Fc inert), which lacks effector function (Lo et al., J. Biol. Chem., 292: 3900-3908, 2017); mIgG2b, which engages activating and inhibitory FcγR with comparable affinities; and mIgG2a, which preferentially engages activating FcγR (Nimmerjahn et al., Immunity, 24:19-28, 2006).

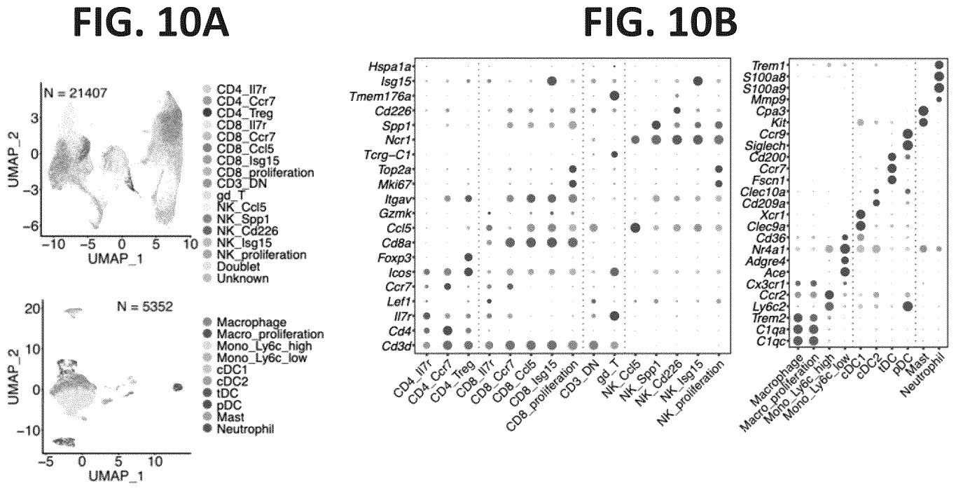

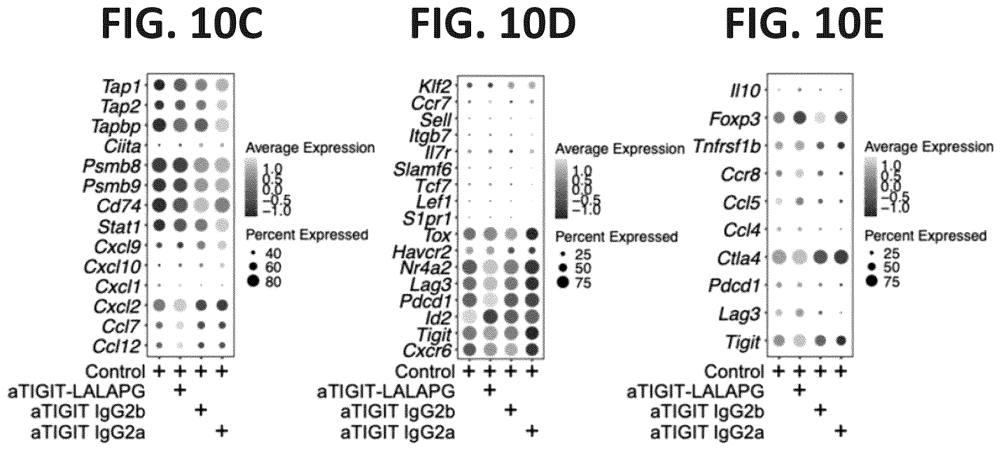

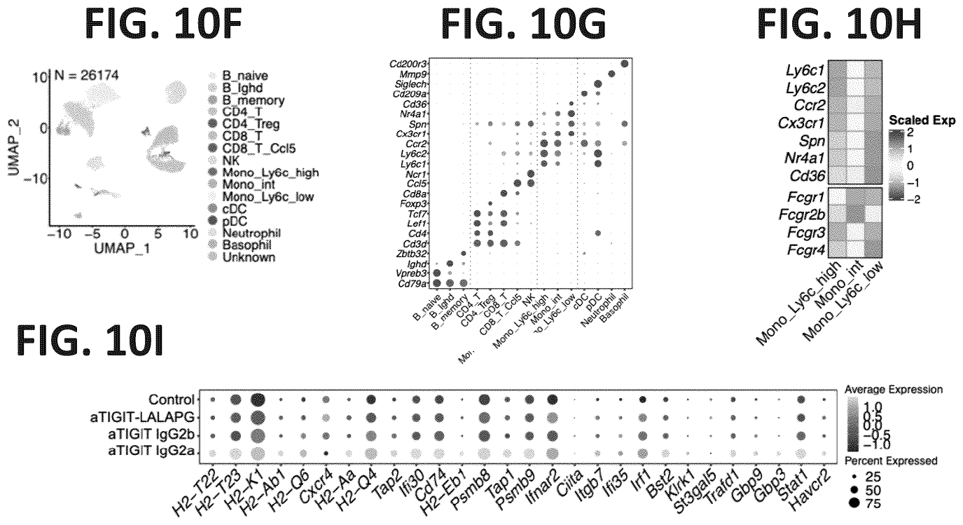

From within the tumors, 21,407 T and NK cells and 5,352 myeloid cells were characterized (FIGS. 10A and 10B). Gene expression analysis of macrophages and monocytes revealed that TIGIT-IgG2a and TIGIT-IgG2b both modulated the expression of MHC and cytokine genes, with TIGIT-IgG2a having a larger impact (FIG. 10C), consistent with a role for activating FcγR. Additionally, in the CD8+ T cells, it was observed that TIGIT Fc inert slightly increased the expression of exhaustion genes such as Pdcd1 and Lag3, whereas the expression of those genes was reduced by TIGIT-IgG2b and further by TIGIT-IgG2a (FIG. 10D). TIGIT-IgG2a also increased the expression of naïve- and memory-like genes in CD8+ T cells (FIG. 10D). In the CD4+ Tregs, TIGIT-IgG2a reduced the expression of immunosuppressive and exhaustion genes including Il10, Ccr8, Ctla4, Pdcd1, and Tigit, again with a larger effect size than TIGIT-IgG2b (FIG. 10E).

Circulating blood immune cells (n=26,174) were also characterized and annotated (FIGS. 10F and 10G). Treatment effects were specially evaluated on non-classical monocytes that expressed high levels of Fcgr4 (FIG. 10H), the activating FcγR engaged by IgG2a Fc. Compared with isotype control, TIGIT Fc inert and TIGIT-IgG2b had minimal effect on the expression of MHC and interferon-response genes in contrast to TIGIT-IgG2a (FIG. 10I). Together, these data indicate that anti-TIGIT mAbs can drive tumor macrophage activation, CD8+ and CD4+ T cell modulation, and blood monocyte activation via activating FcγR.

### Example 6. Fc-Active Anti-TIGIT Synergizes with Anti-PD-L1 to Remodel the Tumor Microenvironment and Activate Peripheral Monocytes

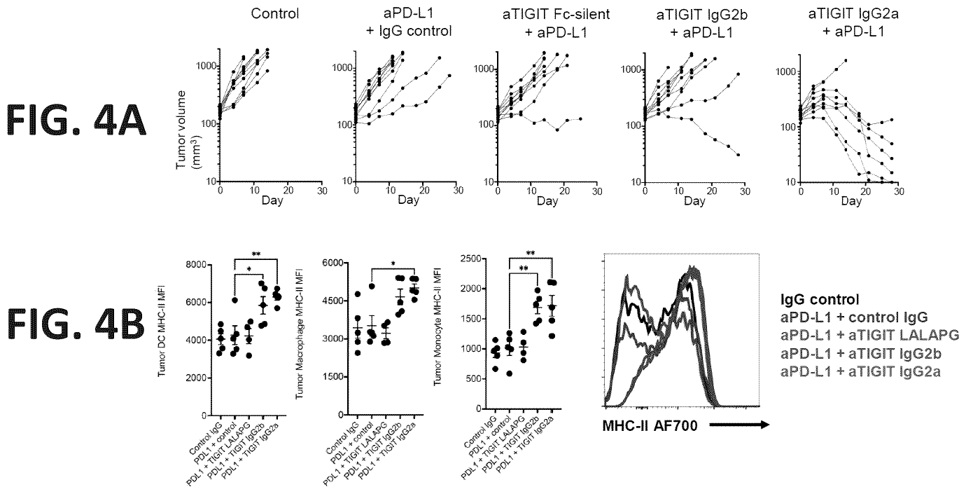

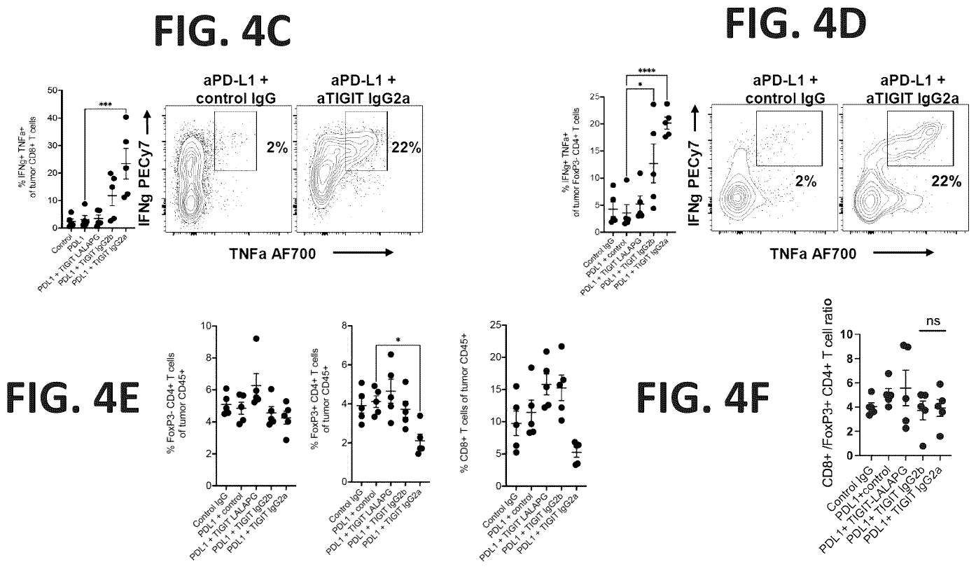

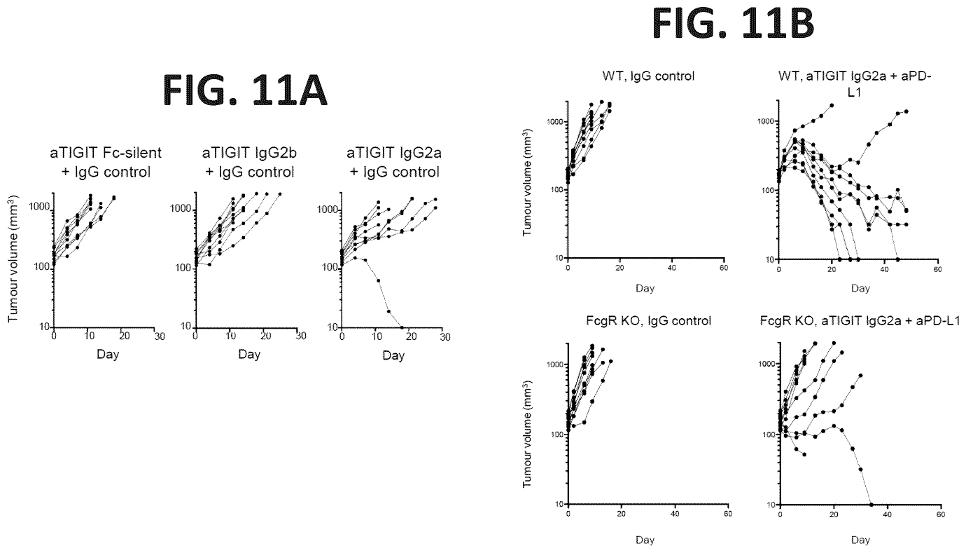

Next co-blockade of PD-L1 and TIGIT and its efficacy in tumor growth control was investigated. When combined with an mIgG2a-LALAPG mouse surrogate anti-PD-L1 mAb, mIgG2a, but not mIgG2b or mIgG2a-LALAPG anti-TIGIT, was capable of inducing tumor rejection (FIG. 4A). Anti-TIGIT monotherapies, including a mIgG2a-formatted mAb, exhibited limited effect on tumor growth (FIG. 11A). The combination of mIgG2a anti-TIGIT and anti-PD-L1 failed to control tumor growth in FcγR knockout mice, validating a requirement for Fc-FcγR engagement for therapeutic activity of anti-TIGIT mAbs in the CT26 mouse tumor model (FIG. 11B).

Ex vivo flow-cytometry analysis of tumor-infiltrating leukocytes revealed that Fc-active TIGIT mAb drove increased cell surface expression of MHC-II on myeloid cells including dendritic cells, macrophages, and monocytes (FIG. 4B). Activating FcγR engagement was also required for anti-TIGIT-mediated enhancement of CD8+ and CD4+ T cell competency to co-produce IFNγ and TNFα, with IgG2a anti-TIGIT driving the strongest effects (FIGS. 4C and 4D). IgG2a anti-TIGIT also induced a moderate decrease in CD4+ Treg cell frequency and a similar trend in CD8+ T cell frequency (FIG. 4E), though the ratio of Treg to CD8+ T cells was unchanged (FIG. 4F).

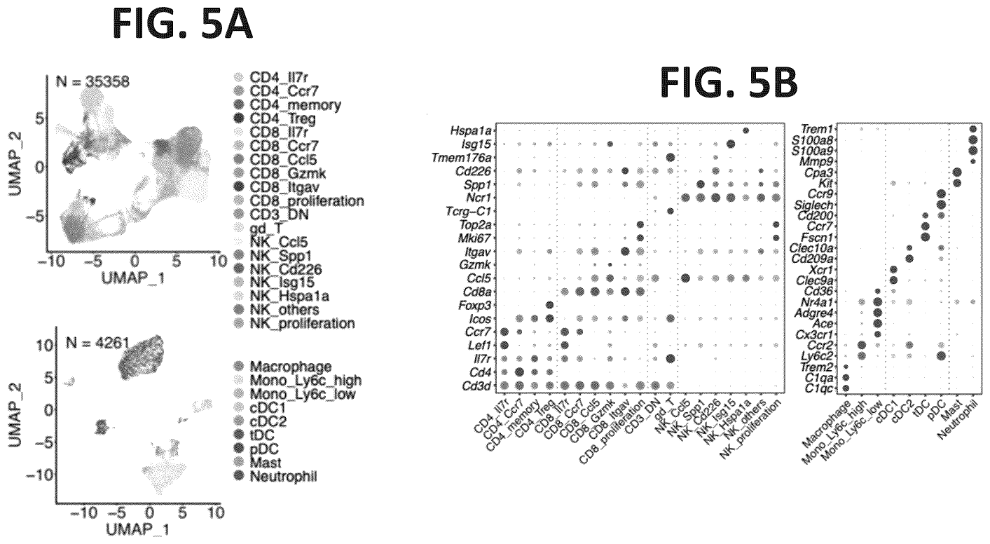

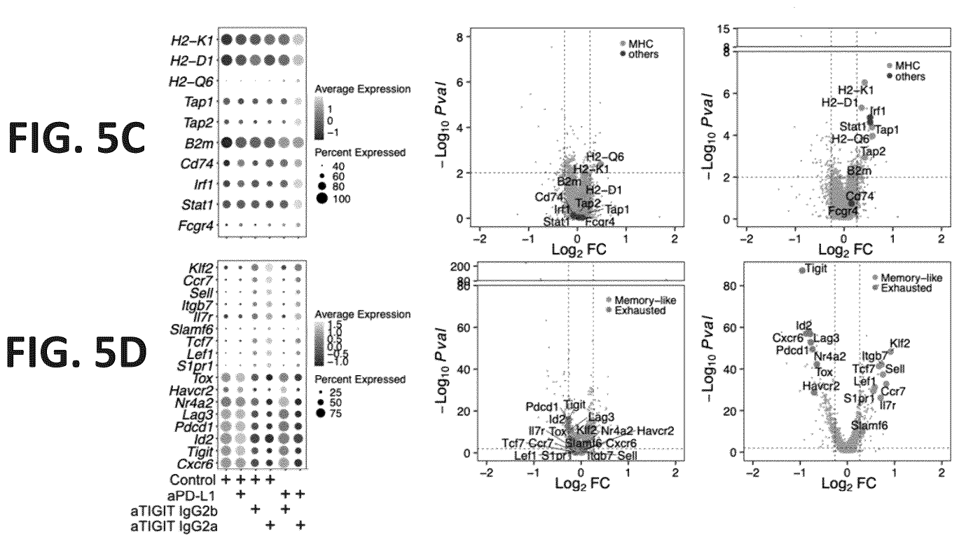

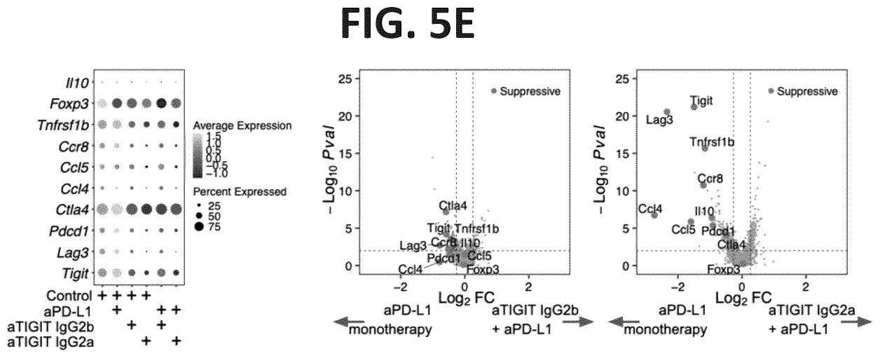

Tumor-infiltrating leukocytes were captured and sequenced by scRNA-seq (n=35,358 for tumor T and NK cells; n=4,261 for tumor myeloid cells) in mice treated with anti-PD-L1±IgG2b and IgG2a anti-TIGIT (FIGS. 5A and 5B). Relative to single agent treatments, anti-PD-L1 plus anti-TIGIT IgG2a synergistically inflamed tumor macrophages, inducing a gene program of high expression of MHC antigen presentation-related genes similar to that observed with anti-TIGIT alone, but to a much greater extent (FIG. 5C). However, the anti-PD-L1 plus anti-TIGIT IgG2b combination did not elicit a comparable effect.

In tumor CD8+ T cells, treatment with anti-PD-L1 sustained expression of a T cell exhaustion gene program characterized by the transcriptional regulators Tox, Nr4a2, and Id2 as well as the co-inhibitory receptors Pdcd1, Tigit, Lag3, and Havcr2. In contrast, treatment with anti-TIGIT drove a shift in tumor CD8+ T cells away from exhaustion and towards a memory- or progenitor-like gene program, with elevated expression of Tcf7, K/f2, Ccr7, Lef1, Il7r, and SelI (FIG. 5D). Treatment with IgG2a anti-TIGIT continued to drive this conversion towards progenitor-like cells, even in combination with anti-PD-L1, while treatment with IgG2b anti-TIGIT plus anti-PD-L1 led to expression of an anti-PD-L1-like exhaustion gene program (FIG. 5D). In tumor Tregs, both anti-TIGIT isotypes drove downregulation of immunosuppressive and Treg-associated genes such as 1110, Ctla4, and Tnfrsf1b relative to treatment with anti-PDL1 or control, and sustained those effects in combination with anti-PD-L1 (FIG. 5E).

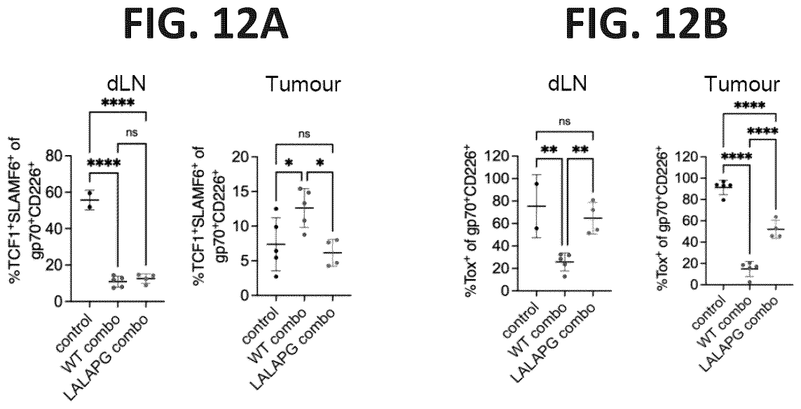

To confirm these effects on tumor antigen-specific immune responses, CD8+ T cells expressing T cell receptors (TCR) specific for the CT26 tumor antigen gp70 were analyzed (Huang et al., Proc. Natl. Acad. Sci. USA, 93: 9730-9735, 1996). As CD226 expression and functional activity may be important for CD8+ T cell anti-tumor responses to anti-PD-L1 plus anti-TIGIT (Banta et al., Immunity, 55: 512-526, 2022), analyses focused on the gp70+CD226+ fraction of CD8+ T cells. In combination with anti-PD-L1, mIgG2a anti-TIGIT drove tumor-resident gp70-specific CD8+ T cells to downregulate TOX while upregulating TCF1 and SLAMF6, consistent with a shift from exhaustion towards a memory-like state (FIGS. 12A and 12B). Fc inert mIgG2a-LALAPG anti-TIGIT drove a lesser downregulation of TOX and did not induce upregulation of TCF1 and SLAMF6 (FIGS. 12A and 12B).

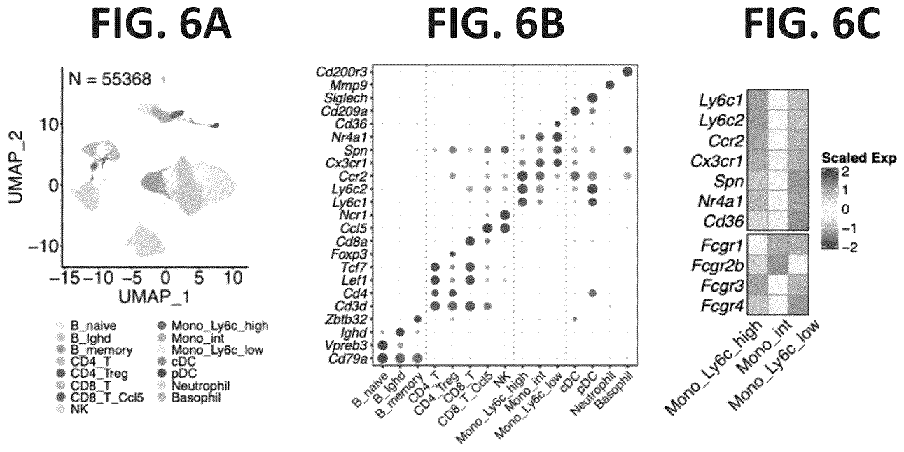

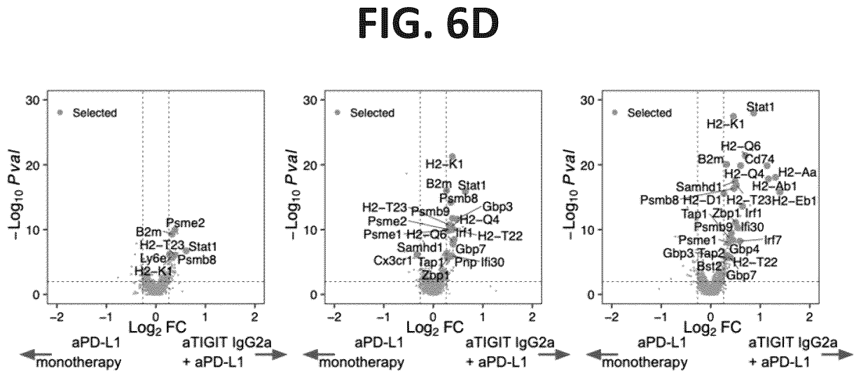

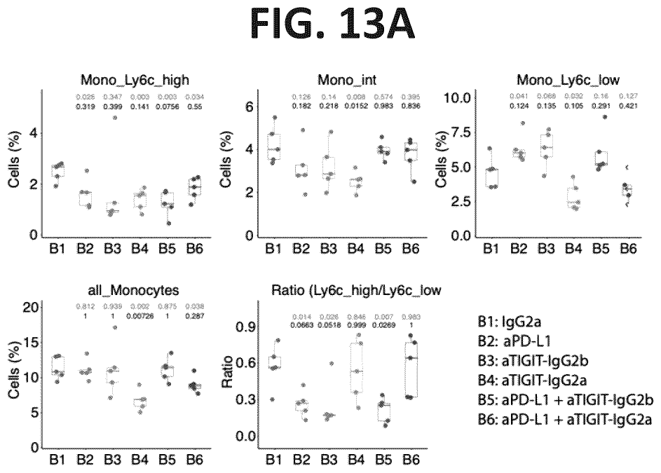

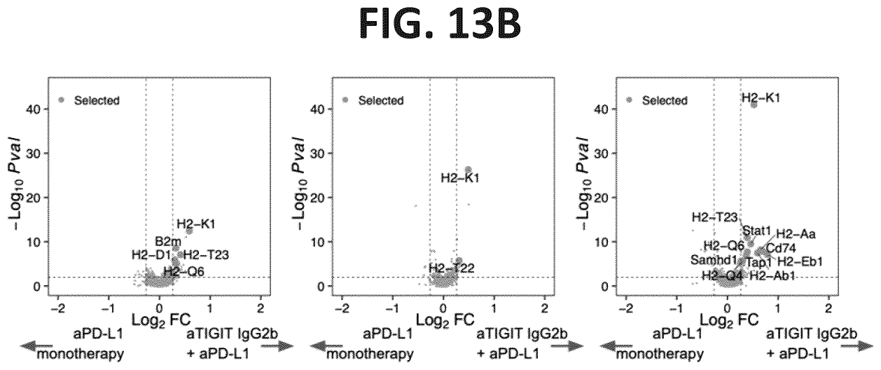

In the blood, a total of 55,368 cells were single-cell sequenced and annotated (FIGS. 6A and 6B). Treatment with IgG2a anti-TIGIT alone or in combination with anti-PD-L1 unexpectedly led to as much as a 50% decrease in the frequency of circulating monocytes relative to control (FIG. 13A). Non-classical monocytes appeared to be most responsible, with decreased prevalence in mice treated with IgG2a anti-TIGIT but increased prevalence in mice treated with anti-PD-L1 and/or IgG2b anti-TIGIT (FIG. 13A). Relative to treatment with anti-PD-L1 alone, combination treatment with IgG2a anti-TIGIT plus anti-PD-L1 led to general induction of antigen presentation programs in all monocyte subsets as well as more specific induction of interferon response gene signatures in non-classical monocytes and intermediate monocytes, which express higher levels of activating FcγR than do classical monocytes (FIGS. 6C and 6D). Similar monocyte modulation was observed in the IgG2b anti-TIGIT plus anti-PD-L1 treatment, but to a much smaller effect size (FIG. 13B).

### C. Conclusions

Tiragolumab is a mAb designed to bind to TIGIT and to prevent it from binding to its ligands, which include the high-affinity ligand CD155 or poliovirus receptor (PVR) (Manieri et al., Trends Immunol., 38: 20-28, 2017, Johnston et al., Annu. Rev. Cancer Biol., 5: 203-219, 2021). In mouse models, co-blockade of TIGIT and PD-L1 has been shown to synergistically elicit anti-tumor T cell responses. It has recently emerged that the TIGIT and PD-1 pathways are mechanistically interdependent, working via the activating receptor and TIGIT family member CD226 (Banta et al., Immunity, 55: 512-526, 2022; Johnston et al., Annu. Rev. Cancer Biol., 5: 203-219, 2021). Several mechanisms of action have been proposed for TIGIT targeted therapies, including canonical co-inhibitory receptor blockade, Fc-dependent depletion of TIGIT expressing regulatory T cells (Tregs), and Fc-dependent myeloid cell modulation (Johnston et al., Cancer Cell, 26: 923-937, 2014; Banta et al., Immunity, 55: 512-526, 2022; Waight et al., Cancer Cell, 33: 1033-1047, 2018; Han et al., Front. Immunol., 11: 658, 2020; Preillon et al., Mol. Cancer Ther., 20: 121-131, 2021; Yu et al., Nat. Immunol., 10: 48-57, 2008; Stanietsky et al., Proc. Nat. Acad. Sci. USA, 106: 17858-17863, 2009). It is not yet known which of these mechanisms are relevant in the clinical blockade of TIGIT, and as a result, the functionality of the anti-TIGIT Fc domain has been the subject of much debate. While therapeutic PD-1 and PD-L1 mAbs possess an inert or attenuated Fc, the Fc strategies of TIGIT mAbs in clinical development are diverse, and variously intended to maintain, enhance, or eliminate Fc gamma receptor (FcγR) engagement (Dolgin et al., Nat. Biotechnol., 38: 1007-1009, 2020).

Checkpoint inhibitors provide therapeutic benefit by enhancing anti-tumor T cell responses, and benefit is concentrated in patients whose tumors are rich in T cells and T cell-driven inflammation (Ribas et al., Science, 359: 1350-1355, 2018). In contrast, intratumoral macrophages and immature monocytes are typically understood to suppress anti-tumor T cell responses and therefore resist the effects of checkpoint inhibitor treatment (Morad et al., Cell, 184: 5309-5337, 2021). In CITYSCAPE, adding tiragolumab to atezolizumab resulted in significantly improved ORR, PFS, and OS relative to atezolizumab monotherapy in patients with NSCLC (Cho et al., Lancet Oncology, 23: 781-792, 2022). The biomarker analysis presented herein uncovered an unexpected positive association of high TAMs and Tregs in tumors with improved clinical outcome following combination treatment, but not atezolizumab monotherapy treatment, suggesting a differentiated mechanism of action in which typically suppressive tumor myeloid cells potentiate rather than limit tiragolumab activity. Serum peptide and scRNA-seq of patient PBMCs suggested monocyte activation, along with T cell and NK cell activation 2-3 weeks after treatment initiation.

In preclinical models, both Fc-silent and Fc-active tiragolumab surrogate mAbs drove T cell and NK cell responses, similar to what is observed with other checkpoint inhibitors. However, Fc-active TIGIT mAbs further activated TAMs and other myeloid cells and did so synergistically in combination with anti-PD-L1. A key effect of this interaction was induction of a memory-like gene program and downregulation of a terminal differentiation gene program in tumor CD8+ T cells. Interestingly, anti-PD-L1 appeared to oppose this effect of anti-TIGIT. In combination treatment, the degree of anti-TIGIT activating FcγR engagement determined the T cell fate, with IgG2b anti-TIGIT yielding to anti-PD-L1 and terminal differentiation and IgG2a anti-TIGIT dominantly driving a memory-like program. Tumor Tregs also responded to Fc-active anti-TIGIT with downregulation of an immunosuppressive gene program.

As a mechanism of action, activation of myeloid cells is differentiated from other approved checkpoint inhibitors that may not meaningfully engage myeloid cells including antibodies targeting PD-L1, PD-1 and LAG-3. Modulatory effects on myeloid cells potentiated by the Fc portion of anti-TIGIT mAb and/or PVR signaling may have far-reaching impacts in the tumor setting. An immunosuppressive network between myeloid-derived suppressor cells (MDSCs) and Treg cells and NK T cells has been described (Lindau et al., Immunology, 138: 105-115, 2013). There is cross-talk between MDSCs and Tregs, with MDSCs promoting development and induction of Tregs. NK T cells, on the other hand, are capable of abrogating MDSC suppressive activity and converting them into antigen-presenting cells (APCs). Thus, manipulation of one component of the immunosuppressive tumor microenvironment (TME) may have cascading effects that result in reshaping of the entire TME. In addition to shaping the TME by promoting proinflammatory myeloid cell subsets supportive of anti-tumor T cell activity, modulation of myeloid cells can influence T cell priming, activation, trafficking, and survival through the elaboration of cytokines and chemokines (Callister et al., J. Proteome Res., 5: 277-286, 2006). As different myeloid cell subsets can produce different repertoires of cytokines and chemokines, tipping the balance in the cellular composition may be a key factor in generating productive anti-tumor immunity (Banchereau et al., J. Immunother. Cancer, 9: e002231, 2021; Louie et al., Biotechnol. Bioeng., 114: 632-644, 2017). Indeed, cDC1s, monocytes, and macrophages have been shown to have differential roles in influencing T cell fate decisions (Leek et al., PLoS Genet., 3: 1724-1735, 2007). Future studies dissecting the complex network of myeloid cell interactions within the tumor immune contexture will provide more insight into the mechanistic contributions mediated by the anti-TIGIT Fc. This myeloid activating Fc mechanism is likely applicable beyond anti-TIGIT; a recent report described similar myeloid cell-activating effects of Fc-active ipilimumab and surrogate anti-CTLA-4 antibodies (Yofe et al., Nat. Cancer, 3:1336-1350, 2022).

To date, the importance of FcγR engagement to TIGIT antibodies has been uniquely controversial in the checkpoint inhibitor field, with antibodies in clinical development running the gamut from Fc inert to highly Fc competent isotypes (Chiang and Mellman, J. Immunother. Cancer, 10: e004711, 2022). The present CITYSCAPE and nonclinical findings now reveal a clear positive role for FcγR engagement in anti-TIGIT immunotherapy, suggesting that TIGIT antibodies that are able to engage FcγR may deliver greater therapeutic benefit than those that cannot.

### Example 7. ScRNA Seq Analysis of Tumor-Infiltrating CD45+ Immune Cells

Tumor-infiltrating CD45+ immune cells collected from BALB/c mice having syngeneic CT26 tumors were analyzed by scRNA-seq. Samples were collected three days after the start of treatment with the control or anti-PD-L1 and/or anti-TIGIT antibody. Five mice were analyzed per group.

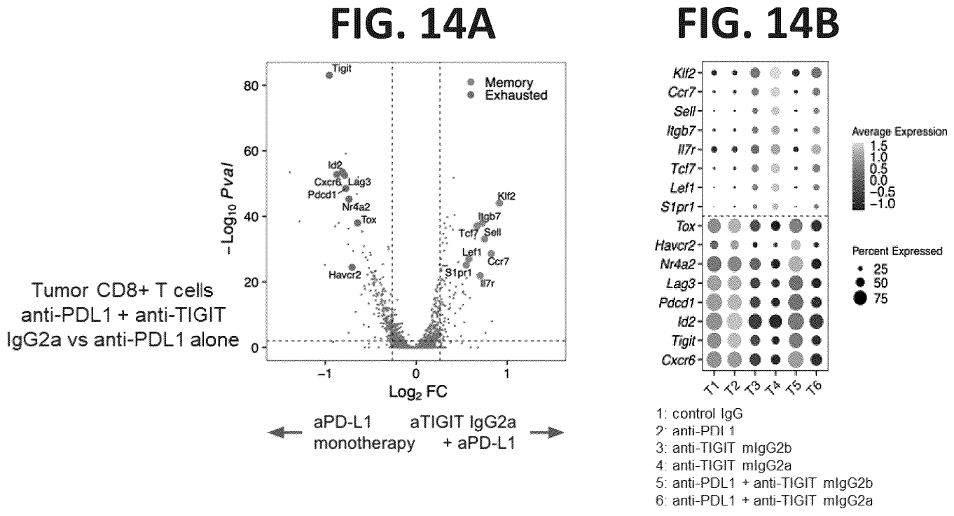

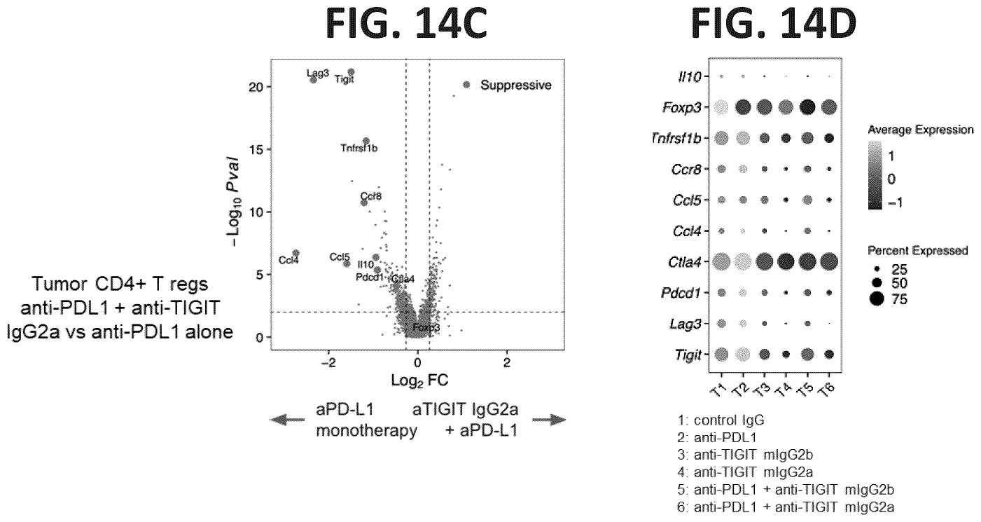

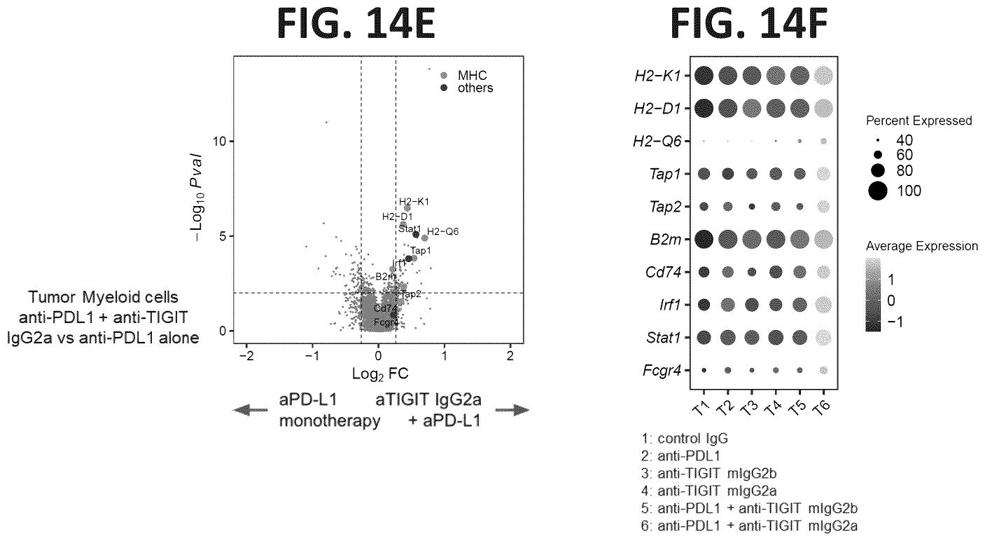

scRNA-seq analysis of tumor-infiltrating CD8+ T cells showed that the Fc-active mIgG2a anti-TIGIT antibody combined with an anti-PD-L1 antibody increased T effector memory gene expression (Klf2, Itgb7, Tcf7, SelI, Lef1, Ccr7, S1pr1, and Il7r) and suppressed exhaustion-related genes (Tigit, Id2, Cxcr6, Lag3, Pdcd1, Nr4a2, Tox, and Havcr2) (FIGS. 14A and 14B). CD4+ Tregs showed reduced immune suppressive gene expression (Lag3, Tigit, Tnfrsf1b, Ccr8, Ccl4, Ccl5, Il10, Pdcd1, Ctla4, and Foxp3) and increased proinflammatory gene expression with the combination therapy (FIGS. 14C and 14D). Monocytes showed increased MHC-related antigen presenting gene expression with the combination therapy (FIGS. 14E and 14F).

### VII. Other Embodiments

Some embodiments of the technology described herein can be defined according to any of the following numbered embodiments:
1. A method of treating an individual having a cancer, the method comprising administering an anti-TIGIT antagonist antibody to the individual, wherein the anti-TIGIT antagonist antibody is capable of Fc-dependent activation of myeloid cells, optionally wherein the myeloid cell is a cell selected from the group consisting of intratumoral type 1 conventional dendritic cells (cDC1s), macrophages, neutrophils, and circulating monocytes.
2. Use of an anti-TIGIT antagonist antibody in the manufacture of a medicament for treating cancer, wherein the anti-TIGIT antagonist antibody is capable of Fc-dependent activation of myeloid cells, optionally wherein the myeloid cell is a cell selected from the group consisting of intratumoral cDC1s, macrophages, neutrophils, and circulating monocytes.
3. A method of treating an individual having a cancer, the method comprising administering an anti-TIGIT antagonist antibody to the individual, wherein the anti-TIGIT antagonist antibody is capable of interacting with the Fc gamma receptor (FcγR) on myeloid cells and is capable of inducing CD8+ T cell mobilization in the blood and/or an expansion of proliferating CD8+ T cells within the tumor bed.
4. Use of an anti-TIGIT antagonist antibody in the manufacture of a medicament for treating cancer, wherein the anti-TIGIT antagonist antibody is capable of interacting with the FcγR and is capable of inducing CD8+ T cell mobilization in the blood and/or an expansion of proliferating CD8+ T cells within the tumor bed.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-3. (canceled)

4. A method of treating an individual having a cancer, the method comprising:
(a) detecting an expression level of each of C1QC, MSR1, MRC1, VSIG4, SPP1, and MARCO in a sample from the individual and determining a TAM signature score therefrom, wherein the TAM signature score is above a reference TAM signature score and thereby identifies the individual as one who may benefit from a treatment comprising a PD-1 axis binding antagonist and an anti-TIGIT antagonist antibody; and
(b) administering an effective amount of a PD-1 axis binding antagonist and an anti-TIGIT antagonist antibody to the individual.

5. A method of treating an individual having a cancer, the method comprising administering a PD-1 axis binding antagonist and an anti-TIGIT antagonist antibody to the individual, wherein the individual has been determined to have a TAM signature score that is above a reference TAM signature score, thereby identifying the individual as one who may benefit from a treatment comprising a PD-1 axis binding antagonist and an anti-TIGIT antagonist antibody, and wherein the TAM signature score is based on the expression level of each of C1QC, MSR1, MRC1, VSIG4, SPP1, and MARCO detected in a sample from the individual.

6. The method of claim 5, wherein the sample is obtained from the individual prior to treatment with the PD-1 axis binding antagonist and the anti-TIGIT antagonist antibody.

7. The method of claim 5, wherein the benefit is an increase in progression-free survival (PFS), objective response rate (ORR), or overall survival (OS).

8. The method of claim 5, wherein the reference TAM signature score is a pre-assigned TAM signature score.

9. The method of claim 5, wherein the reference TAM signature score is a TAM signature score in a reference population.

10. The method of claim 9, wherein the TAM signature score in the reference population is a median TAM signature score of the reference population.

11. The method of claim 9, wherein the reference population is a population of individuals having the cancer.

12. The method of claim 5, wherein the TAM signature score is an average of the expression levels of C1QC, MSR1, MRC1, VSIG4, SPP1, and MARCO in the sample from the individual.

13. The method of claim 12, wherein the TAM signature score is an average of the normalized expression levels of C1QC, MSR1, MRC1, VSIG4, SPP1, and MARCO in the sample from the individual.

14. The method of claim 5, wherein the expression level of one or more of ACP5, MCEMP1, CYP27A1, OLR1, GRN, GLIPR2, ARRDC4, APOE, FOLR2, and CTSD has been detected in the sample from the individual and wherein the TAM signature score is an average of the expression levels of C1QC, MSR1, MRC1, VSIG4, SPP1, MARCO, and one or more of ACP5, MCEMP1, CYP27A1, OLR1, GRN, GLIPR2, ARRDC4, APOE, FOLR2, and CTSD in the sample from the individual.

15. (canceled)

16. The method of claim 14, wherein the expression level of each of ACP5, MCEMP1, CYP27A1, OLR1, GRN, GLIPR2, ARRDC4, APOE, FOLR2, and CTSD has been detected in the sample from the individual, and wherein the TAM signature score is an average of the expression levels of C1QC, MSR1, MRC1, VSIG4, SPP1, MARCO, ACP5, MCEMP1, CYP27A1, OLR1, GRN, GLIPR2, ARRDC4, APOE, FOLR2, and CTSD in the sample from the individual.

17-19. (canceled)

20. A method for monitoring the response of an individual having a cancer to a treatment comprising a PD-1 axis binding antagonist and an anti-TIGIT antagonist antibody, the method comprising detecting an expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 in a sample from the individual at a time point during or after administration of the PD-1 axis binding antagonist and the anti-TIGIT antagonist antibody, wherein an increase in the expression level of one or more of MARCO, CAMP, CD5L, CD163, NGAL, CSF1R, CD44, APOC2, APOC3, APOC4, APOA2, APOE, TRFL, VCAM1, PERM, B2MG, LYSC, LYAM1, LCAT, and LIRA3 relative to a respective reference expression level is predictive of an individual who is likely to respond to the treatment comprising the PD-1 axis binding antagonist and the anti-TIGIT antagonist antibody.

21-28. (canceled)

29. A method of treating an individual having a cancer, the method comprising:
(a) detecting the expression level of each of FOXP3, CTLA4, IL10, TNFRSF18, CCR8, IKZF4, and IKZF2 in a sample from the individual and determining a Treg signature score therefrom, wherein the Treg signature score is above a reference Treg signature score and thereby identifies the individual as one who may benefit from a treatment comprising a PD-1 axis binding antagonist and an anti-TIGIT antagonist antibody; and
(b) administering an effective amount of a PD-1 axis binding antagonist and an anti-TIGIT antagonist antibody to the individual.

30-51. (canceled)

52. The method of claim 5, wherein the sample has been determined to have a PD-L1-positive tumor cell fraction by an immunohistochemical (IHC) assay.

53-57. (canceled)

58. The method of claim 5, wherein the cancer is a lung cancer.

59. The method of claim 58, wherein the lung cancer is a non-small cell lung cancer (NSCLC).

60. The method of claim 5, wherein the anti-TIGIT antagonist antibody comprises the following hypervariable regions (HVRs):
(a) an HVR-H1 comprising the amino acid sequence of SNSAAWN (SEQ ID NO: 1);
(b) an HVR-H2 comprising the amino acid sequence of KTYYRFKWYSDYAVSVKG (SEQ ID NO: 2);
(c) an HVR-H3 comprising the amino acid sequence of ESTTYDLLAGPFDY (SEQ ID NO: 3);
(d) an HVR-L1 comprising the amino acid sequence of KSSQTVLYSSNNKKYLA (SEQ ID NO: 4);
(e) an HVR-L2 comprising the amino acid sequence of WASTRES (SEQ ID NO: 5); and
(f) an HVR-L3 comprising the amino acid sequence of QQYYSTPFT (SEQ ID NO: 6).

61-64. (canceled)

65. The method of claim 5, wherein the anti-TIGIT antagonist antibody comprises:
(a) a VH domain comprising an amino acid sequence having at least 95% sequence identity to the amino acid sequence of EVQLQQSGPGLVKPSQTLSLTCAISGDSVSSNSAAWNWIRQSPSRGLEWLGKTYYRFKWYSDYAVSVKGRI TINPDTSKNQFSLQLNSVTPEDTAVFYCTRESTTYDLLAGPFDYWGQGTLVTVSS (SEQ ID NO: 17) or QVQLQQSGPGLVKPSQTLSLTCAISGDSVSSNSAAWNWIRQSPSRGLEWLGKTYYRFKWYSDYAVSVKGRI TINPDTSKNQFSLQLNSVTPEDTAVFYCTRESTTYDLLAGPFDYWGQGTLVTVSS (SEQ ID NO: 18);
(b) a VL domain comprising an amino acid sequence having at least 95% sequence identity to the amino acid sequence of DIVMTQSPDSLAVSLGERATINCKSSQTVLYSSNNKKYLAWYQQKPGQPPNLLIYWASTRESGVPDRFSGS GSGTDFTLTISSLQAEDVAVYYCQQYYSTPFTFGPGTKVEIK (SEQ ID NO: 19); or
(c) a VH domain as in (a) and a VL domain as in (b).

66-67. (canceled)

68. The method of claim 5, wherein the anti-TIGIT antagonist antibody comprises:
(a) a heavy chain comprising the amino acid sequence of SEQ ID NO: 33; and
(b) a light chain comprising the amino acid sequence of SEQ ID NO: 34.

69. The method of claim 5, wherein the anti-TIGIT antagonist antibody is a monoclonal antibody, a human antibody, a full-length antibody, or an IgG class antibody.

70-71. (canceled)

72. The method of claim 5, wherein the anti-TIGIT antagonist antibody exhibits effector function or comprises an Fc domain that is able to interact with an Fc gamma receptor (FcγR).

73-74. (canceled)

75. The method of claim 72, wherein the IgG class antibody is an IgG1 subclass antibody.

76. The method of claim 5, wherein the anti-TIGIT antagonist antibody is tiragolumab.

77-84. (canceled)

85. The method of claim 5, wherein the PD-1 axis binding antagonist is atezolizumab.

86-186. (canceled)

187. The method of claim 5, wherein the anti-TIGIT antagonist antibody is capable of Fc-dependent activation of myeloid cells.

188. The method of claim 5, wherein the anti-TIGIT antagonist antibody is capable of interacting with the Fc gamma receptor (FcγR) on myeloid cells and is capable of inducing CD8+ T cell mobilization in the blood or an expansion of proliferating CD8+ T cells within the tumor bed.

189-200. (canceled)

201. The method of claim 5, wherein the cancer is hepatocellular cancer.


SEQUENCE LISTING (34 sequences)

SEQ ID NO: 1 - protein, 7 aa, synthetic construct
SNSAAWN

SEQ ID NO: 2 - protein, 18 aa, synthetic construct
KTYYRFKWYSDYAVSVKG

SEQ ID NO: 3 - protein, 14 aa, synthetic construct
ESTTYDLLAGPFDY

SEQ ID NO: 4 - protein, 17 aa, synthetic construct
KSSQTVLYSSNNKKYLA

SEQ ID NO: 5 - protein, 7 aa, synthetic construct
WASTRES

SEQ ID NO: 6 - protein, 9 aa, synthetic construct
QQYYSTPFT

SEQ ID NO: 7 - protein, 23 aa, synthetic construct
DIVMTQSPDSLAVSLGERATINC

SEQ ID NO: 8 - protein, 15 aa, synthetic construct
WYQQKPGQPPNLLIY

SEQ ID NO: 9 - protein, 32 aa, synthetic construct
GVPDRFSGSGSGTDFTLTISSLQAEDVAVYYC

SEQ ID NO: 10 - protein, 10 aa, synthetic construct
FGPGTKVEIK

SEQ ID NO: 11 - protein, 30 aa, synthetic construct
  VARIANT 1: note = MISC_FEATURE - Xaa is Gln or Glu
XVQLQQSGPGLVKPSQTLSLTCAISGDSVS

SEQ ID NO: 12 - protein, 14 aa, synthetic construct
WIRQSPSRGLEWLG

SEQ ID NO: 13 - protein, 32 aa, synthetic construct
RITINPDTSKNQFSLQLNSVTPEDTAVFYCTR

SEQ ID NO: 14 - protein, 11 aa, synthetic construct
WGQGTLVTVSS

SEQ ID NO: 15 - protein, 30 aa, synthetic construct
EVQLQQSGPGLVKPSQTLSLTCAISGDSVS

SEQ ID NO: 16 - protein, 30 aa, synthetic construct
QVQLQQSGPGLVKPSQTLSLTCAISGDSVS

SEQ ID NO: 17 - protein, 126 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 18 - protein, 126 aa, synthetic construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 19 - protein, 113 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 20 - protein, 10 aa, synthetic construct
GFTFSDSWIH

SEQ ID NO: 21 - protein, 18 aa, synthetic construct
AWISPYGGSTYYADSVKG

SEQ ID NO: 22 - protein, 9 aa, synthetic construct
RHWPGGFDY

SEQ ID NO: 23 - protein, 11 aa, synthetic construct
RASQDVSTAVA

SEQ ID NO: 24 - protein, 7 aa, synthetic construct
SASFLYS

SEQ ID NO: 25 - protein, 9 aa, synthetic construct
QQYLYHPAT

SEQ ID NO: 26 - protein, 118 aa, synthetic construct [sequence omitted]

SEQ ID NO: 27 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 28 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 31 - protein, 223 aa, Homo sapiens [sequence omitted]

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 456 aa, Homo sapiens
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, Homo sapiens
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC